(19) United States
(12) Patent Application Publication
(10) Pub. No.: US 2022/0411821 A1
(43) Pub. Date: Dec. 29, 2022

(54) GENE THERAPY VECTORS

(71) Applicant: University of Florida Research Foundation, Incorporated, Gainesville, FL (US)

(72) Inventors: Eric Tzy-Shi Wang, Gainesville, FL (US); John Andrew Berglund, Menands, NY (US); Elizabeth Delgado, Gainesville, FL (US); Jana Rose Jenquin, Gainesville, FL (US)

(73) Assignee: University of Florida Research Foundation, Incorporated, Gainesville, FL (US)

(21) Appl. No.: 17/772,266

(22) PCT Filed: Oct. 28, 2020
(86) PCT No.: PCT/US2020/057796

§ 371 Date: Apr. 27, 2022

Related U.S. Application Data
(60) Provisional application No. 62/927,087, filed on Oct. 28, 2019.

(51) Int. Cl. C12N 15/86; A61K 48/00; A61P 35/00

(52) U.S. Cl. CPC C12N 15/86; A61K 48/0066; A61P 35/00; C12N 2830/42; C12N 2750/14143

(57) ABSTRACT

Provided herein, in some embodiments, are nucleic acid constructs encoding RNA molecules comprising one or more introns that can be regulated (e.g., autoregulated), and that are useful for delivery in a recombinant viral vector. Aspects of the application provide compositions and methods for delivering regulated (e.g., auto-regulated) gem expression constructs to a subject.

### RELATED APPLICATIONS

This application claims the benefit under 35 U.S.C 119(e) of the filing date of U.S. Provisional Application Ser. No. 62/927,087, filed Oct. 28, 2019, the entire contents of which are incorporated herein by reference.

### Reference to a Sequence Listing Submitted as a Text File

This application contains a Sequence Listing which has been submitted in ASCII format and is hereby incorporated by reference in its entirety. Said ASCII copy, created on Oct. 28, 2020 is named U119670080WO00-SEQ-PRW and is 348 kilobytes in size.

### STATEMENT AS TO FEDERALLY SPONSORED RESEARCH

This invention was made with government support under grant number R01 GM 121862 awarded by the National Institutes of Health. The government has certain rights in the invention.

### BACKGROUND

Recombinant viruses can be used to deliver therapeutic gene expression constructs to patients as a form of genetic therapy. For example, a patient suffering from a disease or condition associated with a particular genetic mutation can be treated with a recombinant virus that delivers a therapeutic gene construct to the patient to supplement or replace the function of the mutated gene.

In some cases, a wild-type copy of a defective gene can be delivered to a patient to restore normal physiological function. However, viral vectors typically have size limitations. As a result, recombinant gene expression constructs for viral vector delivery often only include protein coding sequences (e.g., minigenes with no introns) and limited 5′ and 3′ regulatory sequences, making it difficult to appropriately regulate expression of the encoded protein.

### SUMMARY OF THE INVENTION

Aspects of the application provide compositions and methods for delivering regulated (e.g., auto-regulated) gene expression constructs to a subject. Aspects of the application relate to methods and compositions for regulating nucleic acid expression in recombinant adeno-associated virus (rAAV) vectors. In some aspects, an rAAV comprises a recombinant AAV genome that includes a nucleic acid that encodes an RNA (e.g., an mRNA) comprising one or more introns. In some embodiments, splicing of at least one intron is regulated by one or more intracellular factor(s). Regulation of intron splicing can control the expression level of the RNA and/or of the type of RNA (e.g., of an RNA splice alternative) inside a cell.

In some aspects, intron splicing regulation can be used to help control the expression of one or more therapeutic transcripts that are encoded by an rAAV. Accordingly, compositions and methods described herein can be useful to regulate expression of therapeutic transcripts in the context of rAAV based treatments for diseases or disorders.

Certain aspects of the present invention contemplate an rAAV comprising a nucleic acid encoding an RNA, wherein the RNA comprises a first intron. In some embodiments, splicing of the first intron is regulated by an intracellular factor. In some embodiments, a nucleic acid is a recombinant AAV genome that comprises a DNA molecule comprising sequences that encode the RNA. In some embodiments, the rAAV further comprises a second intron.

In some embodiments, the nucleic acid encodes the intracellular factor. In some embodiments, the splicing of the intron is regulated by the encoded intracellular factor. In some embodiments, the splicing of the intron is not regulated by the encoded intracellular factor. In some embodiments, the intracellular factor is a protein, an RNA, or a protein-RNA complex.

In some embodiments, the protein is a tissue-specific RNA binding protein, an autoregulatory RNA binding protein, or a condition-specific RNA binding protein. In some embodiments, the intracellular protein is an MBNL protein, an SR protein, an hnRNP protein, an RbFox protein, a CELF protein, a Nova protein, or a PTB protein. In some embodiments, the protein is any one of MBNL1, MBNL2, MBNL3, hnRNP A1, hnRNP A2B1, hnRNP C, hnRNP D, hnRNP DL, hnRNP F, hnRNP H, hnRNP K, hnRNP L, hnRNP M, hnRNP R, hnRNP U, FUS, TDP43, PABPN1, ATXN2, TAF15, EWSR1, MATR3, TIA1, FMRP, MTM1, KIF5A, microdystrophin, C9ORF72, HTT, DNM2, B1N1, RYR1, NEB, ACTA, TPM3, TPM2, TNNT2, CFL2, KBTBD13, KLHL40, KLHL41, LMOD3, MYPN, SEPN1, TTN, SPEG, MYH7, TK2, POLG1, GAA, AGL, PYGM, SLC22A5, OCTN2, ETF, ETFH, PNPLA2, cytochrome b/cytochrome c oxidase, CLCN 1, SCN4A, DMPK, CNBP, MYOT, LMNA (Lamin A/C), CAV3, DNAJB6, DES, TNPO3, HNRPDL, CAPN3, DYSF, alpha-sarcoglycan, beta-sarcoglycan, gamma-sarcoglycan, delta-sacroglycan, TCAP, TRIM32, FKRP, POMT1, FKTN, POMT2, POMGnT1, DAG1, ANO5, PLEC1, TRAPPC11, GMPPB, 1SPD, LIMS2, POPDC1, TOR1AIP1, POGLUT2, LAMA2, COL6A1, POMT1, POMT2, DUX4, EMD, PAX7, PMP22, MPZ, MFN2, SMCHD 1, or GJB 1, or a truncated version thereof. Accordingly, in some embodiments, a nucleic acid encodes an RNA that comprises (e.g., in an intron and/or in an exon) one or more binding sites (e.g., 1, 2, 3, 4, 5, or more) for one or more of these RNA binding proteins and/or any other RNA binding proteins that can regulate intron splicing.

In some embodiments, the RNA comprises a regulatory RNA molecule, a short hairpin RNA molecule, a micro RNA molecule, or a transfer RNA molecule. In some embodiments, the protein-RNA complex comprises a ribosome, snRNP complex, or other macromolecular complex that contains at least one protein bound to at least one RNA.

In some embodiments, the first and/or second intron flanks an alternatively regulated exon and/or prevents RNAs from exiting the nucleus. In some embodiments, the first and/or second intron is a truncated version of a naturally occurring intron. In some embodiments, the first and/or second intron is or is derived from any one or more of: an NMD exon-flanking intron of SmB/B′, an exon 2b-flanking intron of SMN, an intron 3 of SMN, a 3′ UTR intron of hnRBP A2B1, an NMD exon-flanking intron of Tia1, an exon 7-flanking intron of Bin1, an exon 11-flanking intron of Bin1, an alternative exon-flanking intron of hnRNP D, an exon 13-flanking intron of FMRP, an exon 14-flanking intron of FMRP, an exon 15-flanking intron of FMRP, an alternative exon-flanking intron of Lamin A/C, an exon 11-flanking intron of S77, an alternative exon-flanking intron of Matrin 3, an alternative exon-flanking intron of NEXN, an alternative exon-flanking intron of NRAP, an alternative exon-flanking intron of MTM1, an exon 9-flanking intron of CACNA1C, an exon T2-flanking intron of MBNL1, an exon 1-flanking intron of MBNL1, an exon 2-flanking intron of MBNL1, an exon 3-flanking intron of MBNL1, an exon 4-flanking intron of MBNL1, an exon 5-flanking intron of MBNL1, an exon 6-flanking intron of MBNL1, an exon 7-flanking intron of MBNL1, an exon 9-flanking intron of MBNL1, an intron 6 of PABPN1, an intron 6 of TDP43, an intron 7 of TDP43, an intron 6 of FUS, an intron 7 of FUS, an intron 10 of hnRNP A1, and/or an exon 22-flanking intron of ATP2A1.

In some embodiments, the first and/or second intron comprises a 5′ splice donor site, optionally wherein the 5′ splice donor site is a GU or an AU. In some embodiments, the first and/or second intron comprises a 3′ splice acceptor site, optionally wherein the 3′ splice acceptor site is an AG or an AC. In some embodiments, the first and/or second intron comprises a region that regulates intron splicing. In some embodiments, the region that regulates intron splicing comprises one or more binding sites for a protein that regulates intron splicing.

In some embodiments, the rAAV further comprises an RNAi that targets a chromosomal allele encoding a gene encoding the intracellular factor. In some embodiments, the rAAV further comprises an exon.

In some embodiments, the exon is flanked by at least the first intron, optionally wherein the exon is flanked by the first and second intron. In some embodiments, the intracellular factor is a protein, wherein the exon comprises an open reading frame that encodes a portion of the protein. In some embodiments, the exon is naturally occurring. In some embodiments, the exon is a recombinant exon. In some embodiments, the recombinant exon comprises two or more naturally-occurring exons that are fused together without any intervening introns. In some embodiments, the rAAV further comprises a regulatory exon, wherein the regulatory exon comprises an in-frame stop codon that is at least 50 nucleotides upstream of the 5′ splice junction of the regulatory exon.

In some embodiments, the exon is or is derived from any one or more of: an NMD exon of SmB/B′, an exon 2b of SMN, an exon 3 of SMN, an exon 4 of SMN, an hnRBP A2B1 exon, an NMD exon of Tia1, an exon 7 of Bin1, an exon 11 of Bin1, an alternative exon of hnRNP D, an exon 13 of FMRP, an exon 14 of FMRP, an exon 15 of FMRP, an alternative exon of Lamin A/C, an exon 11 of ST7, an alternative exon of Matrin 3, an alternative exon of NEXN, an alternative exon of NRAP, an alternative exon of MTM1, an exon 9 of CACNA1C, an exon T2 of MBNL1, an exon 1 of MBNL1, an exon 2 of MBNL1, an exon 3 MBNL1, an exon 4 of MBNL1, an exon 5 of MBNL1, an exon 6 of MBNL1, an exon 7 of MBNL1, an exon 9 MBNL1, an exon 6 of PABPN1, an exon 7 of PABPN1, an exon 6 of TDP43, an exon 7 of TDP43, an exon 8 of TDP43, an exon 6 of FUS, an exon 7 of FUS, an exon 8 of FUS, an exon 10 of hnRNP A1, an exon 11 of hnRNP A1, and/or an exon 22 of ATP2A1.

In some embodiments, all introns and all exons are from the same gene. In some embodiments, at least one intron and at least one exon are from different genes. In some embodiments, the gene(s) comprises any one or more of: MBNL1, MBNL2, MBNL3, hnRNP A1, hnRNP A2B1, hnRNP C, hnRNP D, hnRNP DL, hnRNP F, hnRNP H, hnRNP K, hnRNP L, hnRNP M, hnRNP R, hnRNP U, FUS, TDP43, PABPN1, ATXN2, TAF15, EWSR1, MATR3, TIA1, FMRP, MTM1, KIF5A, a microdystrophin-encoding gene, C90RF72, HTT, DNM2, B1N1, RYR1, NEB, ACTA, TPM3, TPM2, TNNT2, CFL2, KBTBD13, KLHL40, KLHL41, LMOD3, MYPN, SEPN1, TTN, SPEG, MYH7, TK2, POLG1, GAA, AGL, PYGM, SLC22A5, OCTN2, ETF, ETFH, PNPLA2, a cytochrome b oxidase-encoding gene, a cytochrome c oxidase-encoding gene, CLCN1, SCN4A, DMPK, CNBP, MYOT, LMNA, CAV3, DNAJB6, DES, TNPO3, HNRPDL, CAPN3, DYSF, an alpha-sarcoglycan-encoding gene, a beta-sarcoglycan-encoding gene, a gamma-sarcoglycan-encoding gene, a delta-sacroglycan-encoding gene, TCAP, TRIM32, FKRP, POMT1, FKTN, POMT2, POMGnT1, DAG1, ANUS, PLEC1, TRAPPC11, GMPPB, ISPD, LIMS2, POPDC1, TOR1AIP1, POGLUT2, LAMA2, COL6A1, POMT1, POMT2, DUX4, EMD, PAX7, PMP22, MPZ, MFN2, SMCHD1, and/or GJB1.

In some embodiments, the rAAV further comprises a promoter. In some embodiments, the promoter is a constitutive promoter or a regulated promoter. In some embodiments, the regulated promoter is an inducible promoter. In some embodiments, the promoter comprises any one of: CMV, EF1alpha, CBh, synapsin, enolase, MECP2, MHCK7, Desmin, GFAP.

In some embodiments, the nucleic acid is flanked by adeno-associated virus (AAV) inverted terminal repeat (ITR) sequences. In some embodiments, the AAV ITR sequences comprise AAV1, AAV2, AAV5, AAV7, AAV8, or AAV9 ITR sequences.

Some aspects of the present invention contemplate a method of treating a disease or condition in a subject, comprising administering an rAAV according to any embodiment of the present disclosure to the subject. In some embodiments, the subject is a human subject.

In some embodiments, the disease or condition is selected from the group consisting of: a repeat expansion disease, a laminopathy, a cardiomyopathy, a muscular dystrophy, a neurodegenerative disease, a cancer, an intellectual disability, and/or premature aging. In some embodiments, the disease or condition is selected from the group consisting of: Dentatorubral-pallido-luysian atrophy (DRPLA), myotonic dystrophy type 1 (DM1), myotonic dystrophy type 2 (DM2), Fragile X syndrome of mental retardation (FMR1), Fragile X tremor ataxia syndrome (FXTAS), FRAXE mental retardation (FMR2), Friedreichs ataxia (FRDA), Huntington disease (HD), Huntington disease-like 2 (HDL2), Oculopharyngeal muscular dystrophy (OPMD), Myoclonic epilepsy type 1, Alzheimer's disease, ALS/FTD, spinocerebellar ataxia type 1 (SCA1), spinocerebellar ataxia type 2 (SCA2), spinocerebellar ataxia type 3 (SCA3), spinocerebellar ataxia type 6 (SCA6), spinocerebellar ataxia type 7 (SCAT), spinocerebellar ataxia type 8 (SCA8), spinocerebellar ataxia type 10 (SCA10), spinocerebellar ataxia type 12 (SCA12), spinocerebellar ataxia type 17 (SCA17), Syndromic/non-syndromic X-linked mental retardation, Emery-Dreifuss muscular dystrophy type 2, familial partial lipodystrophy, limb girdle muscular dystrophy type 1B, dilated cardiomyopathy, familial partial lipodystrophy, Charcot-Marie-Tooth disorder type 2B 1, mandibuloacral dysplasia, childhood progeria syndrome (Hutchinson-Gilford syndrome), Werner syndrome, Dilated cardiomyopathy (DCM), Hypertrophic cardiomyopathy (HCM), Restrictive cardiomyopathy (RCM), Left Ventricular Non-compaction (LVNC), Arrhythmogenic Right Ventricular Dysplasia (ARVD), takotsubo cardiomyopathy, Duchenne muscular dystrophy, Becker muscular dystrophy, Limb-girdle muscular dystrophy, Facioscapulohumeral muscular dystrophy, Congenital muscular dystrophy, Oculopharyngeal muscular dystrophy, Distal muscular dystrophy, Emery-Dreifuss muscular dystrophy, dementia, Parkinson's disease (PD), a PD-related disorder, Prion disease, a motor neuron disease (MND), Progressive bulbar palsy (PBP), Progressive muscular atrophy (PMA), Primary lateral sclerosis (PLS), Spinal muscular atrophy (SMA), a bladder cancer, a breast cancer, a colorectal cancer, a kidney cancer, a lung cancer, a lymphoma, a melanoma, an oral cancer, an ovarian cancer, an oropharyngeal cancer, a pancreatic cancer, a prostate cancer, a thyroid cancer, a uterine cancer, Down syndrome, Prader-Willi Syndrome (PWS), Bloom Syndrome, Cockayne Syndrome Type I-216400, Cockayne Syndrome Type III, Cockayne Syndrome Type I, Hutchinson-Gilford Progeria Syndrome, Mandibuloacral Dysplasia with Type A Lipodystrophy, Progeria, Adult Onset Progeroid Syndrome, Neonatal Rothmund-Thomson Syndrome, Seip Syndrome, Werner Syndrome, Replication Focus-Forming Activity 1, and/or centronuclear myopathy.

Accordingly, in some embodiments a recombinant nucleic acid described in this application (e.g., in an rAAV) is administered to a subject (e.g., a human patient) having one or more signs or symptoms of a disease or disorder (e.g., described herein) in order to treat or assist in the treatment of the disease or disorder.

In some embodiments, the rAAV is administered to the subject at least one time, optionally wherein the rAAV is administered to the subject multiple times. In some embodiments, the rAAV is administered by intravenous injection, intramuscular injection, intrathecal injection, or intravitreal injection.

Certain aspects of the present invention encompass an rAAV comprising a recombinant MBNL gene, wherein the recombinant MBNL gene comprises an MBNL protein coding sequence, and at least one truncated intron of the MBNL gene. In some embodiments, splicing of the truncated intron is regulated by an intracellular protein. In some embodiments, the MBNL gene is MBNL1, MBNL2, or MBNL3.

In some embodiments, the rAAV further comprises at least one exon. In some embodiments, the at least one exon is exon 1 and/or exon 5 of the MBNL gene. In some embodiments, the at least one truncated intron is a truncated exon 1-flanking intron and/or a truncated exon 5-flanking intron of the MBNL gene. In some embodiments, the at least one truncated intron comprises: SEQ ID NOs: 2 and 4, but does not comprise SEQ ID NO: 3; SEQ ID NOs: 6 and 8, but does not comprise SEQ ID NO: 7: SEQ ID NOs: 11 and 13, but does not comprise SEQ ID NO: 12; or SEQ ID NOs: 15 and 17, but does not comprise SEQ ID NO: 16. In some embodiments, the at least one exon is exon 3 or exon 9 of the MBNL gene. In some embodiments, the at least one exon is exon 8 and/or exon 10 of the MBNL gene. In some embodiments, the at least one exon is exon 22 of the ATPA1 gene, optionally wherein the rAAV further comprises one or more exon 22-flanking introns of the ATPA1 gene.

In some embodiments, the rAAV further comprises a promoter. In some embodiments, the promoter is an endogenous or an exogenous promoter. In some embodiments, the exogenous promoter is any one of an: EF1 alpha promoter, beta actin promoter, MHCK7, CBh, synapsin, MECP2, enolase, GFAP, Desmin, and CAG promoter. In some embodiments, the rAAV further comprises an endogenous or an exogenous 3′ untranslated region (UTR). In some embodiments, the exogenous 3′ UTR is the 3′ UTR from bovine growth hormone, SV40, EBV, or Myc.

In some embodiments, the expression construct comprises the second 5′ UTR of the MBNL gene, and does not include the first or the third 5′ UTR of the MBNL gene. In some embodiments, the recombinant MBNL gene is flanked by adeno-associated virus (AAV) inverted terminal repeat (ITR) sequences. In some embodiments, the AAV ITR sequences comprise AAV 1, AAV2, AAV5, AAV7, AAV8, or AAV9 ITR sequences.

Some aspects of the present invention describe a method of treating an MBNL-related disease or condition in a subject, comprising administering an rAAV according to any embodiment of the present disclosure to the subject. In some embodiments, the rAAV is administered to the subject at least one time, optionally wherein the rAAV is administered to the subject multiple times. In some embodiments, the rAAV is administered by intravenous injection, intramuscular injection, intrathecal injection, or intravitreal injection.

In some embodiments, the MBNL-related disease or disorder is any one of Fuch's endothelial corneal dystrophy, Huntington Disease, or a cancer.

Some aspects of the present invention contemplate an rAAV comprising a nucleic acid having the sequence of any one or more of SEQ ID NOs: 1, 2, 4-6, 8-11, 13-15, and/or 17-49.

Accordingly, in some embodiments, recombinant gene expression constructs that encode one or more recombinant introns are provided. Such gene expression constructs can be packaged in a recombinant virus to provide regulated (e.g., auto-regulated) gene expression vectors that can be delivered to a patient.

In some embodiments, a recombinant intron comprises a regulatory region that can regulate splicing of the intron. In some embodiments, the regulatory region contains one or more nucleic acid (e.g., RNA) binding sites for a protein (e.g., an RNA binding protein) that regulates splicing of the intron. In some embodiments, a recombinant intron is shorter than a naturally-occurring intron that contains the regulatory region. In some embodiments, a recombinant intron contains a 5′ splice donor site. In some embodiments, a recombinant intron contains a 3′ splice acceptor site. In some embodiments, the regulatory region, the 5′ splice donor site, and/or the 3′ splice acceptor site can be from a naturally-occurring intron. In some embodiments, the naturally-occurring intron can be truncated (e.g., by removing regions between the 5′ splice donor site, the regulatory region, and/or the 3′ splice acceptor site). In some embodiments, the recombinant intron contains one or more of a synthetic 5′ splice donor site, regulatory region, and/or 3′ splice acceptor site (e.g., that have sequences that vary from naturally-occurring sequences). In some embodiments, the recombinant intron contains a combination of a 5′ splice donor site, a regulatory region, and a 3′ splice acceptor site that are from different naturally-occurring introns and/or synthetic.

In some embodiments, a recombinant intron flanks an exon in a gene expression construct to allow for regulated splicing of the intron to control whether the exon is included or not in the translated coding sequence of the spliced RNA product. In some embodiments, an exon is flanked by two recombinant introns to allow for alternative splicing that either includes or excludes the exon from a protein coding spliced RNA product. In some embodiments, at least one of the recombinant introns includes a regulatory (e.g., auto-regulatory) sequence that regulates intron splicing.

A recombinant intron coding sequence can be designed or provided to be sufficiently small to fit in a recombinant viral genome so that it can be efficiently packaged in a viral vector. In some embodiments, a recombinant intron coding sequence can be between around 20 bases long and around 2,000 bases long. In some embodiments, a recombinant intron is around 50 bases, around 100 bases, around 250 bases, around 500 bases, around 1,000 bases, around 1,500 bases, or around 2,000 bases long. However, longer recombinant introns also can be used in some embodiments.

Accordingly, in some embodiments a recombinant viral genome comprises a gene expression construct that includes at least one exon coding sequence flanked by a recombinant intron coding sequence, wherein the recombinant intron a) comprises a region that regulates intron splicing, and b) is shorter than an intron that flanks the at least one exon in a naturally-occurring gene. In some embodiments, the recombinant intron is a truncated naturally-occurring intron.

In some embodiments, a recombinant viral genome comprises a gene expression construct that includes at least one exon coding sequence flanked by two recombinant intron coding sequences, wherein at least one of the two recombinant intron coding sequences comprises a region that regulates intron splicing.

In some embodiments, a region in an intron that regulates intron splicing comprises one or more binding sites for a protein (e.g., an RNA binding protein) that regulates intron splicing. In some embodiments, the protein that regulates intron splicing is encoded by the gene expression construct (e.g., by the construct that contains the one or more introns that are regulated). Accordingly, in some embodiments, intron splicing of the gene expression construct is auto-regulated by the protein that is expressed from the construct.

In some embodiments, the regulatory protein (e.g., the protein that binds to and regulates splicing of the recombinant intron) is a tissue-specific RNA binding protein. In some embodiments, the regulatory protein is an MBNL protein, an SR protein, an hnRNP protein, an RbFox protein, a CELF protein, a Nova protein, or a PTB protein. In some embodiments, the MBNL protein is an MBNL1, MBNL2 or MBNL3 protein. In some embodiments, the hnRNP protein is an hnRNP A1, hnRNP A2, or hnRNP C protein.

In some embodiments, a recombinant intron comprises a 5′ splice donor site (e.g., comprising a 5′ GU). In some embodiments, a recombinant intron comprises a 3′ splice acceptor site (e.g., comprising a 3′ AG).

In some embodiments, the gene expression construct in a recombinant viral genome is flanked by viral terminal repeat sequences. In some embodiments, the viral terminal repeat sequences are adeno-associated virus (AAV) inverted terminal repeat (ITR) sequences.

In some embodiments, the gene expression construct comprises a constitutive promoter operatively linked to a protein coding sequence that includes one or more recombinant intron coding sequences. In some embodiments, the gene expression construct comprises a regulated promoter operatively linked to a protein coding sequence that includes one or more recombinant intron coding sequences. In some embodiments, the regulated promoter is an inducible promoter.

In some embodiments, the gene expression construct encodes only one recombinant intron. In some embodiments, the gene expression construct encodes only two recombinant introns, and the two recombinant introns flank an open reading frame (e.g., a portion of a protein coding sequence) that encodes a portion of a protein. In some embodiments, the open reading frame is a naturally-occurring exon. In some embodiments, the open reading frame comprises two or more naturally-occurring exons that are fused together without any intervening introns in the gene expression construct.

In some embodiments, a gene expression construct encodes one or two recombinant introns for which splicing is regulated. In some embodiments, splicing of the one or two recombinant introns is auto-regulated (e.g., by a protein encoded by the gene expression construct).

In some embodiments, a gene expression construct includes additional exons (e.g., all or a subset of the other exons from a naturally-occurring gene) in addition to the one or more exons that are flanked by regulated recombinant introns. Accordingly, in some embodiments a gene expression construct encodes 1-10 or more additional exons.

In some embodiments, the additional exons are fused without any intervening introns (e.g., without the naturally-occurring introns) in a gene expression construct. However, in some embodiments, the gene expression construct encodes one or more additional introns (e.g., 1-10 or more additional introns) between the additional exons and for which splicing is not regulated. The additional introns can be recombinant introns that are shorter than their corresponding naturally occurring introns.

In some embodiments, a recombinant virus is provided comprising a recombinant viral genome containing one or more recombinant intron coding sequences as described herein. In some embodiments, the recombinant virus is a recombinant AAV virus.

In some embodiments, a recombinant virus is used to deliver a gene expression construct for which splicing is regulated (e.g., auto-regulated) to a subject. In some embodiments, the subject is a human subject. In some embodiments, the subject has a disease or condition for which delivery of a regulated (e.g., auto-regulated) gene expression construct can be helpful to alleviate or treat one or more symptoms. In some embodiments, the disease or condition is selected from the group consisting of myotonic dystrophy or any disease involving dysregulation and/or altered activity of an RNA binding protein (e.g., spinocerebellar ataxias, Huntington's Disease, Fuch's Endothelial Corneal Dystrophy, Amyotrophic Lateral Sclerosis, Frontotemporal Dementia, Fragile X Tremor-Associated Syndrome, Spinal Muscular Atrophy, certain types of cancer, or other diseases or disorders). In some embodiments, the disease or disorder is due to one or more repeat expansions or other mutations.

In some embodiments, methods and compositions comprising a recombinant intron can be used to deliver an MBNL expression construct that is auto-regulated. Accordingly, in some embodiments a recombinant MBNL expression construct comprises an MBNL gene and one or more truncated introns of the MBNL gene. In some embodiments, the MBNL gene is MBNL1, MBNL2, or MBNL3.

In some embodiments, the recombinant MBNL expression construct comprises exon 1 of the MBNL gene. In some embodiments, the recombinant MBNL expression construct comprises exon 5 of the MBNL gene. In some embodiments, the recombinant MBNL expression construct comprises exon 1 and exon 5 of the MBNL gene. Accordingly, in some embodiments the recombinant MBNL expression construct encodes one or more truncated introns selected from the group of exon 1-flanking introns and exon 5-flanking introns of the MBNL gene.

In some embodiments, a truncated intron comprises SEQ ID NOs: 2 and 4, but does not comprise SEQ ID NO: 3. In some embodiments, a truncated intron comprises SEQ ID NOs: 6 and 8, but does not comprise SEQ ID NO: 7. In some embodiments, a truncated intron comprises SEQ ID NOs: 11 and 13, but does not comprise SEQ 1D NO: 12. In some embodiments, a truncated intron comprises SEQ ID NOs: 15 and 17, but does not comprise SEQ ID NO: 16.

Accordingly, in some embodiments a recombinant MBNL expression construct encodes exon 1 and/or exon 5 of the MBNL gene, each flanked by one or two recombinant (e.g., truncated) introns. In some embodiments, a recombinant MBNL expression construct further encodes one or more additional MBNL exons (e.g., all the exons of a wild-type MBNL gene). However, in some embodiments, a recombinant MBNL expression construct does not encode all the exons of an MBNL gene. In some embodiments, the recombinant MBNL expression construct comprises exon 3 of the MBNL gene. In some embodiments, the recombinant MBNL expression construct does not include exon 9 of the MBNL gene. In some embodiments, the recombinant MBNL expression construct comprises exon 8 of the MBNL gene. In some embodiments, the recombinant MBNL expression construct comprises exon 10 of the MBNL gene. In some embodiments, the recombinant MBNL expression construct comprises exons 8 and 10 of the MBNL gene.

In some embodiments, a recombinant gene expression construct (e.g., a recombinant MBNL expression construct) comprises an exogenous promoter. In some embodiments, the exogenous promoter is selected from the group consisting of an EF1 alpha promoter, a beta actin promoter, and a CAG promoter.

In some embodiments, a recombinant gene expression construct (e.g., a recombinant MBNL expression construct) comprises an exogenous 3′ untranslated region (UTR). In some embodiments, the exogenous 3′ UTR is the 3′ UTR from bovine growth hormone. In some embodiments, the expression construct comprises the second 5′ UTR of an MBNL gene, and does not include the first or the third 5′ UTR of the MBNL gene.

In some embodiments, a recombinant gene expression construct (e.g., a recombinant MBNL expression construct) is flanked by viral terminal repeat sequences (e.g., AAV ITRs).

In some embodiments, a recombinant expression construct (e.g., a recombinant MBNL expression construct), for example flanked by viral terminal repeat sequences (e.g., in a recombinant viral particle) is administered to a subject. In some embodiments, the subject administered the recombinant expression construct (e.g., MBNL expression construct) has one or more of the following non-limiting list of conditions: myotonic dystrophy, Fuch's Endothelial Corneal Dystrophy, Huntington's Disease, any number of spinocerebellar ataxias, and/or other conditions in which increased MBNL levels would be beneficial. In some embodiments, an auto-regulated RNA binding protein construct (e.g., a recombinant MBNL expression construct) is administered to a subject having any one of a number of diseases in which RNA binding protein homeostasis is perturbed. In a non-limiting example, an auto-regulated RNA binding construct is used to alter the ratio of non-mutated versus mutated versions of the RNA binding protein in disease (e.g., amyotrophic lateral sclerosis, frontotemporal dementia, Spinal Muscular Atrophy, other repeat expansion diseases, or cancer).

The details of one or more embodiments of the invention are set forth in the description below. Other features or advantages of the present invention will be apparent from the following drawings and detailed description of several embodiments, and also from the appended claims.

### BRIEF DESCRIPTION OF DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1D show non-limiting examples of an MBNL gene therapy approach. FIG. 1A shows schematics of coding sequences for MBNL1 and MBNL2. Constitutive exons are in white and alternative exons in gray. Protein domains (zinc fingers, bipartite nuclear localization sequences, and the unstructured C-terminal tail) are indicated. FIG. 1B shows splicing patterns for MBNL1. FIG. 1C illustrates a non-limiting example of an MBNL (e.g., MBNL1) gene construct having truncated introns flanking exons 1 and 5, allowing the construct to fit the cargo inside the AAV packaging limit of 4.9 kb. FIG. 1D shows a non-limiting example of the proposed functionality of AAV-MBNL1_{spliced} as compared to a version that cannot splice.

FIG. 2 shows a graph and schematic depicting mini-gene constructs in which introns 4 and 5 have been truncated retain auto-regulatory activity. MiniAR5.1 is the “full length” construct that contains endogenous human intron 4, exon 5, and intron 5 of MBNL1. Subsequent mini-genes contain similar sequence but intronic sequences have been removed. All mini-gene constructs were transfected with DT480, empty, or EGFP-MBNL1 plasmids into Hela cells. The inclusion level of exon 5 in the mini-gene constructs were assessed by RT-PCR using primers targeting the flanking exons.

FIG. 3 shows a graph and schematic depicting AAV constructs containing full length MBNL1 coding sequence and truncated or optimized versions of intron 4, intron 5, and other sequences. AR5 constructs were tested in Hela cells by co-transfection with DT480, EGFP, or EGFP-MBNL1. The inclusion level of exon 5 in the AR5 constructs was assessed by RT-PCR across the exon. AR5.1 is the “truncated” intron 4-exon 5-intron 5 cassette derived from miniAR5.4. AR5.2 contains a mutation to a cryptic splice site in exon 6. AR5.3-AR5.5 contain additional endogenous sequence from intron 5 that has been re-introduced into the constructs, along with the mutation made in AR5.2.

FIG. 4 shows results of optimization of AAV AR5 constructs. Hela cells were transfected with AR5.2-AR5.5, along with DT480, empty plasmid, or EGFP-MBNL 1. All isoforms generated, including intron retention isoforms, are illustrated. Intron retention is reduced in AR5.4 and AR5.5 relative to the other constructs.

FIG. 5 shows a graph of mini-gene constructs in which the introns between T2 and exon 1 and between exon 1 and exon 2 have been truncated retain auto-regulatory activity. MiniAR1.1 contains the entirety of exon 1, whereas miniAR 1.2 and miniAR 1.3 contain versions of exon 1 in which additional sequences have been removed. All mini-gene constructs were transfected with DT480, empty, or pCI-MBNL1-260aa plasmids into Hela cells. The inclusion level of exon 1 in the mini-gene constructs were assessed by RT-PCR using primers targeting the flanking exons.

FIG. 6 shows constructs showing auto-regulatory activity of exon 1. AR1.1 and AR1.2 are cloned in the context of AAV ITRs, and AR1.3-AR1.6 are in a pcDNA expression backbone. All constructs were transfected with DT480, empty, or pCI-MBNL1-260aa plasmids into Hela cells. Protein was harvested and subjected to Western blotting against the HA tag that is found at the end of each AR1 construct, as well as GAPDH as a normalizing control.

FIG. 7 shows cryptic splice sites in AR1.1 and AR1.2 that are used upon MBNL over-expression. In addition to complete skipping of exon 1, e.g. joining of the 5′ splice site of T2 to the 3′ splice site of exon 2, cryptic splice sites within exon 1 are frequently used. In AR1.1, use of this cryptic splice site upon MBNL over-expression leads to translation of a short peptide that would be expected to elicit nonsense-mediated decay. In AR1.2, use of a different cryptic splice site upon MBNL over-expression leads to an “ATG-STOP” sequence, which was also expected to result in mRNA decay. Cryptic splice site usage was determined by RT-PCR and Sanger sequencing.

FIG. 8 shows a graph of some isoforms generated by AR1.1 and AR1.2 increase in relative abundance upon UPF1 knockdown. AR1.1Q (a variation of AR1.1 in which synonymous mutations were made in exon 10 in order to distinguish AR1.1 from endogenous MBNL1 by RT) and AR1.2Q (an analogous variation of AR1.2) were co-transfected with or without a short hairpin RNA against UPF1, and DT480, empty plasmid, or EGFP-MBNL1- 269aa. Isoforms that are subject to NMD show increased abundance upon UPF knockdown and are lowest in the DT480 condition.

FIG. 9 shows that AAV constructs containing both AR1 and AR5 cassettes maintain auto-regulatory behavior. The exon 5 portion of these constructs was assessed by RT-PCR following transfection of each construct with DT480, EGFP, or EGFP-MBNL1-269aa. Similar to constructs shown in FIG. 8, these also have synonymous mutations in exon 10 to distinguish the exogenous construct from endogenous MBNL1.

FIGS. 10A-10D show proposed AAV constructs that exhibit auto-regulation. Each RNA binding protein contains naturally occurring regulatory mechanisms that allow it to respond to its own levels and the levels of other RNA binding proteins. The specific introns and exons important for this behavior are outlined here; truncation and/or incorporation of these introns into AAV enables expression of a therapeutic cargo that can self-regulate. Designs are shown for PABPN1 (FIG. 10A), TDP43 (FIG. 10B), FUS (FIG. 10C), and hnRNP A1 (FIG. 10D).

FIG. 11 shows a Western blot of protein from Hela cells transfected with an auto-regulated AAV encoding hnRNP A1. In lanes 1-3, increasing amounts of AAV-HA-hnRNP A1 are transfected in the presence of decreasing amounts of AAV-HA-GFP (to maintain similar total plasmid transfected across conditions). In lanes 4-6, AAV-HA-hnRNP A1 is kept constant, but increasing amounts of pcDNA hRNP A1 coding sequence with a V5 tag are transfected in the presence of decreasing amounts of pcDNA LacZ with a V5 tag (to maintain total plasmid transfected across conditions).

FIG. 12 shows a Western blot of protein from Hela cells transfected with an auto-regulated AAV encoding TDP43. In lanes 1-3, increasing amounts of AAV-HA-TDP43 are transfected in the presence of decreasing amounts of AAV-HA-GFP (to maintain similar total plasmid transfected across conditions). In lanes 4-6, AAV-HA-TDP43 is kept constant, but increasing amounts of pcDNA TDP43 coding sequence with a V5 tag are transfected in the presence of decreasing amounts of pcDNA LacZ with a V5 tag (to maintain total plasmid transfected across conditions).

FIGS. 13A-13B show a design for a heterologous auto-regulated AAV construct that uses NMD to down-regulate its own activity. FIG. 13A shows inclusion levels of ATP2A 1 exon 22 in healthy and DM1 human tibialis biopsies; splicing from each biopsy is ranked according to inferred MBNL activity—figure adapted from Wagner et al, PLOS Genetics 2016. FIG. 13B shows a design for this cassette exon and flanking introns in the context of MBNL1 coding sequence. The cassette exon was modified to contain a stop codon in the MBNL1 reading frame, and additional sequence was added to ensure that the distance from the stop codon to the downstream splice junction was sufficient to elicit nonsense-mediated decay (>50 nucleotides).

FIGS. 14A-14C show a design and proof of concept for a heterologous auto-regulated AAV construct. FIG. 14A shows inclusion levels of MBNL1 exon 5 in healthy and DM1 human tibialis biopsies; splicing from each biopsy is ranked according to inferred MBNL activity —figure adapted from Wagner et al, PLOS Genetics 2016. This exon is mostly excluded in healthy muscle, and mostly included in severe DM1. FIG. 14B shows a design for this construct using exon 5 and flanking introns, moved from their natural position to the beginning of MBNL 1 coding sequence. The natural start codon was removed from exon 1, and an “A” was added before a “TG” at the very end of exon 5, to generate a new “ATG”. Thus, when exon 5 was included, protein was generated, whereas when it was excluded, no protein was generated. FIG. 14C shows proof of concept for this construct. AR5.5 was used as the alternative splicing cassette, which uses optimized/truncated introns. The construct was co-transfected into Hela with DT480, empty plasmid, or EGFP-MBNL1-269aa, and the splicing pattern was assessed by RT-PCR across the alternative exon. The exon responded as expected, being included with DT480 and excluded with EGFP-MBNL1-269aa. Western blotting of protein from these cells also showed the expected pattern of protein expression, where more protein was expressed with DT480 and less with EGFP-MBNL1-269aa.

FIG. 15 shows that auto-regulated AAV-MBNL1 can rescue mis-splicing in a DM1 mouse model. WT and HSALR mice were dosed with AAV encoding CBh promoter driven MBNL1-40 coding sequence, AR1.2, AR5.2, or a mixture of MBNL1-40 and AR1.2 by intramuscular injection into the tibialis anterior: PBS was dosed into the contralateral leg. Tissue was harvested ˜28 days later and splicing patterns for 3 different MBNL-dependent splicing events was studied. The black dots show PSI in the PBS-dosed leg, and the colored dots show PSI in the AAV-dosed leg. Generally, all constructs show splicing rescue in the WT direction, or the direction of rescue, supporting the expression and activity of auto-regulated constructs.

FIGS. 16A-16C show that auto-regulated AAV-MBNL1 responds to the overall concentration of MBNL in various tissues in a mouse model of DM1. WT and HSALR mice were dosed with AAV encoding CBh-driven AR5.2 by tail vein injection (FIG. 16A) or intramuscular injection (FIG. 16B). In liver, with low or high doses, the inclusion level of exon 5 in AR5.2 was not appreciably different between WT and HSALR mice, because the liver was not diseased in this mouse model. However, in muscle, the inclusion level of exon 5 of AR5.2 showed a difference between WT and HSALR mice, because there was MBNL depletion in HSALR muscle. The endogenous splicing pattern of MBNL1 exon 5 was also assayed (FIG. 16C) and mirrored that of AR5.2, indicating that the alternative splicing behavior of AR5.2 behaves similarly to endogenous exon 5, even though AR5.2 contained truncated sequence and was delivered by AAV.

### DETAILED DESCRIPTION

The present disclosure relates to methods and compositions that are useful for delivering gene constructs to treat diseases or disorders that involve regulated intron splicing (e.g., auto-regulated intron splicing). Aspects of the application relate to methods and compositions for regulating nucleic acid expression in recombinant adeno-associated virus (rAAV) vectors. In some aspects, an rAAV comprises a recombinant AAV genome that includes a nucleic acid that encodes an RNA (e.g., an mRNA) comprising one or more introns. In some embodiments, splicing of at least one intron is regulated by one or more intracellular factor(s). Regulation of intron splicing can control the expression level of the RNA and/or of the type of RNA (e.g., of an RNA splice alternative) inside a cell.

In some aspects, intron splicing regulation can be used to help control the expression of one or more therapeutic transcripts that are encoded by an rAAV. Accordingly, compositions and methods described herein can be useful to regulate expression of therapeutic transcripts in the context of rAAV based treatments for diseases or disorders (e.g., diseases or disorders that involve regulated intron splicing, such as auto-regulated intron splicing). Abnormal cellular regulation (e.g., abnormal regulation of intron splicing of one or more genes) can lead to changes in gene regulation and subsequent protein expression associated with a disease state. In some aspects, the present application provides compositions and methods that are useful for delivering genes that retain or restore therapeutically effective levels of regulation (e.g., therapeutically effective regulation of intron splicing).

In some embodiments, a recombinant nucleic acid comprises one or more introns and/or exons that include one or more RNA protein binding motifs such that splicing of at least one intron can be regulated (e.g., in response to a level of an RNA binding protein, for example in a tissue-specific fashion, in response to intracellular levels of an autoregulated protein, in response to one or more intracellular factors and/or levels of intracellular factors, or in response to an external stimulus). In some embodiments, splicing results in expression of a transcript. In some embodiments, splicing results in the production of a protein coding sequence that is translated. In some embodiments, splicing results in an alternative protein coding sequence being produced. Accordingly, by including one or more regulated introns in a recombinant nucleic acid for delivery in a recombinant virus (e.g., for delivery in an rAAV), expression of a transcript (e.g., of a protein coding transcript) encoded by the nucleic acid can be regulated in a host cell (e.g., in a subject) transduced by the virus (e.g., rAAV). In some embodiments, a recombinant nucleic acid is designed to include only a single intron or a subset of introns that are found in a natural gene. The one or more introns can be designed to be sufficiently small to be efficiently packaged in a recombinant virus (e.g., in an rAAV) as part of the recombinant nucleic acid (e.g., recombinant rAAV genome) that also can include one or more exons in some embodiments.

### Introns

In some embodiments, an rAAV of the present invention comprises a nucleic acid encoding an RNA, wherein the RNA comprises at least one intron (e.g., 1-5, 5-10, for example 1, 2, 3, 4, 5, 6, 7, 8, 9, 10, 10-15, or more) that may be regulated by an intracellular factor.

In some aspects, an intron for which splicing may be regulated is an intron for which splicing levels differ by at least 5%, for example at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% under two different conditions (e.g., in different tissues, in response to intracellular levels of one or more RNA binding proteins, in the context of an autoregulated gene, etc.). By “splicing levels differ by 5%”, it is meant that the splicing levels for an intron of interest are measured in two different conditions, and the splicing level is compared between the conditions and expressed as a percentage change. For example, if the splicing level in condition A is 80%, and the splicing level in condition B is 85%, the splicing levels between conditions A and B differ by 5%. Likewise, if the splicing level in condition A is 80%, and the splicing level in condition B is 75%, the splicing levels between conditions A and B also differ by 5%.

In some embodiments, an intron contains functional splice donor and acceptor sites (e.g., naturally occurring or engineered splice donor and/or acceptor sites), and one or more regulatory regions that can control intron splicing (e.g., whether splicing occurs or whether which of one or more alternative splicing events occurs). In some embodiments, a regulatory region can bind to one or more intracellular factors (e.g., RNA binding proteins) that regulate intron splicing. In some embodiments, an intracellular factor comprises a protein, an RNA, or a protein-RNA complex. In some embodiments, the protein comprises an RNA-binding protein. In some embodiments, an intron for which splicing is regulated comprises one or more RNA binding protein sites (e.g., 2, 3, 4, 5, 6, 7, 8, 9, 10, or 10-20). As used herein, an “RNA binding protein site” refers to an RNA sequence or structure that interacts with an “RNA binding protein” as defined below. In some embodiments, the binding site(s) can be in an intron, an exon, or both. An “RNA binding protein” (RBP) refers to a protein that binds to double- or single-stranded RNA in cells and participates in forming ribonucleoprotein complexes. RBPs contain various structural motifs, such as RNA recognition motif (RRM), dsRNA binding domain, zinc finger and others.

In some embodiments, an RNA binding protein is a sequence-specific RNA binding protein. In some embodiments, a useful sequence-specific RNA binding protein binds to a target sequence with a binding affinity (e.g., Kd) of 0.01-1000 nM or less (e.g., 0.01 to 1, 1-10, 10-50, 50-100, 100-500, 500-1,000 nM). In some embodiments, an RNA binding protein has serine/arginine domains that act as splicing enhancers, or glycine-rich domains that act as splicing repressors. In some embodiments, an RNA binding protein acts as an intronic splicing enhancer, intronic splicing silencer, exonic splicing enhancer, or exonic splicing silencer.

Different types of sequence-specific RNA binding proteins can be used. In some embodiments, a sequence-specific RNA binding protein is one that contains zinc fingers, RNA recognition motifs, KH domains, deadbox domains, or dsRBDs. Non-limiting examples of RBPs that contain zinc fingers include: MBNL, T1S 11, or TTP. Non-limiting examples of RBPs that contain RNA recognition motifs include hnRNPs and SR proteins, RbFox, PTB, Tra2beta. Non-limiting examples of RNA binding proteins that contain KH domains include Nova, SF1, and FBP. Non-limiting examples of RNA binding proteins that contain deadbox domains are DDX5, DDX6, and DDX17. Non-limiting examples of RNA binding proteins that contain dsRBDs include ADAR, Staufen, and TRBP.

Further examples of these types of RNA binding proteins and their respective sequence specific binding motifs are known in the art, and can be found, for example, in Perez-Perri, J. I., et al., (2018), Nat. Comm., 9:4408; Van Nostrand, E. L., et al., (2020), Nature, 583, 711-19; and Corley, M., et al., (2020), Cell, (20): 30159-3, the contents of which are hereby incorporated by reference with respect to RNA protein binding sites and RNA binding proteins.

In some embodiments, splicing of a regulated intron (e.g., an auto-regulated intron) in a gene of a subject can be affected by the presence in the subject of one or more mutations (e.g., genomic mutations) that alter binding of splice regulatory proteins to a regulatory region of the regulated intron. In some embodiments, such mutations can include the presence of other sequences that can alter (e.g., increase or decrease) binding to splice regulatory proteins. Accordingly, in some embodiments a composition of the application can be delivered to a subject that has a condition associated with aberrant splice regulation of one or more genes in order to restore, at least partially, normal levels of splice regulation. In some embodiments, a gene comprising one or more introns for which splicing is appropriately regulated is provided in an rAAV. In some embodiments, the rAAV also encodes an inhibitory molecule (e.g., an inhibitory RNA, for example an siRNA) that inhibits expression (e.g., transcription and/or translation) of the aberrant transcript (the transcript for which splicing is aberrant) expressed from the genome of the subject. In some embodiments, an inhibitory RNA targets one of the genomic alleles in a subject. In some embodiments, an inhibitory RNA targets both genomic alleles in a subject.

In some embodiments, an intron is an engineered intron. In some embodiments, the engineered intron comprises a donor and acceptor splice site, and a functional branch point to which the donor splice site can be joined in the first trans-esterification reaction of splicing. In some embodiments, an intron comprises a truncated version of a natural intron. By “truncated natural intron”, it is meant that the naturally-occurring, full-length intron is shortened (e.g., truncated) via the removal of nucleotides. In some embodiments, a recombinant intron (e.g., a synthetic intron) can be used. In some embodiments, a recombinant intron is a truncated version of a natural intron. However, in some embodiments a recombinant intron can be designed to include functional splice donor and acceptor sites and a functional branch point in addition to one or more regulatory regions that are derived from different introns, or that are non-naturally occurring sequences (e.g., sequence variants of naturally-occurring sequences, consensus sequences, or de-novo designed sequences). Accordingly, in some embodiments a recombinant intron is not a truncated version of a naturally occurring intron, but contains one or more sequences from a naturally occurring intron.

In some embodiments, a truncated intron is truncated at its 5′ end. In some embodiments, 1-10,000 nucleotides are truncated from the 5′ end (e.g., 1-50, 50-100, 100-500, 500-1,000, 1,000-5,000, 5,000-10,000, 10,000-20,000, 20,000-50,000, or 50,000-100,000 nucleotides are truncated from the 5′ end). In some embodiments, the 5′ splice site is retained in the truncated intron. In some embodiments, a different 5′ splice site is included in the truncated intron.

In some embodiments, a truncated intron is truncated at its 3′ end. In some embodiments, 1-10,000 nucleotides are truncated from the 3′ end (e.g., 1-50, 50-100, 100-500, 500-1,000, 1,000-5,000, 5,000-10,000, 10,000-20,000, 20,000-50,000, or 50,000-100,000 nucleotides are truncated from the 3′ end). In some embodiments, the 3′ splice site is retained in the truncated intron. In some embodiments, a different 3′ splice site is included in the truncated intron.

In some embodiments, a truncated intron is truncated at one or more internal locations. In some embodiments, 1-10,000 internal nucleotides are removed (e.g., 1-50, 50-100, 100-500, 500-1,000, 1,000-5,000, 5,000-10,000, 10,000-20,000, 20,000-50,000, or 50,000-100,000 internal nucleotides are removed). In some embodiments, the splice regulatory region is retained in the truncated intron. In some embodiments, a different splice regulatory region is included in the truncated intron.

In some embodiments, a truncated intron comprises one or more 5′, 3′, and/or internal deletions. It should be understood that the extent of truncation may depend on the size of the intron and the size of the gene. A truncation may require removal of sufficient intron sequences to result in a recombinant gene construct that is small enough to be packaged in a recombinant virus of interest (e.g., in an rAAV virus).

However, an engineered (e.g., truncated) intron typically includes one or more sequences required for efficient splicing and/or regulated (e.g., autoregulated) splicing. In some embodiments, a recombinant (e.g., truncated) intron retains a donor site (e.g., towards the 5′ end of the truncated intron), a branch site (e.g., towards the 3′ end of the truncated intron), an acceptor site (e.g., at the 3′ end of the truncated intron), and a splice regulatory sequence. In some embodiments, the intron comprises a 5′ splice donor site. In some embodiments, the 5′ splice donor site is a GU or an AU. In some embodiments, the intron comprises a 3′ splice acceptor site. In some embodiments, the 3′ splice acceptor site is an AG or an AC. In some embodiments, a regulatory sequence comprises a response element within an AG exclusion zone of the truncated intron. In some embodiments, the truncated intron retains sequence motifs bound by the encoded protein (e.g., YGCY motifs for MBNL1, or GCAUG for RBFOX, or YCAY for NOVA, etc.).

In some embodiments, an engineered (e.g., truncated) intron may include one or more human, non-human primate, and/or other mammalian or non-mammalian intron splice-regulatory sequences. In some embodiments, the regulatory sequences may have 80%-100% (e.g., 80-85%, 85%-90%, greater than 90%, 90%-95%, or 95%-100%) sequence identify with a wild-type regulatory sequence.

In some embodiments, an engineered intron is approximately 50 to 4000 nucleotides long. In some embodiments, an engineered intron is approximately 50 to 100, 75-125, 100-150, 125-175, 200-250, 225-275, 300-350, 325-375, 400-450, 425-475, 500-550, 525-575, 600-650, 625-675, 700-750, 725-775, 800-850, 825-875, 900-950, 925-975, 950-1000, 1025-1075, 1050 to 1100, 1075-1125, 1100-1150, 1125-1175, 1200-1250, 1225-1275, 1300-1350, 1325-1375, 1400-1450, 1425-1475, 1500-1550, 1525-1575, 1600-1650, 1625-1675, 1700-1750, 1725-1775, 1800-1850, 1825-1875, 1900-1950, 1925-1975, 1950-2000, 2025-2075, 2050 to 2100, 2075-2125, 2100-2150, 2125-2175, 2200-2250, 2225-2275, 2300-2350, 2325-2375, 2400-2450, 2425-2475, 2500-2550, 2525-2575, 2600-2650, 2625-2675, 2700-2750, 2725-2775, 2800-2850, 2825-2875, 2900-2950, 2925-2975, 2950-3000, 3025-3075, 3050 to 3100, 3075-3125, 3100-3150, 3125-3175, 3200-3250, 3225-3275, 3300-3350, 3325-3375, 3400-3450, 3425-3475, 3500-3550, 3525-3575, 3600-3650, 3625-3675, 3700-3750, 3725-3775, 3800-3850, 3825-3875, 3900-3950, 3925-3975, or 3950-4000 nucleotides long, or any integer contained therein (e.g., 51, 52, 53, 54, 55, etc.).

Accordingly, in some embodiments a recombinant nucleic acid for which splicing is regulated is a construct that can produce either i) a protein coding transcript of interest (e.g., a functional protein coding sequence) or ii) a transcript that that does not encode a full length protein coding sequence depending on whether an intron is removed or not (e.g., if the splicing event results in inclusion of a start codon, or if the splicing event results in inclusion of a stop codon). However, in some embodiments, a recombinant nucleic acid for which splicing is regulated can produce alternative splice products each of which can encode a protein, but the proteins have different functions (e.g., the alternatively spliced RNA products encode protein isomers). For example, in some embodiments the splicing events could generate cardiac- versus skeletal muscle-specific isoforms, or isoforms with differing functions or properties. In some embodiments, inclusion of MBNL1 exon 5 results in a more nuclear localized protein than the isoform lacking exon 5. In some embodiments, exon 12 in NRAP is skeletal muscle specific and completely skipped in heart. In some embodiments, the Chloride Channel 1 mRNA that contains exon 7a is subject to nonsense-mediated decay, but the mRNA that does not contain exon 7a generates full length protein. Accordingly, in some embodiments one or more tissue specific RNA protein binding motifs can be included in a recombinant nucleic acid (e.g., within an intron or an exon) in order to regulate splicing (e.g., to promote splicing in a first tissue or cell type relative to a second tissue or cell type).

In some embodiments, a recombinant nucleic acid for which splicing is regulated is a synthetic construct configured to regulate expression of an RNA by including a nonsense mediated decay (NMD) exon within the RNA, wherein the NMD exon is flanked by introns for which alternative splicing is regulated. In some embodiments, an NMD exon is an exon that encodes at least one stop codon that is in frame with a previous exon, wherein the stop codon is at least 50 nucleotides upstream (5′) from the 5′ splice site of the exon. In some embodiments, if the NMD exon is included in the spliced RNA, it causes degradation of the RNA via nonsense-mediated decay. In some embodiments, if the NMD exon is spliced out, the resulting transcript is stable, and in some embodiments encodes a functional (e.g., full-length) protein of interest.

In some embodiments, a recombinant nucleic acid for which splicing is regulated is a synthetic construct configured to regulate expression of a protein by including a 5′ exon comprising an amino terminal amino acid encoding sequence (e.g., an ATG or part of the ATG) and/or translation control sequences, wherein the 5′ exon is separated from subsequent exon(s) by an intron for which splicing is regulated. In some embodiment, if the intron is spliced out of the RNA transcript, the recombinant 5′ exon is spliced in frame to the subsequent exon(s) and the resulting spliced transcript encodes a protein that is expressed. In some embodiments, if the intron is not spliced out of the RNA transcript, the recombinant 5′ exon is not spliced to the subsequent exon(s) and as a result a protein is not expressed from the transcript.

In some embodiments, an intron for which splicing is regulated can be included within a gene that encodes a regulatory RNA (e.g., an siRNA). In some embodiments, intron(s) for which splicing is regulated and that encode regulatory RNA(s) can be included in a recombinant nucleic acid encoding an RNA transcript.

It will be understood by those of skill in the art that a nucleic acid may comprise a single intron or multiple introns. In some embodiments, the nucleic acid comprises one intron. In some embodiments, the nucleic acid comprises two introns. In some embodiments, the nucleic acid comprises 3, 4, 5, 6, 7, 8, 9, 10, 11, 12, 13, 14, 15, or more than 15 introns. In some embodiments, an intron is or is derived from any one or more of: an NMD exon-flanking intron of SmB/B′, an exon 2b-flanking intron of SMN, an intron 3 of SMN, a 3′ UTR intron of hnRBP A2B1, an NMD exon-flanking intron of Tia1, an exon 7-flanking intron of Bin1, an exon 11-flanking intron of Bin 1, an alternative exon-flanking intron of hnRNP D, an exon 13-flanking intron of FMRP, an exon 14-flanking intron of FMRP, an exon 15-flanking intron of FMRP, an alternative exon-flanking intron of Lamin A/C, an exon 11-flanking intron of ST7, an alternative exon-flanking intron of Matrin 3, an alternative exon-flanking intron of NEXN, an alternative exon-flanking intron of NRAP, an alternative exon-flanking intron of MTM1, an exon 9-flanking intron of CACNA1C, an exon T2-flanking intron of MBNL1, an exon 1-flanking intron of MBNL1, an exon 2-flanking intron of MBNL1, an exon 3-flanking intron of MBNL1, an exon 4-flanking intron of MBNL1, an exon 5-flanking intron of MBNL1, an exon 6-flanking intron of MBNL1, an exon 7-flanking intron of MBNL1, an exon 9-flanking intron of MBNL1, an intron 6 of PABPN 1, an intron 6 of TDP43, an intron 7 of TDP43, an intron 6 of FUS, an intron 7 of FUS, an intron 10 of hnRNP A1, and/or an exon 22-flanking intron of ATP2A1, or a combination thereof.

### Exons

In some embodiments, a nucleic acid comprises an exon. In some embodiments, the exon is flanked by one or more introns (e.g., one or more auto-regulated introns), that may in some embodiments be truncated as described herein. In some embodiments, the exon is naturally occurring. In some embodiments, the exon is a recombinant exon. In some embodiments, the exon is an alternatively regulated exon. In some embodiments, the alternatively regulated exon is flanked by one or more introns. In some embodiments, the retention of one or more introns causes the transcript to remain in the nucleus and unable to be exported into the cytoplasm, preventing translation of any protein product.

In some embodiments, exons that are naturally flanked by non-regulated introns are fused into continuous coding regions (e.g., a recombinant exon) without any intervening introns. In some embodiments, each exon that is auto-regulated has two or more flanking introns, which may or may not be truncated as described herein. As used herein, “flanking” refers to an intron located immediately upstream (5′) or immediately downstream (3′) of an associated exon.

In some embodiments, the exon is a regulatory exon. In some embodiments, the regulatory exon is a nonsense-mediated decay (NMD) exon. In some embodiments, the NMD exon comprises an in-frame stop codon that is at least 50 nucleotides upstream of the 3′ splice junction of the NMD exon.

It will be understood by those of skill in the art that a nucleic acid may comprise a single exon or multiple exons. In some embodiments, the nucleic acid comprises one exon. In some embodiments, the nucleic acid comprises two exons. In some embodiments, the nucleic acid comprises 3, 4, 5, 6, 7, 8, 9, 10, 11, 12, 13, 14, 15, or more than 15 exons. In some embodiments, the exon is or is derived from any one or more of: an NMD exon of SmB/B′, an exon 2b of SMN, an exon 3 of SMN, an exon 4 of SMN, an hnRBP A2B1 exon, an NMD exon of Tia1, an exon 7 of Bin1, an exon 11 of Bin1, an alternative exon of hnRNP D, an exon 13 of FMRP, an exon 14 of FMRP, an exon 15 of FMRP, an alternative exon of Lamin A/C, an exon 11 of ST7, an alternative exon of Matrin 3, an alternative exon of NEXN, an alternative exon of NRAP, an alternative exon of MTM 1, an exon 9 of CACNA1C, an exon T2 of MBNL1, an exon 1 of MBNL1, an exon 2 of MBNL1, an exon 3 MBNL1, an exon 4 of MBNL1, an exon 5 of MBNL1, an exon 6 of MBNL1, an exon 7 of MBNL1, an exon 9 MBNL1, an exon 6 of PABPN1, an exon 7 of PABPN 1, an exon 6 of TDP43, an exon 7 of TDP43, an exon 8 of TDP43, an exon 6 of FUS, an exon 7 of FUS, an exon 8 of FUS, an exon 10 of hnRNP A1, an exon 11 of hnRNP A1, and/or an exon 22 of ATP2A1, or a combination thereof.

In some embodiments, one or more tissue-specific alternative exons are included in a recombinant nucleic acid (e.g., in a rAAV). Non-limiting examples of tissue-specific alternative exons are described in Supplemental Table S4 from Wang, E. T., et al., (2008), Nature, 456, 470-76, incorporated herein by reference. Other tissue-specific exons can be identified from transcriptome data. Non-limiting examples of RNA sequence motifs that can exhibit tissue-specific activity, thereby controlling the inclusion or exclusion of tissue-specific exons, are described in Badr, E., et al., (2016), PLOS One, 11(11): e0166978, incorporated herein by reference.

### Intracellular Factors

In some embodiments, a regulatory region can bind to one or more intracellular factors (e.g., RNA binding proteins) that regulate intron splicing. In some embodiments, the nucleic acid expresses or encodes the intracellular factor. In some embodiments, the splicing of the intron is regulated by the expressed or encoded intracellular factor. In some embodiments, the splicing of the intron is not regulated by the expressed or encoded intracellular factor. In some embodiments, an intracellular factor comprises a protein, an RNA, or a protein-RNA complex.

In some embodiments, wherein the intracellular factor comprises a protein, the protein may comprise a tissue-specific RNA binding protein, an autoregulatory RNA binding protein, or a condition-specific RNA binding protein. In some embodiments, the protein is encoded by the nucleic acid and auto-regulates splicing of the mRNA encoded by the nucleic acid. In some embodiments, splicing of a recombinant intron can be regulated by two or more different splice regulatory proteins that bind to regulatory region(s) of the recombinant intron. For example, in some embodiments. NRAP exon 12 is highly included in skeletal muscle but absent in heart. In some embodiments, TPM2 exon 2 is low in heart but high in smooth muscle. In some embodiments, SLC25A3 is very high in heart but low in brain. Many other examples can be found in the literature and one example of a list of such “switch-like exons” can be found in Wang, E. T., et al., (2008), Nature, 456(7221):470-6.

In some embodiments, wherein the intracellular factor comprises a protein, the protein comprises an MBNL protein, an SR protein, an hnRNP protein, an RbFox protein, a CELF protein, a Nova protein, or a PTB protein. In some embodiments, wherein the intracellular factor comprises a protein, the protein is any one of MBNL1, MBNL2, MBNL3, hnRNP A1, hnRNP A2B1, hnRNP C, hnRNP D, hnRNP DL, hnRNP F, hnRNP H, hnRNP K, hnRNP L, hnRNP M, hnRNP R, hnRNP U, FUS, TDP43, PABPN1, ATXN2, TAF15, EWSR1, MATR3, TIA1, FMRP, MTM1, KIF5A, microdystrophin, C9ORF72, HIT, DNM2, BIN1, RYR1, NEB, ACTA, TPM3, TPM2, TNNT2, CFL2, KBTBD13, KLHL40, KLHL41, LMOD3, MYPN, SEPN1, TTN, SPEG, MYH7, TK2, POLG1, GAA, AGL, PYGM, SLC22A5, OCTN2, ETF, ETFH, PNPLA2, cytochrome b/cytochrome c oxidase, CLCN1, SCN4A, DMPK, CNBP, MYOT, LMNA (Lamin A/C), CAV3, DNAJB6, DES, TNPO3, HNRPDL, CAPN3, DYSF, alpha-sarcoglycan, beta-sarcoglycan, gamma-sarcoglycan, delta-sacroglycan, TCAP, TRIM32, FKRP, POMT1, FKTN, POMT2, POMGnT1, DAG1, ANO5, PLEC1, TRAPPC11, GMPPB, ISPD, LIMS2, POPDC1, TOR1A1P1, POGLUT2, LAMA2, COL6A1, POMT1, POMT2, DUX4, EMD, PAX7, PMP22, MPZ, MFN2, SMCHD 1, or GJB 1, or a truncated version thereof. Other examples of regulatory proteins are known in the art, and may be found, for example, in González-Jamett, A. M., et al., (2018), Muscle Cell and Tissue—Current Status of Research Field: Hereditary Myopathies (edited by Sakuma, K.), published online at DOI: 10.5772/intechopen.76076.

In some embodiments, wherein the intracellular factor comprises an RNA, the RNA comprises a regulatory RNA molecule, a short hairpin RNA molecule (shRNA), a microRNA molecule, or a transfer RNA molecule (tRNA). In some embodiments, wherein the intracellular factor comprises an RNA, the RNA comprises a DMPK-targeting shRNA or microRNA. In some embodiments, wherein the intracellular factor comprises an RNA, the RNA comprises a repeat-targeting shRNA or microRNA (e.g., a CUG shRNA, CAG shRNA, or GGGGCC shRNA). In some embodiments, wherein the intracellular factor comprises an RNA, the RNA comprises an shRNA or microRNA that targets an RNA binding protein. In some embodiments, wherein the intracellular factor comprises an RNA, the RNA comprises an shRNA or microRNA that targets other members of a related biological pathway. In some embodiments, wherein the intracellular factor comprises an RNA, the RNA comprises a specific tRNA that might be missing or mutated in disease (e.g. mitochondrial tRNAs in MERFF or MELAS).

In some embodiments, wherein the intracellular factor comprises a protein-RNA complex, the protein-RNA complex comprises a ribosome, snRNP complex, or other macromolecular complex that can interact with RNA to regulate splicing decisions. In some embodiments, wherein the intracellular factor comprises a protein-RNA complex, a snRNP complex comprises U1 snRNP or U2 snRNP. In some embodiments, wherein the intracellular factor comprises a protein-RNA complex, the RNA comprises a ribozyme that targets one or more CUG repeats. In some embodiments, wherein the intracellular factor comprises a protein-RNA complex, the RNA comprises a ribozyme that targets specific mRNAs.

Non-limiting examples of RNA binding protein motifs and RNA target sequences that can confer or regulate spicing activity are described, for example, in Ray, D., et al., (2014), Nature, 499(7457): 172-77; Lambert, N., et al., (2014), Mol. Cell., 54(5): 887-900; and Van Nostrand, E. L., et al., (2020), Nature, 583, 711-19, the disclosures of which are incorporated herein by reference.

### Nucleic Acids

In some embodiments, a nucleic acid (e.g., a recombinant gene) is provided in a viral vector (e.g., an rAAV vector). In some embodiments, the nucleic acid comprises a promoter and sequence corresponding to an RNA molecule that is capable of being expressed from the nucleic acid. In some embodiments, the nucleic acid is a rAAV genome comprising a DNA molecule, wherein the DNA molecule comprises sequences that encode an RNA molecule.

In some embodiments, the nucleic acid is sufficiently small to be effectively packaged in an AAV viral particle (e.g., the gene construct may be around 0.5-5 kb long, for example around 4.9 kb, 4.8 kb, 4.7 kb, 4.6 kb, 4.5 kb, 4.4 kb, 4.3 kb, 4.2 kb, 4.1 kb, 4 kb, 3.5 kb, or 3 kb long). So as to fit into the AAV viral particle, in some embodiments a nucleic acid comprises one or more truncated and/or recombinant introns. Accordingly, a recombinant intron for an rAAV vector is typically shorter than 4 kb, but can be between around 20 bases long and around 2,000 bases long to provide space for other components (e.g., exons, regulatory sequences, other introns, viral packaging sequences) in the nucleic acid (e.g., recombinant gene) construct. In some embodiments a recombinant intron is around 50 bases, around 100 bases, around 250 bases, around 500 bases, around 1,000 bases, around 1,500 bases, or around 2,000 bases long. In some embodiments, a recombinant intron is shorter than 4 kb, shorter than 3 kb, shorter than 2 kb, shorter than 1 kb, 100-900 bases long, or shorter than 500 bases long.

In some embodiments, the only intron that is retained in a nucleic acid (e.g., recombinant gene) construct is the truncated regulated intron. In some embodiments, two or more regulated introns are retained and truncated in the nucleic acid construct. In some embodiments, all other (e.g., non-regulatory) introns have been removed. However, in some embodiments, one or more of the other introns (e.g., the introns that are not subject to regulated splicing, for example that are not subject to auto-regulated splicing) may be retained (and optionally truncated) depending on the size of the nucleic acid and the size limitations of the virus, respectively. In some embodiments, the only introns in a nucleic acid (e.g., recombinant gene) construct are truncated introns (e.g., only one, 2, 3, 4, 5, 6, 7, 8, 9, 10 truncated auto-regulated introns). In some embodiments, the nucleic acid (e.g., recombinant gene) does not contain any full-length introns. In some embodiments, the nucleic acid (e.g., recombinant gene) does not contain any truncated introns that are not regulated (e.g., that are not auto-regulated).

In some embodiments, the nucleic acid comprises an RNAi that targets a chromosomal allele encoding a gene encoding the intracellular factor. As used herein, an “RNAi”, or inhibitory RNA, refers to RNAs that reduce the level of their binding targets for example via an Argonaute-mediated pathway. Examples of RNAi are known in the art, and may include, for example, short hairpin RNAs that are cleaved by Drosha and Dicer and recruit Argonaute to their targets for subsequent cleavage and/or regulation of translation. Examples of RNAi are known in the art, and a review of RNAi can be found, for example, in is reviewed in Wilson, R. C. and Doudna, J. A., (2013), Annual Review of Biophysics: Molecular Mechanisms of RNA Interference, 42:1, 217-39.

In some embodiments, the nucleic acid comprises a recombinant gene. In some embodiments, all the introns and exons of the nucleic acid construct are from the same gene. In some embodiments, at least one intron and at least one exon of the nucleic acid construct are from different genes. In some embodiments, the gene(s) comprises any one or more of: MBNL1, MBNL2, MBNL3, hnRNP A1, hnRNP A2B1, hnRNP C, hnRNP D, hnRNP DL, hnRNP F, hnRNP H, hnRNP K, hnRNP L, hnRNP M, hnRNP R, hnRNP U, FUS, TDP43, PABPN1, ATXN2, TAF15, EWSR1, MATR3, TIA1, FMRP, MTM1, KIF5A, a microdystrophin-encoding gene, C9ORF72, HTT, DNM2, BIN. RYR1, NEB, ACTA, TPM3, TPM2, TNNT2, CFL2, KBTBD13, KLHL40, KLHL41, LMOD3, MYPN, SEPN1, TTN, SPEG, MYH7, TK2, POLG1, GAA, AGL, PYGM, SLC22A5, OCTN2, ETF, ETFH, PNPLA2, a cytochrome b oxidase-encoding gene, a cytochrome c oxidase-encoding gene, CLCN1, SCN4A, DMPK, CNBP, MYOT, LMNA, CAV3, DNAJB6, DES, TNPO3, HNRPDL, CAPN3, DYSF, an alpha-sarcoglycan-encoding gene, a beta-sarcoglycan-encoding gene, a gamma-sarcoglycan-encoding gene, a delta-sacroglycan-encoding gene, TCAP, TRIM32, FKRP, POMT1, FKTN, POMT2, POMGnT 1, DAG 1, ANO5, PLEC 1, TRAPPC 11, GMPPB, ISPD, LIMS2, POPDC1, TOR1AIP1, POGLUT2, LAMA2, COL6A1, POMT1, POMT2, DUX4, EMD, PAX7, PMP22, MPZ, MFN2, SMCHD 1, and/or GJB 1.

In some embodiments, wherein all the introns and exons are from the same gene, the gene comprises any one of: SMN, hnRNP A1, hnRNP A2B1, Tia1, Bin1, hnRNP D, FMRP, Lamin A/C (LAMN), TDP43, FUS, or MBNL (e.g., MBNL1, 2, or 3), or any other gene that may be regulated or autoregulated.

The survival motor neuron (SMN) gene provides instructions for making the SMN protein and is found throughout the body, with highest levels in the spinal cord. In some embodiments, to prevent uncontrolled SMN over-expression, the NMD exon and flanking introns from SmB/B′ are incorporated into the SMN gene. In some embodiments, the incorporation results in regulation, such that high levels of SMN protein lead to inclusion of the NMD exon in the SMN gene construct. In some embodiments, inclusion of the NMD exon decreases in SMN expression (see, e.g., Saltzman, A. L., et al., (2011) Genes & Dev., 25: 373-84). In some embodiments, SMN exons 2b and intron 3 are incorporated into the SMN gene construct. In some embodiments, the incorporation leads to the proper generation of the axonal isoform of SMN (see, e.g., Setola, V., et al., (2007), PNAS, 104:(6), 1959-64).

Heterogeneous Nuclear Ribonucleoprotein A2/B 1 (hnRNPA2B 1) is a protein coding gene; the HNRNPA2 and HNRNPB1 proteins are involved in packaging nascent mRNA, in alternative splicing, and in cytoplasmic RNA trafficking, translation, and stabilization. In some embodiments, to prevent uncontrolled over-expression, the 3′ UTR introns from the endogenous hnRNPA2B 1 gene are incorporated into the hnRNPA2B 1 gene construct. In some embodiments, the incorporation results in regulation, such that NMD is activated when A2B1 protein levels are too high (see, e.g., McGlincy, N. J., et al., (2010), BMC Genomics, 11: 565).

T1A1 or Tia1 is a protein coding gene; TIA1 or Tia1 cytotoxic granule-associated RNA binding protein is a 3′UTR mRNA binding protein that can bind the STOP sequence of 5′TOP mRNAs. It is associated with programmed cell death and regulates alternative splicing of the gene encoding the Fas receptor, an apoptosis-promoting protein. In some embodiments, to limit the overexpression of Tia1 protein, the auto-regulated NMD exon (Jangi, M., et al., (2014), Genes & Dev., 28:637-51) is incorporated into the Tia1 gene construct, with flanking introns.

Bridging integrator 1 (Bin1) is a protein coding gene. Myc box-dependent-interacting protein 1, also known as Bridging Integrator-1 and Amphiphysin-2 is a protein that in humans is encoded by Bin1. In some embodiments, exon 7 of Bin1 is included in the Bin1 gene construct. In some embodiments, exon 7 of Bin1 is not included in the Bin1 gene construct. In some embodiments, exon 11 of Bin1 is included in the Bin 1 gene construct (e.g., in skeletal muscle). In some embodiments, exon 11 of Bin1, with flanking introns, is included in the Bin1 gene construct (e.g., in genetic disease with mutations in Bin). In some embodiments, exon 7 of Bin1 is not included in the Bin1 gene construct (e.g., in heart tissue). In some embodiments, the incorporation results in regulation such that appropriate isoforms can be expressed in each tissue.

Heterogeneous Nuclear Ribonucleoprotein D (Hnrnp D) is a protein coding gene that encodes Heterogeneous nuclear ribonucleoprotein DO, also known as AU-rich element RNA-binding protein 1. In some embodiments, to control hnRNP D protein overexpression, an alternative exon, and, in some embodiments, the flanking intron, at the start of the Hnrnp D 3′ UTR is incorporated (see, e.g., Kemmerer., K., et al., (2018), RNA, 24(3): 324-31).

The Fragile X Mental Retardation (FMRP) gene encodes the FMRP protein, which is an RNA-binding protein that also interacts with numerous cytoplasmic and nuclear proteins. FMRP has multiple isoforms that play distinct roles in axonal biology (see, e.g., Zimmer, S., et al., (2017), Dev. Neurobiol., 77(6): 738-52). In some embodiment, to preserve proper regulation of these isoforms, the alternative splicing patterns are incorporated near of exons 13-15 of the FMRP gene.

The Lamin A/C (LMNA) gene provides instructions for making several slightly different proteins called lamins. The two major proteins produced from this gene, lamin A and lamin C, are made in most of the body's cells. These proteins are made up of a nearly identical sequence of protein building blocks (amino acids). The small difference in the sequence makes lamin A longer than lamin C. The Lamin A/C gene has 2 distinct isoforms with alternative last exons. In some embodiments, to maintain appropriate ratios of both isoforms, the alternative last exons of Lamin A/C are incorporated the Lamin A/C gene construct.

In some embodiments, wherein TDP43 activity is low, the nucleic acid construct comprises intron 7 of TDP43 and a polyadenylation site within the retained intron. In some embodiments, retaining intron 7 of TDP43 results in normal TDP43 expression and mRNA export. In some embodiments, wherein TDP43 activity is high, the nucleic acid construct does not comprise intron 7 of TDP43.

In some embodiments, wherein FUS activity is low, the nucleic acid construct does not comprise FUS introns 6 and 7. In some embodiments, wherein FUS activity is high, the nucleic acid construct comprises FUS introns 6 and 7.

In some embodiments, wherein hnRNP A1 activity is low, the nucleic acid construct does not comprise hnRNP A1 intron 10. In some embodiments, wherein hnRNP A1 activity is high, the nucleic acid construct comprises hnRNP A1 intron 10.

In some embodiments, a nucleic acid comprises a recombinant nucleic acid encoding at least one gene that contains at least one recombinant (e.g., truncated) intron that supports sufficient splice regulation (e.g., auto-regulation) of the at least one gene to be therapeutically effective. In some embodiments, a recombinant nucleic acid is an RNA molecule (e.g., a pre-mRNA) that contains one or more (e.g., two or more) recombinant introns flanking one or more exons. In some embodiments, a recombinant nucleic acid is a DNA molecule that encodes the RNA molecule containing one or more recombinant introns. In some embodiments, the nucleic acid molecule contains other regulatory sequences (e.g., promoters, 5′ or 3 UTRs, or other regulatory sequences) in addition to the gene coding (e.g., protein coding) sequences and the at least one recombinant intron for which splicing can be regulated.

Some embodiments, of the present invention contemplate heterologous gene constructs, wherein introns and exons from different genes are integrated into a single nucleic acid construct. In some embodiments, at least one intron and at least one exon of the nucleic acid construct are from different genes. In some embodiments, wherein at least one intron and at least one exon of the nucleic acid construct are from different genes, the gene comprises any one or more of: SMN, Matrin 3, ST7, GAA, NEXN, NRAP, MTM1, RBM20, CACNA1C, MBNL (e.g., MBNL 1, 2, or 3), TDP43, and/or ATP2A1, or any other gene that may be regulated or autoregulated.

The Matrin 3 (MATR3) gene provides instructions for making the matrin 3 protein, which is found in the nucleus of the cell as part of the nuclear matrix. The nuclear matrix is a network of proteins that provides structural support for the nucleus and aids in several important nuclear functions. In some embodiments, to prevent uncontrolled over-expression of the matrin 3 protein, exon 11 from ST7 is incorporated into the Matrin 3 gene construct. In some embodiments, to prevent uncontrolled over-expression of the matrin 3 protein, any robustly regulated Matrin 3 splicing target is incorporated into the Matrin 3 gene construct (see, e.g., Coelho, M. B., et al., (2015), EMBO J., 34(5): 653-68). In some embodiments, the incorporate results in regulation such that expression of Matrin 3 ceases when its levels are too high.

The GAA gene provides instructions for producing acid alpha-glucosidase (also known as acid maltase), which is active in lysosomes. The levels of GAA required to rescue symptoms in Pompe disease may have different thresholds in muscle versus heart (see, e.g., Raben, et al., (2002), Mol. Ther., 6:(5), 601-08). In some embodiments, to titrate the amount of protein produced in each tissue, alternative exons that are included at different levels in the heart versus muscle are incorporated into the GAA gene construct. In some embodiments, for example, an alternative exon, with flanking introns, is placed at the start of the GAA gene and the ATG start codon of GAA is moved to the end of the exon in the GAA gene construct. In some embodiments, the alternative exon is an alternative exon of NEXN or NRAP. In some embodiments, the modifications of the GAA construct result in regulation, such that total GAA protein output is determined by the inclusion of the exon in the tissue of interest (e.g., heart tissue, muscle tissue).

The MTM 1 gene provides instructions for producing the enzyme myotubularin, which is thought to be involved in the development and maintenance of muscle cells. The levels of MTM 1 protein desired in myotubular myopathy may be different in skeletal muscle versus heart. In some embodiments, to titrate the amount of protein produced in each tissue, alternative exons that are included at different levels in the heart versus muscle are incorporated into the MTM 1 gene construct. In some embodiments, for example, an alternative exon, with flanking introns, is placed at the start of the MTM 1 gene and the ATG start codon of MTM 1 is moved to the end of the exon in the MTM 1 gene construct. In some embodiments, the alternative exon is an alternative exon of NEXN or NRAP. In some embodiments, the modifications of the MTM 1 construct result in regulation, such that total MTM1 protein output is determined by the inclusion of the exon in the tissue of interest (e.g., heart tissue, muscle tissue).

RNA-binding motif 20 (RBM20) gene is a protein coding gene that encodes RBM20 protein; mutations in the RBM20 gene are known to cause dilated cardiomyopathy (DCM). In some embodiments, to control the levels of RBM20 protein, one of its splicing targets and exon 9 of CACNA1C, with flanking introns, are incorporated into the RBM20 construct. In some embodiments, exon 9 of CACNA 1C is converted into an NMD exon by introducing stop codons and placing the exon, and flanking introns, into the middle of the RBM20 gene construct. In some embodiments, the modifications result in regulation such that high levels of RBM20 protein can be prevented by eliciting NMD.

In some embodiments, a nucleic acid construct comprises introns and exons from MBNL and ATP2A1. In some embodiments, the MBNL-ATP2A1 construct comprises exon 22 of ATP2A 1. In some embodiments, the MBNL-ATP2A 1 construct comprises exon 22 of ATP2A 1 and flanking intron(s). In some embodiments, the MBNL-ATP2A 1 construct comprises SEQ ID NO: 20.

In some embodiments, regulatory proteins and/or target sequences can be included from one or more RNA binding proteins that auto-regulate via NMD. Non-limiting examples include Lareau, L. F., et al., (2007), Nature, 446(7138): 926-29; Lareau, L. F., et al., (2007) Chapter 12: The Coupling of Alternative Splicing and Nonsense Mediated mRNA Decay, Alternative Splicing in the Postgenomic Era, edited by Benjamin J. Blencowe and Brenton R. Graveley; and Pervouchine, D., et al., (2019), Nucleic Acids Res., 47(10): 5293-306, the disclosures of which are incorporated herein by reference.

In some embodiments, the rAAV further comprises promoter. In some embodiments, the promoter is a constitutive promoter or a regulated promoter. In some embodiments, the regulated promoter is an inducible promoter. In some embodiments, the promoter comprises any one of: CMV, EF1alpha, CBh, synapsin, enolase, MECP2, MHCK7, Desmin, GFAP.

In some embodiments, an rAAV is provided comprising a nucleic acid having the sequence of any one or more of SEQ ID NOs: 1, 2, 4-6, 8-11, 13-15, and/or 17-49.

### MBNL Constructs

In some embodiments, a nucleic acid that comprises one or more introns that are subject to regulated splicing (e.g., for which splicing is autoregulated) is provided. Examples of genes that contain regulated (e.g., autoregulated) introns include MBNL genes (e.g., MBNL1 or MBNL2 or MBNL3). Other non-limiting examples of genes that contain regulated introns include SR proteins (e.g., SRSF1, SRSF2, etc.), hnRNP proteins (e.g., hnRNP A1, hnRNP A2, hnRNP C, etc.), other tissue-specific RNA binding proteins (e.g., other proteins in the MBNL family, RbFox family, CELF family, Nova family, PTB family, etc.), and/or any additional genes described in Lareau and Brenner, 32(4) Mol Bio Evol. 1072-1079 (2015)²⁰, the contents of which are incorporated herein by reference in their entirety.

In some embodiments, methods and compositions described in this application can be used to deliver recombinant MBNL genes that are auto-regulated. Accordingly, in some embodiments recombinant MBNL constructs are useful for delivery in viral vectors. In some embodiments, an MBNL construct comprises truncated MBNL gene sequences. In some embodiments, an MBNL expression construct comprises an MBNL coding sequence and one or more truncated introns as described herein. In some embodiments, one or more of exons 1, 2, 3, 4, 5, 6, 7, 8, 9, 10, and 11 are included in the MBNL coding sequence. In some embodiments, a truncated intron flanking exon 1 is included. In some embodiments, a truncated intron flanking exon 2 is included. In some embodiments, a truncated intron flanking exon 3 is included. In some embodiments, a truncated intron flanking exon 4 is included. In some embodiments, a truncated intron flanking exon 5 is included. In some embodiments, a truncated intron flanking exon 6 is included. In some embodiments, a truncated intron flanking exon 7 is included. In some embodiments, a truncated intron flanking exon 8 is included. In some embodiments, a truncated intron flanking exon 9 is included. In some embodiments, a truncated intron flanking exon 10 is included. In some embodiments, a truncated intron flanking exon 11 is included.

In some embodiments, one or more additional intron sequences are included (e.g., one or more additional introns or one or more additional truncated introns). In some embodiments, the truncated MBNL gene does not contain any full-length introns. In some embodiments, the truncated MBNL gene does not contain any truncated introns that are not regulated (e.g., that are not autoregulated). Accordingly, MBNL exons that are naturally flanked by other introns are fused into continuous coding regions without any introns.

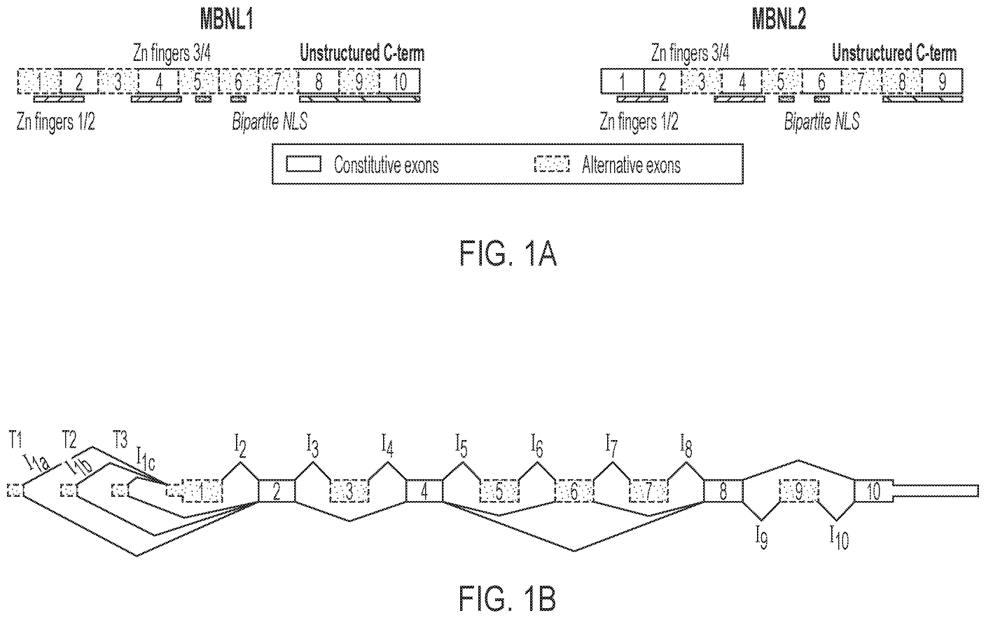

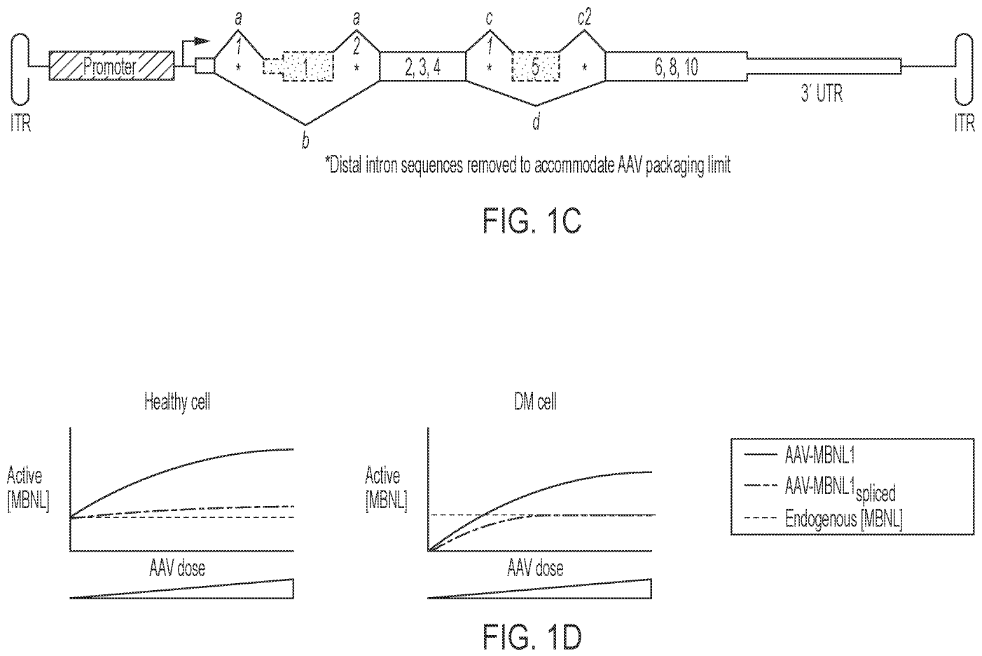

In some embodiments, the MBNL construct is a recombinant MBNL1 or MBNL2 expression construct comprising exon 1 and one or two truncated exon 1-flanking introns of MBNL1 or MBNL2. In some embodiments, one flanking intron is truncated. In some embodiments, two flanking introns are truncated. In some embodiments, one flanking intron is truncated, and one flanking intron is unmodified. In some embodiments, one flanking intron is truncated, and one flanking intron is removed. In some embodiments, the introns are flanking a fusion of exons (e.g., a series of exons with no introns between exons), wherein the introns between exons have been removed. In some embodiments, the flanking intronic sequences can be truncated forms of intron I1c and/or intron 12 of MBNL1 or MBNL2, for example, as depicted in FIG. 1B and FIG. 1C. In some embodiments, the splicing event includes exon 1 (see, e.g., FIG. 1C, a1 and a2). In some embodiments, the splicing event excludes exon 1 (see, e.g., FIG. 1C, b).

In some embodiments, the MBNL construct is a recombinant MBNL1 or MBNL2 expression construct comprising exon 1 and a truncated intron I1c of MBNL1 or MBNL2, for example, as depicted in FIGS. 1B and 1C. In some embodiments, the MBNL construct is a recombinant MBNL1 or MBNL2 expression construct comprising exon 1 and a truncated intron I2 of MBNL1 or MBNL2, for example, as depicted in FIGS. 1B and 1C. In some embodiments, the MBNL construct is a recombinant MBNL1 or MBNL2 expression construct comprising exon 1 and both a truncated intron Ile and a truncated intron I2 of MBNL1 or MBNL2.

In some embodiments, intron I1c (e.g., the intron located between exon T2 and exon 1 in MBNL1) is truncated by removing the portion of nucleotides shown in SEQ ID NO: 12. In some embodiments, intron 12 (e.g., the intron located between exon 1 and exon 2 in MBNL1) is truncated by removing the portion of nucleotides shown in SEQ ID NO: 16. In some embodiments, intron Ile (e.g., the intron located between exon T2 and exon 1 in MBNL1) comprises the nucleotides shown in SEQ ID NOs: 11 and 13, but does not include SEQ ID NO: 12. In some embodiments, intron I2 (e.g., the intron located between exon 1 and exon 2 in MBNL1) comprises the nucleotides shown in SEQ ID NOs: 15 and 17, but does not include SEQ ID NO: 16. The sequences described herein should be construed as non-limiting, and optionally smaller and/or larger sequences or portions thereof could be used to create the gene construct.

In some embodiments, the MBNL construct is a recombinant MBNL1 or MBNL2 expression construct comprising exon 5 and one or both truncated exon 5-flanking intron of MBNL1 or MBNL2, for example, as depicted in FIGS. 1B and 1C. In some embodiments, the flanking intronic flanking sequences can be truncated forms of introns 15 and/or I6 of MBNL1 or MBNL2, for example, as depicted in FIGS. 1B and 1C. In some embodiments, the splicing event includes exon 5 (see, e.g., FIG. 1C, c1 and c2). In some embodiments, the splicing event excludes exon 5 (see, e.g., FIG. 1C, d).

In some embodiments, intron I5 (e.g., the intron located between exon 4 and exon 5 in MBNL 1) is truncated by removing the portion of nucleotides shown in SEQ ID NO: 3. In some embodiments, intron I6 (e.g., the intron located between exon 5 and exon 6 in MBNL1) is truncated by removing the portion of nucleotides shown in SEQ ID NO: 7. In some embodiments, intron I5 (e.g., the intron located between exon 4 and exon 5 in MBNL1) comprises the nucleotides shown in SEQ ID NOs: 2 and 4, but does not include SEQ ID NO: 3. In some embodiments, intron I6 (e.g., the intron located between exon 5 and exon 6 in MBNL1) comprises the nucleotides shown in SEQ ID NOs: 6 and 8, but does not include SEQ ID NO: 7. The sequences described herein should be construed as non-limiting, and optionally smaller and/or larger sequences or portions thereof could be used to create the gene construct.

In some embodiments, the MBNL construct is a recombinant MBNL1 or MBNL2 expression construct comprising exon 5 and a truncated intron 5 of MBNL1 or MBNL2. In some embodiments, the MBNL1 or MBNL2 construct is a recombinant MBNL1 expression construct comprising exon 5 and a truncated intron I5 of MBNL1 or MBNL2. In some embodiments, the MBNL construct is a recombinant MBNL1 or MBNL2 expression construct comprising exon 5 and both a truncated intron I5 and a truncated intron I6 of MBNL1 or MBNL2.

In some embodiments, the MBNL construct is a recombinant MBNL1 or MBNL2 expression construct comprising exon 1 and one or two truncated exon 1-flanking introns of MBNL1 or MBNL2, and exon 5 and one or two truncated exon 5-flanking introns of MBNL1 or MBNL2, for example, as depicted in FIGS. 1B and 1C. In some embodiments, the MBNL construct is a recombinant MBNL1 or MBNL2 expression construct comprising exon 1 and a truncated intron I1c and a truncated intron 12 of MBNL1 or MBNL2, and exon 5 and a truncated intron I5 and a truncated intron I6 of MBNL1 or MBNL2.

In some embodiments, the MBNL construct further comprises exon 2. In some embodiments, the MBNL construct further comprises exon 3. In some embodiments, the MBNL construct further comprises exon 4. In some embodiments, the MBNL construct further comprises exon 6. In some embodiments, the MBNL construct further comprises exon 8. In some embodiments, the MBNL construct further comprises exon 10. In some embodiments, the MBNL construct further comprises exon 11.

In some embodiments, the MBNL construct only includes a truncated intron I1c, exon 1, a truncated intron I2, exon 2, and exon3. In some embodiments, the MBNL construct only includes a truncated intron I1c, exon 1, a truncated intron I2, exon 2, exon3, and exon 4. In some embodiments, the MBNL construct only includes a truncated intron I1c, exon 1, a truncated intron I2, exon 2, exon3, exon 4, and exon 6. In some embodiments, the MBNL construct only includes a truncated intron I1c, exon 1, a truncated intron I2, exon 2, exon3, exon 4, exon 6, and exon 10. In some embodiments, the MBNL construct only includes a truncated intron I1c, exon 1, a truncated intron I2, exon 2, exon3, and exon 4, exon 6, exon 10, and exon 11.

In some embodiments, the MBNL construct only includes a truncated intron I1c, exon 1, a truncated intron I2, exon 2, exon 3, a truncated intron 5, exon 5, and a truncated intron 6. In some embodiments, the MBNL construct only includes a truncated intron I1c, exon 1, a truncated intron I2, exon 2, exon 3, exon 4, a truncated intron 5, exon 5, and a truncated intron 6.

In some embodiments, the MBNL construct only includes a truncated intron I1c, exon 1, a truncated intron I2, exon 2, exon 3, exon 4, a truncated intron 5, exon 5, a truncated intron 6, and exon 6. In some embodiments, the MBNL construct only includes a truncated intron I1c, exon 1, a truncated intron I2, exon 2, exon 3, exon 4, a truncated intron 5, exon 5, a truncated intron 6, exon 6, and exon 10. In some embodiments, the MBNL construct only includes a truncated intron I1c, exon 1, a truncated intron I2, exon 2, exon 3, exon 4, a truncated intron 5, exon 5, a truncated intron 6, exon 6, exon 10, and exon 11. In some embodiments, exon 7 and/or exon 9 is/are also included.

In some embodiments, a truncated intron comprises about 20%, 30%, 40%, 50%, 60%, 70%, 80%, 90%, or more of the intron. In some embodiments, a truncated intron comprises a truncation of 5-10%, 10-20%, 20-30%, 30-40%, 40-50%, 50-60%, 60-70%, 70-80%, or 80-90% of the intron. In some embodiments, the truncated portion of the intron (e.g., introns I1c, I2, I5 and 16 of MBNL 1) comprises a truncation of 0.01-0.1 kb, 0.1-0.5 kb, 0.5-1.0 kb, 1-5 kb, 5-25 kb, or 25-100 kb. In some embodiments, the truncation is a 5′ truncation, an internal truncation, a 3′ truncation, or a combination thereof.

In some embodiments, the MBNL construct comprises “full length” exon 4, intron 4, exon 5, intron 5, and exon 6 (miniAR5.1). In some embodiments, the MBNL construct comprises “full length” exon 4, exon 5, intron 5, and exon 6, wherein intron 4 was truncated from 1164 bases to 491 bases (mAR5.2). In some embodiments, the MBNL construct comprises “full length” exon 4, intron 4, exon 5, and exon 6, wherein intron 5 was truncated from 862 bases to 121 bases (mAR5.3). In some embodiments, the MBNL construct comprises “full length” exon 4, exon 5, and exon 6, wherein both introns 4 and 5 were truncated (miniAR5.4). In some embodiments, miniAR5.2, miniAR5.3, and miniAR5.4 exhibit splicing regulatory activity in response to CTG480 and MBNL over-expression that largely mirrors that exhibited by full length “miniAR5.1” conditions.

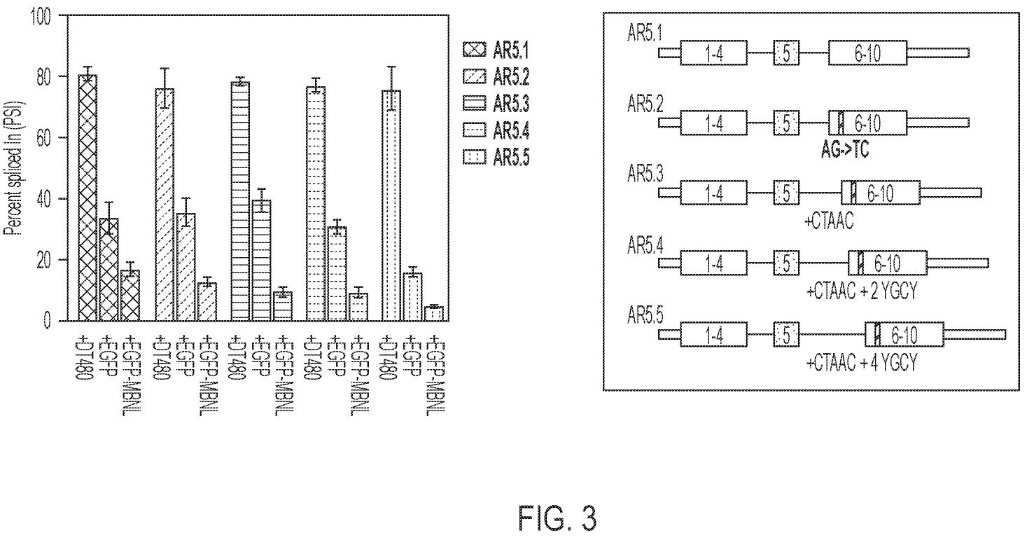

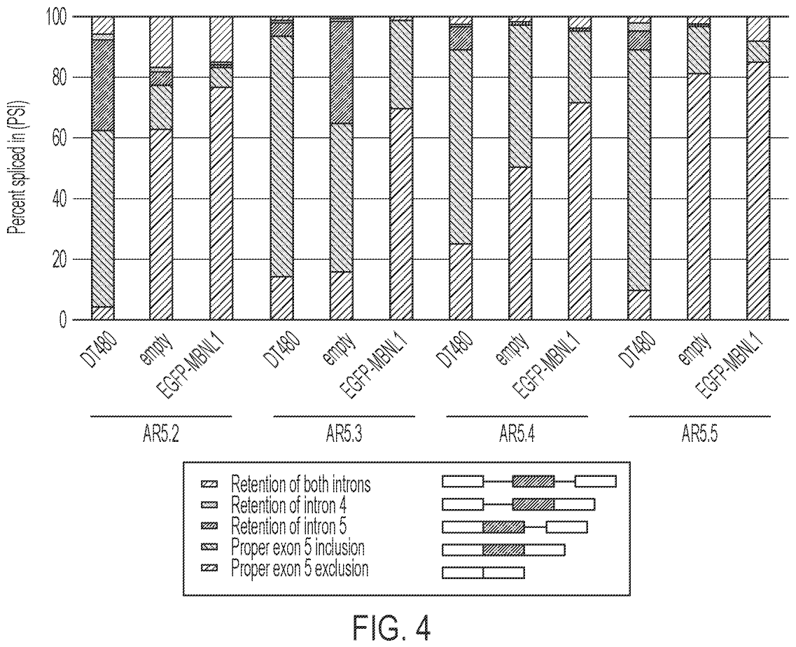

In some embodiments, the MBNL construct comprises the truncated alternative splicing cassette, cloned from miniAR5.4, into the context of a full length MBNL1 coding sequence in the context of AAV ITRs (AR5.1) (FIG. 3). In some embodiments, expression of the AR5.1 construct results in an additional isoform that uses a cryptic 3′ splice site within exon 6. In some embodiments, the MBNL construct comprises a mutation such that the “AG” in the AR5.1 construct cryptic splice site becomes a “TC” (e.g., to eliminate the unwanted isoform: AR5.2). In some embodiments, the MBNL construct comprises the AR5.2 construct with additional endogenous MBNL intron 5 sequence with strong branch points, and no copies of the YGCY motif to which MBNL binds, added back to the construct (AR5.3). In some embodiments, the MBNL construct comprises the AR5.2 construct with additional endogenous MBNL intron 5 sequence with strong branch points, and two copies of the YGCY motif to which MBNL binds, added back to the construct (AR5.4). In some embodiments, the MBNL construct comprises the AR5.2 construct with additional endogenous MBNL intron 5 sequence with strong branch points, and four copies of the YGCY motif to which MBNL binds, added back to the construct (AR5.5). In some embodiments, expression of the AR5.5 construct eliminates intron retention of intron 5 (FIG. 4).

In some embodiments, the MBNL construct comprises a truncated version of the intron before exon 1 and the intron after exon 1 of MBNL (miniAR1.1). In some embodiments, the final intron before exon 1 in miniAR1.1 is 259 bases and the final intron between exon 1 and 2 is 173 bases. In some embodiments, the MBNL construct comprises miniAR1.1 with additional deletions in MBNL exon 1 (miniAR1.2; miniAR1.3). In some embodiments, the MBNL construct comprises miniAR 1.1 cloned into a full length MBNL1 coding sequence, including AAV ITRs (AR1.1). In some embodiments, the MBNL construct comprises AR1.1 with the “AG” of a cryptic splice site mutated to “TC” (AR1.2). In some embodiments, the MBNL construct comprises AR1.1, wherein additional sequence from the intron before exon 1 was added back to the construct (AR1.3-AR1.6).

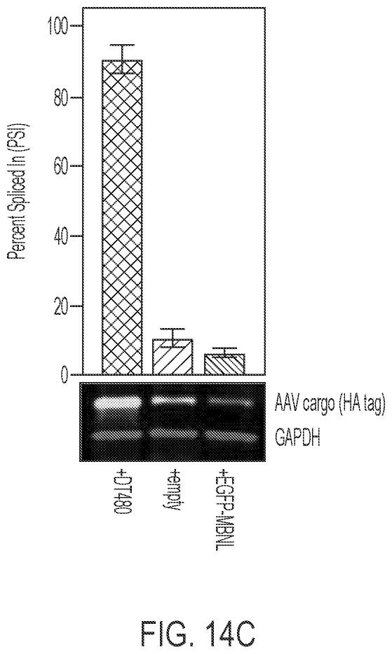

In some embodiments, the MBNL construct comprises AR1.2 and AR5.5 were combined into a single construct. In some embodiments, the MBNL construct comprises exon 22 and flanking introns from ATP2A1. In some embodiments, the MBNL construct comprises exon 22 and flanking introns from ATP2A1, with premature stop codons introduced into exon 22. In some embodiments, the MBNL construct comprises AR5.5, wherein the intron 4-exon 5-intron 5 cassette from AR5.5 is moving to the beginning of the MBNL1 coding sequence, the start ATG codon from the MBNL 1 coding sequence is removed, and an “A” is added to exon 5 of the AR5.5 cassette such that the last 3 bases of exon 5 encode “ATG” (FIG. 14B) In some embodiments, the recombinant MBNL1 or MBNL2 expression construct includes an exogenous promoter. Non-limiting examples of exogenous promoters include EF1 alpha promoter, beta actin promoter, CAG promoter, Desmin promoter, muscle creatine kinase promoter, synthetic C5-12 muscle promoter, synapsin promoter, GFAP promoter, or other promoters.

In some embodiments, the recombinant MBNL1 or MBNL2 expression construct includes an exogenous 3′ untranslated region (UTR). In some embodiments, the exogenous 3′ UTR is the 3′ UTR from bovine growth hormone.

In some embodiments, the recombinant MBNL1 or MBNL2 expression construct includes the second 5′ UTR of the MBNL1 or MBNL2 gene, and does not include the first or the third 5′ UTR of the MBNL1 or MBNL2 gene.

In some aspects, this disclosure provides a recombinant adeno-associated virus (rAAV) comprising the recombinant MBNL1 or MBNL2 expression constructs described herein. In some embodiments, the rAAV includes an MBNL1 or MBNL2 expression construct that is flanked by AAV ITRs.

Packaging Nucleic Acids into rAAV Particles

According to some aspects of the present disclosure nucleic acids molecules are designed and/or modified (e.g., truncated) so as to be packaged into viral particles (e.g., rAAV particles). In some embodiments, the nucleic acid is truncated relative to a corresponding wild-type nucleic acid to allow for efficient packaging in a recombinant viral vector (e.g., in a recombinant AAV, lentiviral, retrovirus, foamyvirus, or other vector). In some embodiments, the nucleic acid construct contains one or more deletions relative to the corresponding wild-type nucleic acid. In some embodiments, the one or more deletions are within an intron for which splicing is regulated (e.g., auto-regulated).

In some embodiments, a nucleic acid construct (e.g., an RNA or DNA molecule) is provided as a recombinant viral genome that contains sufficient viral sequences for packaging in a viral vector (e.g., an rAAV particle). For example, in some embodiments a nucleic acid construct is flanked by viral sequences (for example, terminal repeat sequences) that are useful to package the nucleic acid construct in a viral particle (e.g., encapsidated by viral capsid proteins). In some embodiments, the flanking terminal repeat sequences are rAAV ITRs. In some embodiments, the AAV ITR sequences comprise AAV 1, AAV2, AAV5, AAV7, AAV8, or AAV9 ITR sequences.

Methods of Delivering rAAV

Some aspects of the invention contemplate a method of treating a disease or condition in a subject comprising administering an rAAV of the present disclosure to a subject. Accordingly, provided herein is a method of delivering the disclosed rAAV particles. In some embodiments, rAAV particles are delivered by administering any one of the compositions disclosed herein to a subject. In some embodiments, “administering” or “administration” means providing a material to a subject in a manner that is pharmacologically useful. In some embodiments, rAAV particles are delivered to one or more tissues and cell types in a subject. In some embodiments, rAAV particles are delivered to one or more of muscle, heart, CNS, and immune cells. In some embodiments, delivery of an rAAV particle restores transcriptome homeostasis.

In some embodiments, an rAAV particle is administered to the subject parenterally. In some embodiments, an rAAV particle is administered to a subject subcutaneously, intraocularly, intravitreally, subretinally, intravenously (IV), intracerebro-ventricularly, intramuscularly, intrathecally (IT), intracisternally, intraperitoneally, enterally, via inhalation, topically, or by direct injection to one or more cells, tissues, or organs. In some embodiments, an rAAV particle is administered to the subject by injection into the hepatic artery or portal vein.

To “treat” a disease as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of rAAV particles may be an amount of the particles that are capable of transferring an expression construct to a host organ, tissue, or cell. A therapeutically acceptable amount may be an amount that is capable of treating a disease, e.g., DM1 and/or DM2. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

In some embodiments, a single composition comprising rAAV particles as disclosed herein is administered only once. In some embodiments, a subject may need more than 1 administration of an rAAV composition (e.g., 2, 3, 4, 5, 6, 7, 8, 9, or 10 or more times). For example, a subject may need to be provided a second administration of any one of the rAAV compositions as disclosed herein 1 day, 1 week, 1 month, 1 year, 2 years, 5 years, or 10 years after the subject was administered a first composition. In some embodiments, a first composition of rAAV particles is different from the second composition of rAAV particles.

In some embodiments, the administration of the composition is repeated at least once (e.g., at least once, at least twice, at least thrice, at least four times, at least five times, at least six times, at least 10 times, at least 25 times, or at least 50 times), and wherein the time between a repeated administration and a previous administration is at least 1 month (e.g., at least 1 month, at least 2 months, at least 3 months, at least 4 months, at least 5 months, at least 6 months, or at least 12 months). In some embodiments, the administration of the composition is repeated at least once, and wherein the time between a repeated administration and a previous administration is at least 1 year (e.g., at least 1, at least 2, at least 3, at least 4, at least 5, at least 10, or at least 20 years).

In some embodiments, the administration of the composition is facilitated by AAV capsids such as AAV 1-9, e.g., with AAV2 ITRs, or other capsids that sufficiently deliver to affected tissues.

### Subjects

Aspects of the disclosure relate to methods for use with a subject (e.g., a mammal). In some embodiments, a mammalian subject is a human, a non-human primate, or other mammalian subject. In some embodiments, the subject has one or more mutations associated with aberrant auto-regulated intron splicing of a gene encoding an RNA binding protein.

In some embodiments, a subject suffers from or is at risk of developing a disease or condition associated with aberrant splice regulation (e.g., in an auto-regulated gene encoding an RNA binding protein) resulting in one or more symptoms of disease or disorder. Non-limiting examples of these diseases include instances in which the homeostasis of RNA binding proteins is altered (e.g., other repeat expansion diseases), or diseases in which there are mutations in RNA binding protein sequences. In some embodiments, the disease or condition is selected from: a repeat expansion disease, a laminopathy, a cardiomyopathy, a muscular dystrophy, a neurodegenerative disease, a cancer, an intellectual disability, and/or premature aging.

In a non-limiting example, compositions or methods of this application are administered to a subject resulting in regulated overexpression of the RNA binding protein exhibiting aberrant activity. In another non-limiting example, compositions or methods of this application are administered to a subject resulting in the regulated addition of additional non-mutated, non-aberrant RNA binding protein.

In some embodiments, the disease or condition is selected from the group consisting of: Dentatorubral-pallido-luysian atrophy (DRPLA), myotonic dystrophy type 1 (DM1), myotonic dystrophy type 2 (DM2), Fragile X syndrome of mental retardation (FMR1), Fragile X tremor ataxia syndrome (FXTAS), FRAXE mental retardation (FMR2), Friedreichs ataxia (FRDA), Huntington disease (HD), Huntington disease-like 2 (HDL2), Oculopharyngeal muscular dystrophy (OPMD), Myoclonic epilepsy type 1, Alzheimer's disease, ALS/FTD, spinocerebellar ataxia type 1 (SCA1), spinocerebellar ataxia type 2 (SCA2), spinocerebellar ataxia type 3 (SCA3), spinocerebellar ataxia type 6 (SCA6), spinocerebellar ataxia type 7 (SCAT), spinocerebellar ataxia type 8 (SCA8), spinocerebellar ataxia type 10 (SCA10), spinocerebellar ataxia type 12 (SCA12), spinocerebellar ataxia type 17 (SCA17), Syndromic/non-syndromic X-linked mental retardation, Emery-Dreifuss muscular dystrophy type 2, familial partial lipodystrophy, limb girdle muscular dystrophy type 1B, dilated cardiomyopathy, familial partial lipodystrophy, Charcot-Marie-Tooth disorder type 2B 1, mandibuloacral dysplasia, childhood progeria syndrome (Hutchinson-Gilford syndrome), Werner syndrome, Dilated cardiomyopathy (DCM), Hypertrophic cardiomyopathy (HCM), Restrictive cardiomyopathy (RCM), Left Ventricular Non-compaction (LVNC), Arrhythmogenic Right Ventricular Dysplasia (ARVD), takotsubo cardiomyopathy, Duchenne muscular dystrophy, Becker muscular dystrophy, Limb-girdle muscular dystrophy, Facioscapulohumeral muscular dystrophy, Congenital muscular dystrophy, Oculopharyngeal muscular dystrophy, Distal muscular dystrophy, Emery-Dreifuss muscular dystrophy, dementia, Parkinson's disease (PD), a PD-related disorder, Prion disease, a motor neuron disease (MND), Progressive bulbar palsy (PBP), Progressive muscular atrophy (PMA), Primary lateral sclerosis (PLS), Spinal muscular atrophy (SMA), a bladder cancer, a breast cancer, a colorectal cancer, a kidney cancer, a lung cancer, a lymphoma, a melanoma, an oral cancer, an ovarian cancer, an oropharyngeal cancer, a pancreatic cancer, a prostate cancer, a thyroid cancer, a uterine cancer, Down syndrome, Prader-Willi Syndrome (PWS), Bloom Syndrome, Cockayne Syndrome Type I-216400, Cockayne Syndrome Type III, Cockayne Syndrome Type I, Hutchinson-Gilford Progeria Syndrome, Mandibuloacral Dysplasia with Type A Lipodystrophy, Progeria, Adult Onset Progeroid Syndrome, Neonatal Rothmund-Thomson Syndrome, Seip Syndrome, Werner Syndrome, Replication Focus-Forming Activity 1, and/or centronuclear myopathy.

Non-limiting examples of symptoms of these diseases include neurodevelopmental, neurofunctional, or neurodegenerative changes (e.g., ALS, FTD, Spinocerebellar Ataxias, FXTAS, or Huntington's Disease symptoms) or abnormal proliferation or migration of cells (e.g., as in cancer). For example, myotonic dystrophy type 1 and type 2 (dystrophia myotonica, DM1 and DM2, respectively) are caused by expanded CTG repeats in the DMPK gene and CCTG repeats in the CNBP gene, respectively. Both diseases are highly multi-systemic with symptoms in skeletal muscles, cardiac tissue, gastrointestinal tract, endocrine system, and central nervous system, among others¹.

In some aspects, the present disclosure relates to methods and compositions that are useful for treating myotonic dystrophy type 1 and type 2 (dystrophia myotonica, DM1 and DM2, respectively), for example by delivering rAAV particles comprising nucleic acid constructs (e.g., containing one or more truncated introns) to cells or tissue in a subject. In addition to the symptoms described above, DM1 can also manifest in a severe form called congenital DM1, in which profound developmental delays occur. A 25% chance of death before the age of 18 months and 50% chance of survival into mid-30s has been reported². Methods and compositions of the application can be useful to treat one or more symptoms of DM1.

Accordingly, in some embodiments one or more nucleic acid constructs can be delivered to a subject having one or more symptoms of myotonic dystrophy. In some embodiments, an rAAV composition provided herein is administered to a subject having congenital DM1 or DM2. In some embodiments, wherein in the nucleic acid construct is an MBNL construct, the MBNL constructs ameliorate one or more symptoms associated with DM1 and/or DM2. In some embodiments, wherein in the nucleic acid construct is an MBNL construct, treatment with the disclosed MBNL constructs reduces muscle weakness in a subject. In some embodiments, wherein in the nucleic acid construct is an MBNL construct, treatment with the disclosed MBNL constructs reduces muscle loss or muscle wasting in a subject. In some embodiments, wherein in the nucleic acid construct is an MBNL construct, treatment with the disclosed MBNL constructs reduces prolonged muscle contractions in a subject. In some embodiments, wherein in the nucleic acid construct is an MBNL construct, treatment with the disclosed MBNL constructs improves speech and/or swallowing in a subject. In some embodiments, treatment reduces or corrects one or more other symptoms of myotonic dystrophy.

In some embodiments, splicing of a recombinant intron is sufficiently regulated (e.g., through auto-regulation) to be therapeutically effective. In some embodiments, levels of autoregulation are measured by perturbing the baseline gene product (e.g., MBNL) activity in the cell. In some embodiments, levels of autoregulation are measured by the activity of the protein that is mimicked by the protein delivered via virus. In some embodiments, the level of autoregulation measured by either perturbing the baseline protein (e.g., MBNL) activity in the cell or the activity of the protein that is mimicked by the protein delivered via virus is compared to phenotypic symptoms to determine treatment efficacy.
Methods of Producing rAAV Particles

Methods of producing rAAV particles and nucleic acid vectors are known in the art and commercially available (see, e.g., Zolotukhin et al. Production and purification of serotype 1, 2, and 5 recombinant adeno-associated viral vectors. Methods 28 (2002) 158-167; and U.S. Patent Publication Nos. US 2007/0015238 and US 2012/0322861, which are incorporated herein by reference; and plasmids and kits available from ATCC and Cell Biolabs, Inc.). For example, a plasmid containing the nucleic acid vector sequence may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected into a producer cell line such that the rAAV particle can be packaged and subsequently purified.

In some embodiments, the one or more helper plasmids includes a first helper plasmid comprising a rep gene and a cap gene and a second helper plasmid comprising a E1a gene, a E1 b gene, a E4 gene, a E2a gene, and a VA gene. In some embodiments, the rep gene is a rep gene derived from AAV2 and the cap gene is a cap gene derived from AAV2 and includes modifications to the gene in order to produce a modified capsid protein described herein. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, Pa.; Cellbiolabs, San Diego, Calif.; Agilent Technologies, Santa Clara, Ca; and Addgene, Cambridge, Mass.; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081; Grimm et al. (2003), Helper Virus-Free, Optically Controllable, and Two-Plasmid-Based Production of Adeno-associated Virus Vectors of Serotypes 1 to 6, Molecular Therapy,Vol. 7, 839-850; Kronenberg et al. (2005), A Conformational Change in the Adeno-Associated Virus Type 2 Capsid Leads to the Exposure of Hidden VP1N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO4-mediated transfection, lipids or polymeric molecules such as Polyethylenimine (PEI) with the helper plasmid(s) and a plasmid containing a nucleic acid vector described herein. The HEK293 cells are then incubated for at least 60 hours to allow for rAAV particle production. Alternatively, in another example Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known the art or described herein, e.g., by iodixanol step gradient, CsCl gradient, chromatography, or polyethylene glycol (PEG) precipitation.

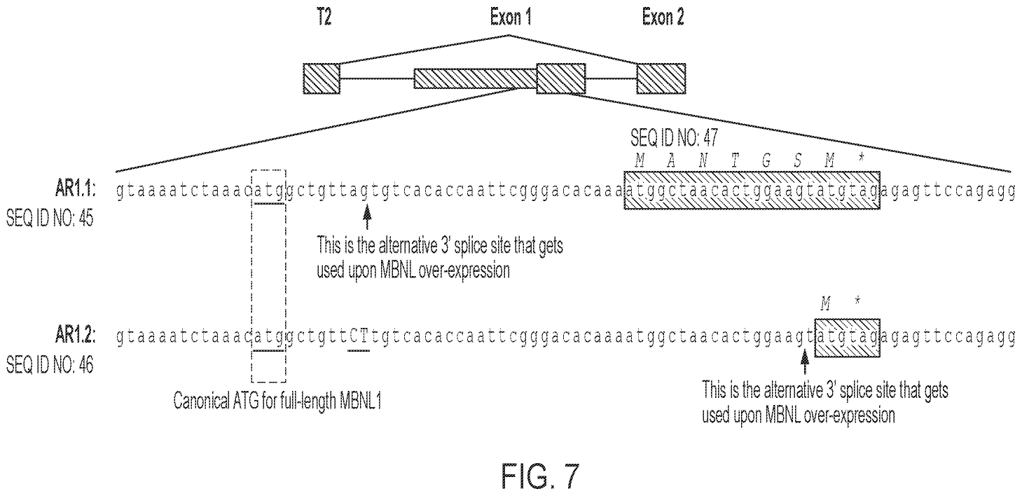

TABLE 1
Non-limiting example of
sequences of the disclosure

| | SEQ ID |
|---|---|
| Description | NO: |
| 3′ end of | 1 |
| MBNL1 exon 4 | |
| Truncated | 2 |
| MBNL1 | |
| intron I5 | |
| Deleted | 3 |
| portion of | |
| MBNL1 | |
| intron I5 | |
| Truncated | 4 |
| MBNL1 | |
| intron I5 | |
| MBNL1 | 5 |
| exon 5 | |
| Truncated | 6 |
| MBNL1 | |
| intron I6 | |
| Deleted | 7 |
| portion of | |
| MBNL1 | |
| intron I6 | |
| Truncated | 8 |
| MBNL1 | |
| intron I6 | |
| 5′ end of | 9 |
| MBNL1 | |
| exon 6 | |
| 3′ end of | 10 |
| MBNL1 | |
| exon T2 | |
| Abbreviated | 11 |
| MBNL1 intron | |
| I1c between T2 | |
| and exon 1 | |
| Deleted | 12 |
| portion of | |
| MBNL1 intron | |
| I1c between T2 | |
| and exon 1 | |
| Abbreviated | 13 |
| MBNL1 intron | |
| I1c between T2 | |
| and exon 1 | |
| MBNL1 exon 1 | 14 |
| Truncated | 15 |
| MBNL intron | |
| I2 between | |
| exon 1 and | |
| exon 2 | |
| Deleted | 16 |
| portion | |
| MBNL1 intron | |
| I2 between | |
| exon l and | |
| exon 2 | |
| Truncated | 17 |
| MBNL1 intron | |
| I2 between | |
| exon 1 and | |
| exon 2 | |
| 5′ end of | 18 |
| MBNL exon 2 | |
| AAV-hnRNP | 19 |
| A1 construct | |
| AAV-MBNL- | 20 |
| ATP2A1-NMD | |
| construct | |
| AAV-TDP43 | 21 |
| construct | |
| AR1.1 MBNL | 22 |
| construct | |
| AR1.1-AR5.5Q | 23 |
| MBNL | |
| construct | |
| AR1.1Q MBNL | 24 |
| construct | |
| AR1.2 MBNL | 25 |
| construct | |
| AR1.2-AR5.5Q | 26 |
| MBNL | |
| construct | |
| AR1.2Q MBNL | 27 |
| construct | |
| AR1.3 MBNL | 28 |
| construct | |
| AR1.4 MBNL | 29 |
| construct | |
| AR1.5 MBNL | 30 |
| construct | |
| AR1.6 MBNL | 31 |
| construct | |
| AR5.1 MBNL | 32 |
| construct | |
| AR5.2 MBNL | 33 |
| construct | |
| AR5.3 MBNL | 34 |
| construct | |
| AR5.4 MBNL | 35 |
| construct | |
| AR5.5 MBNL | 36 |
| construct | |
| Heterologous | 37 |
| MBNLex5 | |
| miniAR1.1 | 38 |
| MBNL | |
| construct | |
| miniAR1.2 | 39 |
| MBNL | |
| construct | |
| miniAR1.3 | 40 |
| MBNL | |
| construct | |
| miniAR5.1 | 41 |
| MBNL | |
| construct | |
| miniAR5.2 | 42 |
| MBNL | |
| construct | |
| miniAR5.3 | 43 |
| MBNL | |
| construct | |
| miniAR5.4 | 44 |
| MBNL | |
| construct | |
| Fragment of | 45 |
| AR1.1 MBNL | |
| construct | |
| (FIG. 7) | |
| Fragment of | 46 |
| ARI.2 MBNL | |
| construct | |
| (FIG. 7) | |
| Fragment of | 47 |
| AR1.1 MBNL | |
| construct | |
| (FIG. 7) | |
| AAV-FUS | 48 |
| construct | |
| AAV-PABPN1 | 49 |
| construct | |

In some embodiments, nucleic acids can include one or more of these sequences or fragments thereof and/or sequences that are at least 80%, at least 85%, at least 90%, 90-95%, or 95-100% identical to one or more of these sequences or fragments thereof.

These and other aspects of the application are illustrated by the following non-limiting examples.

### EXAMPLES

### Example 1. Tissue-Specific Delivery of Synthetic MBNL Cargoes Using AAV to Patients

CTG/CCTG repeats in DM1/DM2 are transcribed into RNA and sequester members of the Muscleblind-like (MBNL) family of RNA binding proteins³. This prevents them from interacting with their normal RNA substrates in the cell. Because the MBNL proteins regulate alternative splicing⁴, alternative polyadenylation⁵, and RNA localization^{6.7}, sequestration of MBNL proteins leads to systemic defects in DM1 and DM2. There are 3 MBNL proteins in mammals—MBNL1, MBNL2, and MBNL3⁸. MBNL1 and 2 are expressed ubiquitously across tissues and cell types, but are most highly expressed in muscle, heart, CNS, and immune cells⁹. MBNL1 and 2 are lowly expressed early in development and rise throughout postnatal development. MBNL1 levels are higher than MBNL2 in skeletal muscle, and MBNL2 levels are higher than MBNL1 in CNS cell types. MBNL3 is more highly expressed during embryonic development but is also expressed in adult placenta and muscle satellite cells. MBNL1 and 2 each have two pairs of C3H zinc fingers and are both highly alternatively spliced¹⁰; at least 9 isoforms have been documented for MBNL1 and at least 6 for MBNL2 (FIGS. 1A, 1B). Some isoforms exhibit more nuclear localization and some exhibit more cytoplasmic localization; the presence of both nuclear localization sequences as well as ubiquitination sites¹¹ may be partly responsible for these subcellular localization patterns. The cytoplasmic roles for MBNL proteins are distinct from splicing and may be important for neuronal function to allow for proper RNA localization and local translation of neurotransmitter receptors. In fact, cytoplasmic depletion of MBNL is an early event in DM pathogenesis and is associated with neuronal and behavioral phenotypes in CNS mouse models of DM1¹².

Because symptoms in DM are in part due to a loss of MBNL function, exogenous addition of MBNL to DM tissues may compensate for MBNL sequestration and restore transcriptome homeostasis. In biochemical and cell-based assays, MBNL over-expression by plasmid or virus has mitigated the effects of expanded CTG repeat expression in model systems. Use of an AAV2/1 (AAV2 ITR, AAV 1 capsid) to overexpress the 41 kD isoform of MBNL1 tagged with myc in the tibialis anterior of 4-week-old HSALR mice resulted in a ˜2-fold increase in total MBNL1 levels as assessed by Western blot, reductions in CUG RNA foci, rescue of alternative splicing changes, and rescue of myotonia as assessed by EMG^{13,14}. Although most of these metrics were assessed 23 weeks after injection, myotonia was reduced as early as 4 weeks post-injection. Histopathological abnormalities in HSALR such as centralized nuclei, split fibers, and fiber size heterogeneity were not rescued by AAV-MBNL1. At 43 weeks of age, myotonia began to return.

Transgenic mice have been engineered to over-express the 40 kD isoform of MBNL 1 driven by a beta actin promoter, resulting in two different mouse lines¹⁵. In one line (14686), over-expression was achieved across multiple tissues, resulting in protein levels of ˜3-fold (brain), ˜9-fold (heart), and ˜17-fold (gastrocnemius) higher than endogenous MBNL1. In some tissues, a smaller proteolytically cleaved fragment of the transgene was observed. This line did not show any overt differences from WT animals except that they developed more frequent eye infections that could be resolved by antibiotics, and mortality was increased at 76 weeks of age (but not at 60 weeks). The deaths in this context were sudden and of unknown cause. The other mouse line (14685) showed prominent over-expression in the skeletal muscle (˜9-fold in gastrocnemius and quadriceps) but not in other tissues, and showed a significant ˜15-20% reduced body mass relative to WT. Such effects may be due to MBNL1 over-expression or an insertion effect of the transgene(s). Muscle in both lines appeared healthy with no histopathological or functional abnormalities, but premature splicing patterns were observed for MBNL targets in muscle, heart, and brain. The distribution of exogenous MBNL1 in muscle was nuclear, sometimes appearing as large puncta. The 14685 line was bred to the HSALR, which expresses expanded CUG repeats only in skeletal muscle, to determine whether constitutive over-expression of MBNL1 in muscle can rescue DM phenotypes. The resulting line showed decreased myotonia, improved muscle histopathology (fewer centralized nuclei and fewer ringed fibers) and normalized splicing patterns. Nuclear RNA foci were still present, consistent with the idea that MBNL1 is still sequestered but in sufficient quantities to also regulate other mRNA targets. Phenotypic rescue was observed through at least 1 year of age.

Over-expression of MBNL1 in the spinal cord delivered by a local injection has also been shown to result in paralysis.

This data suggests that over-expression of MBNL(s) can rescue DM-associated phenotypes by compensating for the MBNL protein(s) that are sequestered by expanded CUG repeats. Evidence also suggests that over-expression of MBNL in an uncontrolled manner may be toxic.

### The AAV Landscape

Developments in AAV technology have enabled clinical programs to be developed, most notably in the Spinal Muscular Atrophy space and the Duchenne muscular dystrophy space. An important improvement is in the composition of AAV capsids. AAV9 is used to target muscle, heart, and CNS, and AAV capsids derived from rhesus monkey are also in development. Given recent data, it appears that AAV delivery technology is no longer limited to delivering cargoes to the muscle and CNS. While these advances provide reasonable opportunities to deliver a gene therapy cargo, they do not provide the ability to regulate MBNL expression in a controlled manner, which is important to avoid potentially toxic levels.

### Strategies to Control MBNL Levels in the Context of AAV Delivery

The MBNL1 and 2 loci undergo extensive RNA processing; half of the exons in these genes are alternatively spliced, and these splicing patterns are regulated by the activity of numerous RNA binding proteins, including MBNLs themselves¹⁶. For example, the intronic sequence surrounding exon 5 in both MBNL1 and 2 are classified as 2 of 481 “ultra-conserved” sequences in the genome¹⁷, showing >200 basepairs of perfect sequence conservation between human, mouse, and rat. This extreme level of conservation may reflect a selection for sequences that provide the capacity for many RNA binding proteins to regulate the splicing of this exon. Exon 5 contains one half of a bi-partite nuclear localization sequence, and therefore MBNLs containing exon 5 exhibit a nuclear distribution whereas those that lack exon 5 are more cytoplasmic. The appropriate ratio of nuclear/cytoplasmic MBNL may be important to balance its splicing and RNA localization functions in multiple cell types.

In addition to exon 5, exon 1 appears to play a critical role in regulating the levels of MBNL1¹⁸. Exon 1 is actually the fourth exon in the MBNL locus but the first exon that has a start codon is referred to as exon 1 in the literature. High levels of MBNLs result in skipping of exon 1, leading to usage of a downstream alternative start codon, producing an MBNL1 protein that only has one pair of zinc fingers, which is unstable and rapidly degraded. In contexts of low or depleted MBNL, e.g., DM1, the inclusion level of exon 1 is increased, to compensate for loss of MBNL activity. In DM1, widespread splicing changes may occur after the autoregulatory capacity of exon 1 is outstripped by increased repeat load during disease progression.

These autoregulatory feedback loops provide opportunities to control the expression of exogenously administered MBNLs. In some embodiments, for example, AAV cargoes are designed such that exon 1 and exon 5 are incorporated as alternative splicing cassettes with their associated flanking introns. In a non-limiting example, FIG. 1C shows proposed AAV cargo that would allow for alternative splicing of exon 1 and exon 5. A significant portion of the introns would need to be removed in order to fit the cargo inside the AAV packaging limit of 4.9 kb. In some instances, the splicing event includes exon 1 (see, e.g., FIG. 1C, a1 and a2). In other instances, the splicing event excludes exon 1 (see, e.g., FIG. 1C, b). In some instances, the splicing event includes exon 5 (see, e.g., FIG. 1C, c1 and c2). In other instances, the splicing event excludes exon 5 (see, e.g., FIG. 1C, d). AAV ITR sequences, which allow for AAV replication and packaging, are shown. A generic promoter and 3′ UTR are also shown. In this non-limiting example, the levels of protein produced from the AAV genome would be regulated appropriately by the set of RNA binding proteins in the infected cell, in a manner that preserves proper MBNL stoichiometry in both nucleus and cytoplasm. For example, an unaffected or mildly affected DM1 cell that does not have a high repeat load would contain relatively normal MBNL activity and therefore protein production from the AAV cargo would be suppressed. In contrast, a DM1 cell that contains a very high repeat load would contain very low MBNL activity and therefore protein production from the AAV cargo would be permissive.

In some embodiments, the intronic regions contain the appropriate cis-elements to allow for regulation by not only MBNL but also other RNA binding proteins. As a consequence, in some embodiments proper cell type-specific expression of MBNLs would occur, no matter the amount of AAV-MBNL present in the cell. For example, a glial cell may require different levels of MBNL than a neuron, and/or different nuclear/cytoplasmic ratios. An MBNL gene cargo that contains alternative exons would allow for production of multiple isoforms in a regulated manner, depending on the trans-factor environment in that given cell.

The endogenous introns flanking exon 1 and exon 5 are much too large to fit within the AAV packaging limit of 4.9 kb. However, many alternative splicing events have been studied in the context of plasmid mini-genes, in which large segments of distal intronic sequence are omitted in the construction of the minigene. Therefore, a key activity to enable AAV-MBNL gene therapy is to identify the exon-intron sequences that preserve appropriate regulatory activity yet can still fit within the size of an AAV genome. Extensive work has been performed to understand elements surrounding exon 5 of MBNL 1¹⁹; the manner in which this exon responds to MBNL activity depends on a number of sequence features in the upstream intron.

MBNL1 and MBNL2 are highly similar at the amino acid level, and perturbations to either MBNL1 or 2 result in cross-regulation to normalize the levels of the other, such that the total MBNL activity is maintained in the cell. However, the subcellular distribution of MBNL1 and MBNL2 can differ in some cell types. For example, in most neurons, MBNL1 is predominantly cytoplasmic and MBNL2 is predominantly nuclear. This may allow for division of labor, for MBNL2 to primarily regulate splicing and for MBNL1 to primarily regulate RNA localization to synapses. In any case, a choice must be made as to which MBNL to package into an AAV gene therapy. Because the two proteins are so similar, they extensively cross-regulate each other, and extensive work has been performed to understand the splicing of MBNL1 exons 1 and 5. Accordingly, in some embodiments, an AAV-MBNL1 vector is developed with truncated versions of the introns flanking exons 1 and 5, that behave similarly to the normal genomic context. In some embodiments, exon 3 is included, because it is important for efficient RNA binding activity. In some embodiments, exon 7 is included. In some embodiments, exon 7 is excluded, because it has been proposed to influence dimerization activity. In some embodiments, exon 9 is included. In some embodiments, exon 9 is excluded because it is included relatively infrequently in tissues and alters the C-terminal sequence in exon 10.

In some embodiments, preclinical work is performed to compare the effects of AAV-MBNL1 to AAV-MBNL1_{spliced} in both WT mice as well as DM1 mouse models. In some embodiments, the expression and activity of AAV-MBNL 1_{spliced} in single cells of the central nervous system are studied. MBNL levels may need to be tightly regulated in CNS cells to avoid deleterious consequences. For example, it has been observed that over-expression of exogenous MBNL leads to premature adult splicing patterns¹⁵. In some embodiments, administration of AAV-MBNL1_{spliced} would not perturb splicing patterns in WT mice but would still rescue splicing patterns in DM1 mice. As a non-limiting example, FIG. 1D shows the proposed functionality of AAV-MBNL1_{spliced}as compared to a version that cannot splice. In a healthy cell, the amount of MBNL activity increases as a function of AAV-MBNL1 dose, but stays constant if dosed with AAV-MBNL1_{spliced}. In a DM cell, both AAV-MBNL1 and AAV-MBNL1_{spliced} can rescue the depletion of MBNLs by expanded CUG repeat RNAs, but the AAV-MBNL 1 that cannot splice has risk of overshooting the target concentration. AAV-MBNL 1_{spliced} would supplement the MBNL in the cell to compensate for what is sequestered by CUG repeat RNA, and then remain at that level even if higher doses of AAV are achieved. This has the advantage of using endogenous splicing machinery to maintain appropriate levels of MBNL no matter what dose is achieved or what cell type is infected.

### Promoter and UTR Considerations

While an AAV-MBNL1_{spliced} gene therapy cargo will contain exonic and intronic sequences from the MBNL1 locus, promoter type and UTRs can be variable. The MBNL1 coding sequence alone is ˜1.2 kb and therefore ˜3.7 kb remains for other regulatory elements, including introns. There are 3 alternative 5′ UTRs for MBNL1 (FIG. 1B), and the second is most commonly used in heart and muscle¹⁸. Since these exons are relatively short, it is possible to use the second exon and splice it to “exon 1” as described above. The nomenclature can be confusing in that “exon 1” as referred to above is actually the fourth exon; the first 3 exons are alternative first exons that all splice to “exon 1” as referred to above.

The endogenous 3′ UTR of MBNL1 is ˜3.4 kb and would be too large to include in an AAV vector. While there are likely additional regulatory mechanisms in the cell that act through the 3′ UTR, because of its large size, it may be more favorable to simply use a 3′ UTR that favors high message stability, such as the bovine growth hormone 3′ UTR. Other non-limiting examples of additional regulatory sequences could include endogenous or synthetic 5′ UTR or 3′ UTR sequences that confer appropriate regulatory activity.

A ubiquitous promoter should be selected, since splicing and regulation of translation may limit MBNL expression where appropriate. Non-limiting examples of small, effective promoters would be EF 1 alpha, beta actin, and the CAG promoter. In the context of Duchenne muscular dystrophy, the following non-limiting, exemplary promoters have been proposed: CK8, MHCK7, miniMCK, and SPc5-12. These are muscle-specific and therefore would not achieve expression in other tissues affected in myotonic dystrophy.

### Target Patient Population

All types of DM patients, including DM1 and DM2, would benefit from an AAV-MBNL1 gene therapy treatment. Targeting congenital DM1 cases may offer several advantages: 1) the mortality rate due to congenital DM1 is ˜25% before the age of 18 months, 2) gene therapy approaches from SMA have shown beneficial effects in other settings (e.g., in infants), and 3) during postnatal development, MBNL activity rises and the splicing network is actively forming and therefore there may be lower risks to exogenously introducing RNA binding proteins. In some embodiments, small molecule, antisense, and gene therapy approaches are used in combination to provide greater benefit to this patient population.

### Example 2. Measuring Splicing Behavior of Synthetic MBNL Constructs 1-4

In this example, four constructs were separately transfected into HeLa cells to measure their splicing behavior:
1) A full version of a minigene that includes part of exon 4, all of intron 5, exon 5, all of intron 6, and part of exon 6;
2) A minigene with part of intron I5 deleted;
3) A minigene with part of intron I6 deleted; or
4) A minigene with parts of intron I5 and intron I6 deleted.

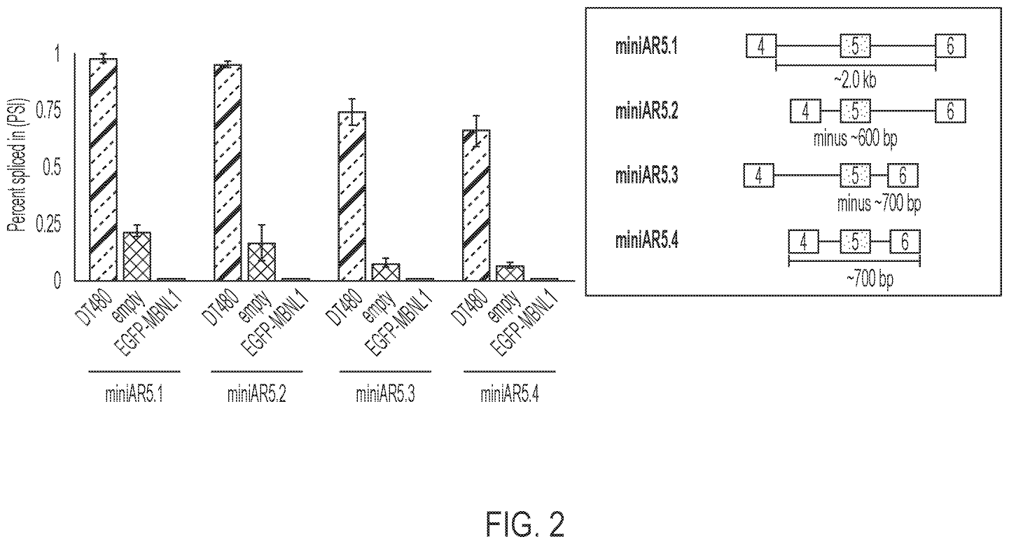

The four constructs above were either tested alone, with a plasmid expressing 480 CTG repeats, or with a plasmid encoding MBNL1 coding sequence. RT-PCR was performed to measure inclusion levels of exon 5. For all constructs, it was observed that MBNL1 over-expression reduces exon 5 inclusion, and CTG repeats increase exon 5 inclusion, as shown in FIG. 2. These data show that regulation of exon 5 splicing in response to MBNL levels can be performed despite these deletions of intron I5 and intron I6.

Exon 4, intron I5, exon 5, intron I6, and exon 6 sequences used in the above constructs are found below. The noted portions of the intron I5 and intron I6 sequences were deleted to make constructs 2-4 (deleted portions are shown as struck-through).

3′ end of exon 4:
(SEQ ID NO: 1)
AGCACAATGATTGACACCAATGACAACACAGTCACTGTGTGTATGGATTA
CATCAAAGGGAGATGCTCTCGGGAAAAGTGCAAATACTTTCATCCCCCTG
CACATTTGCAAGCCAAGATCAAGGCTGCCCAATACCAGGTCAACCAGGCT
GCAGCTGCACAGGCTGCAGCCACCGCAGCTGCCATG
Truncated Intron 5:
(SEQ ID NO: 2)
GTGAGTAGAGATATCAGCTCTCTCCTTGTTAGCAGTCAGAAAAGCAAAGT
GAGCAACTATATCTGACTACAAGCTATTCATTTAGTAACCTTTTTAAAAA
AATTGCTGAAGATATGTTTGTTCAGGTATCCCAGAC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

(SEQ ID NO: 3 is deleted)

(SEQ ID NO: 4)
CACGAGAGAGATCTTTTCTGTGTTTATGGATACTTGAGCAAAAATACAGA
AGGCAGACTCTCTCCTCCTCTCTTCCTTTCACTCTTTTTTTTTTCTGTTA
GAGTATCTTGTTTGTAATTAACTACAAAGAGGAGTTATCCTCCCAATAAC
AACTCAGTAGTGCCTTTATTGTGCATGCTTAGTCTTGTTATTCGTTGTAT
ATGGCATTCCGATGATTTGTTTTTTTATTTGTTTTTTCTCACCTACCCAA
AAATGCACTGCTGCCCCCATGATGCACCTCTGCTTGCTGTTTATGTTAAT
GCGCTTGAACCCCACTGGCCCATTGCCATCATGTGCTCGCTGCCTGCTAA
TTAAG
Exon 5:
(SEQ ID NO: 5)
ACTCAGTCGGCTGTCAAATCACTGAAGCGACCCCTCGAGGCAACCTTTGA
CCTG
Truncated Intron 16:
(SEQ ID NO: 6)
GTACTATGACCTTTCACCTTTTAGCTTGGCATGTAGCTTTATTGTAG

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

(SEQ ID NO: 7 is deleted)

(SEQ ID NO: 8)
GCCTTCGACTGATTTTTCTTTTTTCTTTTTCTCTTTTTACTGGTATTTGT
TTTTTATACTCATTCACTAAACAG
5′ end of exon 6:
(SEQ ID NO: 9)
GGAATTCCTCAAGCTGTACTTACC

Each of the sequences described herein should be construed as non-limiting, and optionally smaller and/or larger sequences or portions thereof could be used to make the gene construct (e.g., sequences that are 1-10% shorter, for example around 1%, around 2%, around 3%, around 4%, around 5%, around 6%, around 7%, around 8%, around 9%, or around 10% shorter, or around 1-10% longer, for example around 1%, around 2%, around 3%, around 4%, around 5%, around 6%, around 7%, around 8%, around 9%, or around 10% longer).

### Example 3. Measuring Splicing Behavior of Synthetic MBNL Constructs 5-8

In this example, four constructs were separately transfected into HeLa cells to measure their splicing behavior:
1) A full version of a minigene that includes part of exon T2, all of intron I1c, exon 1, all of intron I2, and part of exon 2;
2) A minigene with part of intron I1c deleted;
3) A minigene with part of intron I2 deleted; or
4) A minigene with parts of intron I1c and intron 12 deleted.

The four constructs above were either tested alone, with a plasmid expressing 480 CTG repeats, or with a plasmid encoding MBNL1 coding sequence. RT-PCR was performed to measure inclusion levels of exon 1.

Exon T2, intron I1c, exon 1, intron I2, and exon 2 sequences used in the above constructs are found below. The bold-faced and underlined portions of the intron I1c and intron I2 sequences were deleted to make constructs 2-4.

3′ end of exon T2:
(SEQ ID NO: 10)
AGCTGAATGAGTTGTGGCGCCCACAATGCTCCCATGACAAGGAGCTGACA
AGTTCCATTTTCCGTCGCGGGCATCTTGGAATCATGACTCCCACAATGCC
TTGGGCACTTGGTCGACAGTGGGGCCGCCTCTGAAAAAAAAATGTGAGAG
Abbreviated intron lie between
T2 and exon 1:
(SEQ ID NO: 11)
GTAAGTTTCCATTTTCACAGTTTCCCCGCGCCGCTTCATTGTTCGGACTC
CGGCGGGTCTGCCCGTGGCTGAAGGAGGAAGTGCGAGGAGGTGCTCGCCG
GCCGCGGTTCTCCCGGGCAGGGGCGGGCCGCTCGCGGTAGTTGGTTTCGC
(SEQ ID NO: 12 is deleted)
(SEQ ID NO: 13)
AGGCTGACTTGAATATTACTTAGTCTGGTATCACATGGCAAGGTTGTGAT
ACTGTAGGACATCAGTGAAGTGCTACTTAGTAAATCTTAACCTATCTCTT
TTTTCTACAG
Exon 1:
(SEQ ID NO: 14)
GTTGGTACTAAGAAGTGCCTTTCCTGACGTCTCTGCTGCTTGGAACCGCT
TCTAGAGCAGTCTCTGCTTTTGCCTTGCTTGCTGCCAGCTAGACTGTGAC
GACAGCACATCCACCCTCCACCTCTAGCCCAGACACCCCCATTTCTACTT
ATAATCAAGAGAAAAGCTCTAAGTATCTGGCATTGCCCTAGGCTGCTTTA
GTGTTAAAAGAAAAGTTTGCTGAAAAAGTAAGATATCTTCTGCCAGGAAA
TCAAGGAGGAAAAAAAAAATCATTTTCTCGATTTTGCTCTAAACTGCTGC
ATCTGTCTATGCCAAACTAATCAATACCGATTGCACCACCAAACTCCATT
GCAAATTCAGCTGTGAGGAGATTCCCTTTCAGACAACTTTGCTGAAAGCA
GCTTGGAAATTCGGTGTCGAAGGGTCTGCCACGTTTTCATGCTTGCATTT
TGGGCTCCAAATTGGCACTGGGAAGGGGTTACTGAGAGCACAAGGCTGAT
ACCAGGCCCTACTTTTAAACGTTCATCTACTTACAATCCTAGTATTTCTC
TAAAAACCAAAACCTCTTTGAATTAACAGTTTCATGCTGTGAATTTCTAG
TGGGAGATCTTTTCCTTGATATTGACGACACAATTTTCCATGTACTTTTA
AAGCAGGGAGTGGGGAAAAGTATTTTGAGGGGACATTTTCATCATCAGTT
CAGCTTTTTTTTTTTGGTTGTTGCTCTTTTTTGGGGGGGTTGGGTTTGTT
GGTTTCACTGAAACATTTAACTACCTGTAAAATCTAAACATGGCTGTTAG
TGTCACACCAATTCGGGACACAAAATGGCTAACACTGGAAGTATGTAGAG
AGTTCCAGAGGGGGACTTGCTCACGGCCAGACACGGAATGTAAATTTGCA
CATCCTTCGAAAAGCTGCCAAGTTGAAAATGGACGAGTAATCGCCTGCTT
TGATTCATTGAAA
Truncated intron 12 between exon 1
and exon 2:
(SEQ ID NO: 15)
GTGAGTAACTATTATATTCTTTTAAGGATATTCAATGATTGAATGAGGGG
TATATGGACATACAGTTCTCTGC
(SEQ ID NO: 16 is deleted)
(SEQ ID NO: 17)
CCAATGATACATTCAAGTGGGTGGTTTGCATTATATTTTTTTCATAATTT
GCTGATTTTATTCTTTTCTAAGATGATGTTTGCTGCTTTTGTTTCTCTAG
5′ end of exon 2:
(SEQ ID NO: 18)
GGCCGTTGCTCCAGGGAGAACTGCAAATATCTTCATCCACCCCCACATTT
AAAAACGCAGTTGGAGATAAATGGACGCAATAACTTGATTCAGCAGAAGA
ACATGGCCATGTTGGCCCAGCAAATGCAACTAGCCAATGCCATGATGCCT
GGTGCCCCATTACAACCCGTG

Each of the sequences described herein should be construed as non-limiting, and optionally smaller and/or larger sequences or portions thereof could be used to make the gene construct (e.g., sequences that are 1-10% shorter, for example around 1%, around 2%, around 3%, around 4%, around 5%, around 6%, around 7%, around 8%, around 9%, or around 10% shorter, or around 1-10% longer, for example around 1%, around 2%, around 3%, around 4%, around 5%, around 6%, around 7%, around 8%, around 9%, or around 10% longer).

### Example 4. Truncation and Design of Naturally Occurring Introns for Use in an AAV Cargo that Contains MBNL Coding Sequence

Most mammalian introns are ˜1 kilobase in length or more (reviewed in (21)), and therefore use of introns that flank an alternative exon may take up a large part of the AAV packaging capacity, leaving little room for additional sequence. Generally, the approach to truncate introns by removing their distal sequences has been taken, using phylogenetic conservation as a guide as to which sequences to maintain in a final cargo.

### MBNL1 Exon 5 and Flanking Introns

First, MBNL1 exon 5 and flanking introns 4 and 5 were used as a test case. MBNL1 exon 5 was a nuclear localization signal-containing alternative exon whose inclusion can be regulated by MBNL proteins (22). In conditions of high MBNL concentration, MBNL proteins bind to intron 4 and cause skipping of exon 5. This results in a protein isoform that is less nuclear relative to MBNL1 isoforms that contain exon 5. In conditions of low MBNL concentration (e.g. in myotonic dystrophy), exon 5 is more highly included, generating protein isoforms that are more nuclear. Therefore, splicing regulation of exon 5 forms a regulatory loop that allows MBNL1 to regulate its own nuclear versus cytoplasmic levels.

For the assays described below, three conditions in which the behavior of exon 5 inclusion can be tested are described: 1) in the presence of CTG480 over-expression, 2) in the presence of no other plasmid or just a “filler” plasmid, or 3) in the presence of a separate MBNL1 expression cassette. Plasmids are transfected into Hela cells; RNA is harvested 24-48 hours later, and RT-PCR is performed to assay the inclusion level of the alternative exon.

First, data is shown for a mini-gene containing “full length” exon 4, intron 4, exon 5, intron 5, and exon 6 (miniAR5.1). The inclusion level of exon 5 in condition (1) is >90%, (2) is ˜20%, and (3) is ˜1%. Subsequently, constructs were tested in which intron 4 was truncated from 1164 bases to 491 bases (mAR5.2), intron 5 was truncated from 862 bases to 121 bases (mAR5.3), or both introns 4 and 5 were truncated (miniAR5.4). miniAR5.2, miniAR5.3, and miniAR5.4 exhibit splicing regulatory activity in response to CTG480 and MBNL over-expression that largely mirrors that exhibited by full length “miniAR5.1” conditions.

Upon obtaining these results, the truncated alternative splicing cassette was cloned from miniAR5.4 into the context of a full length MBNL1 coding sequence in the context of AAV ITRs (AR5.1) (FIG. 3). When this cargo was expressed in Hela cells by transfection, the expected behavior was observed in response to CTG480 and MBNL over-expression. However, an additional isoform that uses a cryptic 3′ splice site within exon 6 was also observed. Subsequently, the “AG” in the cryptic splice site was mutated to a “TC” to eliminate this isoform (AR5.2). This change did not disrupt the splicing regulation that occurs in response to CTG480 or MBNL over-expression.

While AR5.2 eliminated isoforms that use the cryptic splice site internal to exon 6, AR5.2 showed some intron retention, in particular of truncated intron 5. This intron retention was reflected both at the RNA level as well as at the protein level; Western blotting showed this isoform that results from translation of exons 1-5 and into intron 5, terminating at a stop codon within intron 5. AR5.2 was further optimized by adding back additional endogenous MBNL intron 5 sequence; the major changes included adding back sequences that included a strong branch point (CTAAC), along with 0 (AR5.3), 2 (AR5.4), or 4 (AR5.5) copies of the YGCY motif to which MBNL binds. All constructs largely reflected similar behavior in response to CTG480 and MBNL over-expression, but AR5.5 showed behavior most similar to a “full-length” AR5.1 construct, and also eliminated intron retention of intron 5 (FIG. 4). Thus, a construct was engineered that behaves similarly to “full length” MBNL1 with an alternative exon 5 splicing cassette.

### MBNL1 Exon 1 and Flanking Introns

In the genomic context, MBNL1 exon 1 (which contains a large part of the 5′ UTR and the start of the coding sequence) is alternatively spliced. There are 3 transcription start sites (T1, T2, and T3), and each can be spliced to the 5′ splice site of exon 1¹⁸. T2 is most commonly used in tissues in which MBNL expression is high, and T2 can either splice to the 5′ splice site of exon 1 or can splice to the 5′ splice site of exon 2, effectively skipping exon 1. Skipping of exon 1 leads to usage of a translation start codon that generates a truncated protein that is rapidly degraded. Exon 1 skipping is regulated by MBNL itself, as MBNL proteins can bind to exon 1 and cause skipping. This comprises an auto-regulatory loop that allows MBNL1 to regulate its own protein levels.

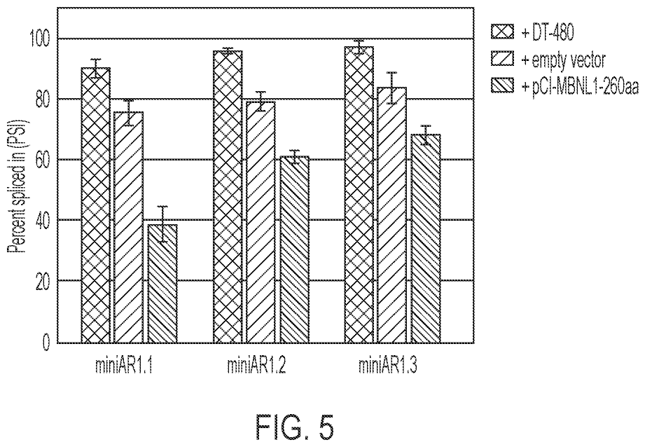

The length of sequence from the beginning of T2 to the end of exon 2 is ˜150 kilobases —too large to fit within the AAV packaging limit. Therefore, the intron was truncated before exon 1 and the intron after exon 1 to generate minigene “miniAR 1.1”; the final intron before exon 1 in this construct was 259 bases and the final intron between exon 1 and 2 was 173 bases. This mini-gene was subjected to CTG480 or MBNL over-expression to observe its behavior in Hela cells (FIG. 5). It was observed that exon 1 was included at ˜90% with CTG480, ˜75% with a filler plasmid, and ˜40% with MBNL over-expression, validating auto-regulatory behavior. Several additional constructs were tested in which parts of exon 1 were deleted, e.g. “miniAR 1.2”, “miniAR 1.3”, but these did not show the same extent of auto-regulatory behavior.

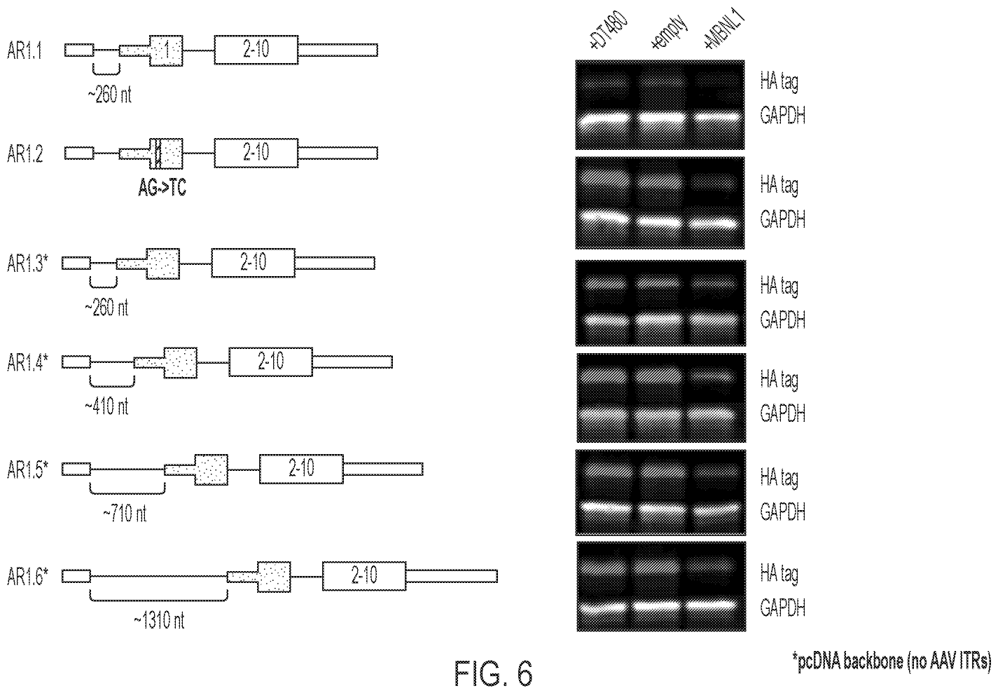

Subsequently, this cassette was cloned into a full length MBNL 1 coding sequence, in the context of AAV ITRs (AR1.1). Inspection of protein levels following co-transfection of CTG480 or EGFP-MBNL revealed prominent down-regulation of the AR1.1 cargo in the presence of MBNL over-expression and up-regulation in the presence of CTG480 (FIG. 6). Assessment of RNA isoforms revealed several isoforms: 1) splicing from the 3′ splice site of T2 to the 3′ splice site of exon 1, and splicing from the 5′ splice site of exon 1 to the 3′ splice site of exon 2, resulting in “full length” MBNL1 protein; and 2) splicing from the 3′ splice site of T2 to a cryptic 3′ splice site just downstream of the canonical ATG start codon, and splicing from the 5′ splice site of exon 1 to the 3′ splice site of exon 2, essentially resulting in an mRNA substrate that encodes a short peptide, followed by the exon 1-exon 2 exon junction complex (FIG. 7).

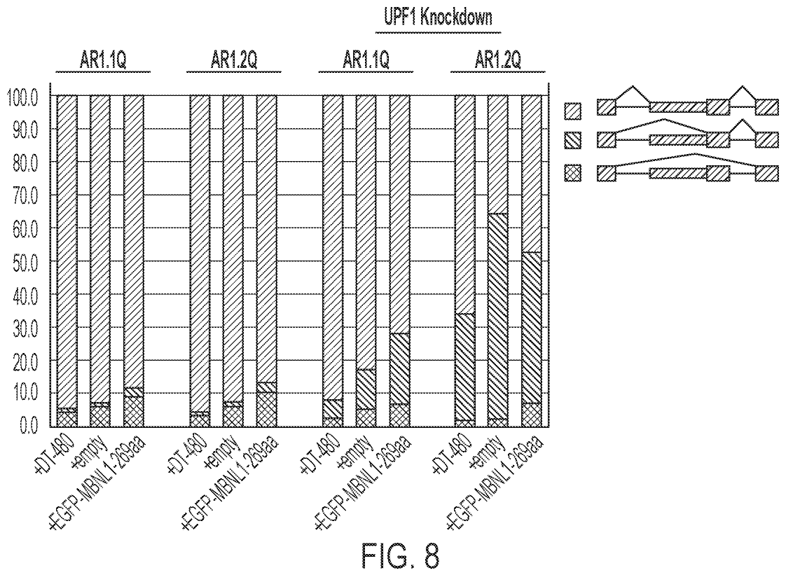

This second isoform was expected to undergo nonsense-mediated decay, because the stop codon is >50 nucleotides away from the downstream exon junction. Indeed, when the NMD machinery was knocked down by shRNA against UPF1, it was observed that this second isoform increases in abundance in the MBNL over-expression condition (FIG. 8). To test the robustness of this mechanism and the ability of MBNL to regulate its own exon 1 splicing patterns, the “AG” of this cryptic splice site was mutated to “TC” (AR1.2). This new construct was tested and also revealed strong protein down-regulation in response to MBNL over-expression.

Upon analysis of RNA isoforms, use of another cryptic splice site downstream of the first cryptic splice site was observed. The two predominant isoforms where one where exon 1 was fully included, and one where the 5′ end of exon 1 was skipped such that the 5′ splice site of T2 was spliced to this newly observed cryptic splice site. This new isoform generated an ATG-TAG sequence that was >50 nucleotides upstream of the exon 1-exon 2 splice junction and was also expected to result in mRNA degradation. Indeed, this additional isoform was also increased in abundance upon knockdown of UPF1 and over-expression of MBNL (FIG. 7). To further test the robustness of auto-regulatory activity of AR1 constructs, additional expression plasmids were generated in which additional sequence from the intron before exon 1 was added back (AR1.3-AR1.6). All of these constructs retained their auto-regulatory activity (FIG. 6). Thus, constructs that exhibit the auto-regulatory behavior conferred by exon 1 of MBNL1, that fit within the AAV packaging limit, were generated.

MBNL1 Exon 1 and Exon 5 with Flanking Introns in the Same AAV Cargo

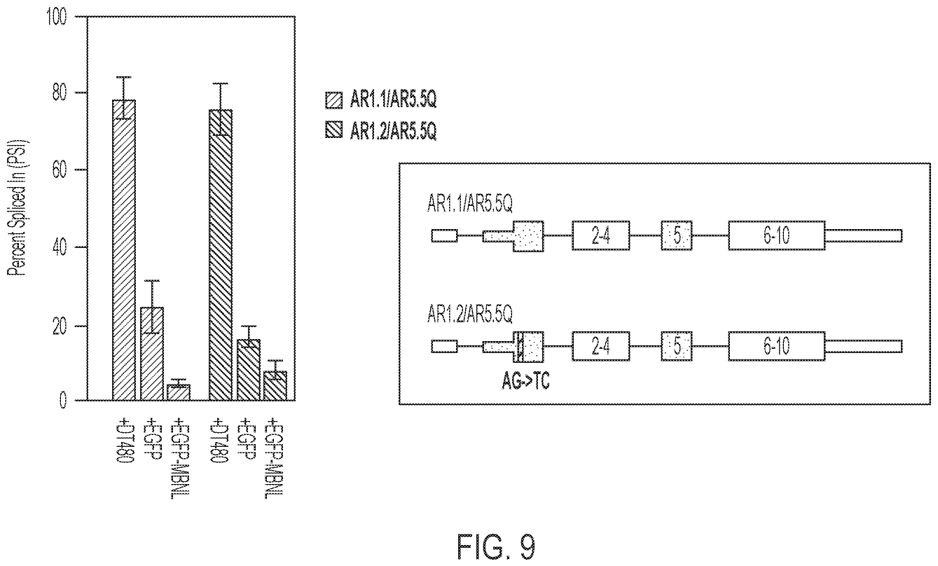

AAV constructs were generated in which the alternative splicing cassettes from AR1.1 and AR5.5 were combined into a single construct, or AR1.2 and AR5.5 were combined into a single construct. Similar to what was observed for AR5.5 alone, the inclusion levels of exon 5 in the AAV constructs exhibits auto-regulation in response to DT480 or EGFP-MBNL1 co-transfection (FIG. 9).

### Example 5. Truncation and Optimization of Additional Alternative Splicing Cassettes for Use in Other AAV Cargoes

Beyond MBNL, gene therapies that can self-regulate or sense their intracellular surroundings are highly desirable. These properties mitigate dose-limiting toxicities as well as facilitate desired spatiotemporal aspects of gene regulation (e.g., provide cargo expression in the right place at the right time). Described herein is a general framework for how auto- or cross-regulatory properties can be built into gene therapy cargoes, either using naturally occurring (e.g. phylogenetically conserved) elements or using hybrid/synthetic elements.

### Oculopharyngeal Muscular Dystrophy (OPMD)

OPMD is an adult onset, autosomal dominant muscle disease caused by a GCN repeat expansion within the N-terminus of the PABPN1 gene (23). PABPN1 is a critical protein that shuttles between the nucleus and cytoplasm and has roles in regulating polyA tail length, mRNA stability, and RNA export (24). Normally, there is a polyalanine repeat of 10 units in the N-terminus of the protein but in individuals with OPMD, the repeat can be up to 17 units. This expansion results in both loss and gain of various molecular functions, which also are both thought to contribute to disease (25-27). Some of the interactors of PABPN1 include well established RNA binding proteins also implicated in disease, such as Matrin3 (28), and expanded, but not WT, PABPN1, was shown to interact with TDP43(29).

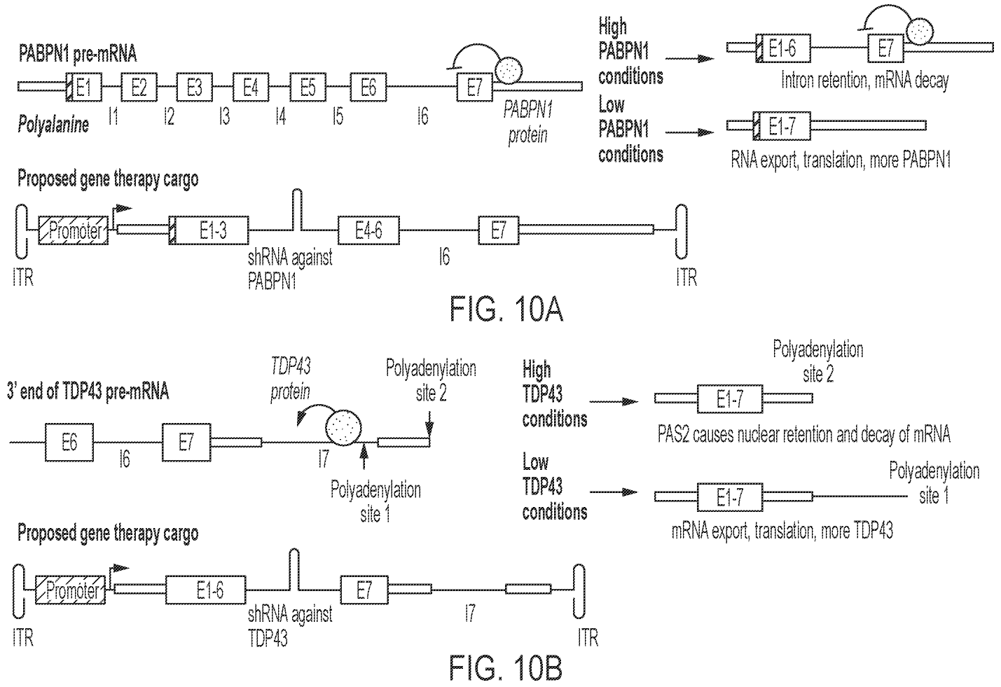

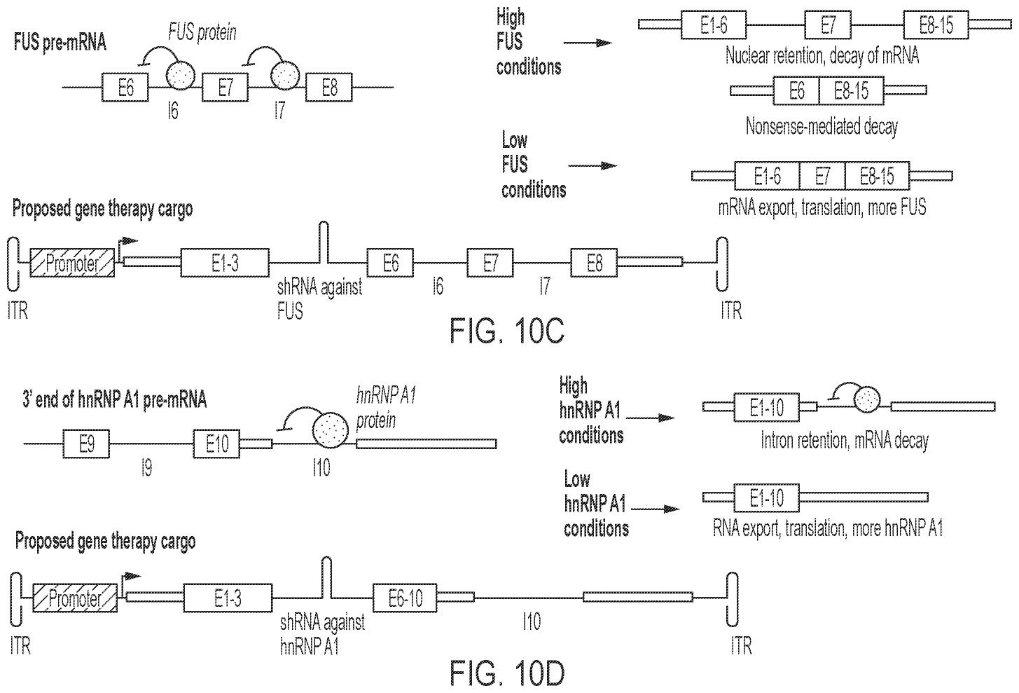

Various approaches to treat this disease have been taken, including a knock-down and replacement approach in which RNAi was used to knockdown the endogenous copies of PABPN1 and an RNAi-resistant version of PABPN1 was added (30-32). One shortcoming of current knockdown and replacement approaches is that the levels of the exogenous PABPN1 cannot be effectively controlled. PABPN1 over-expression has been shown to alter numerous gene expression changes (33) and as indicated above may be toxic when over-expressed outside a certain range. The endogenous PABPN1 is subject to high degrees of RNA processing and regulatory control, including an intron retention event that leads to nuclear RNA decay of the PABPN 1 transcript (34). In conditions of low PABPN 1 activity, the intron was efficiently spliced, but in conditions of high PABPN 1 activity, the intron was retained through binding of PABPN 1 to a stretch of adenosines in exon 7: this intron retention event leads to decay of the RNA in a nuclear exosome-dependent manner (FIG. 10A). This auto-regulatory loop has been studied and recapitulated in a mini-gene transfection-based context, providing a blueprint for how an auto-regulated AAV-mediated gene therapy could be developed.

A straightforward strategy was to include intron 6 (the retained intron) within the context of the full-length coding sequencing of PABPN1 with 10 polyalanine repeats, and package this into the AAV along with an RNAi cassette against endogenous PABPN 1. The RNAi cassette was included as a U6-driven shRNA or a shRNA that was processed from an intron which were incorporated into an upstream constitutive intron (FIG. 10A). Synonymous codon or 3′ UTR mutations were made such that the exogenous copy was insensitive to the RNAi cassette.

### Familial and Sporadic Forms of Genetically Defined ALS

A number of genetically defined forms of ALS are caused by mutations in RNA binding proteins (35-37). These RNA binding proteins include TDP43, FUS, hnRNP A1, hnRNP A2B1, TIA1, EWSR1, MATR3, ATXN2, TAF15, and others. An emerging hypothesis is that normal RBP metabolism requires frequent association and dissociation, and that mutations found in these RBPs either alter their stoichiometries in certain cellular compartments, or affect their ability to undergo reversible liquid-liquid phase separations (38). Aggregation or localization to stress granules are related processes that may also play a role in disease pathogenesis (39). Many of these proteins have low complexity or intrinsically disordered domains that seem crucial to normal function (40), but are hotspots for mutations that lead to altered biophysical behavior. Overall, the prevailing consensus is that some of these newly acquired interactions set off a cascade of molecular events leading to cell dysfunction and death.

Although a knockdown and replacement strategy seems reasonable in these contexts, the dosage of RBPs must be tightly controlled. Indeed, ˜2-fold over-expression of hnRNP A1 can cause cytotoxicity (41), and over-expression of wild type TDP43 23 or wild type FUS 24 both cause motor deficits and are used as models to study ALS disease pathogenesis. Conveniently, most, if not all of the RBPs listed above contain potent auto-regulatory circuitry that operates at the genomic level, and these auto-regulatory capabilities are critical for normal healthy function. For example, the auto-regulatory loop that preserves appropriate nuclear/cytoplasmic levels of FUS is “broken” by ALS-associated mutations (44), and certain mutations in TDP43 that enhance cytoplasmic localization are also associated with ALS (45).

TDP43: Although sequences that confer auto-regulatory capabilities have been extensively studied, they have not been incorporated into an exogenous gene therapy. The TDP43 auto-regulatory loop allows for effective control of total TDP43 levels primarily via an intron retention event and regulation of polyadenylation. In conditions of low TDP43 activity, intron 7 was retained and a polyadenylation site within this retained intron was chosen, resulting in normal TDP43 expression and mRNA export. In conditions of high TDP43 activity, TDP43 bound intron 7 and caused it to be spliced out. The transcript was left with less efficiently recognized polyadenylation sites, RNA PolII stalling, and transcript degradation. Thus, TDP43 controlled the levels of its own mRNA via splicing regulation. A gene therapy that included these regulatory elements would then allow for auto-regulation of TDP43 levels (FIG. 10B).

FUS: FUS is regulated via a similar auto-regulatory loop, in which it binds to its own introns 6 and 7. Retention of these introns in conditions of high nuclear FUS causes 1) retention and degradation of the mRNA in the nucleus (46) or alternatively 2) skipping of exon 7, generating a nonsense-mediated decay isoform (47). In low FUS conditions, productive splicing occurred, and the mRNA was exported, generating a FUS protein that normally contains a nuclear localization signal (NLS). The presence of the NLS was essential for the auto-regulatory loop; multiple ALS mutations disrupt this NLS and therefore cause high cytoplasmic FUS to accumulate without a change in splicing patterns or degradation of FUS mRNA⁴⁴. Thus, FUS also controls the levels of its own mRNA via splicing regulation, and a gene therapy could also include such relevant regulatory elements (FIG. 10C).

hnRNP A1: Notably, in addition to ALS, mutations in hnRNP A1 are also linked to multisystem proteinopathy (48). hnRNP A 1 is a highly expressed protein with roles in regulation of transcription, splicing, RNA export, RNA stability, and microRNA biogenesis (49). Mutations in its low complexity domain have been associated with ALS. This protein also possesses a potent auto-regulatory loop (41). Intron 10 was bound by hnRNP A 1, leading to retention of the intron, and degradation of the mRNA, potentially via a mechanism similar to what occurs for TDP43. Similar approaches can be taken to develop a gene therapy (FIG. 10D).

As described above, excessive over-expression of some RBPs may be toxic, and therefore gene therapies that lack auto-regulation would likely be toxic. Following knock-down and replacement with an auto-regulated cargo, regardless of AAV dose, the final level of each gene therapy would be indistinguishable from normal healthy conditions. The feasibility of this was determined by the overall buffering capacity of each auto-regulated cargo. In multiple contexts, over-expression of an exogenous RBP resulted in almost complete down-regulation of its endogenous mirror copy; by recapitulating the robustness of this auto-regulatory capacity, the total dose would be decoupled from the total copy number of AAV episomes within the cell. Finally, some of these genetically defined forms of ALS have especially aggressive disease courses with no viable treatment options. In some cases, death occurs 2 years following diagnosis. At this time, a rationally designed gene therapy approach may be the most effective way to intervene; at the same time, it would provide learnings of how robust this approach is and how broadly it might be employed across multiple disease areas.

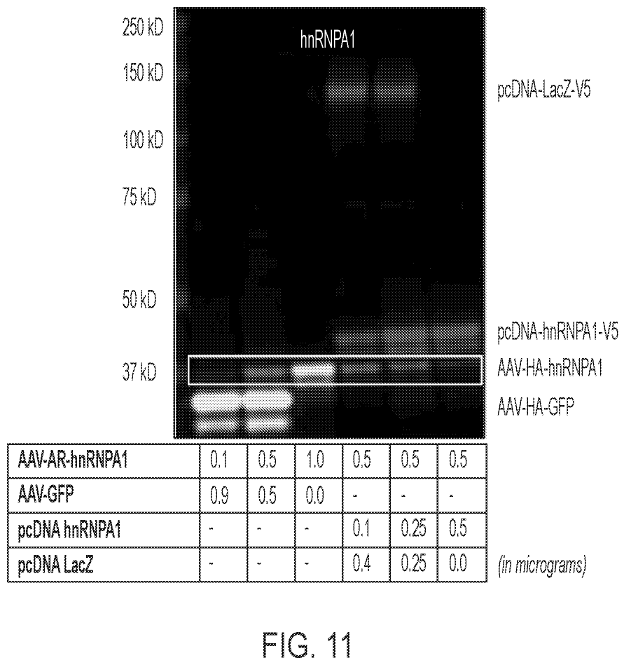

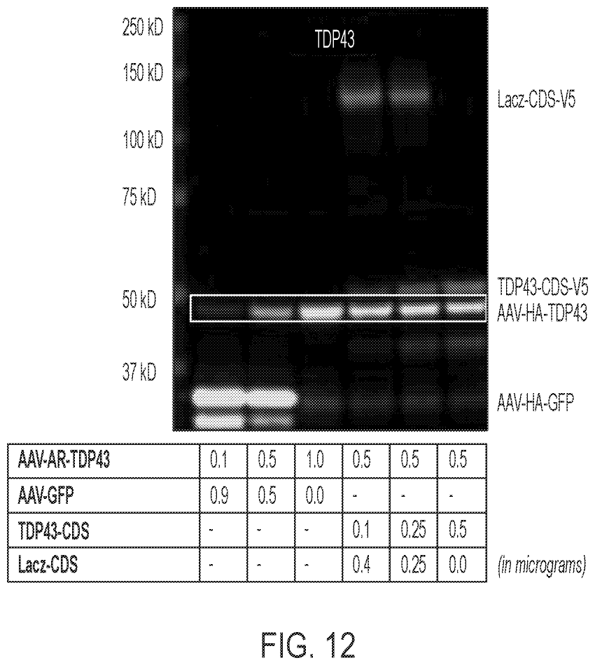

Data for hnRNP A1 was generated, in which an HA-tagged coding sequence for hnRNP A1 was cloned into an AAV backbone; intron 10 of hnRNP A1 was also included in the construct, as this sequence is required for hnRNP A1 auto-regulation. A fixed amount of the AAV construct was co-transfected with increasing amounts of an expression plasmid encoding a V5-tagged coding sequence of hnRNP A1 and decreasing amounts of an expression plasmid encoding LacZ (into Hela cells). Protein was harvested 24 hours later and a Western blot was performed against the HA tag and the V5 tag. High expression of the hnRNP A1 coding sequence resulted in down-regulation of hnRNP A1 derived from the AAV auto-regulated plasmid (FIG. 11).

Data for TDP43 was also generated, in which an HA-tagged coding sequence for TDP43 was cloned into an AAV backbone; introns 6 and 7 of TDP43 were also included in the construct, as these sequences are required for TDP43 auto-regulation. A fixed amount of the AAV construct was co-transfected with increasing amounts of an expression plasmid encoding a V5-tagged coding sequence of TDP43 and decreasing amounts of an expression plasmid encoding LacZ (into Hela cells). Protein was harvested 24 hours later and a Western blot was performed against the HA tag and the V5 tag. High expression of the TDP43 coding sequence resulted in down-regulation of TDP43 derived from the AAV auto-regulated plasmid (FIG. 12).

### Example 6. Heterologous Constructs to Confer Novel Regulatory Behavior for Use in an AAV Cargo

Provided herein are approaches to build a cargo that incorporates intron-exon structures in their non-natural contexts to achieve desired regulatory behaviors.

Control of MBNL Protein Levels by Incorporating ATP2A1 Exon 22 Along with New Stop Codons to Elicit NMD when Included

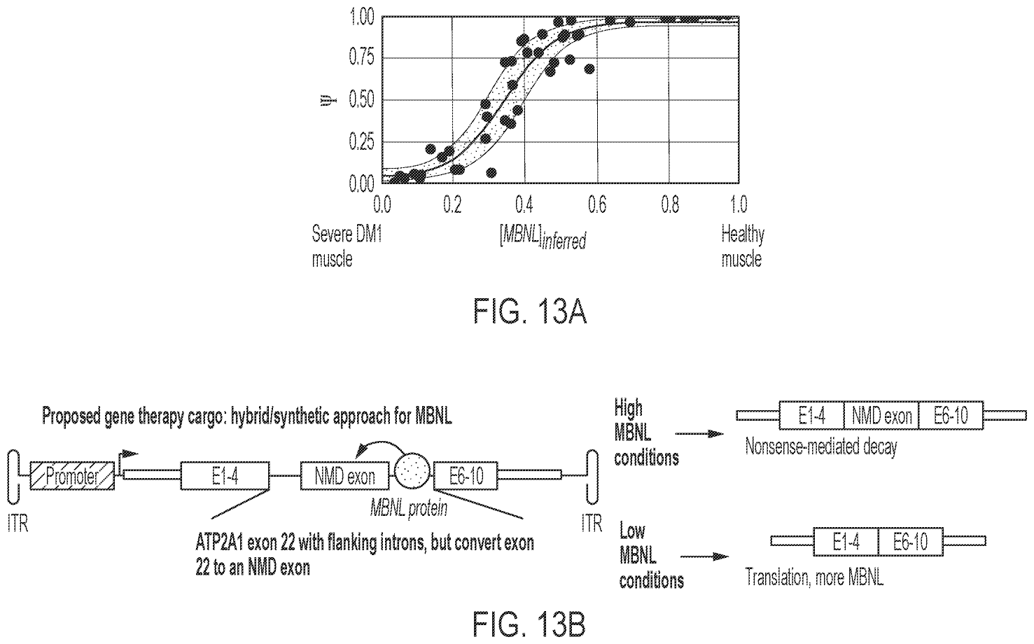

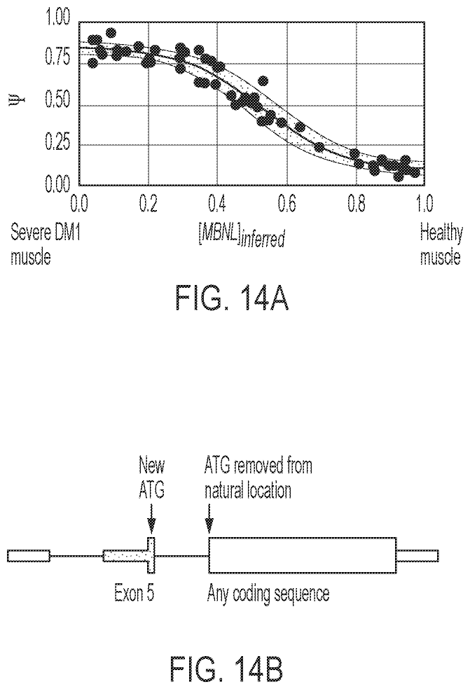

Exon 22 and flanking introns from ATP2A1 were used to elicit nonsense mediated decay (NMD). Exon 22 showed full inclusion in healthy muscle and full exclusion in severe DM1 muscle 31 (FIG. 13A). These exons and their flanking introns were placed into the coding sequence of MBNL, and premature stop codons were introduced into this exon. As a result, production of 2 distinct isoforms was achieved; one in which the exon was skipped, leading to full length functional MBNL, and the second in which the exon was included, leading to nonsense-mediate decay and destruction of the mRNA (FIG. 13B). This synthetic circuit then allowed endogenous MBNL activity to dictate the extent of inclusion of the poison cassette exon, and therefore the total amount of MBNL that was produced from the AAV episome. This approach to elicit NMD can be taken in the context of any AAV cargo, using any combination of coding sequences and/or intron-exon structures.

### Control of MBNL Protein Levels by Moving MBNL1 Exon 5 and Flanking Introns to the 5′ End of the Transgene and Altering the Location of the Start Codon

MBNL1 exon 5 was highly responsive to MBNL activity and it can be used in the context of an AAV backbone as demonstrated above. It is highly excluded in human tibialis muscle and highly included in DM1 tibialis muscle (FIG. 14A). The intron 4-exon 5-intron 5 cassette from AR5.5 was repurposed by moving it to the beginning of the MBNL1 coding sequence, removing the start ATG codon from the MBNL1 coding sequence, and adding an “A” to exon 5 of the AR5.5 cassette such that the last 3 bases of exon 5 then encoded “ATG” (FIG. 14B). The overall effect of these modifications was to cause full length MBNL1 protein to be translated only when exon 5 was included. This construct was tested in the presence of DT480, empty plasmid, or EGFP-MBNL1-269aa in Hela cells. It was observed that the cassette exon was mostly included with DT480 and mostly skipped with EGFP-MBNL1-269aa. Protein output also followed this trend such that high levels of MBNL resulted in little protein production, and low endogenous MBNL activity yields high protein output (FIG. 14C).

### Example 7. Proof of Concept for an Auto-Regulated AAV Cargo in an Animal Model of Disease

AR1.2 and AR5.2 cargoes were tested in a mouse model of myotonic dystrophy, in which expanded CTG repeats were expressed exclusively in skeletal muscle (14). The cargo was packaged into an AAV9 capsid and delivered either systemically by tail vein (intravenous) injection or directly to the muscle by intramuscular injection. This was also compared to coding sequence MBNL1 (the 40 kilodalton isoform, MBNL1-40).

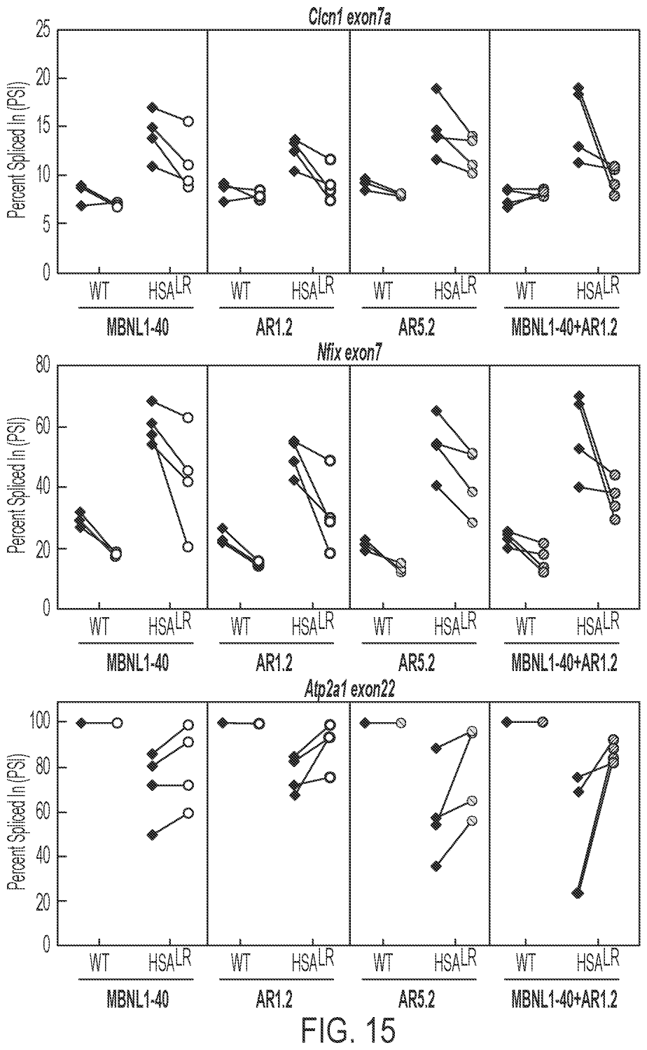

AAV9-MBNL1-40, AAV9-AR1.2, AAV9-AR5.2, and a mixture of AAV9-MBNL1-40 and AAV9-AR1.2 was delivered to WT and HSALR tibialis anterior by intramuscular injection. The contralateral leg was injected with PBS. Tissues were harvested ˜28 days later and processed them to study RNA splicing patterns of MBNL-dependent exons. It was observed that all constructs were capable of splicing rescue, validating their activity in vivo (FIG. 15).

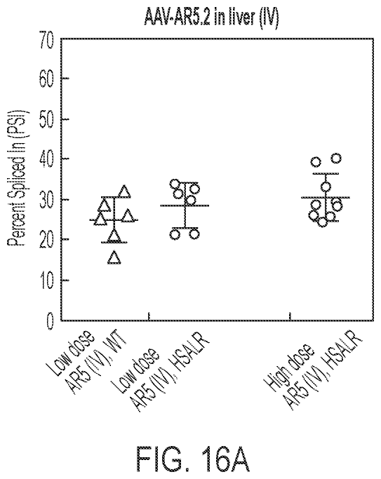

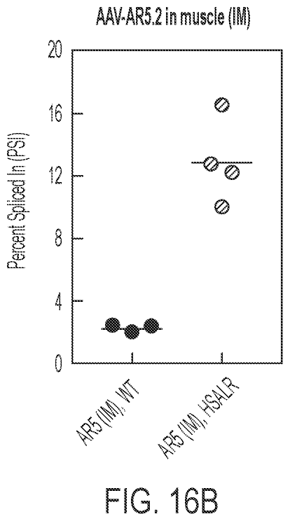

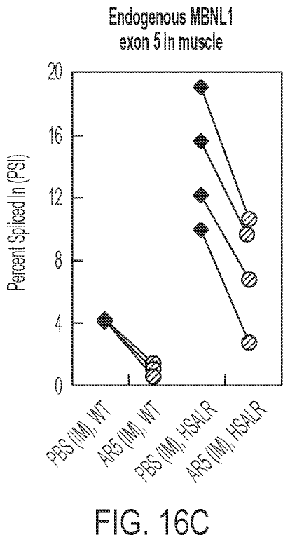

To assess auto-regulatory behavior in vivo, AAV9-AR5.2 was tested. AAV9-AR5.2 was dosed systemically and then harvested liver tissue ˜28 days later. In WT mice, the inclusion levels of the AR5.2 exon 5 cassette were similar to those observed in HSALR mice (FIG. 16A), because the liver did not express the disease transgene in the HSALR model. In the mice in which AAV9-AR5.2 was delivered intramuscularly, inclusion levels of ˜2% were observed in WT and ˜13% in HSALR (FIG. 16B). Inclusion levels of endogenous exon 5 were also assayed and observed that they were ˜2% in WT and ˜9% in HSALR (FIG. 16C). This suggested that the human synthetic AAV cargo exhibited regulation similar to that of the endogenous mouse locus. Furthermore, in the legs injected with AR5.2, splicing rescue of endogenous MBNL1 exon 5 was observed (FIG. 16C), illustrating that similarly to an MBNL coding sequence, AR5.2 produces MBNL protein that is capable of exerting therapeutic effects in a mouse model of myotonic dystrophy.

Overall, these data indicate that auto-regulated AAV constructs not only function as expected in cell culture but also do so in vivo.

### Example 8. Overall Process to Develop Auto-Regulated AAV Cargoes

The auto-regulatory behavior of intron-exon cassettes was confirmed in vitro and in vivo. This process could be broadly applied to a variety of cargoes.

1) First, intron-exon cassettes were identified by analysis of publicly available datasets. Some of these datasets may be RNAseq datasets of different human tissues, cells, or conditions. For example, if expression of a transgene in only one particular tissue or condition is desirable, exons that are either exclusively included or excluded in those tissues or conditions can be identified. This search may result in a shorter list of exons that are amenable to further engineering.

2) Analysis of the intron-exon cassettes was performed to determine the extent of phylogenetic conservation within and around the relevant sequences, because phylogenetic conservation can be used to a) determine how to truncate introns and b) provide confidence that regulatory behavior may be conserved across species. Additionally, splice site strength analysis and analyses of exonic splicing silencers, exonic splicing enhancers, intronic splicing silencers, and intronic splicing enhancers were performed to make informed decisions about where to make truncations or mutations.

3) A minigene that contains either the full-length intron-exon cassette or a truncated version was cloned into an expression cassette. Additional expression plasmids were created that express proteins that are hypothesized to regulate the splicing pattern of the minigene. For example, MBNL1 expression plasmids were used in order to over-express MBNL1 to regulate MBNL1 exon 5 or MBNL1 exon 1. Plasmids that allow for knockdown of relevant proteins may also be useful, e.g. shRNA, microRNA, or CRISPR based approaches down-regulate proteins that may regulate the minigene. In the case of DM1, expression plasmids encoding expanded CTG repeats reduce MBNL levels. The minigene was subjected to perturbations to assess splicing regulatory behavior.

4) Optimization of minigene sequences may be necessary in order to achieve the desired activity. This may involve mutation of splicing regulatory sequences including splice sites or altering intron or exon lengths. Standard assays such as RT-PCR to study isoforms generated and/or potential protein sequences generated should be applied. An iterative process should be performed to achieve the desired regulatory behavior. Library based approaches in which randomized or semi-randomized intronic/exonic sequence (associated with a barcode that can be recovered by RT-PCR and deep sequencing) may be taken, where deep sequencing or amplicon sequencing is used to identify mutations that confer the desired behavior.

5) The alternative splicing cassette may be incorporated into a full-length coding sequence of choice. Protein tags may be added onto the coding sequence to facilitate measurement and detection. The behavior of the alternative splicing cassette and now full-length coding sequence can be assessed in response to over-expression and/or knock down perturbations, or other biological perturbations. Further optimization of the cassette may be necessary to achieve the desired behavior. Again, library-based approaches could be taken in the context of a full-length coding sequence to identify desired sequences.

6) The AAV transgene with optimized cassettes can be tested in vivo by packaging the cargo into the capsid of choice. These viruses can be introduced by various routes to WT or disease animal models. Pharmacodynamic effects can be assessed as appropriate, and auto-regulatory behavior can be assessed by analyzing the appropriate tissues, cell types, or conditions in which the behavior is expected to operate. Auto-regulated cargoes in particular should generate a different dose response curve than non-auto-regulated cargoes. Cargoes that contain cassettes with tissue-specific regulation should also show appropriate activity when comparing tissues. Library based approaches can also be taken in vivo, in which a pool of AAVs encoding distinct intron-exon sequences are introduced; appropriate tissues or cell types can be harvested and splicing or expression behavior of different transgenes can be recovered via deep sequencing across associated barcodes and transgene sequences. This process can be repeated to further optimize cargoes and to identify new behaviors that are desired.

### REFERENCES

1. Heatwole, C., Bode, R., Johnson, N., Quinn, C., Martens, W., McDermott, M. P., Rothrock, N., Thornton, C., Vickrey, B., Victorson, D., & Moxley, 3rd, R. (2012) Patient-reported impact of symptoms in myotonic dystrophy type 1 (PRISM-1). Neurology 79, 348-57.
2. Reardon, W., Newcombe, R., Fenton, I., Sibert, J., & Harper, P. S. (1993) The natural history of congenital myotonic dystrophy: mortality and long-term clinical aspects. Arch Dis Child 68, 177-81.
3. Miller, J. W., Urbinati, C. R., Teng-Umnuay, P., Stenberg, M. G., Byrne, B. J., Thornton, C. A., & Swanson, M. S. (2000) Recruitment of human muscleblind proteins to (CUG)(n) expansions associated with myotonic dystrophy. EMBO J 19, 4439-48.
4. Ho, T. H., Charlet-B, N., Poulos, M. G., Singh, G., Swanson, M. S., & Cooper, T. A. (2004) Muscleblind proteins regulate alternative splicing. EMBO J 23, 3103-12.
5. Batra, R., Charizanis, K., Manchanda, M., Mohan, A., Li, M., Finn, D. J., Goodwin, M., Zhang, C., Sobczak, K., Thornton, C. A., & Swanson, M. S. (2014) Loss of MBNL leads to disruption of developmentally regulated alternative polyadenylation in RNA-mediated disease. Mol Cell 56, 311-322.
6. Taliaferro, J. M., Vidaki, M., Oliveira, R., Olson, S., Than, L., Saxena, T., Wang, E. T., Graveley, B. R., Gertler, F. B., Swanson, M. S., & Burge, C. B. (2016) Distal Alternative Last Exons Localize mRNAs to Neural Projections. Mol Cell 61, 821-33.
7. Wang, E. T., Cody, N. A. L., Jog, S., Biancolella, M., Wang, T. T., Treacy, D. J., Luo, S., Schroth, G. P., Housman, D. E., Reddy, S., Lécuyer, E., & Burge, C. B. (2012) Transcriptome- wide regulation of pre-mRNA splicing and mRNA localization by muscleblind proteins. Cell 150, 710-24.
8. Pascual, M., Vicente, M., Monferrer, L., & Artero, R. (2006) The Muscleblind family of proteins: an emerging class of regulators of developmentally programmed alternative splicing. Differentiation 74, 65-80.
9. Kanadia, R. N., Urbinati, C. R., Crusselle, V. J., Luo, D., Lee, Y.-J., Harrison, J. K., Oh, S. P., & Swanson, M. S. (2003) Developmental expression of mouse muscleblind genes Mbn11, Mbn12 and Mbn13. Gene Expr Patterns 3, 459-62.
10. Tran, H., Gourrier, N., Lemercier-Neuillet, C., Dhaenens, C.-M., Vautrin, A., Fernandez- Gomez, F. J., Arandel, L., Carpentier, C., Obriot, H., Eddarkaoui, S., Delattre, L., Van Brussels, E., Holt, I., Morris, G. E., Sablonniére, B., Buée, L., Charlet-Berguerand, N., Schraen-Maschke, S., Furling, D., Behm-Ansmant, I., Branlant, C., Caillet-Boudin, M.-L., & Sergeant, N. (2011) Analysis of exonic regions involved in nuclear localization, splicing activity, and dimerization of Muscleblind-like-1 isoforms. J Biol Chem 286, 16435-46.
11. Wang, P.-Y., Chang, K.-T., Lin, Y.-M., Kuo, T.-Y., & Wang, G.-S. (2018) Ubiquitination of MBNL 1 Is Required for Its Cytoplasmic Localization and Function in Promoting Neurite Outgrowth. Cell Rep 22, 2294-2306.
12. Wang, P.-Y., Lin, Y.-M., Wang, L.-H., Kuo, T.-Y., Cheng, S.-J., & Wang, G.-S. (2017) Reduced cytoplasmic MBNL1 is an early event in a brain-specific mouse model of myotonic dystrophy. Hum Mol Genet 26, 2247-2257.
13. Kanadia, R. N., Shin, J., Yuan, Y., Beattie, S. G., Wheeler, T. M., Thornton, C. A., & Swanson, M. S. (2006) Reversal of RNA missplicing and myotonia after muscleblind overexpression in a mouse poly(CUG) model for myotonic dystrophy. Proc Natl Acad Sci USA 103, 11748-53.
14. Mankodi, A., Logigian, E., Callahan, L., McClain, C., White, R., Henderson, D., Krym, M., & Thornton, C. A. (2000) Myotonic dystrophy in transgenic mice expressing an expanded CUG repeat. Science 289, 1769-73.
15. Chamberlain, C. M. & Ranum, L. P. W. (2012) Mouse model of muscleblind-like 1 overexpression: skeletal muscle effects and therapeutic promise. Hum Mol Genet 21, 4645-54.
16. Konieczny, P., Stepniak-Konieczna, E., & Sobczak, K. (2018) MBNL expression in autoregulatory feedback loops. RNA Biol 15, 1-8.
17. Bejerano, G., Pheasant, M., Makunin, 1., Stephen, S. Kent, W. J., Mattick, J. S., & Haussler, D. (2004) Ultraconserved elements in the human genome. Science 304, 1321-5.
18. Konieczny, P., Stepniak-Konieczna, E., Taylor, K., Sznajder, L. J., & Sobczak, K. (2017) Autoregulation of MBNL1 function by exon 1 exclusion from MBNL1 transcript. Nucleic Acids Res 45, 1760-1775.
19. Wagner, S. D., Struck, A. J., Gupta, R., Farnsworth, D. R., Mahady, A. E., Eichinger, K., Thornton, C. A., Wang, E. T., & Berglund, J. A. (2016) Dose-Dependent Regulation of Alternative Splicing by MBNL Proteins Reveals Biomarkers for Myotonic Dystrophy. PLoS Genet 12, a 1006316.
20. Lareau, L. F. and Brenner, S. E. (2015) Regulation of Splicing Factors by Alternative Splicing and NMD Is Conserved between Kingdoms Yet Evolutionarily Flexible. 32(4) Mol Bio Evol., 1072-79.
21. Hubé, F. & Francastel, C. (2015). Mammalian introns: when the junk generates molecular diversity. Int J Mol Sci 16, 4429-52.
22. Lin, X., Miller, J. W., Mankodi, A., Kanadia, R. N., Yuan, Y., Moxley, R. T., Swanson, M. S., & Thornton, C. A. (2006). Failure of MBNL1-dependent post-natal splicing transitions in myotonic dystrophy. Hum Mol Genet 15, 2087-97.
18. Konieczny, P., Stepniak-Konieczna, E., Taylor, K., Sznajder, L. J., & Sobczak, K. (2017). Autoregulation of MBNL1 function by exon 1 exclusion from MBNL1 transcript. Nucleic Acids Res 45, 1760-1775.
23. Brais, B., Bouchard, J. P., Xie, Y. G., Rochefort, D. L., Chrétien, N., Tomé, F. M., Lafreniére, R. G., Rommens, J. M., Uyama, E., Nohira, O., Blumen, S., Korczyn, A. D., Heutink, P., Mathieu, J., Duranceau, A., Codére, F., Fardeau, M., Rouleau, G. A., & Korcyn, A. D. (1998). Short GCG expansions in the PABP2 gene cause oculopharyngeal muscular dystrophy. Nat Genet 18, 164-7.
24. Banerjee, A., Apponi, L. H., Pavlath, G. K., & Corbett. A. H. (2013). PABPN1: molecular function and muscle disease. FEBS J 280, 4230-50.
25. Riaz, M., Raz, Y., van Putten, M., Paniagua-Soriano, G., Krom, Y. D., Florea, B. 1., & Raz, V. (2016). PABPN1-Dependent mRNA Processing Induces Muscle Wasting. PLoS Genet 12, e1006031.
26. Apponi, L. H., Leung, S. W., Williams, K. R., Valentini, S. R., Corbett, A. H., & Pavlath, G. K. (2010). Loss of nuclear poly(A)-binding protein 1 causes defects in myogenesis and mRNA biogenesis. Hum Mol Genet 19, 1058-65.
27. Chartier, A., Benoit, B., & Simonelig, M. (2006). A Drosophila model of oculopharyngeal muscular dystrophy reveals intrinsic toxicity of PABPN1. EMBO J 25, 2253 62.
28. Banerjee, A., Vest, K. E., Pavlath, G. K., & Corbett, A. H. (2017). Nuclear poly(A) binding protein 1 (PABPN 1) and Matrin3 interact in muscle cells and regulate RNA processing. Nucleic Acids Res 45, 10706-10725.
29. Banerjee, A., Phillips, B. L., Deng, Q., Seyfried, N. T., Pavlath, G. K., Vest, K. E., & Corbett, A. H. (2019). Proteomic analysis reveals that wildtype and alanine-expanded nuclear poly(A)- binding protein exhibit differential interactions in skeletal muscle. J Biol Chem 294, 7360-7376.
30. Malerba, A., Klein, P., Lu-Nguyen, N., Cappellari, O., Strings-Ufombah, V., Harbaran, S., Roelvink, P., Suhy, D., Trollet, C., & Dickson, G. (2019). Established PABPN1 intranuclear inclusions in OPMD muscle can be efficiently reversed by AAV-mediated knockdown and replacement of mutant expanded PABPN 1. Hum Mol Genet 28, 3301-3308.
31. Abu-Baker, A., Kharma, N., Perreault, J., Grant, A., Shekarabi, M., Maios, C., Dona, M., Neri, C., Dion, P. A., Parker, A., Varin, L., & Rouleau, G. A. (2019). RNA-Based Therapy Utilizing Oculopharyngeal Muscular Dystrophy Transcript Knockdown and Replacement. Mol Ther Nucleic Acids 15, 12-25.
32. Malerba, A., Klein, P., Bachtarzi, H., Jarmin, S. A., Cordova, G., Ferry, A., Strings, V., Espinoza, M. P., Mamchaoui, K., Blumen, S. C., St Guily, J. L., Mouly, V., Graham. M., Butler- Browne, G., Suhy, D. A., Trollet, C., & Dickson, G. (2017). PABPN 1 gene therapy for oculopharyngeal muscular dystrophy. Nat Commun 8, 14848.
33. Corbeil-Girard, L.-P., Klein, A. F. Sasseville, A. M.-J., Lavoie, H., Dicaire, M.-J., Saint- Denis, A., Pagé, M., Duranceau, A., Codére, F., Bouchard, J.-P., Karpati, G., Rouleau, G. A., Massie, B., Langelier, Y., & Brais, B. (2005). PABPN 1 overexpression leads to upregulation of genes encoding nuclear proteins that are sequestered in oculopharyngeal muscular dystrophy nuclear inclusions. Neurobiol Dis 18, 551-67
34. Bergeron, D., Pal, G., Beaulieu, Y. B., Chabot, B., & Bachand, F. (2015). Regulated Intron Retention and Nuclear Pre-mRNA Decay Contribute to PABPN 1 Autoregulation. Mol Cell Biol 35, 2503-17.
35. Kapeli, K., Martinez, F. J., & Yeo, G. W. (2017). Genetic mutations in RNA-binding proteins and their roles in ALS. Hum Genet 136, 1193-1214.
36. Purice, M. D. & Taylor, J. P. (2018). Linking hnRNP Function to ALS and FTD Pathology. Front Neurosci 12, 326.
37. Zhao, M., Kim, J. R., van Bruggen, R., & Park, J. (2018). RNA-Binding Proteins in Amyotrophic Lateral Sclerosis. Mol Cells 41, 818-829.
38. Verdile, V., De Paola, E., & Paronetto, M. P. (2019). Aberrant Phase Transitions: Side Effects and Novel Therapeutic Strategies in Human Disease. Front Genet 10, 173.
39. Li, Y. R., King, O. D., Shorter, J., & Gitler, A. D. (2013). Stress granules as crucibles of ALS pathogenesis. J Cell Biol 201, 361-72.
40. Calabretta, S. & Richard, S. (2015). Emerging Roles of Disordered Sequences in RNABinding Proteins. Trends Biochem Sci 40, 662-672.
41. Suzuki, H. & Matsuoka, M. (2017). hnRNPA1 autoregulates its own mRNA expression to remain non-cytotoxic. Mol Cell Biochem 427, 123-131.
42. Xu, Y.-F., Gendron, T. F., Zhang, Y.-J., Lin, W.-L., D'Alton, S., Sheng, H., Casey, M. C., Tong, J., Knight, J., Yu, X., Rademakers, R., Boylan, K., Hutton, M., McGowan, E., Dickson, D. W., Lewis, J., & Petrucelli, L. (2010). Wild-type human TDP-43 expression causes TDP-43 phosphorylation, mitochondrial aggregation, motor deficits, and early mortality in transgenic mice. J Neurosci 30, 10851-9.
43. Mitchell, J. C., McGoldrick, P., Vance, C., Hortobagyi, T., Sreedharan, J., Rogelj, B., Tudor, E. L., Smith, B. N., Klasen, C., Miller, C. C. J., Cooper, J. D., Greensmith, L., & Shaw, C. E. (2013). Overexpression of human wild-type FUS causes progressive motor neuron degeneration in an age- and dose-dependent fashion. Acta Neuropathol 125, 273-88.
44. Ling, S.-C., Dastidar, S. G., Tokunaga, S., Ho, W. Y., Lim, K., Ilieva, H., Parone, P. A., Tyan, S. H., Tse, T. M., Chang, J.-C., Platoshyn, O., Bui, N. B., Bui, A., Vetto, A., Sun, S., McAlonis- Downes, M., Han, J. S., Swing, D., Kapeli, K., Yeo, G. W., Tessarollo, L., Marsala, M., Shaw. C. E., Tucker-Kellogg, G., La Spada, A. R. Lagier-Tourenne, C., Da Cruz, S., & Cleveland, D. W. (2019). Overriding FUS autoregulation in mice triggers gain-of-toxic dysfunctions in RNA metabolism and autophagy-lysosome axis. Elife 8.
45. Barmada, S. J., Skibinski, G., Korb, E., Rao, E. J., Wu, J. Y., & Finkbeiner. S. (2010). Cytoplasmic mislocalization of TDP-43 is toxic to neurons and enhanced by a mutation associated with familial amyotrophic lateral sclerosis. J Neurosci 30, 639-49.
46. Humphrey, J. ( ). FUS ALS-causative mutations impact FUS autoregulation and the processing of RNA-binding proteins through intron retention. https://doi.org/10.1101/567735.
47. Zhou, Y., Liu, S., Liu, G., Oztürk, A., & Hicks, G. G. (2013). ALS-associated FUS mutations result in compromised FUS alternative splicing and autoregulation. PLoS Genet 9, e1003895.
48. Kim, H. J., Kim, N. C., Wang, Y.-D., Scarborough, E. A., Moore, J., Diaz, Z., MacLea, K. S., Freibaum, B., Li, S., Molliex, A., Kanagaraj, A. P., Carter, R., Boylan, K. B., Wojtas, A. M., Rademakers, R., Pinkus, J. L., Greenberg, S. A., Trojanowski, J. Q., Traynor, B. J., Smith, B. N., Topp, S., Gkazi, A. S., Miller, J., Shaw, C. E., Kottlors, M., Kirschner, J., Pestronk, A., Li, Y. R., Ford, A. F., Gitler, A. D., Benatar, M., King, O. D., Kimonis, V. E., Ross, E. D., Weihl, C. C., Shorter, J., & Taylor, J. P. (2013). Mutations in prion-like domains in hnRNPA2B 1 and hnRNPA1 cause multisystem proteinopathy and ALS. Nature 495, 467-73.
49. Jean-Philippe, J., Paz, S., & Caputi, M. (2013). hnRNP A 1: the Swiss army knife of gene expression. Int J Mol Sci 14, 18999-9024.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential characteristics of the present disclosure, and without departing from the spirit and scope thereof, can make various changes and modifications of the present disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined. i.e., elements that are conjunctively present in some cases and disjunctively present in other cases. Multiple elements listed with “and/or” should be construed in the same fashion. i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B): in another embodiment, to B only (optionally including elements other than A); in yet another embodiment, to both A and B (optionally including other elements): etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. An rAAV comprising a nucleic acid encoding an RNA, wherein the RNA comprises a first intron, wherein splicing of the first intron is regulated by an intracellular factor.

2. The rAAV of claim 1, further comprising a second intron.

3. The rAAV of claim 1 or claim 2, wherein the nucleic acid encodes the intracellular factor.

4. The rAAV of claim 3, wherein the splicing of the intron is regulated by the encoded intracellular factor.

5. The rAAV of claim 3, wherein the splicing of the intron is regulated by an intracellular factor that is not encoded by the RNA.

6. The rAAV of any one of claims 1-5, wherein the intracellular factor is a protein, an RNA, or a protein-RNA complex.

7. The rAAV of claim 6, wherein the protein comprises a tissue-specific RNA binding protein, an autoregulatory RNA binding protein, or a condition-specific RNA binding protein.

8. The rAAV of claim 6 or claim 7, wherein the intracellular protein comprises an MBNL protein, an SR protein, an hnRNP protein, an RbFox protein, a CELF protein, a Nova protein, or a PTB protein.

9. The rAAV of any one of claims 6-8, wherein the protein is any one of MBNL1, MBNL2, MBNL3, hnRNP A1, hnRNP A2B1, hnRNP C, hnRNP D, hnRNP DL, hnRNP F, hnRNP H, hnRNP K, hnRNP L, hnRNP M, hnRNP R, hnRNP U, FUS, TDP43, PABPN1, ATXN2, TAF15, EWSR1, MATR3, TIA1, FMRP, MTM1, KIF5A, microdystrophin, C9ORF72, HTT, DNM2, BIN1, RYR1, NEB, ACTA, TPM3, TPM2, TNNT2, CFL2, KBTBD13, KLHL40, KLHL41, LMOD3, MYPN, SEPN1, TTN, SPEG, MYH7, TK2, POLG1, GAA, AGL, PYGM, SLC22A5, OCTN2, ETF, ETFH, PNPLA2, cytochrome b/cytochrome c oxidase, CLCN 1, SCN4A, DMPK, CNBP, MYOT, LMNA (Lamin A/C), CAV3, DNAJB6, DES, TNPO3, HNRPDL, CAPN3, DYSF, alpha-sarcoglycan, beta-sarcoglycan, gamma-sarcoglycan, delta-sacroglycan, TCAP, TRIM32, FKRP, POMT1, FKTN, POMT2, POMGnT1, DAG 1, ANO5, PLEC 1, TRAPPC 11, GMPPB, ISPD, LIMS2, POPDC1, TOR 1 A1P1, POGLUT2, LAMA2, COL6A1, POMT1, POMT2, DUX4, EMD, PAX7, PMP22, MPZ, MFN2, SMCHD1, or GJB1, or a truncated version thereof.

10. The rAAV of claim 6, wherein the RNA comprises a regulatory RNA molecule, a short hairpin RNA molecule, a microRNA molecule, a transfer RNA molecule, or a ribosome.

11. The rAAV of claim 6, wherein the protein-RNA complex comprises a ribosome, snRNP complex, or other macromolecular complex that contains at least one protein bound to at least one RNA, optionally wherein the snRNP complex is U1 snRNP or U2 snRNP.

12. The rAAV of any one of claims 1-11, wherein the first and/or second intron flanks an alternatively regulated exon and/or prevents RNAs from exiting the nucleus.

13. The rAAV of any one of claims 1-12, wherein the first and/or second intron is a truncated version of a naturally occurring intron.

14. The rAAV of any one of claims 2-13, wherein the first and/or second intron is or is derived from any one or more of: an NMD exon-flanking intron of SmB/B′, an exon 2b-flanking intron of SMN, an intron 3 of SMN, a 3′ UTR intron of hnRBP A2BJ, an NMD exon-flanking intron of Tia1, an exon 7-flanking intron of Bin1, an exon 11-flanking intron of Bin1, an alternative exon-flanking intron of hnRNP D, an exon 13-flanking intron of FMRP, an exon 14-flanking intron of FMRP, an exon 15-flanking intron of FMRP, an alternative exon-flanking intron of Lamin A/C, an exon 11-flanking intron of S77, an alternative exon-flanking intron of Matrin 3, an alternative exon-flanking intron of NEXN, an alternative exon-flanking intron of NRAP, an alternative exon-flanking intron of MTM1, an exon 9-flanking intron of CACNA1C, an exon T2-flanking intron of MBNL1, an exon 1-flanking intron of MBNL1, an exon 2-flanking intron of MBNL1, an exon 3-flanking intron of MBNL1, an exon 4-flanking intron of MBNL1, an exon 5-flanking intron of MBNL1, an exon 6-flanking intron of MBNL1, an exon 7-flanking intron of MBNL1, an exon 9-flanking intron of MBNL1, an intron 6 of PABPN1, an intron 6 of TDP43, an intron 7 of TDP43, an intron 6 of FUS, an intron 7 of FUS, an intron 10 of hnRNP A1, and/or an exon 22-flanking intron of ATP2A1.

15. The rAAV of any one of claims 1-14, wherein the first and/or second intron comprises a 5′ splice donor site, optionally wherein the 5′ splice donor site is a GU or an AU.

16. The rAAV of any one of claims 1-15, wherein the first and/or second intron comprises a 3′ splice acceptor site, optionally wherein the 3′ splice acceptor site is an AG or an AC.

17. The rAAV of any one of claims 1-16, wherein the first and/or second intron comprises a region that regulates intron splicing.

18. The rAAV of claim 17, wherein the region that regulates intron splicing comprises one or more binding sites for a protein that regulates intron splicing.

19. The rAAV of any one of claims 1-18, further comprising an RNAi that targets a chromosomal allele encoding a gene encoding the intracellular factor.

20. The rAAV of any one of claims 1-19, further comprising an exon.

21. The rAAV of claim 20, wherein the exon is flanked by at least the first intron, optionally wherein the exon is flanked by the first and second intron.

22. The rAAV of claim 21, wherein the intracellular factor is a protein, wherein the exon comprises an open reading frame that encodes a portion of the protein.

23. The rAAV of any one of claims 19-22, wherein the exon is naturally occurring.

24. The rAAV of any one of claims 19-22, wherein the exon is a recombinant exon.

25. The rAAV of claim 24, wherein the recombinant exon comprises two or more naturally-occurring exons that are fused together without any intervening introns.

26. The rAAV of any one of claims 19-25, further comprising a regulatory exon, wherein the regulatory exon comprises an in-frame stop codon that is at least 50 nucleotides upstream of the 5′ splice junction of the regulatory exon.

27. The rAAV of any one of claims 19-26, wherein the exon is or is derived from any one or more of: an NMD exon of SmB/B′, an exon 2b of SMN, an exon 3 of SMN, an exon 4 of SMN, an hnRBP A2B1 exon, an NMD exon of Tia1, an exon 7 of Bin1, an exon 11 of Bin1, an alternative exon of hnRNP D, an exon 13 of FMRP, an exon 14 of FMRP, an exon 15 of FMRP, an alternative exon of Lamin A/C, an exon 11 of ST7, an alternative exon of Matrin 3, an alternative exon of NEXN, an alternative exon of NRAP, an alternative exon of MTM1, an exon 9 of CACNA1C, an exon T2 of MBNL1, an exon 1 of MBNL1, an exon 2 of MBNL1, an exon 3 MBNL1, an exon 4 of MBNL1, an exon 5 of MBNL1, an exon 6 of MBNL1, an exon 7 of MBNL1, an exon 9 MBNL1, an exon 6 of PABPN1, an exon 7 of PABPN1, an exon 6 of TDP43, an exon 7 of TDP43, an exon 8 of TDP43, an exon 6 of FUS, an exon 7 of FUS, an exon 8 of FUS, an exon 10 of hnRNP A1, an exon 11 of hnRNP A1, and/or an exon 22 of ATP2A1.

28. The rAAV of any one of claims 19-27, wherein all introns and all exons are from the same gene.

29. The rAAV of any one of claims 19-27, wherein at least one intron and at least one exon are from different genes.

30. The rAAV of claim 28 or claim 29, wherein the gene(s) comprises any one or more of: MBNL1, MBNL2, MBNL3, hnRNP A1, hnRNP A2B1, hnRNP C, hnRNP D, hnRNP DL, hnRNP F, hnRNP H, hnRNP K, hnRNP L, hnRNP M, hnRNP R, hnRNP U, FUS, TDP43, PABPN1, ATXN2, TAF15, EWSR1, MATR3, TIA1, FMRP, MTM1, KIF5A, a microdystrophin-encoding gene, C9ORF72, HIT, DNM2, BIN1, RYR1, NEB, ACTA, TPM3, TPM2, TNNT2, CFL2, KBTBD13, KLHL40, KLHL41, LMOD3, MYPN, SEPN1, TTN, SPEG, MYH7, TK2, POLG1, GM, AGL, PYGM, SLC22A5, OCTN2, ETF, ETFH, PNPLA2, a cytochrome b oxidase-encoding gene, a cytochrome c oxidase-encoding gene, CLCN1, SCN4A, DMPK, CNBP, MYOT, LMNA, CAV3, DNAJB6, DES, TNPO3, HNRPDL, CAPN3, DYSF, an alpha-sarcoglycan-encoding gene, a beta-sarcoglycan-encoding gene, a gamma-sarcoglycan-encoding gene, a delta-sacroglycan-encoding gene, TCAP, TRIM32, FKRP, POMT1, FKTN, POMT2, POMGnT1, DAG1, ANO5, PLEC1, TRAPPC11, GMPPB, ISPD, LIMS2, POPDC1, TOR1AIP1, POGLUT2, LAMA2, COL6A1, POMT1, POMT2, DUX4, EMD, PAX7, PMP22, MPZ, MFN2, SMCHD1, and/or GJB1.

31. The rAAV of any one of claims 1-30, further comprising a promoter.

32. The rAAV of claim 31, wherein the promoter is a constitutive promoter or a regulated promoter.

33. The rAAV of claim 32, wherein the regulated promoter is an inducible promoter.

34. The rAAV of any one of claims 31-33, wherein the promoter comprises any one of: CMV, EF1alpha, CBh, synapsin, enolase, MECP2, MHCK7, Desmin, GFAP.

35. The rAAV of any one of claims 1-34, wherein the nucleic acid is flanked by adeno-associated virus (AAV) inverted terminal repeat (ITR) sequences.

36. The rAAV of claim 35, wherein the AAV ITR sequences comprise AAV 1, AAV2, AAV5, AAV7, AAV8, or AAV9 ITR sequences.

37. A method of treating a disease or condition in a subject comprising administering the rAAV of any one of claims 1-36 to the subject.

38. The method of claim 37, wherein the subject is a human subject.

39. The method of claim 37 or 38, wherein the disease or condition is selected from the group consisting of: a repeat expansion disease, a laminopathy, a cardiomyopathy, a muscular dystrophy, a neurodegenerative disease, a cancer, an intellectual disability, and/or premature aging.

40. The method of any one of claims 37-39, wherein the disease or condition is selected from the group consisting of: Dentatorubral-pallido-luysian atrophy (DRPLA), myotonic dystrophy type 1 (DM1), myotonic dystrophy type 2 (DM2), Fragile X syndrome of mental retardation (FMR1), Fragile X tremor ataxia syndrome (FXTAS), FRAXE mental retardation (FMR2), Friedreichs ataxia (FRDA), Huntington disease (HD), Huntington disease-like 2 (HDL2), Oculopharyngeal muscular dystrophy (OPMD), Myoclonic epilepsy type 1, Alzheimer's disease, ALS/FTD, spinocerebellar ataxia type 1 (SCA1), spinocerebellar ataxia type 2 (SCA2), spinocerebellar ataxia type 3 (SCA3), spinocerebellar ataxia type 6 (SCA6), spinocerebellar ataxia type 7 (SCAT), spinocerebellar ataxia type 8 (SCA8), spinocerebellar ataxia type 10 (SCA10), spinocerebellar ataxia type 12 (SCA12), spinocerebellar ataxia type 17 (SCA17), Syndromic/non-syndromic X-linked mental retardation, Emery-Dreifuss muscular dystrophy type 2, familial partial lipodystrophy, limb girdle muscular dystrophy type 1B, dilated cardiomyopathy, familial partial lipodystrophy, Charcot-Marie-Tooth disorder type 2B1, mandibuloacral dysplasia, childhood progeria syndrome (Hutchinson-Gilford syndrome), Werner syndrome, Dilated cardiomyopathy (DCM), Hypertrophic cardiomyopathy (HCM), Restrictive cardiomyopathy (RCM), Left Ventricular Non-compaction (LVNC), Arrhythmogenic Right Ventricular Dysplasia (ARVD), takotsubo cardiomyopathy, Duchenne muscular dystrophy, Becker muscular dystrophy, Limb-girdle muscular dystrophy, Facioscapulohumeral muscular dystrophy, Congenital muscular dystrophy, Oculopharyngeal muscular dystrophy, Distal muscular dystrophy, Emery-Dreifuss muscular dystrophy, dementia, Parkinson's disease (PD), a PD-related disorder, Prion disease, a motor neuron disease (MND), Progressive bulbar palsy (PBP), Progressive muscular atrophy (PMA), Primary lateral sclerosis (PLS), Spinal muscular atrophy (SMA), a bladder cancer, a breast cancer, a colorectal cancer, a kidney cancer, a lung cancer, a lymphoma, a melanoma, an oral cancer, an ovarian cancer, an oropharyngeal cancer, a pancreatic cancer, a prostate cancer, a thyroid cancer, a uterine cancer, Down syndrome, Prader-Willi Syndrome (PWS), Bloom Syndrome, Cockayne Syndrome Type I-216400, Cockayne Syndrome Type III, Cockayne Syndrome Type I, Hutchinson-Gilford Progeria Syndrome, Mandibuloacral Dysplasia with Type A Lipodystrophy, Progeria, Adult Onset Progeroid Syndrome, Neonatal Rothmund-Thomson Syndrome, Seip Syndrome, Werner Syndrome, Replication Focus-Forming Activity 1, and/or centronuclear myopathy.

41. The method of any one of claims 37-40, wherein the rAAV is administered to the subject at least one time, optionally wherein the rAAV is administered to the subject multiple times.

42. The method of any one of claims 37-41, wherein the rAAV is administered by intravenous injection, intramuscular injection, intrathecal injection, or intravitreal injection.

43. An rAAV comprising a recombinant MBNL gene, wherein the recombinant MBNL gene comprises:
a. an MBNL protein coding sequence, and
b. at least one truncated intron of the MBNL gene,
wherein splicing of the truncated intron is regulated by an intracellular protein.

44. The rAAV of claim 43, wherein the MBNL gene is MBNL1, MBNL2, or MBNL3.

45. The rAAV of claim 43 or claim 44, further comprising at least one exon.

46. The rAAV of claim 45, wherein the at least one exon is exon 1 and/or exon 5 of the MBNL gene.

47. The rAAV of any one of claims 43-46, wherein the at least one truncated intron is a truncated exon 1-flanking intron and/or a truncated exon 5-flanking intron of the MBNL gene.

48. The rAAV of any one of claims 43-47, wherein the at least one truncated intron comprises:
a. SEQ ID NOs: 2 and 4, but does not comprise SEQ ID NO: 3;
b. SEQ ID NOs: 6 and 8, but does not comprise SEQ ID NO: 7;
c. SEQ ID NOs: 11 and 13, but does not comprise SEQ ID NO: 12; or
d. SEQ ID NOs: 15 and 17, but does not comprise SEQ ID NO: 16.

49. The rAAV of claim 45, wherein the at least one exon is exon 3 or exon 9 of the MBNL gene.

50. The rAAV of claim 45, wherein the at least one exon is exon 8 and/or exon 10 of the MBNL gene.

51. The rAAV of claim 45, wherein the at least one exon is exon 22 of the ATPA1 gene.

52. The rAAV of claim 51, further comprising one or more exon 22-flanking introns of the ATPA1 gene.

53. The rAAV of any one of claims 43-52, further comprising a promoter.

54. The rAAV of claim 53, wherein the promoter is an endogenous or an exogenous promoter.

55. The rAAV of claim 54, wherein the exogenous promoter is any one of an: EF1 alpha promoter, beta actin promoter, MHCK7, CBh, synapsin, MECP2, enolase, GFAP, Desmin, and CAG promoter.

56. The rAAV of any one of claims 43-55, further comprising an endogenous or an exogenous 3′ untranslated region (UTR).

57. The rAAV of claim 56, wherein the exogenous 3′ UTR is the 3′ UTR from bovine growth hormone, SV40, EBV, or Myc.

58. The rAAV of any one of claims 43-57, wherein the expression construct comprises the second 5′ UTR of the MBNL gene, and does not include the first or the third 5′ UTR of the MBNL gene.

59. The rAAV of any one of claims 43-58, wherein the recombinant MBNL gene is flanked by adeno-associated virus (AAV) inverted terminal repeat (ITR) sequences.

60. The rAAV of claim 59, wherein the AAV ITR sequences comprise AAV 1, AAV2, AAV5, AAV7, AAV8, or AAV9 ITR sequences.

61. A method of treating an MBNL-related disease or condition in a subject, comprising administering an rAAV according to any one of claims 43-60 to the subject.

62. The method of claim 61, wherein the rAAV is administered to the subject at least one time, optionally wherein the rAAV is administered to the subject multiple times.

63. The method of claim 61 or 62, wherein the rAAV is administered by intravenous injection, intramuscular injection, intrathecal injection, or intravitreal injection.

64. The method of any one of claims 61-63, wherein the MBNL-related disease or disorder is any one of Fuch's endothelial corneal dystrophy, Huntington Disease, or a cancer.

65. An rAAV comprising a nucleic acid having the sequence of any one or more of SEQ ID NOs: 1, 2, 4-6, 8-11, 13-15, and/or 17-49.


SEQUENCE LISTING (49 sequences)

SEQ ID NO: 1 - DNA, 186 bp, Homo Sapiens
agcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatg

SEQ ID NO: 2 - DNA, 136 bp, Homo Sapiens
gtgagtagagatatcagctctctccttgttagcagtcagaaaagcaaagtgagcaactatatctgactacaagctattcatttagtaacctttttaaaaaaattgctgaagatatgtttgttcaggtatcccagac

SEQ ID NO: 3 - DNA, 673 bp, Homo Sapiens
aatatataaagaagttactttcacagaagtgagggtaactcctcaaatggttcagattgctcctactcttggcctaactcttaatctgtcttattgtggatgtagggtttctgaaatctctgctttgtaaagaaccacctgtctccttttcaacttaacgatattgccattttcagtcttcttatttctgtgtgccatctaatcagccattgttttcctttggtgtgttcttttgcacatcaaggctcttctttacatatatcttgcttttaacaataacatgatctggaactatttaggagaattgttcaacaaaattgttcttagaattagctacaggattttgagtagtatactatctagattgtctgaaataatattgtggcataaacataatagtaacactagagaagcttgactatgccattatatagacaggagagagacagcagtactcaaggagttagaaattggatcattaagaatagctactaaactatattctcaaggtaaattttaatgtgatctagtagctgattttaatgagcccattctttgttctttgcttacctgttttaatgttgtcatttttttacataaatatatgacttaaatcaatgaaatactcattttaaaagtcactgtgtagtatttcataacacagtgaaataat

SEQ ID NO: 4 - DNA, 355 bp, Homo Sapiens
cacgagagagatcttttctgtgtttatggatacttgagcaaaaatacagaaggcagactctctcctcctctcttcctttcactcttttttttttctgttagagtatcttgtttgtaattaactacaaagaggagttatcctcccaataacaactcagtagtgcctttattgtgcatgcttagtcttgttattcgttgtatatggcattccgatgatttgtttttttatttgttttttctcacctacccaaaaatgcactgctgcccccatgatgcacctctgcttgctgtttatgttaatgcgcttgaaccccactggcccattgccatcatgtgctcgctgcctgctaattaag

SEQ ID NO: 5 - DNA, 54 bp, Homo Sapiens
actcagtcggctgtcaaatcactgaagcgacccctcgaggcaacctttgacctg

SEQ ID NO: 6 - DNA, 47 bp, Homo Sapiens
gtactatgacctttcaccttttagcttggcatgtagctttattgtag

SEQ ID NO: 7 - DNA, 741 bp, Homo Sapiens
atacaagttttttttttaaatcaactttaaaatatatatccttttttctgttatagagttgtaaagtacaatgaaaaaactgagtgtggtttcctgacaaaattagtagaaagactataatctaagtacatagatggatatcatacaataaaagattctgaaagcccagcagcccacattcagtttaactacattgtagaatgttctgaagaaatagtcggaggacataaagagtgtatttttgactgtatatttatgtacctgtttggttagcatggctttagtggtagaattatttacattttaatatttatagttcagtatttaaggaaaaaaacttgcatgctctgggacatttgcatgtctaggtagatctggggggaggagcagagtgggttagtttttattttatctgtttcagtcactctgtacaattaactattaacattgcttatacttgtcaatcctcatggagctcgcagtgcggcttttcttcttttcccaaatcaatggcttctcacagaaagcctgaagcattatgggacataaagcactttaacacattataacatgtgctcttgtactagtgagattttgttttggacaaaaataatgcttcctttaaagcttttatcttgctttttttttttttttcctccttaattttttgttcattggattttttccctcgggtagttaagtgctctgctgcttgcttgctcatgcttcctaacaatttta

SEQ ID NO: 8 - DNA, 74 bp, Homo Sapiens
gccttcgactgatttttcttttttctttttctctttttactggtatttgttttttatactcattcactaaacag

SEQ ID NO: 9 - DNA, 24 bp, Homo Sapiens
ggaattcctcaagctgtacttacc

SEQ ID NO: 10 - DNA, 150 bp, Homo Sapiens
agctgaatgagttgtggcgcccacaatgctcccatgacaaggagctgacaagttccattttccgtcgcgggcatcttggaatcatgactcccacaatgccttgggcacttggtcgacagtggggccgcctctgaaaaaaaaatgtgagag

SEQ ID NO: 11 - DNA, 150 bp, Homo Sapiens
gtaagtttccattttcacagtttccccgcgccgcttcattgttcggactccggcgggtctgcccgtggctgaaggaggaagtgcgaggaggtgctcgccggccgcggttctcccgggcaggggcgggccgctcgcggtagttggtttcgc

SEQ ID NO: 12 - DNA, 30052 bp, Homo Sapiens
gtccctcagcacctcctgcccgggggaaggcgggagggtgagccgcagccgctcctgcgcccttccctgccgcgggctctgcggacgctgtcacggcacagcccgcgcacggcttggcagcccagcgggacccagggagaaacaggggggcgagaagagaaacaggtgcgtgcaagtgtactcacaacttaatgacctttgtcgggggaaggtgtgagtccctctgagtcgagcggcgcgcgggtcttcccggcggctcccgcatccctggccccggggctgccatcccgcccggttttcatctgggccaaatatgtgcacgcaaaggttaattctctgcttgctacccataaattacgcatgacatgtttagctgggctgccttatatggagagtgctgtatttcaggccagtttgccttttgccaagtttacagggcggaagacgccctgtaggagagggctgcctcctccttttcttcttcttcttttttttttcatcgttttgtagtgcgtcactcaaaaactgtaagaaaagtaagtatagaagctctttgggtggagccgcagttggttgaattgctttcatgaaaggttgaaaagaatgaaaagtagttagtacttgggttctggtttttagacaaaagcaggacaaatatgtagccaccccccaacttttttttttttttttaaacggagccggaatctgttattggtagtaatgtgtcatgtgagaatactttgattccagagcctgtaacacgagcaggtatatggtggcgcttatgccttcccctcgcacagcactttacaactacaggggtgttgctgccagagtaatattgtattcttaaggctctctggggccgggagagtatgcggctgttgggattctggggtgtatttccgtggagtatgtctccccaggattaaagttaagaagctgccggaaaggttgttgccgctgacttcactttgtgtgcccaccagcacaggcacaagtcatagcagatggatggcaaactctgccatatactctgaggagtttcttaaattaatacgctaggagagaagtcataattaatgcttttagactttcgagccctctgcagcgtgctttgaaatactacaccggtggacactatgtcccttcctcctgggtttgaacacagtgtctgtttatacacagagggtgtttagtacctaagtattctataccagcacaagagagccactccacagttatctctctgtgtggattcctaagtgtgttggtgtttctccttgctgttgagataaaatattatcacccagagagaaaatgaaagttcaacgactgtaaaatttatcccaggaatgtgcagttcaatacttagaatgtcttagatgtagttatagatagaggcactgaataagttattttgtagcctgagaatgcacatcgctgtcacttgtatcttgctttcttaaagttggtgacaactaaggctaaacttctttagaaaacctaatgttctagtttttatctgttttgtgtatttgcatgggagttactttattattattattatattgttaggtcttctttgaaaatgtaggcatagcttttttagtcttatttgtttaatggggggtttccttgctgaagactacaaaagttgccttttgtgttttaaggaacaggttgggtcagcaacagaaaacctgttcatctataattatagtgtcacttaagacataataatcagatttcattattactaagagactcagaaaaacctcacatatgcctagtttaatttttttccttatgtaactgtgaacaaatttgtgtgtagatacacatcaagtgtggtactctgcgcagtcatgccaaagagaaactgaagtgttatatggtttgagatattattgcttgtgtttacatttccacatttgaggcaaagttagactccaagttgaattcattaaattttgattccaacattctgttcttcctttgggcacagaattcctatgaaatgaaacactgggttttgttccagaacaagaacagactaacccaaaatttctcctgggggaaaaaaaaagagtaaaattatatttctcattgaaaattgtatttctcattgaggaagggtgtgttactttattatataaaatgccttatcttcattataagaaaaaaatgcccttgcatttgtcactgttgccatacattaggcctgtatttgatagatctatatggaaacagagaataaataatcttgctgcaccttctggctttctgcttggtgatagatgtgcggattagagaacatctcaggtcttggttaggagaagctgaaaagaataggtgattgggtttttaaaattctgtatgcaaaggcattagggtgggtagtagatatgagagtcaaaagagtggaggaaagaagtgaaataggttcacgaagtgattggcccaaaataaaatcccatcaaccatatggatcatagagaatgtgacatagaaagattttaaagtgtatggtttttttaggccaaaaagcttcatatgtttggaaatctagaatgtgctgtccagtacagtagccacatgtggctgttgagcacttgaaatgtgtcttgtccagattgagaagtgtaaaatactaccggttttgaaaactttatatgaggggaagaaagtaaaacatctgattaatatttttatatttatgatatgttttaatgataatttttgatatattggttctaatagaagatcttactagattaacttcacctgtttcttttctctctctctctctctctctctctctctctttcatggtgtctagaaaatataacaaatgtggctaacatcgtatttattttggacagtgttgttctaggataaagcaggaaaacaatattctgggagaaagcttaaagattttgctggtcaaaacaataaaattggcttattttattatacatagaaatacctcgtctttcttatccattgtaaccaaaatttgagaaaacacaccacctaacatctatttttatttcctcaaagctgctatttagtttagagatttgatggtgaggtagtaataatagagaaaaacttataattagagaatgagtcttaacacctctcaaaggccaaaatagagtatgattgttacttaggaacattttagagacactgtagttatcattgtcctgaagttaccaaacataaacgagtcactaaaattattttaaaatagagattttagtaaatagcaattatcttcaaatacttaatatgtgttttcttttgctagaactttctttagaaagttaaaaaaaaaaacccatatccaaagtcctgagcaaatgttaataaaagattgctgcattcataagaatgaaagatttttacctgcagataggagggtattgttttgaatattagcaaaggtgaaaatgtgtttctaacgtaacattatgggctctgaagaatttctatacatggagacactgtctttataatttgaacacattcttgagaaggcatctgactggtaaacaaagcacaatggcataggtcctggaaacattcctttcagatattttcataactgaagtacattttgccattcacagtgagtgttaagctgaaaaaaatgtggttcttttctagccatataaagtatgcttactattattttctcgaaggatgccaaaaaatgtaaaatggatgttgtggattcttcgtggggaggttagctagtttgtatatagagcctcaagaagagttgcctcagcttggtgccatcttgagtctgtcttccactagatgaggcatagcttccattagtgtcagtgcgtgtgtggagggtgaggggaagaatgatccccattaactttgtatattgtttctgatgagtcatggagcatattcactaatggttggagaattattgattgcaaggcagaggattcaagaagagctattggcatatggcattaactgtctctagacatattttttatttttaaatttgaggtagagaaatcggtttttacgtagtgtttttcatattataaaaaataaacgtttatataagcattacagatacatccttgaggaaaaaaatagtaaatacatggttatagttttctacatattaccagtttttttcataacagatccagaacatctctttcagataatcgttcatgtttttagagaggcatctcatcatatgtgcctgtgaaatctgtaaaaatatgtgtaacacacatcttgaaatatttgtgttaggagaattaagtcttacacatttggttctttttctactgtttttgattatgttctctaggttactatgattatcttttgttaaaacagttcaggaaatattaatctcaaaacgaaatgtagccatattggacagagtagagaattgcttttccacagagaccagttattccaaaaaggtgaagatgtttctgaaataatatcatgctcatatgctccaaaagaaaaaccatcatctcttttgacattcttttaggaaactgagagatgtatgttttatataccaaaagaggtgaataatagttgataaaggtatgcaaaatacatactcctgtgaagtgtgaacatacaatttgtatgtaacgcatgcatgagcatataactttccccaagacaatgccaatttattcaaatcacctgaatgttaaataatatcatactattaagaagaattgtggtaaatatgaaatctaaataccagtgatgatgtccatgctgatgtcaactccattccagtcctgctttagctaaattcacaaaatgaagagatgtctttctccaaagtagttttccaaaaatttgtatttgacactggattttttcctacgttttcacttttaaaagtttagaagctattaactaaaaatgaacttgttttagagagctaagaagattctagatttatggggacatttagttctatgaaataagcattacgttgctgacttgaaatattattctatcatataaagttgatttataagtgaatacatattgtgaaaaaataccacagtagacttagtgaaatctaccaaacttaatcaagatagatatacgttgtcctcaaaaaaataagaatctggtagggaaaataagagcttcgaaaataactataaagtagaaagtgatatttaataaacaatggctgggtaacatggtatgaaagttcagataagggaaagtcttaagataactctgacttgacccacatacatgtaataaagggaacagtccttaaatgcataaataatgggaacaaaaggtattcagtatttttcagccttagcaaaatattattctgttaacgcagtattctattcagcaatggttccatattttccatatgtttatagataacattcagatatcgaattttcatatatgttttaattcatttatttatttcttttatctaggagaaatataatggtttccaaatgtcctatactagattattgatagattttgtcattagttttactattgaataccatgaaatatcagctgttgcagccactaaatagttacaaactgttggtactacagtttactagtcaatccatggttatatgcttgtgctatgctaaatataaaggattcaagatgaggacttaagcagttcatatttgtatttagaatattatcaaagatattttcttaacttgtctcagtattagaatttacagcccattttcagcttattgtatccagaaatttcaatcatatccaataatttatcagcatttgttgatgacagtaaaagtgacacattagcattttatttgtttctgtgttgtttgattacaacagtgacattttagcactctttgtggacggataactcttctagtagaaagaaagcaggagggtggtcaagctaagtgacagatacaaagagttggaggacatagggctttctgtgggtaagaaatggatgagaaaagctttatgtcactgactaggcttgcaattcagtattcccctatcaaaggcccccttactgatgtttctgtttaccagatcttggtttcaagatgccaggttctgaagagatacaccacgagttcttataaacactatatctaaatttggtttgaaagtctaatgtttggctgtgtgcagtggctcacacctgtaatcccagcactttgggaggccgaggcaggcagatcacgaggtcaggagattgaggccgtcttggccaacatggtgaaaccccgtctctactaaaatacaaaaaattagctgggcatggtggcgcatgcctgtaatcccagttacttgggagctgaggcagggggattgcttgaactcgggaggcggaggttgcagtgagctgagatcatgccactgcactccagtctggcaacagagcaagactcttgtctcaaaaaaaaaaaaaaaaaaaaaaaaaaggctaatgtttataccaggcaggtagaaaaacaaaaacggacctcattagaggatacatgaggtttctaaaagtggaactcctttgtaaatgtaagaagtgatatcaatattattattattttaatatacttattttgaagtaactaagtttttttgtttatttcttgaaagaaacaatagattggggaaaaaccatttactcttttcatgccctagtttctaaatctgtgacattataaaggtgtaaatatttatgaaaatagtagaagttgccactggctgtattcttttaagtaaggtatctcatttaatcttttctactgtaaagtagccgtaaagtaataagacttttaccttaaagatcatgactagaacaattttttcatttgcggagatatgaggcttgtcatttttattattccaatgttcatgtttttaaccaatttttcttgtcttgcctccactgattatatataaagaatagcgatgagaatatagctgtaattatgattactaacatctttgaatactttctgtgtgctaagctaaggtttataattaattcatttattcctcagaacaaccctatggtataggtactgttaatattctgattttacagataggggaactgaagcacagacaagtccaattaattgtgcaaaggtacttaattaataagtagcacaataaggagttgaaccaaggtagcccagctttagaatccatgctcctgtgtaaaagaataaaaacagattttaaaaatacttattttggatgctgataggagcttgagaacataaacgtttggaatactttaaaagacttgtactttctacatcatttgaagagattccagaagaatgggcagaaagtaagaggatagagagaaaaggcctggggtgaggggtgggaagcattaataggataggaagaaatgaggaatcaggtgttcaagggagattggtgctacacttttgaggtcattgatgaccagctagtaagtttaataatacgtagtgtggaacataaacagacacttggtgtttagggtagtaaccttacaggagttgtgttttggtgacattttataggggtaggacacatatagaatgaagtagagattgtggttatacagaacagagatgagggactagatgaggggagtggccctagaatgaaaaggaggacatgtatgctgagagaaatgtgataaggttaggctgcataggatgtaccagctgactggatgtggatattaaaggcaatgggggtattaacaattatttttaaaatacagcaagaatttaaaaaacttttaatcaatcaaaaattccttatttaagaattatataaaatatcagtaagtgaaaaacatttaaaattgtattttatcagggattataacttaagtagaagatacatgttaatttcttaaaacatttcatttctgtgctaagctctttcaaagcactggatggtgatctaaaggtcaccaaattctttgtcccaaattggtaattttaaagtaaacataatttcaacgttaggcctattttgttatttgctgagaaatgtctcaatgtcaaccaggcagttctcctctcttattaaagtccatgctcagaatgcctgtgtaacctatccttagtgataaacaaggtggcagatagatacatgataaaacaagtttgccatgtgctcaatgtggctgacatttatacttctcagaccttgctattttagggaaggtatcagtataaccagggcattctttttcctggaactaaaaatccttaataatcagtcactattataaacagaatgatttacatacaggaatcaataaacagttgcttttggttaatggttatctggagtaggaggatagttatatttcaaatagaatagaaagctttattggaatacttacatgtctttttagtttgaggaccttacaagctttttacaaataaatggtttaaaatagtttaaacagaaaatgtcagaacattgatagattatgttttatttaatatacagagaaaaattcccatgaaataaagacagttttcaagaaaaatattctatttataaatatttacaacttattagttatttagattgcccgtgtccataaaaataatacagaaattacactatttatgtctttaaatatagcatgtttctataaactagagaataatgttgacagtattttattttgcctttgttagtattatgtgaaagtcacacttattacatgcctttttctattattagactttttttcctatctagtttttgccttgtgctatacagtatggtgaaaatctatttagctatgtcattaagataatgcttagaaaatgctaatttttaagtaactaagattaagtagtattggatattctttgccttctttattattctgtcattgggctaattttaactagacaaatttattccaaagtattttgagccattatttgaacttcattgctgtaacattcaggaaaaactgcattgtctacctagtggcattcagataaggcatttggtcttaggtacctcttccaaaagacttttaagaggtttgataagcatagcaaaagtgaacgacggaggatacaagaaaatctttgatccaggagtagtctctctgaagcattgcatgacctttccaaaaaataatttgagaagtgcctaacaatagaaaaatatatattttttccttcaataggaaagactttagaaaaaatatactcaagtagaattctgcataatgctttctgggggaccacatttgctgaaaattgcctacgggaaatgactttctcagagtgatatagttttgaggactgagattttaaaagtaaatattttattgggagcaattaatttaggaagaatagtattatgcctattcctctgtagttgattaaagtttactaagtgctgaggtcttttcccttaaaacattactttgccatgttaacaatcaggtcatagaggaatgaattcatcttttgtgttttgctaaataatcagttaacaaacatattctgagcacgtttaaagtcaaaggattgtagtaggtcacaagagggagatcgaaagtataattacatagtgtctttccttctttgacatacaattgggttgaggagaaagacttgaaaacttaatgcaaggcactatacaagttagtaccaaaggaggaaactgaaagagggaaggaaattcaaagaaggaagtttttgtggaactgaatggcaagggaatgctgtggggaagagggggcatctgtcataggcccaaagattaagcactcttttcggaatatggagtgacatagtaagaataaggtggaagtagtgatgaaagctaccatatttgagcagcgtcttttctatgtgaggccttgctagcttatgttgtttttggaaaaaaacagatagacaacctggtagtgatagtagacaatgaatgaattgggccggtgagtcaggaatcatcatcctcttagaggacacagacttagagaaattatcttatttgcctaaatccacatagctggtgatcagccgaactgaacagaagccaggtcttttcatacttcaaaaccagcattccccaaccccatccctccccatcccatgattctgttgggaatgagtgtggcatgtttatgacatagtaattttaaaacttcacccatacaacaactgacataagtcaagaagatagaaattttatttaaaaggctgtcaaatgtattttcatcttttttgctttgatgatgggttacaaatagcgtatcgtttgacaaatgcttatactctgtttcttggatttgtaaaaaaaataaaacaaactaggcctcccatcttcagtagatatggtatggagttgcagtatcatgatgttgtaacagtgcatttcgcagtaggtagacccatttgggattccctcagaggaagcttgtgaagtttgaagataatgtactttctcaccaatagtatgaggctataaaattttcaccaagaaatgataggggcaaaagtttatagatcatggttgtttgattattttatgcttttatgttacatcatctaatttatacatcagtctctgctctctgtaactttacatgtgatgtagcctgtgactttaaaagataagctgttaattgtagagttgtgctgttggagacctgtcactttgctaacaaattcctctcttcatagagctatttagaacatatggcaaataaaattatgtgattatgtgtttttagagaaaaatttatttatataaaatgaaagtctttgcttttggctgcaaaagaaagcatccttaatagcaaatattgattgcagtgccagtgtgccatgtatcatgctataccctttggaggaaaaagaggtaagatgtgcctgttctcaaagtaatttccagtatagtgatagaaatattcacaaataatggacgggcaaaacaaagtgggcacaaagaagccaagaataagctgtttgtgggcagagtgtggaatttgcctaaaaccattcggtagacaggttaactctttagatactttgtatatattggagaagagacaaaatcaactcataatttattaaaatgatgtgatttgatatgtacaccatttagtttttctgttcaagtatttggtaaataagtgtgatgccagcatagtatcatcctggactagacagaagaaattaatttatttctgatgctggcagttttggagaaaggtgggcacatacggttcatctgaatcttcctcatgctaaattcttgattggaagcaggttaaaacactcaggttaacgtttatgttaacagaaaagaaaaatcagtcagagagaggtgcgttgtatttaacattaatgacagagaattttaaatatgcacatgattctctttttcctccagcttttctttctgcacaggaaataaagctgtattttgtaggccagcttccagactttgaaatggtgaaagtgcaactgtgcatattcttctataagaccccattcagctgtgacgtgttgttgtttccaagatttaatctgcattctagtttagaaaattcaggattttattgcttattattattcatttttattcataaatatttgtttaaagcccattacatataaagttttactataagattttaattgaaatttttaagacaaattaagaatatggtttctgattttctgaagtttatggcctatagaaaaaggtccattcataagtgaaataataaaagcaaaacaacaaaagactcgagctttagccattctagtaattattaaaatagctactttcttcgtaggaacttattattttacatgtgtgatcccatagctcttacacaattctgtgtgatttaggcattgctatctttctggctcccaagtgagaagattgaggcttggaaagattaaggttaggggatttgctcaagggtcatatgataaggtactactgtgagagctgagatctcaacccaggagttttgctgtttttctgcttgtttatttccatcagtatggtatgaattaggcacacaaatactcattgcatgtagatttgggacctactccaagttaattctggaaagactcagtgaagaagataagatgtggaagaggttttttttaagcattatggtcatgaaagagcagactatggaatggactttgaagtttcactagctttgaaaggaggggtatatcaaatggcctaatctgcctgtgtggtttgctcagtattagttctactcatacctaataccctctgccccagcactcgccaagatcagcaaaatctacttttagactcaaaatcattaccacctgtgaagtttctgatgtcgcttctgagtgacatagcactctgtcaattgctctgttggatggctttgctgaatggcaccttttaagaagtgccttgagccccaaaagagtttcaaatgctcttgtaatctattcagctgcattattgaagatattagggtaattcaattcattttttcatcactaaaacatagaaatctgttgccaacattaaaatagacaaacatatcattgaaaatttaatagctctacattcacttggagatttccgataaacttttctcactttcccccaaatccttaataattagagcatgtccatgtgtgtatgtgtgcatgtttttgtgtcatatccctatgtgtctatatatatattccataaatgattgtgaagtggtacacaagttgtagctaaaaatgaaatagaataaatgtatgaagctaagggatccattctccaactaatgcgttagttgacaaatttgctctgaaatattgataccattttgtgattccctgtattgaatacaagtatgggcttgtgaaaatatagtatccacagaccctaattagaaacaaaataaaggtattctaatatacctatgcaaatttaaagttttaacttattcatataacagttttcagcttgtcagtgacctgatatcaatggatttgaaaatgattttacctttaagtaccaagaattcttagggacatgtgtgtgtgtgtgtattcagattaaagattccatgctaatatactaattttttggcaagaaaattaagcacctgtatttcagtttaatagtactgtggtaaatgaaatatattaaacatgcagattctgttacattcacctaatattaatttgaatttttaatattctaggcactgtgctagatattgaatacataatggtaaacataaacagatagtaaacagttataaaatttagacatctctcaattttcaaattggccattgcagaaatagtgacattttattaggttttgttgccacaagattttcaataattaaaaaaaaattatttatagatgccagtaaatatttattttacagcatctagatgccagtaaatatttattttacagcatctaccaatgttagatttactatgcacgaaggaaggattttattttcatgtaaagaaaaatgccattttatgcttagaatcaatgctttacaattaaacaagagaaatagcatcacaaaataatacgggaagagaaaactcattttgagtagcagcaaaatattgtgagtttaaaaggtcaggccagtacaggaggctccagttttgccactcactatgcagaatgctgtgacagccagggctgatacagctgaaaatatctgcagctttggaagccttgtaagtttcagaaggtcattttggcatcatgataggagcacagattcacatggaaggtcaaactggacagccaggcaggaagataggttgctgaacccagtgcttgtagattttagagcaatcaccaactgcagtaactgagttttttaattcaatgcagccatctttcattgtaatgtttattggggaatgagctttacttggtggggaaattataaattaccttctctcttctgaaacctaagtgttgcatctacaactttccctggtatcaagacaaatgttactttaattttatttacgtgtgatatataccccatcaagcttacaaaagtaaagtgcacatcagataggactgttgttgttgttggtggtggtgtttttaatcaattagaaggcaaagaaatgaaacaagtacttatgatgaaattgagcactaaatttatctctcagcttcctggcaactgaagcagaagctttaggaagtgaaaaagtcctgagtttatttaataagatttcttcagatcccatacactgtcttattcttagccctttgggtgtttagttaaatatactgactctgcccttgggtgaagtcttgaatgggtgaattccaattactaaaaaagtgaaggctgaattctttagcctgcctttaaggcccttactgatttggcttcagcccaaagttccagggtcatcttctgccagccccattacatacaggtgttccatacagaccagttcttccagtgttgatatgcagtgctttcccacatccctgccattactgtctggatttctacagtttggatttctactcctttttgtctctaaccaaatgaaacttgtccttccagaccaaccttccatttcttatcccttttgaaactaaccttgacctctctctttaacttaccaagctttcttgttacctctactgtgtacttctcgcttccttttttatatatagttatttgtgtgttagttttatacctttaattaaacatcaaattcttaaaggctggatccaagtattctcgctatatctaggctacatataaaaggtggtcaatacacagttaatataatcaatacacatattttatgtgtattatatattgttaatgaatatattcatttaataaagttactgttgaatgtatttgaatttcttgaaggaaaacatttcttttcctaggatagtagggaacaaagaaagaaaacctaaaaaaagaagcacaattagagaaacacactcctgtaatttttctcaaaggaaatattttgagacagcagaaccaagattataattttttttaattgttttgactttgggttttctttttataagtcagcttaatatttgtgatttctctgtgccaaaaagtaaccaatattaatagagaacagctttagtacatgtaaaagaaaaaaatcctttatgtcatttcaaaccctcagtgctttctttctgacagatttttaatggttttatcagctgttaattaggttggtgcaaaagtaattgcagttttaaaaaaagatcgcaaaaactgaaattacttttgcaccaccctgataaaaaattggcctgactatattgctttgaaatgtttcagacagtattctgtctccttcattgctcttcagatactgttttagcagtttatctagatcagtcttactgtgtgcatagtcagcatacatcagttttatacttttaaagcggcattgcattttacctacgtggaagttttctgcagacatattttaagcttctctaagaaaagctctgaagaaaatgtaggctcctgttgctcggagggctgatgaataaatttaaaaaaatcatgctcaaagaccacttccttgacaattaatctttcaaaatcagctacggagtatctagaacttcataatctattcaaaattgattaacaatcttaattgagaagacatttgtatctgaagtgtagaattttcttgtctccaggtatacattattaatgtttagtgtctaccccatggacataaaagagcaatagcatccgttctcagctctgtaaaagagaccatcttatcttctctagaatcaatattcaggggtttctccttcacttacacacacctcactctgagctaaccacaagcaatgcagtagtggtacctccaataaatcccactcaccccctatctctctcccttcttttggcttctgttgtatatattagtagtcattgtcatttctaccaagtttggtattaaattgctagcatgtatcgtgtatcgtgaatgaagttttacatttgaaatcagaagagctgatttggagttctggttcttccatttatttagcatagggattttaatgggtcagtcacttaaccatgcaagcctcgcctcaatttatttgttcacaaatgagcaaaataatataaccaaccatctcttagagttgtgaggaacaaatagtaattaggatgaaaataaattgtatgtcaacatataaaatattatttgctatttttgaataatagaatgtataagttgttgggaaagtaaatttctatgcttggcttcttcagtggaaactttttttttttcaatttttttttttttttttgagacagggtctccctctgttgcccaggctggagtgcagtggcatgatctcatcttactgtggcctcccgggctcaaacaatcctcctgcctcagccttccaagtagcttgtactgcaggcatgcaccaccatgcctggctaatttttgtattttctgtagagacggaggtctcactgtgttgttcaggctggtctcgaactcttgggctcaagcagtactcccgccttggtctctcaaagtgctgcgattataggtatgagccactgtgtccatcctgaaacttttttttttttaattaaagaaccaaatgaacaccttgcagattttatgtataagccagaaagaaagctcagtacatgtatagaatgtagcacaagccaacttttcaaaaattaatctttttaaggggcattatgtaatgaaggagcatacatttttagttttatccaggataaaagtggttctttttcccccattaactttaacacagaggttttactttaatttaaattccaaggactaggtttttgttgtatatgaagaaaacgatttgactaaatccaatgtaaatgtttttccttattctttgggggcacacacatacacacattaatccttattttatattttatttataatacaaatatttaattatattttatttataatacaaatatttaattatattttatttataatataaatatttatattttatttataatataaatatttaattatattttatttataatataaatatttatattttatttataatataaatatttatattttatttataatataaatatattttatttataatataaatatattttatttataatataaatatttatattttatttataatataaatatattttatttataatataaatatttatattttatttataatataaatatttaattatattttataggttgaataggttccttgtttatcttttttgcaaaataattttattgttataaattgttcttattataagcaataaataaggaacctaaatatcacaatctgtaaagcattttatttgtgtgtgattttatatgatagttcattagacattagataggaaagtatggaaagttttgagattgctacatgattgctcttttaatttgtccaatgtattcctttaaatgttgtgagggatttttttaccagtgaagtagcttttgcatgctaagttgtagtgaactgctgggcagaactaaaacataaatggtttgaggcatgattaatagttgtatatggtggccgggcgtggtggctcatgcctgtaatcccagcactttgggaggccgaggcgggtggctcacgaggtcaggagattgagaccatcctggctaacatggtgaaaccccgtctctactgaaaatacaaaaagtagccgggtgtggtggtgggtgcctgtagtcccagctactcaggaggctgaggcaggagaatggcatgaacccaggtggagcttgcagtgagccgagatcacaccactgcactccagcgtgggtgacagtgtgagactccgtctcaaaaaaaaaaaaaaatggttgtatatggtgtagtatcttagagtgatgtgcagtggcatgcatgatttaaaataatattaataatcataacagagcttttttgtaacattttataagcatactaccttgctgaagtagtcgatggaatataatcaaagattgtgtcaccaaatctttaaacaggacattttatcagagaagatactctggtttcttaggtaaagttatagggtagaaacacagtaagcagtctctggtaagaataatattttagccttgattgattatggttgccaaaatttaatggaattaaaaataacctgggaaaacatagattcctggtttctatcctcagtaggtctagggtaggtcccaggaacggtatatatttttaatagttacccacgagtcactgattcagataatccaataatctttctggagcatcagtatcccagagaaacaaatgttgccgttaataaacaatttgcagttatttcagtttattagctagctacgattgcattctgatttgctgtggaaagctggtcattactttttaagagcatatgatgctgatggaaacatgtagagctttaatcgctgtctcattaaaaggatgtagtttattctttgtgtttccttatttgttacagcttttccagaatgcacagaaccttatgtgcaattaaaataattaagaaaaagatattgttttctatttagcaaaggaactaaatctcatgctcccttatagtttctgttcaatatgcattctcttttgaaagtacaaatttgtaataatacaatttttgatctcgtctcccaagaagcctaactttatttatactctgactacacggtaaaatcacattgtcaaaataaaagcgttggtaatgctcacattctgccaaccaaccatatttgaagtcttttaaaaattcactttcaggtactttattctgtagactgttatttaataactaagaaagagagtttgaggaggacaaagtgagggcggaatgtttttattttccaagaggacatacatgcaaaggcataatcatataagagtatatacctatcttggttattttaattagaagtgcatgctgcttcagggagactccccactcctgactcaggaatgactgcgagtggaacccccgcctcagggaaaattactacatgggaaaatatgcttgggcctacatgagttttccagagttctcttgtaccttagctagtttgataatgctttgcattatgtatagcagttgtcagaaaccatttcctttaatcagttagcatccaaaaaaggaaggggtagcagatattttctgacatttgtagatataatgtattaaaaaaagcattaccttactcttatgacatagtgttgtcagagatttcccaaatacaacatgccctgcagattcttaaaactacacattttccttttgttaataagaaaataacagtaaagtatttattgtctattttgataccctgtcaacccaaacatgtattccaaattccagcctctgtttctttgtaaataattacaaaatgcaaatactatgattgttttaggaatgaaatgccatgttttgatgcaacaggagttgaaaaactgattttacaaatgtttccattgattagctgctatatgagggctaacagttgaaaatattttggaaaaatacaaaaacaggctttcatttctatttatagaaaaacagacatactttttttgcatgttcccacaagagaaatttacagtgtagataaagtacagcataggcttaacaaatatagaaaaatatgttattgactcttggtggaagtacatattcgtaaaggttttcttaaagttttttattctagttcaaagttaatacatatatgaatacacttcatgttctaaggatttatattattaaaatcactcataagcatagctttttatatgcaatttcagcattgattaaaagagtgaaaaatgggcattaacttagttgactactgataacaaaatggacatatagtggagtaatatataattgttaaaaagtaattatattgatctgtttctattgacatagaaagatcattaatatattttatattgtggaatattaacataatttgttgtttaatctctggacctagttagatatgtgaattatgtgaaaccttcttccaaggatatttaagttacattgaagtgaaaataggagttgtcaatgcatgtctttgttttgtggcgtatcttagtcttttaaaccagggaaaaacaactgggtcttgagtcagtgttagtcagtgaaaacattattcttgtttgtgagcatatgtgaatttgttatttttttgaaaaaaagtcaagttaaactgatatatcttgttatgaagaatatatattattatgaggaatatattcagttaacattggtatattttttaaaagtatgtttgagattatttgttaaaaagtaatccttaaggtcagttgtataagtcaatagaaatatccataggattgtaatgttaaagtgtgtagcacctatgtggcttgagtatttgtaagtttggaataagaaatatgttagtgaaataatagggacttttactgattatatggaatggaggaaatatttgtgtattacgttgtgtcatgagaagttcactaatttgtttttttaggatacattcgagcaattccatactcattcgtcattggttttagtcattgtattctgaaaaataattattttggtctttttatttaattataggcatagatttttctatactttttttcataatgattaatagttaaatcacatattttgggccatattcaatataaataagctaatacaattttcctgattgaggttatcacaccaggtttaaaatcagacatttggaaattcaaattctatttgttaagataaagcttctgtggtcagatttagtgggtagtactgagggcaattatcacattaaatttttagaaattctgaacagtgtggcaactattaagtttgatcctaatgagatatgctttatattttgggcaggtttttttttaaattatatctgtcatttttttcacatgttgattttgtaatttatcctacttgagtctgctaaaataggaaaaaaataggttaattatggagaagatcttataactattggttgtgagaatgatttctgtgtgagagggaaagttggaatagaggggcaaagaggtacaaaatgcagcagatttcaccaatgtatgaggtgctgtggaaatattttcttagttctgtgatcaattaacttcatacttatttttgtgttgtgcaataatgtcattagggatgcacagatgaatttgctaccattatttaagcagtgaacatcatattttaaatgttttaggcagaggtgccaccaacctcagcaagaagcttggtgatgttcagaagatatagaggttaaatatgaaaactatgattttggcccttagttactgtatcaaataaaataatactcatttaagtaagctttaggatcaaagtctataatcaggacattgtaacgggaataacagtattactgtgtgttgcatttcatttagcgttcacatttttgttgtgttagttctacattgacagattcgggttgggtcactgtaccattcttcctgaaaattgccaagatgatgagatttgatttttctaccatttagctcttgagcacataacagcattttaacttttgctgtagtctgtgttctgattccctatttactattttgaaatattaaatgatatggacagatcagatatgtctacctgaggctaacaagatgatgtctttgtgtaatatataggtaaataaaacactaattaggagtagtgctttctcttgtagaacaattaagtagcattcacagttttatctcattataccttcttgtagaaggaaatgttaaaatacttcctctttgaatagcatcagaaaaaatgcagtgtttcttttgctggaattgaagactggttctgtcagaatttcacagataattatttaaatattttaaatttgcaaagccacataaatctgtttcgaatattccatggctcaaaatatctttcttcaacttcataatcaaggaggtatttaaatatgttgctattttaacttcatcgcctcatgttctctttccaatgagtgacgtttgatgctggtttcattcacttataaaggtgacatccccaatatactaaaccatatagcttaagcattgtaatttgtcgaaaatacaaactagtctgatatactaagaaaaatcttcagttataccacaaatattaattgttcgtttcctctgggccaggcattgttttggtgatctgtttctattaatggagaactagtgtaaccactgccttctctcatggagcttacatttttgtggcaaaggcaaatgataagcagatagcgagataagcgtgtaatattactctgctttcttcacagtactatgtagtattagatggggtttttttgtttgttttgttttttagacgaggtctcactctgttacctaggctagagtgtagtggcttagtcatagcttactgcagtcttgaactcctgggctcaagcagtactcccatttcagcctcccaagttgctgggaccacaagcacatgccatgccatcatgtgtggctaatttttaaaaatttgttttctgtagagatggggtctcactgtgttgctcaggctggaagtgtaatgttatgtacttctctcaaaacgagtctggactgctgttgggaggaacagaaggttgggtgctttggatggtggcctgaaaatcttctaagaagataactttgcagtagggacttggacgaaggcctattggggtatagagctagtatgcaaagtgcagtgattcattgtgataattatcttctagtgagcttaaaccttttacatttttaaaaattttgcctgatacaggaattaattcaaaattcacttttaaatcattgtctgtatagaagctttatgttctgttttgtcctaaatatgaatacttaaggatcaacaatcattttctaattggaatacagtttagttgccactggaattctaatctttggcagtagtgtgtcacctaaccgacactccttcattatccttcctacagtagaccactaggtgatttagggaaccaaactgttgaatggatcagaattctaaaagactgcttcgacagagcttagattacttatttgcgttagtaaacacaatcttcctttcttggcagcagctagtctgaaatgtggcagaccagcaggccacattggcagtctctatgccattccagaggatgtgtgattgaaagttatctttttctttgcagtattgttaaaacttagtcatttacatatctccttcatgattttcatttatgtgtagtctcttaggtatatttgtgtgtatatgtatctattctttaaatcttttttaaaaatttatttaattaatacttttttttttttcagacagggtcttactctgttgcccaggttggagtgcagtggtgtgatcatggcttactgcagccttgacatcctgtgctcaagcaatcctcctgcctcagcctcctgagtaattgggactacaggcatttgtcaccctgcctggctaatttttgtattttttgtagatatagggtcctactatgttgttcaggctggtctcgaacacctggcctcaagcagtcctccacttcagcctcccaaagtgttggattacaggcgtgagccacggtgcccggccctttaaatttgtaaattaaactttaatttcttcctaagttataatattgatgtaatcacagaccggacacactagttttagcattctaatccatctgttaaaattatgttgtttgtgcatcacctgaaattgccatgggtactgctggtagtgtgcttacccctcactgagaaatacttacctctgctttttgctatgtggagttgaatagtgctgaaggctgcattaactcgttcagatggcagttcttcccatataatgtataacatctttgttactctacttagcaccttagtgaatacagagatggctgtacagcctccagaaatgctaagttggttggttgtatatggaaatctctgttaatatttgggacaaaccctgggaataattgtgtgagcaaacttagtttactaaagaactgcattgctaattatggacttgttgacaccagtgagccaggggaagaagttgtggttttgggcaaggggttctttcttatcaactccaaacttacagggtgaagttagccatctctttcagttagattgaaactatcttaataaacaaaaatcataataaacgacctcaaaaaattacttagttaacttcctcagtatataacttatatcaacattaaaagcatattaaataaaattcttcaaaattaccagctaactactctgggatataactggactgattgcctatatttgctttagaagaacttttgcaatgattagtaaacatatgtgagcaagaaatttaaccttgctcatagaggccatccagttctccaaaagagaatttcattttcataccttgtaagcatctctttatcttctatagcagtaggtatgtgactcttaaataggcattcatatgaagccatatatatatatatgtaatttttaagaataattgttgaaagaataagaataatttcattcttctttatccttttctcggtttttattatctctgtcatttcaaattttatttctttcctttttattttctcccttatatttatcttaaagtagaatatatacatgatttctgatatcattcaaaaagaaattggttcttgtggcagaatattagtgttgtagatgatgtcagataaagcaagaaactatagcgtttggaatgtgatcagtaggttaaagagggactgctgttacctattattcagattttaaagtttgattctttttttttttttttttttttttgaggcagagtctcgctcggtcacccaggctggagtgcagtggtgtgatctcggctcactgcaagctctgcctcccaggttcacgccattctcctgcctcaccctcccgagtagctggggctacaggtgcccgccactatgcccagctaattttttgtattttttagtagagatggggtttcaccgtgttagccaggatgatcttgatctcctgacctcgtgatctgcctgcctcggcctcccaaagtgctgggattacaggcgtgagccaccgtgcccggccttaagtttgattcttacaatatttttgttgttcagagaagtttattataatgtattgaaatataggtcagatatttacctcagaagcaatgtacccattagtagaaatctttcctttaataattatttacagacaaaatttattttaggccaaatatgtttgcaagaattctcataatctggacattacccacatatctttaaacattatatctatctgtagaacaaaaccccattacttagttaatgttttgtgaattcagtattgatccaaattctataaaatcttaatttaccaaaagagatttctattttttgaaattctttttttaatatagagaaggaatataaaacaataatctgatatcagcagtttagtgaaattgatctataggatataatttgagtttgttacccacagtgccctatatcaagggcaggcacatacattatcaggtctagctagactaatgcataatggccagtatctattttaattgttcagtgtctatgcctgaactaattattaatactttgattatcatccttgtctatgtcctttacaattaaatggtacaaatctatgttgataatgctttcagtagcacttttgaagtaaatagaatttctctgctacagaaaataaatataatcagaacactgatttgcaagcattttagtaatcaaagtatattttgtacttgagggattgtctgtaacctggcaaaaaggaaaaataatcaactctagaaattaatttttagttgtatttagatatgactaccaagcaatgcagatatttcttggggtaccagtcttcttacatatattgtctggatgccatagatcatgatattaaataatctatttcatcagtgttgggaccacatcctcccatacatgtttatttctgtaagtgtgaatgaagtttactcatttatcaaacattttaaaaaaagatctgttatgttccaagaactgtgtgatggaagacactggacctacaccctgttgtcagggcatatataacattgagggggagaggtacgaagagctacaaagtaccagaaacattctgagatggagtaatgtttagcatagttgacccagtcctatagagaaggtgatttttgaacagcaatttaaacaaggagaaggaatttgccaagttgcaggggtgaggaagtgtgtgtcaaaccaaacagggagttagcatgtaacaagtcccaggagctcacacatgtcttggcacctgccaagaaccctcagcaactcctctccaggggagatacaagaggtacagcactgagagaagcaacaagataaaatgtatcagtttgttaaaagggaaaggttcaagaaagagggagttgtaaatcatgttacaggtcaagtaaaggcaagaacaaaacatagtctttggacttggctttcctgacagcattgatgaactttgctgagcagtaaagtaatgggtgtgaaagtcagactgataaggattcagaagtgaatgggaaaggaagaagcagaaaaacagcacggtagatgactctttagatatttgcttgggaaggatggattaagtgcattagggagaggaggatgctagccagggcagggtcctcttcctcctgccatgtcaccgtctgcatgttggagtttcttcagcacatttggaagctaagagaaggaactgctgaaagacagggcgtgcatgtatagaggaggcagaaaaagaactagatcttgagaatgactgagtcaaggtggaggcttgtggaagcttaactgtgggcaggaggagggagaactgggagaccaagaaagatacagaaagataggttaaagacttgtgagtaggaaggtaagaaaaagttcatgcttaagggctactgttatggctatgaaatattcgtgacgcataatattattatccactattgctaatgaacatggagaaagagtaatgaagttttagaaaggctgccgtgtgcaatgagatagggagctgtccaaggacagggctagattgtgaggcagtgaaagggcctatgtgaccctggaacataataatttatagaggcaatgattgtgtgtgtgtgtgtgtgtgtgtgtgtgttttcaaatgtatgtttcagtgtgttcatgtgttgtattcatatatgtgttcatgtgtgtgtgtttgtatattttgtaccctagaagtaatccaaatcctgatgcagcagttgataatttagtttaccaatatttttggaactccatctattttgcatcattatggctaagccatggggttactgagatgaataatatattttttctgtccttaaaaacatatggtctagtgggtcagatatataaggaaataaaacactgtatagggcataggtgcagagataattcagagaaggcagtattacgatatggatggatagagatggggataatgacatcttggtagctttttggaggaagtaacatcttagctggctttagaggaagaaaatagttacatgtgaggcacttgaagattctgaagagaaggagtaaagacattgttgaccatgcaggtggtccattctcaaggcatgaaatcattgagtaattcttaatgcagaacaaagtttcaggaagatagacacagcttacagtttatgactggacctttgttttttttttttttttttttatagaaactggcattaagacctaggaacttttttgaaaaccaaaatatttcttgaacactggggatgaggtttttttttttttttttttgatggagtctcacactgtcttccggcctggagtgcagtggcgcaatctcggctcactgcaacattcacctcccaggttctaagtgattccaggagaatcacccagcctcccaaggagctgggattacaggtgcctgccaccatgcccggctaattttttgtatttcagtatttttagagacagggttttgccatgttgaccaggttggtctcgaacacctgacctcgtgatctgcctgcctcggcctcccaaagtgctgggattacaggtatgagccactgtgcccagccatgggttttttaaattttttttagacagagtcttgctctgtcacccaagctggagtgcagtggtgcaatctcagctcactgcagcttccgcctcccgggttcaagtgattcttctgcctcagcctcctgagtagctgggattataggtgccaccaccacgcccagctaatttttgtatttttaatagatatggggttttaccatgttggccaggctggtcttgaactcctgaccttgtgatccacccacctcagcctcccaaagtgcggggattacaggcgtgatccaccaagcttgattgattttttaaatctctatcaaaagttgtagtcgtactgtagtattttccaaatagttttggaaagtagttttttaaacgaaatttagtgttaaaatggcaatattaatcttaaacaatttaagttctagatgttcgtgttttttaatgtacgtatgtatttatatcctatactctagtttccaaaaattataaatataccatatgccaatagagcaattttggctcatgaagaagtatcatcaaatactagttatcatactgtgtcattgcctctttaaaatgtgaaaattttactttttaaagccttgttttcctcattggcaacattcaaacaaaaaataaagaagaaaaatcctgatagaaaaagtaagttgttaaaagtaaaaatgccctgctctaagaagtagtcttgtggtgctgagttaactgatcatcaggcaacaggggatccctcatcccccaccctccagctttgtttggaattgtacttctgaagtgcatcacgctgctcaaatcaaattgctcacttaatatttaatgtttctgcgatggaatcactttctcccttgaggttgacttcagatttaccagacacactgacaacactgtactccttattttccccacatgacctcagcttctgctttaaaaatgtacgaataaaggatgcttttgttcatatcttgcctgcctgtgatatattgacaaagtgtctgcgggctcaatggaatagttttacagtatgagtgctgttttcagtggaggggcaggacttgggctttagaaggaagtgaattggataggtcaggagaatgtaccatttgtaaacacccctttccttttttattcacgtgtcaggacagcaggagcaggctgttcagtgcccgtccctgcgcagagccggagggaaagcagcttggaagtcagctgcaatttttaaaccttgctttttattccctggtaatgatcttcaagtgcttagacttgtctgagaagctgttttgaaactaacatggtttagtccactaactgcatgttgggtaaattcaaaacccacatgctcgtcctcttgcagtggaataagtcacatctgatggacattttctgtgcttatagcatagtaatgaacgtctgacaggcgcgacctttcataagacaacccacactattggctttctgcccagaatattgctgcaacactatctgtgattggttatctctctatcatgcattgcttcacaaggggaaacggcccctttttttaataaatctacgatggctggctgcaatatgcctcactgtaagtattttgtgtatgtatgtgtgggagagagagagag

SEQ ID NO: 13 - DNA, 110 bp, Homo Sapiens
aggctgacttgaatattacttagtctggtatcacatggcaaggttgtgatactgtaggacatcagtgaagtgctacttagtaaatcttaacctatctcttttttctacag

SEQ ID NO: 14 - DNA, 963 bp, Homo Sapiens
gttggtactaagaagtgcctttcctgacgtctctgctgcttggaaccgcttctagagcagtctctgcttttgccttgcttgctgccagctagactgtgacgacagcacatccaccctccacctctagcccagacacccccatttctacttataatcaagagaaaagctctaagtatctggcattgccctaggctgctttagtgttaaaagaaaagtttgctgaaaaagtaagatatcttctgccaggaaatcaaggaggaaaaaaaaaatcattttctcgattttgctctaaactgctgcatctgtctatgccaaactaatcaataccgattgcaccaccaaactccattgcaaattcagctgtgaggagattccctttcagacaactttgctgaaagcagcttggaaattcggtgtcgaagggtctgccacgttttcatgcttgcattttgggctccaaattggcactgggaaggggttactgagagcacaaggctgataccaggccctacttttaaacgttcatctacttacaatcctagtatttctctaaaaaccaaaacctctttgaattaacagtttcatgctgtgaatttctagtgggagatcttttccttgatattgacgacacaattttccatgtacttttaaagcagggagtggggaaaagtattttgaggggacattttcatcatcagttcagctttttttttttggttgttgctcttttttgggggggttgggtttgttggtttcactgaaacatttaactacctgtaaaatctaaacatggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaa

SEQ ID NO: 15 - DNA, 73 bp, Homo Sapiens
gtgagtaactattatattcttttaaggatattcaatgattgaatgaggggtatatggacatacagttctctgc

SEQ ID NO: 16 - DNA, 32767 bp, Homo Sapiens
atatgggattatggattgctttgttcatagtttagttatacagtgtgttgatgatgttgggctggggaactttacgagaagcagtagaatcaagtctggccaaactttgttatgaataactttatcagtcctttcactgcatgtttttttttgctagggcagaaagtgttgacatatttttcacttagtaatggcaaagtgtctgtctgtccctggaaatggtaaatgtagctacctgtgagtgtttagcatatgcatatattatatataaggccatggcaatagctgaatgaggatgatttttatttgttggtaaaggcattgtacagttgcaaaagagaagcagatttaaaatcagctaaacttggatagactttttactcttttaatattttgacattttagattttctttttattttgtgggcattttattttcaggccagagccacgcattttatggaaaatgtaactagaagtaaggtgctgaactgctgctacagcaccttacctgaggcacaaactttgttgcatgtgagttatataaataagtagaaactcataagtactattgtttattggatttttccagcttcaaaaattctaatgtgaattagcagtttattttctgttagatcatgatttttttttatttgtaatagtcttagctagaaatgttaaaagttagcttttgtgggcaccataatactaaaatatttactttagtgtttcttgacattcaaaaatgtcattggatttgtaattcaggtatcatatttgaaaatgagtcttttaaaagataacataaatatctttattttgacacacaaggtcaagactagaaatgtgttcctgggtactttcagcctacttggtttaatcaaattgcttttgaatatgaatgtcctaatttaattctttggacctttgaggggaggacactatcacttctacatatgtagagaagtaaaagtctcatagatccatcttgtctttaaaaatatatggtgctataataatcttttctttaataacatcagggtgagttatgagttaattgtttcagttcattgtttcctggtccattttaggctgttcattttgctaaagtttttgccaaagatttcgagaatatgatctttagagttgaacagaaaggaggtattaacaacaaaaatattttaaaatgttagattcttcttaatgtcatagtcctaaagatcaataagcaaaagttataaactcccattgagactttcttcaccctttcctttgagaggcatcagtgaagggcacagtggttgtacctccgtgaatgtgtattcctaaattcatgctggctgaattcaggtgtaattagagactgagacccataaaatgaggggcaaggagctaattactcttgtcggtaaccagctgtggccataagtaaccctgttttgacccaattaatagtaaatccaatttttaatcctacagacagttgatggtgcactcgtagctgcaaaatgaaggacattaaatttggctgtgacattgagtggttacacagaagacctgcttctacaacatttggaaacattgtgaaacattactattcagttgctatttttccatctcaaaatatttttttttagttgctgctcacttatggccacagaacagtccctttttgagtaaagcagcaaggttgcttaatgacatgctatgtggttaacaagattcaactgccacatcaaggactttaaataatttcaagattttaaatgtatttatgaaatgaactcattacagcagctaatcaatgcaaactttgtcctttttctttgtctttctaagagaaactgaatttaatttaaatttaaattacagtgtctaaaacttggtaaaatattttaaatttgaataagaggaaaaaaaaaacatgaatttatttatgatggagcttcatacccaggacagtagcttcttaaatgttagtaaaataattatgttaagcattaaaacaattttgaatatgtaattaagcatttatgttaaaaaacatgtttcttacgtgtgtgtgtatatatttataactgacacatgtgattctttgtacagtatttagaatccttcccttcagatagcagtcctcttgtattcagtcttgtggctaaaatagatcaaaaacatattttaaaactgttaaagtaactgtatgactcatgcttgaagttctgaaatttgggagcgttatgaggtttccatgccattaaccgctagatcatttgcattctctcacagagtaaacggaaaaaaatttagaaaacttgtaataatcctgacttctttaataaaatgtgtcctgtgtattgcttaaatccatagtccagccttttctgagttcttgagccatgagatggaggtggcctgtgtaatctactatttttagaaagttaggaaagcaaaccaaatactctctgagaggaccctatgaaatggctgtacccttcagctgtgctgcttcagactttggccaggctcccaggtgacttacgttaatagcagacttctagttagaagaggagaatttatttcattaaaaaaaaaaaaaataagtcaaacagttcaagaaagtattcatttatgtaaaagaagtcaattggtatgttactaaagataataggaagaaaattagctcactattttagttgcatactgatgattttaaggtttaaataattagatttaaatataccgtatgtaaatgtagaagtctgtatcgtaataaatctgttgctaagtaaaatgggaatacatgacaacgaggaatgtaggtattaagaatgtcagaattagaacgagaacagtaaaagagcaaaagctgtaataatgtgtgctagtatataaacaatactctaaatacttagtttctctaattttccttttacaattgcagttttaatttataatagtctaagagcgtattcaagctgagacttaaattgcttacgtgctatagatttaggtgtattagctatttcagaaatccagaacatttgcaaaggtgcattattttatgccaaagaggattatgttcccctatggcttgtgaaagagataggaaaaaagaaaagaaaaatattaacactgcaagccaccattagaaaattatatttcaaaaatgatgtttcaaatcaatgacaaattgcctattcagtcatgagctctcaactaaaatttggggagtcaatgtaaagaaagcctacagaatgtattttaatatttgccaagttatttataccctcaaaaaaaaagcttttacgatatgatcaacagaaatagacttattgtttactgaattctatttcaaatacccattcatttttcaggtactagggatgggagaaagagaatataacctttatatttactatctattttccaaaaatctagcaaaattcttttacctttgaaaactttagtcacttgttcctttatcaaattgagccattttatttaagataattaaaatttctttttgctatatacagtgatatctatctataaatagcaccccgatcagatttatcatagacacatttgagatcagattccactgcaaagtggcatacaatcaagggtgctgagaacagttttaaagagcctctgcaggcttagcagaatatatgaagtatggaaattaggtattagaaggcacacaaatatatgtacatacaggtaaatgaacaactaaagcactcaaaatcagtaaggcaaacaagtatccagatggtcacatatggattggatggcataactagacatgataaagtagtcccaaaggtggttatagttaaattttagaagatcctctgttgctagcatggtttatcctcaatccagaaattgcctgtgttgctgctgttctaagccccaagcatagacttttcatcatattttaaaaaccagtttcactgataatcctgataaaatatctgcaagttttagactaaatatgcaattttcaattaaaattttgatctattagggtcctaactggaatcactgttaccaggaagtttttttgtggtttgatttttaaccactggtatctcccatgtattaaaactttgacagattattatatatactgtgctggcacatatattcaatgtgtctttcagtgacagattgacatatcacttccttgttttctcctaataacttaggagatgaactaactataaggcataacctgcgcagcagtatgtttcatataaaaggtccctaaggagaaacttcacaactttgtaaagaatatcctgtgaatatgctgcagtgtcatcaactctgacataaacctttctttaatgaaattctcaatagctgcagttgctttggtgaaactagtaattgtattcatttgcctgtttatattagaacttttttagaaaacttttttttatgagtaaaggtttttttagactattagataaattgtaaaccatgtttcgaatcaagctacctgctgcagtaattctagaactatttattttgcttcatattataagattttggtacactgcgtctactttttttctcatgaaatcttgcttcactgtgtgagtgtgacaaactaaaaacaatttagacctcaagcaatgtggcacttacagctgaattcagtttcattgtaattcagatgtctgaagctaatcaaaattccacggagtacccttgaaacaattgcatatataaggctgtttctcacttaaaagagacaatgcagtgtggggtgaattgtcttttgaagaactgtatgacacatgcccagttcaggtgaaaggtgagaaatggaatgtttctcagacctagcacagacacctagggacctagtttgggaagcttatcttgtcattttccttgccaaggataagtagaggatttcagctatcaacattgctaatccgttacctttcatgctgagcaatgtctctgccttcccacagacttagacaaagcttcttattttcttacaaagaggtcctattgaggaaatttgaattttttttttttttttaaacttagcagtactacatcctgtgcttttagataggtgagcaattttccatgttaacagctaggccaaaaatcatgactgccagtagaagtgggtaactagatagatagatagatggcctttgatttagcactgatgatagttattagcatgtagcaacttaagtaaaagcaactggaaaggcttgatagaaattctgagttgtttgaagtataatagattattaggatagtaatttctttgtgaattttactgtaaacaagcttgattctagtgaggacttatgctaattgctcacttatactaacagcaactacagagtttttatttaatactgtttcttatacaaatatcatcaaatgcacatctacctacagcatcatccctacatttttggcacatgtttgaaatattttcactttgagataaagtttgatttttttgctagcgataaaactttctatgctatgagcttgtgttttgaaaattactgataattactaaacagtataacccctgatatcttctaatttggttgacaggctgtttgtcctctggattcctgagggccttggccatctctgaccattacatttcttcttttgttatctctgagttctttccagctttctaattgtccatctttagaactattggttccgatcttaaatatgtctttccatagtcacacagaagtgatttagacattttgtctgtgaagaattagattcagctttgcataacagagacacctcccctaccctccccagcatttggtataaacaaattaggggtttatatttcagtcgtctaggtctatttggcgactctatagtcattggaactcaggttccttctctcttagttcctaaggagcacatggcttccaactttaggattatttcaaaattgcaatgtggccactgcagtgccagccatcatatccttagtctagcaggacgcagaaaaggcaagacaccataactcagcccagccaggctccttttaagcaaaggttctctgagcttcagccagtaatttttcagtctcttattgactgattttggtagcagggattgctaggaagtatttttttgtatgtttgtttgttttgttttgtttttatttattattattttaaaaaactgttgccttgattgaaatcaggagtctggtaggtaagacagaaggggagaatggaaatggatgttcaggagggccatttctggaataaaagagtgtttatttttcctctcataaaactttcagaattaagtaaatctctctggaaagcatctgggtaagggaggtggttgaagagataccacttttgaagagaaactgaagtcttctcatcttgacaaacttgtcatcacttctgtcttgcttatttagtaaataaaacctggcatatcacaaatggatttatttttccttatagagaaaactaaaacagcttcattttttatcttcttataactgtacttatttatttatttatttgagacagagtcttactctgttgtccaggcttgagtgcagtggtgtgatctcggctcattgtaaccttcgcctcccacgttcaagcaattctcctgcctcaacctcccgagtagctggtcctacaggcgtgtatcaccatgcccagctaatttttgcatttttagtagagacagggtttcaccgtgttggccaggctggcttgaactcttgacctcaggtgatcagcccgccttggccttccaaagtgctgggattacaggcgtgagtcaccgcgcccagcctactattcatatttatataaacagtcacatttttataactggaatatagcagaaatgccatgtcattaaaaacaggtcccaagtgattttatatgacaaaagaatctgcactttgatttgtatttctgctgcttccacaaatacactttcttattttttccgtatggagtaaaataacaaggtgagttaatatttattgaaacttaatgtatgtcaatcagtgttttcagtgcttttcatttttttcatagtattttgtccctataaaatctcatcgagatagttactggtgttacttccatttcacagatggacacactgaaatacagagaagttaggtaatacatgcaaggtcagtcagccagtaaaatgtggacttggagcctcgtaatctgatttcacagcctgtactcttaattgttttgttatgtttgctggaaaacattgcttcttaatttccatatgcatattctaaggattcgacaaagaaaatataactaacagcagtacttaagaataaaaatgaatatccaagacaaatttatagtacagggcctaaaacagtaagtactcagaagtaatcactgtattttaagtcaacaaatgagtcaaggagtgaacccaaaatagcagaaagaatgctgccaccaactttcaaagatgttgtatctgttgtaaaatatatggccatataggaaaaattttaaatgaatcatcttagataactagtcactgtataaactatatagctgcctccccagtgcataaaaagccatattcaaaaggtttaattgtttagaaaatgtttttccacagaagcaaggtgattaagtcatcgggtcaaccctgagaagccaaagtagcccagaggatgcttgagttatagttcagtacaaacagtaccttggaaaccctggttattaattaaacccaagaggctccatagaagtgttatcttatcataaatcttgagaactcctagagtcacagaccccagctgcttttgtcaaactatttctgtggcaaagatgtattttaaatttcagtttagggttccagaaattcagcttgcctagtgcaggggaggtggcctttcccaaagtcctggaactcatgctgtgagccagtgtagttttccccagctttaactttgggctgcttctgtttcctgaagctagtctttaatagggcatttgcttagaatcagcaggatctaaagacagggtatgcagtgaggagctggagtccaaagcttcccacctgtgtaacctttagggtgggaaggtgtcgtcaatatgtcttccaagtgacccccatgtttctaaggactcagcctgcctgttttttgaatgtagtgctcattgatcctgggccctgtctactgcagagcttaatgaacagcttctccccattcttccccctctctgaaattttcagctgtgtggttgttgctgttgttttctaaatactagtagccatggggaaacaggtagatcataaaagtatcaaactcaatttttgttttgttttgtttttttttttggcattttatcataggatatggtcaagagaccggttgtaaaaactcagatgtgaggaagaaaaccattaggaggtgataaggattcaacaatttaagtaatataacttatgtacttcctatgtcaaactttagggaggcagatgttaacaatacagtcctcaaggagccggttactaacagggagactagtatattaggtggtggcggcaatggttaggagggggaagtttctcgacagcaattgcagggtcagtgaaagagatacttgaggaagagagaaagaaatggctctggtggtttatctttcctttttccccttccctctctctcacactattaattatgacatcacaagtgggaataaaagaactagagggaatccccagtaaaagacttcacatagccacaggccaccagactgtggcagctgtccctgaatcatcatgggtcagttggtaacgacctgtattttaaccctagatcacaattttaggcccagcaaaattgttctgaagctgtctagatttattaagctatatttatattttagtttatgttaatatgaggttgagatccaagaccctgttttctccagacctcatatgagtacagtatcttaagttagaatttatctctctgcataaaaatcagacctagtgcgtaggcttctggatctcagaatcacttatacttaagtccaggctgttctcaaataaggcaagaagcatctgctgttaatagctgacagtaaattacacaaagtaaaacatggaaaattaaagtcagaaaagctaggaagcttttctatcattttcaattttctgcaaaaatacagacataatcagtgtttaggatctgcttgtgatggataaattacatctgtaattccttcttttccatattactgcattcagacgataatttgctttcagatatctttgctcatctaatcgttcatagactggaaataagtagtaacatctcccaatcctaggaagcatttataactagtctttgcctttttgggtgttgatagactagtggtgattataagctttcgagcttctgaaaagcacaacgaagattaaaataatcataggataataaaatactttaaaacccttctagtctttaattttaaaatgttccagtagaacacaaatttgctcaggtaacacacaagtaagcattaaatgccttcctgtgtatctgagaagtttgttatgaaatattttggaaaccgctgcatagtcagtgtaggaggagcagatgaattttagctgtggttatgtgtgctgtaaaagactatacgtgcttgtattagtcagaatgagtacaccactaatttttgtatggtaagagatttatactaagctcatcatcagtttctataattcagtgagataaaactgagtcagattgatttttaggtagcacatgtagaaacagctaattttattcccctgatttgatcctcatctattgattatataaactaaagaagctaagaacaattaacccttacgaggttacacagtcaggagatgctgaactgagattcagtgtagaaagtctgtcttcagagcctatgcttttagtctttatgctaagtttaacttgtttaaatagcaagattatgaagcactatacagtgacctcgtatagacaaaaatatagtatattgattattagagaaactacatattagactgttgtacatacgtgggcaagtatttgttaaatcatttcagttgcctaaatttaagcaactgtgctgtttaaaacatgctcattcacattttttcttaatctagaaagtcacttctgaataattgcttgtttagattttctcatttggtgtgggaaatttatattaaaattttaactaatattctaacaatacagagtctgaacctaaagtccagaagaattttaagtcatgccgcagacaggatgaacagtatagcaaatcagaataatagactgtttgggggggttggggggaacccatgagaatttcaggatttcaagataaagcttggaattgaggtaaaggcatcagataaggaagtgtttcatttcataactttttttttgcttgaaatataatatattttaaatcacaaaagtagtataactgttatttttctaatgcacagaaccatgagactttttttttttttttttttttttttgagacagagtcccactctgtcgctcaggctggagtgtggtggtgccatctcagcttactgcaacctccgcctcctgggttcaagcgattctcctgcctcagcctcctgagtagctgggattacaggcatgcaccaccatgcccagctaatttttgtatttttactagaggtggggtttcaccatgttggtcaggctggtatcaaactcctgacctcgtgatccgtctgccttggtctcccaaagtgctgggattacaggcgtgagccactgcgcccagctcaaggcattattttataagaggctgagattttattcatcttactaggcatttatttatattatattgtttctaatatatgcctagtaagacagaaatatgcttaaaaacctttgtaaatctttacaagttgtactatattaagttaaaatccagatgaaaccattcaccacctctttgacaatgattcatatatcttattgtatgtcatgcttgtttgtgattttgagtattacatttcagaaattttctaggactaaaatttcagtagtttattttaattttttgtttatttgtaataattatcaataatgctgaaatatttagatctagtaggtagaatttttttcatgtgaatacattaattttaatgatgtatttaaaaatagctcacactattctttttaagacttttgttatatgtaattggacaaaaatttgaccatgtaaatataagaatcgttgattctattttgttcatgatatggcataaagagcatgtttgctctcaaaaaacatgggtttgagaagatgaaatttctgggcatactgggtatttagtagctatcagattgtaatcctgaactcttaagagatgtatctcggatgggcgcggtggctcacgcctgtaatcccagcactttgggaggccgaggcgggcggatcacgaggtcaggagatcgagaccatcccggctaaaaacggtgaaaccccgtctctactaaaaatacaaaaaaattagccgggcgtagtggcgggcgcctgtagtcccagctacttgggaggctgaggcgggagaatggcgtgaacccgggaggcggagcttgcagtgagccgagatcccgccactgcactccagcctgggcgacagagcgagactccgtctcaaaaaaaaaaaaaaaaaaaaagagatgtatctcaaatattagaattgatcatgttatttaaaatactccatgggaaatataattatttttatggaacagttcatttggatgaatggtagcttgaacagaaactttcgataccaatagctagtgttataagtgtcctttcctgaaactcttgccacgcattgtgttttggcgattttcatggttgtacgaatttagtcagacagccaattagaaatatatttttagaaaattaaaaatgaatttatgtggattcaatgagatcagttgggtaaatcccttaattcagtgtcatgctcatgtaaatactccataaatggtagccattagtattgattgttcttatctgaatgccagagtttagtctaagttatctttcttcttatgggaggaactttgtcttaggtcagaataaagattgttatatagattcaaacttggtatgtaattcaaacttattttatgttcaagactaaattgatcatagggaaaagtgactggtgttccttgtcctttcatttcatttctctggacgctttcttcattggtttacattagctatagtgtaactttttattgtgttttggtattttctgagtgtctagattggcctgtatgctcttttctttgggatgaaatttgtaataaaaattattttgtagcaaataaatactggcgacaggaataatgacaatgtgactaccagtggttttcacttcattcccagtgtcacgtttagaattttcatcaaactaggatcttcagaataattgcaaaaagccacatgaaaaaatgtgcaagtgaaaacttttttttttttttgagacggagtctcgccctttcacccaggctggagtgcaatggcacgatctcggctcactgcaacctccacctcctgggttcaagcgattctcctgcctcagcctcctgagtagctggaattacaggcgtgcaccaccacacctggctgatttttttttatctttaatagagacagggtttcaccatgttggccaggctggtctcaaactcctgaccttgttgtctgcctgcctcggcctcccaaagtgctgggattacaggtgtgagccaccgcgcccggccaacattttattttttatggcaaagaatcaagactttgttaagagcctaaaccgtggattcagctggatctgagtttgataccatagtgacttcctagtgacattgtgccaatcactttactgtcccaagctggtttccttatctgtaaaacaagtctagtatcatttgctttcaagatttgtgaggattcaatgagatcagttagatataaatcatttaatgtagtgtcttactccatgtaaatactccatagatactaacggtagccgtttgtattgttcttatctgaatgacagagttcagtctaagttatctttctcttatgggagaaactttgccttaggtcatagtgaatgttatttgtcattatgcacattgcactacaggtctctgtgttgtttgaaaaaattaaaaaggacttaaccttgaatgtatataagtgtgcacatactagcttatttatttacctgttatcaagagaaaataaccccttaacctcttaacatatgtatgacttgactactagctaaaccagtaggtattaatggaataattcagaatttatattgtctgtacctgaaaattcattttgtaaatgcagttaagtatgaatggaaaaatggttgtagccatctgttagcaaatctaaattcatttaaaactaatcctatttagctttatgctggtctacaataaataattcatacttgatatttcaaatgctgtattatttatgtaaattttctcctaaaagactcacctaagacttttctagaaataaaattgtttattcagcaaagtacctgcacagattagaaggttaaatattaaagtttattttttcatgattgcactattcttttctatttttttatacctatataaacttaattggtcaaatatctaccactcacttcctcaaatgtgtaaagaagcatccaaatccataatactctgtttatcacagctttttttttttccccctttcactgtcactcagaatcgatagggatccaattctagcctgttgaaatggtccgttttgctccaagactttttttttttttttgaggtgggagtttagctcttgttgcccaggctggagtacaatagcgtgatcttggctcaccgcagcctccacctcccgggttgaagcgattctcctgcctcagcctcccaagtagctaggattacaggcatgtgctaccacgcccggctaattttgtatttttagtagagacagagtttctccttgttggtcaggctggtcttgaacttccaacctcaggcgatccgcctgtctcagcctcccaaagtgctgagattataggtgtgagccaccatgcctggcctagctccaaaattttaaaaagcatttgcctcatttatgagtttgagacttttaacatcattagaaataggagagcatgtactttttccagcaagtgtaaagggggaaacaaacaccaaaaccccatctccctgatttttttggcttttcagaaataatacagcctagtcctatggccagttctgggtttttcttgacagcgtctgtaactgttcttgttatagtctaattctgggtatgagttatgtgtttacagtttgatttactgagccatgccactgcattcttgttaaggttattgattattgcccattctgttctgctgtgtattattgatttagtgtgtgacaatgagttaattgtattacttgatgttgaagaaggatcaaatatctgttcaaaggggatgcttgtagcctgtgtaattgagttggctatttcaggcttttaacatgttgtgaaacaattaggagaaagtacaggaggacctctgagggcagggaacataaaatttgttttcagctttttcgcagttgccaaaaatggtgccgtaaggctttcctggaattaagtagtcagttggtatttggttagcagatgctttgtaaaacctaatatattcccactatatttcctttaaaaatcaagggggggttaacacctttagattgtcatgttttattgagaaaaatccacttctctgaaactccctattactggatataagaaaaaataacaagcaagatgtgttgtgtatgttggtgcccacattttggaggaaattacagagatagaaaattaagctaaattattaagaatctgatgagttctttttgcttactacagttttgtttcgtatttgataaaaaaatttccatgtaataacatttaagataaagccagtattattttctttgacattattttgcagtttgaattttctaagctttagacaattcacacttaatgtaaaaaaaaaaattaaccttcagaatgagatcatggtgccatggttctctaatgaatgttaccttttgttttagcagctcagttggctctaatccatggaaaagtttttatttcccatcagctccaaagtaggagccaaaccaaagtttaaagaaacgttttccagcagacaagtttctacagataatagtgaaagcgtgtgaacacacacacacacacacacactctctctctctctctctctcactcctctctctctcacacacatatgcgcgcgcacacacccacacgtcttatggaaatattaaatcaggaagaatttctgaagttctcataataaaaataactgtaatattaataaagtactccttatcagttatttctcgtcacattgtggccccaagggtttgcttgacttttcaaaatgtatttgcttcaggataaattgaagaagtaccactctctccctgtattaatacctttcagggcttttgatctgtaacctcttgtcacctctggtttatctggcttagcatatgaattttgaagaatgagggagcataaacaattatgactgccttttaaaaaatgcacgttaaacctgcggcctcaaacttgtcagttctgaactttaacagtagaaaagtgaaaactcagcgttgaaagtagagagtgtaagagattttgccagttggaccactttgctgcaaatacctattagacccaaacctttcttttgcctagtgatcagctatactaatagcaagatccacttaattccatgtgagtcaacagcttgcatggcatgcccactgtgtgcaaatagcttctcaggtacttttccaagcgtcctgcctgcacaaagcagaagagacttggaatatatttatttattctctcaccccactgcttgctctatcacctagaccttcatgctccatcacctagtcctgcttgctccatcacctagttggaatttctaaatcaacagacatgggcagatatatattttaaaagctgtgtgcctctgattaacaaccattcctttaatactgctgagaggttctgggaacatagtatttttgccatgacttctcaatctatagagaagatgtatttatctcctaaggataaaaccatctggaaattttcacatgctccagactttcagatagagctggctttgccttctctttttcttcctcctcttccttctttttcttcttttatttatttatttattttgctttgcttattaaaactatggatgaatttttaaaaagattaagaagataaaataaccttctcacctgcttagtaatttcttattcatagttagaaggtacagtaactgtttcataactgtccccattaaatttctgattcaaaaaatatatatttttctttataaataccgtttcctatgatttttggtccctaaaaagcaaacaaaaaaaagctacttgatggtaccttggttgtagttagcaggtctccattttctacagatcttccattctgctttattaacttcctttattaatgacaaatcagccagttgataaaataatgtgttaacttttaaaagattcttataaatttatgtggagttctgtgactacctgatatgactttgtgactattttagttgtataaagaatttcaaattatttattcctttaaaaaaatttttttttcagagacagtctcactgtcactcaggctggagtgcagtggcgcaatcttagctcactgcaacttccacttcctgggtttaagtgattctcccgcctcaatctcctgagtatctgggattacaggcatgcaccaccatgccgggctaatttttgtattttttgtatagatgaggtttcagcatgttacccaggctggccttgaaatcctgagctcaagcgatccacccaccttggcctctgaaagtgttgggattacaggcgtgagccaccgtgcctggcctattttttgttttatttttttaatcttgaaataactccacagttatctccaagtaagtctaatatcttctctttgaatttctaactgattcttatttataaacccatagaatctgtaaacccacaaacaaatgattcttcatcgatcaagttctgttttcaaatagatgttcaacgagatgttcaaaaagacgataaaggatacattgtaacctaaggactttatttcgttcatttggtcatttaccttttaattgtagtggacttaaatgtatacagattttcatgtacaacaagtagtatcctttagctgcaacaaatgaatagattagcctatgtgaatatagttttgcaaaatattaatgtatgaaagtagtcctaagactgcaagtactgttgctttagaacttcatgcataaattgggtgtgttgttacaggatacagagaaaattgatggtcaaccttagcctgtagtatattttgacagtgtgaatgagtcaagcaagcaattttccagactgtttaatggtacaaaggaaagggctgctaaggaagtcattcttatgactagctgaaaaatatttttcttagtgactgttctagagaaaaacaggtcaccgttggaagaaaaaagcagtctcacgttacaagcattttgaaagtggtttgatcattaaaatgtatttatcttacgacaaaacttaaaacggatactgcttactatatttgcaatgtcttgaaactcaacaagaaattatgtagcatcattacaagttttaattcatggtagaattttggagattatatttattgagctatgttttttatttcattagtacaaataaaactgcattttatttgttctaatgataaaatgttttaagtttataaaattactgcaaatgtattccactgtgtggttctaaactgcatttgaattaaaataggacttgattgatggagaatcatttgtttgtgggtttacagattctcttctaggtagtttgaaaagattcattgtccagcagcaactgttcacccagagacatagtgcttagcatgagcttgatggattcaaaccactgggtttatcattactacatagatctttgaaaagacaggctgcaaatcttgtttctaagctgtgactagctattattatgtcaatttgaagaacagaagtaaaatcaatccagattcaaaaaaagacactctacaggcattcacagactgcaaccagttcaaattgtaatatcctgcctatttaaagtcccctgattattatgattattcaatatcatgttaatatttaaaacttggaatacaacctactaaccttatttttatttgtactactggaagttcctctagcatttttgaagtagcttggaaaaaaattgagattcagttgtggttctacttacatttttactagagtaagttggagagacaaatcagtgagtagtcaagtcttcatataatcaaagcttttaaaatatgataacaaaaacttcaggtgttagatttagcttcacggaaaacctttttttggggggcctaagtttaaaatgtgttttttctttgcattttaaaagattgcttgtgcagtgttcaatggttttgtttagcatttagagatgattttttcctttgtcactttccacattgaactctgctgatttagtgtgttagaattgccatcagaagagtccttagaaatgatgtcatgcagttcattagttttacagatagggaaccaaggagtggtgaggttagggtttaatcatatccataaagatcttgaggtcattaggaccctttaaatgtgactagttaaatcacagcaaaaactagaatatattgtcttcatttcaggaaaatggcatcattgacacacctgacagatgaaaactgctattaaggtcgagtccaagtgacaaaactttcttgactgttctgaggccttggttacggtggcattaaagttctatactgtttttttttttttttttttttttttggctggacaacagactctccaaaaaaatgcatgtgtatacattatgcttattataaaatttactaataaaatgacatggaagacaattaaaattataataaaatatacaatgctcaacatttaaattctatatggccaattgattcttacagaatgcctttttgatttttgtccaactcttgcatctgtagtcaacctgtggtcgcaattgacagaagagtttaattctttctttgtgctcttactcctgggtccctcattgtacagtgatcatcaaatgcaacatccttcatgaaaatgaatagcttcactttttggcatttccttcctttttatgtgtcatagaatacactacattattcttgattttacagttaatttgtaaattgtttctaacccccactagtttataaactccttaagaatgagaagcttgcattaatcacttctatatatatagtgttttgaatatccttggcattcattaaatgtttagtgaattgaatgcgatgtttcttccagactatgaccaataattgtgagaagattttatgtgtttttaaattcaactttagagaatttctttttgtgtaataggacatttgttggacagcaacatgtaattactcataaaatgtttctctgcggagtccagcatccttcatttgtgataggactcatccagttaaattgctcaggcactataatggactatttatgttctgttgatgtaaatttctagaacaggcctttccctggactcatgccatgttttgtatgttatcttgtgcaggcagcacatggggacctagtcaggcttcctgatatgatagctgtatatacagcccatgtctcagaggggaatgataaattggcctgtgcagtgacacaatacacagttattttggtgacagtattaactctcttttcataagggtgctttgttggaaaactattcagcttaacacagaatatttagctttcaaaggttgcatagggagaaatgatactgccaggatagaaacattatcagttttttctttctttttttttttttttaatcttttttgactctaccgagaatattgaaaagagttttgattagtattggtgtttattacgagaggaacattctgggaaccagactattagtataattattattgtttttattatcaaactaatagcactaacaaggacatacggtatttgtcaagggattcctatatctctgttacacctaaatttctctatactctaattttagaaaaaaagttttgtcttccaactgtgaatactttatagtgttgcaacttctgcttctaatctttaatctgaagtagaaaagagaatccaagcttccaagaagtatgtctctagactctgccacttattaacttgctccttattttttctctccaacaattattttaagatctgtatgatataagctatttttagaagaactgaaaagttctagagagtaaaaaaatgttccagattgtgtgttggtgccagtagtactgacaattaaatatttgattgaggagaaacctacatttatccactttgaagaggatcatcaggggagttaaaaatttagtacttcaggctcattaagagttagaatagagtaattatgtagctggtaacaaatgctacaatcaccacttgagcattgtgatatgttactcattgagacttactggccttcttcagggaacttcagattttataaaagtcatttgcttagagagtaaagttctgctaatccattattttagttctgtcatctttttctgcaaatagggtgagttttccgagagggtaagtccctggtaataatcttaaaaatgatacaactttacagaatatctccccttgccatgaagcaatacttaatgtcttgaacagtgagcctcttgttcatgcagcatttaaaagagatgagtgtagaggtcttccttctacattcattttattcgttagtaattgagaagcaggtctaattttctaatcttgcaagttgggaaattattaaaatgctcaagagacagtagatcaagcctggtgtccctttctccatcaaattttaaaggatttttttttcctcagtaattctgcctttgtgtatttccaatcttgcttcctatttgcattgcacacagtggcatgtcaatttcaaaagaaaatgtattcagatctactttcaaactaactgaaagagatagtggtatttattttatcctgaggttttgttgttgttgttatggttgttctttgttttttaaatctggacatatcagtaatctcctaagagaatctattccaaacacagagactgtttattcttgttttagatactgttttaaaagccaggttatgttattatagtatagatattttaaaaaataatgaaaacagagtttttttaaccatatttttgtactgcataaaaatgcagtgacaagagaaaaacattagaactctgtcatactcctaatatagaccattgctgttaggtttctaactccatgttccctgtctggaagctttataaggccgtgccttagtcttcttaagcctaacacattgtagccttccaataaatattactttatttgtattaaatttaaaatggaatagaatgtggtacatcaccagcgttcatagctcctgacccatgccataactttccttgccccatccccaagattaaaaaaacacaaactattagactaattatagtcattacagttgtaaattaggtcatgcatttcattaaaaattgacgcagctccctttttgaaaggctatgtcacggaaatgcaaactagccttgtatatagaacactaaaatcatttaacttgaattattctaattacgataccacaaagattaaaaaatacatctatattatctcagtctgataactgggctgggaaaaaatgtcagttttgatatgtaacaagattggtatgagttattttacgtgacagttttattgattgatttttttcatttataatcactaattttgtgaggttttctcccctgcagcagtggactgaaaaagttaacatttttctggcattttttaagcgctaagagtagaaatcaatagttaaattgacctaataaaagatctgtgcatcctactactgtgacaagaagagcaacagaaagcaacatgtatagactcttaacaaggctcaaggcacattttggatttgttttgcatatgaatttaataagcatgagacagtgctagtgaattacatttgttttattgagtctatttaagccaggatactgttaagtaatagactgacggtttgtgggagccagattcagtaacaggtcagcaacagacttgccacatgccaaaaatggggttatactggtttcagtttgctaactgcctaagtaaacttgtgtagaatttgcattttttgctttgcaattactggttacttctctttaacattacatttgcattctatgtaaaaaatagattttatatttaattcaaaatttatccaaatgtacttactcattggacattatttttaaatgataaaggaacttaaaaaagtgataggatcaatatagtaataattctaactcccttttatacccatataaaattcatttaactagacttattcattcaacaatattgttaaataacctaaaggaatttttttaaagaaatagtgtaaacatacaaatatgaataatttgaactcccttttatataaatatatctgtagtcactaactgtaaataatttaaaaaatatatatgtacacacacgtgcgtgcacacacacacacacgtcatgccttaacaatggggatatattctgtgtgtcattagggtatttcattgttgtgtgtacatcatagagtatacttacacaaacctaaatggtatagcctactacacatctaggctgtatagtacggcctattgctcctaggctgcaaacgtatacagcctgttatcctattgaatactgtaggcagttgtaacataatgttatgtatttgtgtatcaaaacatactcaaacgtagaaaagatattgtgttgtgctatgaggttatgatgtctacaaatgtcactgggtggtaggaatttttcagctccattgtaatcttatgggaccaccatcatatatgtgggtcctttgttgactgaagcgttgttatacagctattgactatagttagtgaaaaaatctagttctatgattggttttgttccctcctttcttatgatcactattcattcaacaccttttatatgacatgcagtttgaaagcacagggtatgcaaaaattatccccgttttcggttgcagtatctgttgctgacccagataacttcatttccttcagctgtgctttcattttcaacttctctagttggaaataaatcctcttgtgttgccccttgagggagaaatcgtttagcaggaaatcacttactaatcacagttcattaatcacatataaagaatactttataaatgtcaactgtcattcatagaaaatgtatgttaccttgaggaatgtctatgaattacttattcattttcattgcctttcacacattatattccttaggagtttattaatttcaaatataaattgactatgacttatacagagtcttgcaaacaagagagatagcagttttgcaccttttctttgatttacttgttatcttcctggtcatgtagttagtctgttaaagtcactctgcctgtatagcttgttgaaggttacaggcagtggagtgctggaactggtttatgtcgagacctcttcccaattccatgttcaatgatgtctttgttgatagcttaaaattggctatggtgagagcgtttacactttggaaattggcagatggcaaccatagttctctcctagacaaccacttattaaacctttagcagcactcactgtttatagtgttctttttcgttttttaaaaatacttttattatgaggaatagccatgtagaattttatttccagatattttgttgtttaaaaccctaagaacatcactcactataccaattgccaactgagtaactgggaaagttacttagcttttctgtgcccaaatgtccttatctgtaagacggggatgataataatagcacgtaccttgggattgatgtgaggttgaataagaaacacatttatttcagtgaatacataatgacctggaatgaatactcaaataatgctacctgttatctctctgtactttaattaatagatatttattgatcccttactaagtacttagcattttgccatgtacagtgggtcatctactgagactcagtacctcccacatcaagttattagccacattgacctgtggcaaatgacttcttttgatgttcccatgtaatgccacatacccgcccccccccgcccgcttttttttcatttgtttttgtttgttttttggtctttctgcctgggactcttttttcttcctacatgagttttgcagaccttttccatgtttatatatggctcatacattccctcctctagaaagcttctcctcactctagacctggccacataactgttttgcctctttgcttatgtagtttggagtatgaatcataataattatgtatcttactccatctctgcatttgattccacagcctacgacttacacctttgtgttcttaacctcttagtaagaaatgcctgacacgtagtagactgactagaagtgtacattgaatgtttgctggttaaaggaatggaacatggacttcactcatagagaccttactatttggttgaagagataatgcatacacatgtgacatgaaggtaaacacaaaatatattgaagatatctgtattttattttttttagtttatagttaatttttacatactatatatttttgtagtaggtaagtcaattattttcatgctacagatatggacatttatgtatttatgtacaaatacattgatttaggttatctaggccagattgtaagtagcaaagccctgtttgtaaaccaggttttgtgacttcaggttagaaatattaaaaaaaaaaaaaaatccttaattgctcttccagatatgaactcattctttttttttttcccctgtgatttctgtcttttttgtttgttaagtttaaaaaatttatggcaatttttaaatgtacataatattagagaaacaatataacaaccccctccatatatctatcagtcagcattttgtatgcatgtatccgtatgtgcatcagccaacattttgccctttttgttacctttcacactccttttacccctgtagcatgttaaaagaagtctcagacatcctatcatgtcacttatacttcttcagtagtatctctgacagttaagaacttattttaaatataactccatttccaaatctatgaaaattgacgacaattttattgtatcatttgatagccactttatgttcaacttttcctgattgtcacagcaatgtccttttttaataggtacgtttttccagttagcatctatggtgtcatttagcttgttcttccatcatccttattttctgtaaactgaaaattagagtcgagtttaactttaatgttatttattattatttggggggaacatttcctggaaggttctgtatactttctatttcatcatatcaagaggacataattttagtaatccctgcttttaatgatgctgcaatctatcagtgaatcagattggcctgggccaatccttataaagttctccatcagacttggatccaatgatttcacctcattgttgatcattttgtagccatgatattttttagagtttgcaaaaatggtgacttttaattatataatttctgctgcgtttattatcaagaatttttttcataaagatcttttcatagtaactaatggtcaacttcaaaacatagtttatatcagaaaaagggaaaaaatgcatgatatttatcaattttcagaatcagatattctcttctaacataagatctaaataagcttacacatataatcaaggatgccagtaaatttttctaaaaaatttttcccttgggaaaattatttatttatttatttatttatttatttatttatttattgagacagagtttcactattgtctcccaggctggagtgcagtggtgcaatcttggctcactgcaacgtctgcctcctgggttcaagtgattctcctggcttagcctcccgagtagctggaattacaggcatgcaccaccatgcccagctaatttttttgtgtgtatttttagtagagatggggtttcatcatattgaccaggctggtcttgaactcctgacctcaaatgatctgcctgccttggcctcccgaagtgctgggattacaggcgtgagccactgcgctcggctgataaattatttgaattgcttctcctaattttttttgattaatatgttttgataacattgttcctatggtaatattatcaaaaaagttttctttccttcctttttgtttatcattaatgtgcctctgtatggttgaatttatttactcagaagatatttattgggcatctgtctttttttatcaagctctgtgctagtccctgaggatgtagatatgagccctgcttttttcttttctttctttctttttttttatttgagacagactctcactctgtcactcaggctgagtgcagtggcatgatcttggctcactgcaaccgccgcctcctaggtttaagtgattctcctgcctcagcctcctaagtaggtgagataacaggcatgggccaccatacccagctaatttttgtatttttggtagagatggggtttcacccatgttggccaggctggtctcaaactcctggcctcaagtgatctgtctgccttggcctcccaaagtgctgggattataggcttaagccactgtgaccggctgcttttttcctagaatatatagtttgggtttcttttccactcgtgatgtgatttaaaaaattgaacctgttgtatatgaatgtggttttgtaaactataatatttggtatcatatactatatttttttctatatatatatatgctactatatagtcatatagtagcatttttttttatagctttctttggagttcttggtcttagtgtctaaaatagtggccataaaggaatttggaagttaagttgttcttgctgacccaaagaaaaatcagatgtttctgtttaggacactgtatgtgacaaacaaggaagtttttattaatttatttttttgaagaagaattgatgagagaataatgagaattcctccaagattaaaagtaaattataagatcctgtgctattttaatgtaagccaacatttgaatttaatttgacttctattttcagtaaattagagtgaaattttagggatgtctttatctcctatagtgtgtgttttgccaagatttattaagggtaattttggattttagaaaatgtacagtttctaaaggcttgtgaacgttttatattgatagaaaatgtaagtaagactagtaacacattattttctccaaaggatcctgtgactgacacaaaaaatatgatataatattgaatctgccaaaagatgcactgttaatctagggttatgttatgttgttattttattaggtatgtgatactatggtgtgaattttagaatataggtaattatttaagtaattactgtaattctaaacatcttctgagatgttttctctaaagcagaaactcagcttttactcatgtttagataatgtgatatagctgttctagatatttgagattagggttgaggcaaattgttgacatataattaaaatgtattttataactaaaatttagaagaatggaaaaagtttacattgtatttataagactttgctaacatgctttatgacattacggagaagtactaggaagaaggagatctttaaaaggagtgatattctgtttgctttgctggagtaccttatcaggaaatttgaaatcttagccctttaattgtgatatctgttaattctaaaacttacactgaatttaactttgaaggtcaacatatggatttttttaaaaacaatatttactctatcaaaagattagttgtatttttgacttgaggaaatagcttaatctgaacagcacgaaacttgacaaaaaagtttgctaatacataaatattggctattttaatctgttgaaaacggaaggctagtaccaagggagacggtagtaaaattatagaaggagctagtgaaaagtaaacattatgcaacatgaaaagaatactaggggaaaaactgacagagtagaataacaggaagattagtcacttctggaaggaaaaaagtaggtaggggcagggggatgaagtactttaaacaggaatgtggaactggaagtgctagggctaaaagacagatgtgagcttatttgggagctaaactgggctttgaaaaaggaataatactggtagcatttaatatcaaaatttaaattttatgttaacaataagaaaaaaatggagaaaaatttttgttagggattataaaatgcctgttaaaataaaaagacttatatttactcctttgtggagctgaattggtttcggtcagacagcaatgtcaaatgcaaatcaagaaaatacatttctgaggccaaggcaggagatttcttgaccccagaaatttgcaacaaccccaaggaacatagtgagactctgtctctaccaaaaagaaaagaaaaggaaaagaaaagaaaaaaaattagccaggcatggtggtgagcacctgtagtcacagctactcaggaggctgaggtgggaggattgcatgagcttgggaagtcaaggatgcagtgagccatgatcagccattgcactccagcctgggcaatgaaacaagaccctgtctcaagaaatatacatatatattttatcttatatatatatatcttatatattatatatatatataatatatatgtcatatataatatataatatatataagaaatatatatataatttctttcaaaaccggtcaaagttcttttactttcaagtgatggataaaataaaaccattgtggatatcatgggatatatgtgctattggtttgaaataattttcacttttgcatttatttatctgtcaaaggagtaccaatctctaaatattttaaggattatttctatgaattctctaaaaaatataaattgtaactgtctatatcatacaaaaatatttagcaatccttataacagaaaacaagatagtaaacattgtgtgtatgttcatgtgtgtgtatgtgtgtttattggtagtctcattaatccctagtctctactgacgttattagggacattagtggaacagattttgctgaacacattttcagccactctgagtgttcttaggtattttatttatttagagactgaatctgtgttgtcctgcctggtctcaaacttctgggttcaagcgatccttctgccttactttcccaagtatctgggactacagatgcacaccactgcccagctaaaatgtactttaaataaagtagataattctggggttatctcaaactcctggcttcaagtgatcctcctgtctcagcctcccaaagtcctgggattactggcttgagccactatgcttgccacctctggggttatattagcatgacaccaggacaaaaatctgcccatttttatatcaggaaaaaaaatgctactttcatttaggtgcagaaaatggcttatgcctattgaaataaaaaattcagtctaatttagacacctaagattattatttgctataacgacatggaatacatcactgtgtgtcactgaattcatggaaatgcagtacagaatgcaatacagtaatccccccttatctgcaatttcactttctgcagtttcagttacccatggtacaacacgataagatattttgagacagagaaaccaagagagagaccatattcacataacttttattacagtataattgttctgttttattatagttattgttgttaatcacttactgtgcctaatttataaattaaactttattgtaggtatgtataggaaaaataatagtatatttaggggttgttactgtccatggtttcaggcatcctctgggggtcttggaatgtattccccatagataaggattatctactatatacagtcaggtaagccattcccattgattttattacaaattgcagtgcttccatggcttgcttacacataatttgctgaggtaacttttgcagaaagtaaccattttacgtgaacaccaaatactgattaaaaaaatctctttttctccttttccctctctttctctcccttctccctctctccttccctgtcttactccctcgttcctgctcttcttctcttttcatactcacacagacactccttcccctacacacacacacatacacacatacacacacacaccctgcccctgggcataactgaataggaaatgtttattttccaaactacagaaaaaaaccttatctcaaacactgaaagtccttccttttcctggtttggaaacactaagtcatactgagtcacttgtagattttctttagccagttttatttaaagcataaagttgtgaggacaaaatgactcaggaatttgtaatgtaccattagagcttttcacttttaggatgctagttcatattcagatgacttagtattaaagattgatgggttgtttactttctagaagaatctgaggagctcactgtaacttcacttattttgacctaagtatggcctagagtttatgcttattaattcatttatttctcggtaaagccatgtccaaggaattaaataaaaggaagcggacccagtaagggttgatgaataagaagctgttaccttactcatttacatgtattacttcaaatgttattgattgattgattgattgattgagatggagtttttgctctgttgcccagactggagtgcagtggcatgatctcagcaacctctgcctcctgagttcaagcgattcttcagtctcagcctcccaagtagctgggattacagcctccccactaccacacccagctaatttttgtatttttagtagagacagggtttcattatgttggccaggctggtcttgaactcttgacctcaagtgatccacccgcctcagcctcccaaagtgctgggattacaggtgtgagccaccgctcccggccttgatacctctttaaaaatgaagtttgttaaaatgatcagaaactttaatgcagctgattgcattgccattgacatcagtgagaaataaaaactatagctataatggttaagttgagcagttggaatctgtatgtctgtatctatctatgtttatttagatgactggataaaagagtcatgattcctcctagtctatcacgtggtaatgccaaggtcagaaaaatcatttgttatgatctatcttatgtaaaagcctgtggagttactttgttttcctcactcataagatgtcaaaatgtgctttctagaatttttgaataatttttgcttaaattcagtgttaaagttggagactgaaacaaatttaaatctgttgttagccaaagagtaaatcaagcttatgtgggtgcaatatataacatgactacccaagaaattctgcaaaaaaatgtagttgtctagcaattaggtacagagttgtcatcttccttctatgataacttttgattataaaaataattgaaggtaaataaactgtaaatgacacaccacctcaagctatgccacatgggcacctaaataagttctttttagtaaatgccaccaaatggctgtatttcttacagcttatggctaaattctgacagaggggaggtgccttcaccataagagtattttctctttgaagttttcatggttttgtgtgtgtgtgtgtgtgtgtgtgtgtgtgtgtgtgtgtgtgtgtgtgcgcgcgcgcatgcgcacacactagtttatattaggttagagtttaaaaaaagctgaagatagccaaaattagtccagagagttagacttgctgccaaactaaagaaatagcttggtttcagtattcttctggcttctcagtcttaacatttggggacctgagtgcttgatttgagctgactgaaattaacccagatggcccactttgggtcctcaagagctaattaaaggaagtcatttcagttgctgaataatcaaggccgtaccttcctccaatgttggtttcagcccacaccttacagatgatcgctggctttctgaagctctctctaaactcataattattgtttggaccctgcatgtaactaaattaatttgccaaggatgggaatgaaatgtaagttttattggcaacaatggtaatcccccaaaacagaatgacagatacagtgatggtaataatgttac

SEQ ID NO: 17 - DNA, 100 bp, Homo Sapiens
ccaatgatacattcaagtgggtggtttgcattatatttttttcataatttgctgattttattcttttctaagatgatgtttgctgcttttgtttctctag

SEQ ID NO: 18 - DNA, 171 bp, Homo Sapiens
ggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtg

SEQ ID NO: 19 - DNA, 5960 bp, Artificial Sequence
  Synthetic
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagccgtgttttagagctagaaatagcaagttaaaataaggctagtccgtagcgcgtgcgccaattctgcagacaaattctagaggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtcaccatgtacccatacgacgtaccagattacgcttctaagtcagagtctcctaaagagcccgaacagctgaggaagctcttcattggagggttgagctttgaaacaactgatgagagcctgaggagccattttgagcaatggggaacgctcacggactgtgtggtaatgagagatccaaacaccaagcgctccaggggctttgggtttgtcacatatgccactgtggaggaggtggatgcagctatgaatgcaaggccacacaaggtggatggaagagttgtggaaccaaagagagctgtctccagagaagattctcaaagaccaggtgcccacttaactgtgaaaaagatatttgttggtggcattaaagaagacactgaagaacatcacctaagagattattttgaacagtatggaaaaattgaagtgattgaaatcatgactgaccgaggcagtggcaagaaaaggggctttgcctttgtaacctttgacgaccatgactccgtggataagattgtcattcagaaataccatactgtgaatggccacaactgtgaagttagaaaagccctgtcaaagcaagagatggctagtgcttcatccagccaaagaggtcgaagtggttctggaaactttggtggtggtcgtggaggtggtttcggtgggaatgacaacttcggtcgtggaggaaacttcagtggtcgtggtggctttggtggcagccgtggtggtggtggatatggtggcagtggggatggctataatggatttggtaatgatggaagcaattttggaggtggtggaagctacaatgattttgggaattacaacaatcagtcttcaaattttggacccatgaagggaggaaattttggaggcagaagctctggcccctatggcggtggaggccaatactttgcaaaaccacgaaaccaaggtggctatggcggttccagcagcagcagtagctatggcagtggcagaagattttaattaggtaagtaagcacctttttgtgtgttgacataattttttaaattgctgatgaacccaataaccctaatgtagctgagcagtgcaacatagttaacattataattgcagtaattgtggatataaagttaatattcagatcagcaaaatttgtgggaaacaaacttgatattggattgtagccttgagtcttaatatgtttagattaacaactttattccatattgttcaacaggaaacaaagcttagcaggagaggagagccagagaagtgacagggaagctacaggttacaacagatttgtgaactcagccaagcacagtggtggcagggcctagctgctacaaagaagacatgttttagacaaatactcatgtgtatgggcaaaaaactcgaggactgtatttgtgactaattgtataacaggttattttagtttctgttctgtggaaagtgtaaagcattccaacaaagggttttaatgtagatttttttttttgcaccccatgctgttgattgctaaatgtaacagtctgatcgtgacgctgaataaatgtcttttttttaatgtgctgtgtaaagttagtctactcttaagccatcttggtaaatttccccaacagtgtgaagttagaattccttcagggtgatgccaggttctatttggaatttatatacaacctgcttgggtggagaagccattgtcttcggaaaccttggtgtagttgaactgatagttactgttgtgacctgaagttcaccattaaaagggattacccaagcaaaatcatggaatggttataaaagtgattgttggcacatcctatgcaatatatctaaattgaataatggtaccagataaaattatagatgggaatgaagcttgtgtatccattatcatgtgtaatcaataaacgatttaattctcttgaatgaaatgacaactgtatggatttgggactggcagagatttggactttccctacccactccccctgataataatgttgaatgcttctatcacaattcaagttcaaagctctgccagggaatagaaactagctgctggctaatgccgctccataaatccgcagatttgaagtgtctgggaggccttcttaagatacattgatgagtttggacaaaccacaactagaatgcagtgaaaaaaatgctttatttgtgaaatttgtgatgctattgctttatttgtaaccattataagctgcaataaacaagttctgattttgtagttaacacgtgcgaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 20 - DNA, 5762 bp, Artificial Sequence
  Synthetic
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagccgtgttttagagctagaaatagcaagttaaaataaggctagtccgtagcgcgtgcgccaattctgcagacaaattctagaggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtcaccatggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaaggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatggtaaggagtgccctctctgtcccaagccctggccccaccacagccccttccccatgacgccgcccccgccccgccccgtactttgcaggtggtaagtttctcagccctggcaggacctgtgtccgccccgttccccctgcgcctgcaggggccacatctccggggcagccccactgcctcctcagcccccacagcccctatagcccccatgccacctccctgccttgataacagtgcctcttgtcctctctggccataggataagtgataagtagtaagtgagtaactgagtaggctgttccccctcctccatctctgaaccgtgtcacaggtatcacccccttcttgccctcagcccagctgctgtgcccctgccacccgcgccccctcagccccttgcgcgtcgcatccaaggtcacttgtgctcgcagctccacctggagccgttgccactgctgctgctgcgcttccagtcagggtgggccgctggcctcccactgggcgtcagtttggctcccaggccctgggcagtgccagcctctgggcccgtctgctgcgctgcgttgcgctggctgtgtgctgggctgtctttgctgtggggctgcagtgggggggggcggggtgtctggggacgcaggtgagtaggggagaaactggcagggtggtaagcttctgagcctccaggtaagtgcgtgcctgggagatgcacctgggagggacctcgctgccctcctgccgcctgcctcatcccttctctttccccttccagggaattcctcaagctgtacttcccccattaccaaagaggcctgctcttgaaaaaaccaacggtgccaccgcagtctttaacactggtattttccaataccaacaggctctagccaacatgcagttacaacagcatacagcatttctcccaccagttcccatggtgcacggtgctacgccagccactgtgtccgcagcaacaacatctgccacaagtgttcccttcgctgcaacagccacagccaaccagatccctatcatcagcgctgagcacctgactagccacaagtatgttacccagatgtacccatacgacgtaccagattacgcttagcttaagatacattgatgagtttggacaaaccacaactagaatgcagtgaaaaaaatgctttatttgtgaaatttgtgatgctattgctttatttgtaaccattataagctgcaataaacaagttctgattttgtagttaacacgtgcgaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 21 - DNA, 10296 bp, Artificial Sequence
  Synthetic
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagccgtgttttagagctagaaatagcaagttaaaataaggctagtccgtagcgcgtgcgccaattctgcagacaaattctagaggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtcaccatgtacccatacgacgtaccagattacgcttctgaatatattcgggtaaccgaagatgagaacgatgagcccattgaaataccatcggaagacgatgggacggtgctgctctccacggttacagcccagtttccaggggcgtgtgggcttcgctacaggaatccagtgtctcagtgtatgagaggtgtccggctggtagaaggaattctgcatgccccagatgctggctggggaaatctggtgtatgttgtcaactatccaaaagataacaaaagaaaaatggatgagacagatgcttcatcagcagtgaaagtgaaaagagcagtccagaaaacatccgatttaatagtgttgggtctcccatggaaaacaaccgaacaggacctgaaagagtattttagtacctttggagaagttcttatggtgcaggtcaagaaagatcttaagactggtcattcaaaggggtttggctttgttcgttttacggaatatgaaacacaagtgaaagtaatgtcacagcgacatatgatagatggacgatggtgtgactgcaaacttcctaattctaagcaaagccaagatgagcctttgagaagcagaaaagtgtttgtggggcgctgtacagaggacatgactgaggatgagctgcgggagttcttctctcagtacggggatgtgatggatgtcttcatccccaagccattcagggcctttgcctttgttacatttgcagatgatcaggtatttttctccttaacgatatgtcccggccgggcgtggtggctcatgcttacaatcccagcactttggaggccgaggcgggtggatcacgaggtcaggagatcaagaccatcctggccaacatggtgaaaccccgtctctattaaaatacaaaaaaaaaaaaaaatagctcggtgtggtggtgcacgcctgtggtcccagctactctggagcctgaggcaggggaatcgcttgaacccaggaggcggaggtggcagtgaactgagatcacaccactgcattccagcccagtgacagagtgagactctgtctcaaaaaaaaaaatatgtcccaggtaattaggtaatttctcttgaacattgcccatgcagctaattgctattcttagctaagttttctgaccttgtaagacgtagggtgtatgggcctagatgtttgtgatacctgtatctgatatcacaaatacaacattttaagaaatctaaaagtaaatatgcctttagataattttcagtatgtggcatttgggaatgttatatactttttttttttttttgagacagagttttgctcttgttgcccgggctggagtgtgatggcgcgatcttggctcgctgcatcctccacctcatgggttcaagcgattgtcttggctcagcctcctgagtagctgggattacaggtgcctgccaccacgcctggctgatttttttgtgcttttagtagagacagggtttcaccatgttggccaggctggtctcgaactcctgacctcaggtgatctgcccatctcagcctcccaaagtgctgggattacaggcatgagccaccacgcctggccagttttttgtatctttagtagataacggggtttcactgttttgatctggctggtcttgaactcctgacctcaggtgatccacccccctcagcctcccaaagtgctgggattacaggcgtgagccactgctcctgctcccggcccattttttaaattattattttgagacagggtctcactctgttgcccaggctggtggaacacagtggtgtaatcatagctcactacaccctagaactcctgggctcagcctccagggggaggatcctccagcttcagcctcccaagtagctaggacagatgcatgccactacgcccagctaatgtggcttttttgtggttttttttgatagaggtggggtctccctgtgttgtctaggctgccaggctagtcttgaactattggcctcacacagtcctcccaccttggcctcccaaagcgctaggattacaggcatgagccaccatgcccagcctatgtcttttgaaaatcgactgaaatatcactgctgctgttaataaaactaaaagctgtattgggggtttaaatgaaatgagtgttcattgcttatttttcctctggctttagataaattaatgcttgtaatctaagttttgttgctactttaaatatatgaatcagtggtttaatcttctttgtttacatcccttatttcttatagattgcgcagtctctttgtggagaggacttgatcattaaaggaatcagcgttcatatatccaatgccgaacctaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatgtagacagtggggttgtggttggttggtatagaatggtgggaattcaaatttttctaaactcatggtaagtatattgtaaaatacatatgtactaagaattttcaaaattggtttgttcagtgtggagtatattcagcagtatttttgacatttttctttagaaaaaggaagagctaaaggaattttataagttttgttacatgaaaggttgaaatattgagtggttgaaagtgaactgctgtttgcctgattggtaaaccaacacactacaattgatatcaaaaggtttctcctgtaatattttatccctggacttgtcaagtgaattctttgcatgttcaaaacggaaaccattgattagaactacattctttaccccttgttttaatttgaaccccaccatatggatttttttccttaagaaaatctccttttaggagatcatggtgtcacagtgtttggttcttttgttttgttttttaacacttgtctcccctcatacacaaaagtacaatatgaagccttcatttaatctctgcagttcatctcatttcaaatgtttatggaagaagcacttcattgaaagtagtgctgtaaatattctgccataggaatactgtctacatgctttctcattcaagaattcgtcatcacgcatcacaggccgcgtctttgacggtgggtgtcccatttttatccgctactctttatttcatggagtcgtatcaacgctatgaacgcaaggctgtgatatggaaccagaaggctgtctgaacttttgaaaccttgtgtgggattgatggtggtgccgaggcatgaaaggctagtatgagcgagaaaaggagagagcgcgtgcagagacttggtggtgcataatggatattttttaacttggcgagatgtgtctctcaatcctgtggctttggtgagagagtgtgcagagagcaatgatagcaaataatgtacgaatgttttttgcattcaaaggacatccacatctgttggaagacttttaagtgagtttttgttcttagataacccacattagatgaatgtgttaagtgaaatgatacttgtactccccctacccctttgtcaactgctgtgaatgctgtatggtgtgtgttctcttctgttactgatatgtaagtgtggcaatgtgaactgaagctgatgggctgagaacatggactgagcttgtggtgtgctttgcaggaggacttgaagcagagttcaccagtgagctcaggtgtctcaaagaagggtggaagttctaatgtctgttagctacccataagaatgctgtttgctgcagttctgtgtcctgtgcttggatgctttttataagagttgtcattgttggaaattcttaaataaaactgatttaaataatatgtgtctttgttttgcagccctgaatgcaaagaattcatagcagttaattccccttttttgacccttttgagatggaactttcataaagtttcttggcagtagtttattttgcttcaaataaacttatttgaaaagttgtctcaagtcaaatggattcatcacctgtcatgcattgacacctgatacccagacttaattggtatttgttcttgcattggccaaagtgaaaatttttttttttcttttgaaatctagttttgaataagtctgggtgaccgcacctaaaatggtaagcagtaccctccggctttttcttagtgcctctgtgcatttgggtgatgttctatttacatggcctgtgtaaatctccattgggaagtcatgccttctaaaaagattcttatttgggggagtgggcaaaatgttgattattttctaatgctttgtagcaaagcatatcaattgaaaagggaatatcagcaccttcctagtttgggatttgaaaagtggaattaattgcagtagggataaagtagaagaaaccacaaattatcttgtgcctgaaatccattaagaggcctgatagctttaagaattagggtgggttgtctgtctggaagtgttaagtggaatgggctttgtcctccaggaggtgggggaatgtggtaacattgaatacagttgaataaaatcgcttacaaaactcacactctcacaatgcattgttaagtatgtaaaagcaataacattgattctctgttgtacttttttgtaactaattctgtgagagttgagctcattttctagttggaagaatgtgatatttgttgtgttggtagtttacctaatgcccttacctaattagattatgataaataggtttgtcattttgcaagttacataaacatttatcaatgaagtcatcctttagacttgtaatcgccacattgtttcattattcagtttcctctgtaaagggatcttgagttgttttaattttttttttctgcatctgaatctgcatgatttccaaaccctgtaccatctgaattttgcattttagcacttgcactattactcagcagcagtaacatggtaacacttaaaatggtactcggggacctccaaagactaaactgacaagccttcaaggagcccaggggtaagttaacttgtcaacggcatggtttaatcccttctttacacttgtgtaaatttcagttactggtcatagaaggctttcaatgttgagtggccttttattaacatgtttatggtactgcatagatacgggtatttattttaccctaagaagattttgaagtttaaaagtacttaaactatttggcaaagatttgtttttaaaaatctatttggtcaatctaaatgcattcattctaaaaaattttttgaaccagataaataaaatttttttttgacaccacagtttagtgtctggagtcttactggaaaaacacgatttctttttatatgtgatatacagatgctggaaagttacctttaaaaattgagtctctaaagaaaaaagaaaataataaaaattgagtctcaagaagttgtacttcatatgcacaagagaaatgaggccaagactcacagtaagcttttacatggaatggtaacttttagttagactgaaaaacttgaatgttagctattaaaaccttttaggaaggaatagccagctaaaaccaaatgagatttttaaagttaaatcagcacttaagttgtgtccttagtaggaaaaagtaggaagttaactactccgtaatccaagagataatgtgtaacagtaagaatgattttgttgttgttgagtatgagcaaagtagcccctaagtgtgtattagggttctgtacttaactgttgtgtgatgtgtgcttttgttaggcatcactgtgcccaagtatttcatgttcattgtaaagaggaaaaatacagatttctctataatgtcaccacttatttctaatgccacttttcatcttgtggaaatgccatgttttgattcagtcttctgaatttgaacattattcaggttatttccaattgctgggaatatccttactgctaaaataaattcttagcattggaattgctaggtcaaagattatgcatgctttttaagggcttttgaaatgtattgccagtctgtggcctgccaccctccctgaacatgcctggtcttgcttaaaatgtattgccaaatagtccttgggaagtttatgttgtccttaagatacattgatgagtttggacaaaccacaactagaatgcagtgaaaaaaatgctttatttgtgaaatttgtgatgctattgctttatttgtaaccattataagctgcaataaacaagttctgattttgtagttaacacgtgcgaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 22 - DNA, 6359 bp, Artificial Sequence
  Synthetic
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagccgtgttttagagctagaaatagcaagttaaaataaggctagtccgtagcgcgtgcgccaattctgcagacaaattctagaggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggagctgaatgagttgtggcgcccacaatgctcccatgacaaggagctgacaagttccattttccgtcgcgggcatcttggaatcatgactcccacaatgccttgggcacttggtcgacagtggggccgcctctgaaaaaaaaatgtgagaggtaagtttccattttcacagtttccccgcgccgcttcattgttcggactccggcgggtctgcccgtggctgaaggaggaagtgcgaggaggtgctcgccggccgcggttctcccgggcaggggcgggccgctcgcggtagttggtttcgcaggctgacttgaatattacttagtctggtatcacatggcaaggttgtgatactgtaggacatcagtgaagtgctacttagtaaatcttaacctatctcttttttctacaggttggtactaagaagtgcctttcctgacgtctctgctgcttggaaccgcttctagagcagtctctgcttttgccttgcttgctgccagctagactgtgacgacagcacatccaccctccacctctagcccagacacccccatttctacttataatcaagagaaaagctctaagtatctggcattgccctaggctgctttagtgttaaaagaaaagtttgctgaaaaataagatatcttctgccaggaaatcaaggaggaaaaaaaaaatcattttctcgattttgctctaaactgctgcatctgtctatgccaaactaatcaataccgattgcaccaccaaactccattgcaaattcagctgtgaggagattccctttcagacaactttgctgaaagcagcttggaaattcggtgtcgaagggtctgccacgttttcatgcttgcattttgggctccaaattggcactgggaaggggttactgagagcacaaggctgataccaggccctacttttaaacgttcatctacttacaatcctagtatttctctaaaaaccaaaacctctttgaattaacagtttcatgctgtgaatttctagtgggagatcttttccttgatattgacgacacaattttccatgtacttttaaagcagggagtggggaaaagtattttgaggggacattttcatcatcagttcagctttttttttttggttgttgctcttttttgggggggttgggtttgttggtttcactgaaacatttaactacctgtaaaatctaaacatggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaagtgagtaactattatattcttttaaggatattcaatgattgaatgaggggtatatggacatacagttctctgcccaatgatacattcaagtgggtggtttgcattatatttttttcataatttgctgattttattcttttctaagatgatgtttgctgcttttgtttctctagggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatgggaattcctcaagctgtacttcccccattaccaaagaggcctgctcttgaaaaaaccaacggtgccaccgcagtctttaacactggtattttccaataccaacaggctctagccaacatgcagttacaacagcatacagcatttctcccaccagttcccatggtgcacggtgctacgccagccactgtgtccgcagcaacaacatctgccacaagtgttcccttcgctgcaacagccacagccaaccagatacccataatatctgccgaacatctgactagccacaagtatgttacccagatgtacccatacgacgtaccagattacgcttagcttaagatacattgatgagtttggacaaaccacaactagaatgcagtgaaaaaaatgctttatttgtgaaatttgtgatgctattgctttatttgtaaccattataagctgcaataaacaagttctgattttgtagttaacacgtgcgaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 23 - DNA, 7056 bp, Artificial Sequence
  Synthetic
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagccgtgttttagagctagaaatagcaagttaaaataaggctagtccgtagcgcgtgcgccaattctgcagacaaattctagaggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggagctgaatgagttgtggcgcccacaatgctcccatgacaaggagctgacaagttccattttccgtcgcgggcatcttggaatcatgactcccacaatgccttgggcacttggtcgacagtggggccgcctctgaaaaaaaaatgtgagaggtaagtttccattttcacagtttccccgcgccgcttcattgttcggactccggcgggtctgcccgtggctgaaggaggaagtgcgaggaggtgctcgccggccgcggttctcccgggcaggggcgggccgctcgcggtagttggtttcgcaggctgacttgaatattacttagtctggtatcacatggcaaggttgtgatactgtaggacatcagtgaagtgctacttagtaaatcttaacctatctcttttttctacaggttggtactaagaagtgcctttcctgacgtctctgctgcttggaaccgcttctagagcagtctctgcttttgccttgcttgctgccagctagactgtgacgacagcacatccaccctccacctctagcccagacacccccatttctacttataatcaagagaaaagctctaagtatctggcattgccctaggctgctttagtgttaaaagaaaagtttgctgaaaaataagatatcttctgccaggaaatcaaggaggaaaaaaaaaatcattttctcgattttgctctaaactgctgcatctgtctatgccaaactaatcaataccgattgcaccaccaaactccattgcaaattcagctgtgaggagattccctttcagacaactttgctgaaagcagcttggaaattcggtgtcgaagggtctgccacgttttcatgcttgcattttgggctccaaattggcactgggaaggggttactgagagcacaaggctgataccaggccctacttttaaacgttcatctacttacaatcctagtatttctctaaaaaccaaaacctctttgaattaacagtttcatgctgtgaatttctagtgggagatcttttccttgatattgacgacacaattttccatgtacttttaaagcagggagtggggaaaagtattttgaggggacattttcatcatcagttcagctttttttttttggttgttgctcttttttgggggggttgggtttgttggtttcactgaaacatttaactacctgtaaaatctaaacatggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaagtgagtaactattatattcttttaaggatattcaatgattgaatgaggggtatatggacatacagttctctgcccaatgatacattcaagtgggtggtttgcattatatttttttcataatttgctgattttattcttttctaagatgatgtttgctgcttttgtttctctagggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatggtgagtagagatatcagctctctccttgttagcagtcagaaaagcaaagtgagcaactatatctgactacaagctattcatttagtaacctttttaaaaaaattgctgaagatatgtttgttcaggtatcccagaccacgagagagatcttttctgtgtttatggatacttgagcaaaaatacagaaggcagactctctcctcctctcttcctttcactcttttttttttctgttagagtatcttgtttgtaattaactacaaagaggagttatcctcccaataacaactcagtagtgcctttattgtgcatgcttagtcttgttattcgttgtatatggcattccgatgatttgtttttttatttgttttttctcacctacccaaaaatgcactgctgcccccatgatgcacctctgcttgctgtttatgttaatgcgcttgaaccccactggcccattgccatcatgtgctcgctgcctgctaattaagactcagtcggctgtcaaatcactgaagcgacccctcgaggcaacctttgacctggtactatgacctttcaccttttagcttggcatgtagctttattgtatgcttgcttgctcatgcttcctaacaattttagccttcgactgatttttcttttttctttttctctttttactggtatttgttttttatactcattcactaaacagggaattcctcaagctgtacttcccccattaccaaagaggcctgctcttgaaaaaaccaacggtgccaccgcagtctttaacactggtattttccaataccaacaagctctagccaacatgcagttacaacagcatacagcatttctcccaccagttcccatggtgcacggtgctacgccagccactgtgtccgcagcaacaacatctgccacaagtgttcccttcgctgcaacagccacagccaaccagatccctatcatcagcgctgagcacctgactagccacaagtatgttacccagatgtacccatacgacgtaccagattacgcttagcttaagatacattgatgagtttggacaaaccacaactagaatgcagtgaaaaaaatgctttatttgtgaaatttgtgatgctattgctttatttgtaaccattataagctgcaataaacaagttctgattttgtagttaacacgtgcgaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 24 - DNA, 6358 bp, Artificial Sequence
  Synthetic
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagccgtgttttagagctagaaatagcaagttaaaataaggctagtccgtagcgcgtgcgccaattctgcagacaaattctagaggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggagctgaatgagttgtggcgcccacaatgctcccatgacaaggagctgacaagttccattttccgtcgcgggcatcttggaatcatgactcccacaatgccttgggcacttggtcgacagtggggccgcctctgaaaaaaaaatgtgagaggtaagtttccattttcacagtttccccgcgccgcttcattgttcggactccggcgggtctgcccgtggctgaaggaggaagtgcgaggaggtgctcgccggccgcggttctcccgggcaggggcgggccgctcgcggtagttggtttcgcaggctgacttgaatattacttagtctggtatcacatggcaaggttgtgatactgtaggaatcagtgaagtgctacttagtaaatcttaacctatctcttttttctacaggttggtactaagaagtgcctttcctgacgtctctgctgcttggaaccgcttctagagcagtctctgcttttgccttgcttgctgccagctagactgtgacgacagcacatccaccctccacctctagcccagacacccccatttctacttataatcaagagaaaagctctaagtatctggcattgccctaggctgctttagtgttaaaagaaaagtttgctgaaaaataagatatcttctgccaggaaatcaaggaggaaaaaaaaaatcattttctcgattttgctctaaactgctgcatctgtctatgccaaactaatcaataccgattgcaccaccaaactccattgcaaattcagctgtgaggagattccctttcagacaactttgctgaaagcagcttggaaattcggtgtcgaagggtctgccacgttttcatgcttgcattttgggctccaaattggcactgggaaggggttactgagagcacaaggctgataccaggccctacttttaaacgttcatctacttacaatcctagtatttctctaaaaaccaaaacctctttgaattaacagtttcatgctgtgaatttctagtgggagatcttttccttgatattgacgacacaattttccatgtacttttaaagcagggagtggggaaaagtattttgaggggacattttcatcatcagttcagctttttttttttggttgttgctcttttttgggggggttgggtttgttggtttcactgaaacatttaactacctgtaaaatctaaacatggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaagtgagtaactattatattcttttaaggatattcaatgattgaatgaggggtatatggacatacagttctctgcccaatgatacattcaagtgggtggtttgcattatatttttttcataatttgctgattttattcttttctaagatgatgtttgctgcttttgtttctctagggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatgggaattcctcaagctgtacttcccccattaccaaagaggcctgctcttgaaaaaaccaacggtgccaccgcagtctttaacactggtattttccaataccaacaggctctagccaacatgcagttacaacagcatacagcatttctcccaccagttcccatggtgcacggtgctacgccagccactgtgtccgcagcaacaacatctgccacaagtgttcccttcgctgcaacagccacagccaaccagatccctatcatcagcgctgagcacctgactagccacaagtatgttacccagatgtacccatacgacgtaccagattacgcttagcttaagatacattgatgagtttggacaaaccacaactagaatgcagtgaaaaaaatgctttatttgtgaaatttgtgatgctattgctttatttgtaaccattataagctgcaataaacaagttctgattttgtagttaacacgtgcgaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 25 - DNA, 6358 bp, Artificial Sequence
  Synthetic
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagccgtgttttagagctagaaatagcaagttaaaataaggctagtccgtagcgcgtgcgccaattctgcagacaaattctagaggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggagctgaatgagttgtggcgcccacaatgctcccatgacaaggagctgacaagttccattttccgtcgcgggcatcttggaatcatgactcccacaatgccttgggcacttggtcgacagtggggccgcctctgaaaaaaaaatgtgagaggtaagtttccattttcacagtttccccgcgccgcttcattgttcggactccggcgggtctgcccgtggctgaaggaggaagtgcgaggaggtgctcgccggccgcggttctcccgggcaggggcgggccgctcgcggtagttggtttcgcaggctgacttgaatattacttagtctggtatcacatggcaaggttgtgatactgtaggaatcagtgaagtgctacttagtaaatcttaacctatctcttttttctacaggttggtactaagaagtgcctttcctgacgtctctgctgcttggaaccgcttctagagcagtctctgcttttgccttgcttgctgccagctagactgtgacgacagcacatccaccctccacctctagcccagacacccccatttctacttataatcaagagaaaagctctaagtatctggcattgccctaggctgctttagtgttaaaagaaaagtttgctgaaaaataagatatcttctgccaggaaatcaaggaggaaaaaaaaaatcattttctcgattttgctctaaactgctgcatctgtctatgccaaactaatcaataccgattgcaccaccaaactccattgcaaattcagctgtgaggagattccctttcagacaactttgctgaaagcagcttggaaattcggtgtcgaagggtctgccacgttttcatgcttgcattttgggctccaaattggcactgggaaggggttactgagagcacaaggctgataccaggccctacttttaaacgttcatctacttacaatcctagtatttctctaaaaaccaaaacctctttgaattaacagtttcatgctgtgaatttctagtgggagatcttttccttgatattgacgacacaattttccatgtacttttaaagcagggagtggggaaaagtattttgaggggacattttcatcatcagttcagctttttttttttggttgttgctcttttttgggggggttgggtttgttggtttcactgaaacatttaactacctgtaaaatctaaacatggctgtttctgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaagtgagtaactattatattcttttaaggatattcaatgattgaatgaggggtatatggacatacagttctctgcccaatgatacattcaagtgggtggtttgcattatatttttttcataatttgctgattttattcttttctaagatgatgtttgctgcttttgtttctctagggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatgggaattcctcaagctgtacttcccccattaccaaagaggcctgctcttgaaaaaaccaacggtgccaccgcagtctttaacactggtattttccaataccaacaggctctagccaacatgcagttacaacagcatacagcatttctcccaccagttcccatggtgcacggtgctacgccagccactgtgtccgcagcaacaacatctgccacaagtgttcccttcgctgcaacagccacagccaaccagatacccataatatctgccgaacatctgactagccacaagtatgttacccagatgtacccatacgacgtaccagattacgcttagcttaagatacattgatgagtttggacaaaccacaactagaatgcagtgaaaaaaatgctttatttgtgaaatttgtgatgctattgctttatttgtaaccattataagctgcaataaacaagttctgattttgtagttaacacgtgcgaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 26 - DNA, 7056 bp, Artificial Sequence
  Synthetic
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagccgtgttttagagctagaaatagcaagttaaaataaggctagtccgtagcgcgtgcgccaattctgcagacaaattctagaggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggagctgaatgagttgtggcgcccacaatgctcccatgacaaggagctgacaagttccattttccgtcgcgggcatcttggaatcatgactcccacaatgccttgggcacttggtcgacagtggggccgcctctgaaaaaaaaatgtgagaggtaagtttccattttcacagtttccccgcgccgcttcattgttcggactccggcgggtctgcccgtggctgaaggaggaagtgcgaggaggtgctcgccggccgcggttctcccgggcaggggcgggccgctcgcggtagttggtttcgcaggctgacttgaatattacttagtctggtatcacatggcaaggttgtgatactgtaggacatcagtgaagtgctacttagtaaatcttaacctatctcttttttctacaggttggtactaagaagtgcctttcctgacgtctctgctgcttggaaccgcttctagagcagtctctgcttttgccttgcttgctgccagctagactgtgacgacagcacatccaccctccacctctagcccagacacccccatttctacttataatcaagagaaaagctctaagtatctggcattgccctaggctgctttagtgttaaaagaaaagtttgctgaaaaataagatatcttctgccaggaaatcaaggaggaaaaaaaaaatcattttctcgattttgctctaaactgctgcatctgtctatgccaaactaatcaataccgattgcaccaccaaactccattgcaaattcagctgtgaggagattccctttcagacaactttgctgaaagcagcttggaaattcggtgtcgaagggtctgccacgttttcatgcttgcattttgggctccaaattggcactgggaaggggttactgagagcacaaggctgataccaggccctacttttaaacgttcatctacttacaatcctagtatttctctaaaaaccaaaacctctttgaattaacagtttcatgctgtgaatttctagtgggagatcttttccttgatattgacgacacaattttccatgtacttttaaagcagggagtggggaaaagtattttgaggggacattttcatcatcagttcagctttttttttttggttgttgctcttttttgggggggttgggtttgttggtttcactgaaacatttaactacctgtaaaatctaaacatggctgtttctgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaagtgagtaactattatattcttttaaggatattcaatgattgaatgaggggtatatggacatacagttctctgcccaatgatacattcaagtgggtggtttgcattatatttttttcataatttgctgattttattcttttctaagatgatgtttgctgcttttgtttctctagggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatggtgagtagagatatcagctctctccttgttagcagtcagaaaagcaaagtgagcaactatatctgactacaagctattcatttagtaacctttttaaaaaaattgctgaagatatgtttgttcaggtatcccagaccacgagagagatcttttctgtgtttatggatacttgagcaaaaatacagaaggcagactctctcctcctctcttcctttcactcttttttttttctgttagagtatcttgtttgtaattaactacaaagaggagttatcctcccaataacaactcagtagtgcctttattgtgcatgcttagtcttgttattcgttgtatatggcattccgatgatttgtttttttatttgttttttctcacctacccaaaaatgcactgctgcccccatgatgcacctctgcttgctgtttatgttaatgcgcttgaaccccactggcccattgccatcatgtgctcgctgcctgctaattaagactcagtcggctgtcaaatcactgaagcgacccctcgaggcaacctttgacctggtactatgacctttcaccttttagcttggcatgtagctttattgtatgcttgcttgctcatgcttcctaacaattttagccttcgactgatttttcttttttctttttctctttttactggtatttgttttttatactcattcactaaacagggaattcctcaagctgtacttcccccattaccaaagaggcctgctcttgaaaaaaccaacggtgccaccgcagtctttaacactggtattttccaataccaacaagctctagccaacatgcagttacaacagcatacagcatttctcccaccagttcccatggtgcacggtgctacgccagccactgtgtccgcagcaacaacatctgccacaagtgttcccttcgctgcaacagccacagccaaccagatccctatcatcagcgctgagcacctgactagccacaagtatgttacccagatgtacccatacgacgtaccagattacgcttagcttaagatacattgatgagtttggacaaaccacaactagaatgcagtgaaaaaaatgctttatttgtgaaatttgtgatgctattgctttatttgtaaccattataagctgcaataaacaagttctgattttgtagttaacacgtgcgaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 27 - DNA, 6358 bp, Artificial Sequence
  Synthetic
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagccgtgttttagagctagaaatagcaagttaaaataaggctagtccgtagcgcgtgcgccaattctgcagacaaattctagaggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggagctgaatgagttgtggcgcccacaatgctcccatgacaaggagctgacaagttccattttccgtcgcgggcatcttggaatcatgactcccacaatgccttgggcacttggtcgacagtggggccgcctctgaaaaaaaaatgtgagaggtaagtttccattttcacagtttccccgcgccgcttcattgttcggactccggcgggtctgcccgtggctgaaggaggaagtgcgaggaggtgctcgccggccgcggttctcccgggcaggggcgggccgctcgcggtagttggtttcgcaggctgacttgaatattacttagtctggtatcacatggcaaggttgtgatactgtaggaatcagtgaagtgctacttagtaaatcttaacctatctcttttttctacaggttggtactaagaagtgcctttcctgacgtctctgctgcttggaaccgcttctagagcagtctctgcttttgccttgcttgctgccagctagactgtgacgacagcacatccaccctccacctctagcccagacacccccatttctacttataatcaagagaaaagctctaagtatctggcattgccctaggctgctttagtgttaaaagaaaagtttgctgaaaaataagatatcttctgccaggaaatcaaggaggaaaaaaaaaatcattttctcgattttgctctaaactgctgcatctgtctatgccaaactaatcaataccgattgcaccaccaaactccattgcaaattcagctgtgaggagattccctttcagacaactttgctgaaagcagcttggaaattcggtgtcgaagggtctgccacgttttcatgcttgcattttgggctccaaattggcactgggaaggggttactgagagcacaaggctgataccaggccctacttttaaacgttcatctacttacaatcctagtatttctctaaaaaccaaaacctctttgaattaacagtttcatgctgtgaatttctagtgggagatcttttccttgatattgacgacacaattttccatgtacttttaaagcagggagtggggaaaagtattttgaggggacattttcatcatcagttcagctttttttttttggttgttgctcttttttgggggggttgggtttgttggtttcactgaaacatttaactacctgtaaaatctaaacatggctgtttctgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaagtgagtaactattatattcttttaaggatattcaatgattgaatgaggggtatatggacatacagttctctgcccaatgatacattcaagtgggtggtttgcattatatttttttcataatttgctgattttattcttttctaagatgatgtttgctgcttttgtttctctagggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatgggaattcctcaagctgtacttcccccattaccaaagaggcctgctcttgaaaaaaccaacggtgccaccgcagtctttaacactggtattttccaataccaacaggctctagccaacatgcagttacaacagcatacagcatttctcccaccagttcccatggtgcacggtgctacgccagccactgtgtccgcagcaacaacatctgccacaagtgttcccttcgctgcaacagccacagccaaccagatccctatcatcagcgctgagcacctgactagccacaagtatgttacccagatgtacccatacgacgtaccagattacgcttagcttaagatacattgatgagtttggacaaaccacaactagaatgcagtgaaaaaaatgctttatttgtgaaatttgtgatgctattgctttatttgtaaccattataagctgcaataaacaagttctgattttgtagttaacacgtgcgaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 28 - DNA, 8102 bp, Artificial Sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatccactagtccagtgtggtggaattgcccttcaccagctgaatgagttgtggcgcccacaatgctcccatgacaaggagctgacaagttccattttccgtcgcgggcatcttggaatcatgactcccacaatgccttgggcacttggtcgacagtggggccgcctctgaaaaaaaaatgtgagaggtaagtttccattttcacagtttccccgcgccgcttcattgttcggactccggcgggtctgcccgtggctgaaggaggaagtgcgaggaggtgctcgccggccgcggttctcccgggcaggggcgggccgctcgcggtagttggtttcgcaggctgacttgaatattacttagtctggtatcacatggcaaggttgtgatactgtaggacatcagtgaagtgctacttagtaaatcttaacctatctcttttttctacaggttggtactaagaagtgcctttcctgacgtctctgctgcttggaaccgcttctagagcagtctctgcttttgccttgcttgctgccagctagactgtgacgacagcacatccaccctccacctctagcccagacacccccatttctacttataatcaagagaaaagctctaagtatctggcattgccctaggctgctttagtgttaaaagaaaagtttgctgaaaaataagatatcttctgccaggaaatcaaggaggaaaaaaaaaatcattttctcgattttgctctaaactgctgcatctgtctatgccaaactaatcaataccgattgcaccaccaaactccattgcaaattcagctgtgaggagattccctttcagacaactttgctgaaagcagcttggaaattcggtgtcgaagggtctgccacgttttcatgcttgcattttgggctccaaattggcactgggaaggggttactgagagcacaaggctgataccaggccctacttttaaacgttcatctacttacaatcctagtatttctctaaaaaccaaaacctctttgaattaacagtttcatgctgtgaatttctagtgggagatcttttccttgatattgacgacacaattttccatgtacttttaaagcagggagtggggaaaagtattttgaggggacattttcatcatcagttcagctttttttttttggttgttgctcttttttgggggggttgggtttgttggtttcactgaaacatttaactacctgtaaaatctaaacatggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaagtgagtaactattatattcttttaaggatattcaatgattgaatgaggggtatatggacatacagttctctgcccaatgatacattcaagtgggtggtttgcattatatttttttcataatttgctgattttattcttttctaagatgatgtttgctgcttttgtttctctagggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatgggaattcctcaagctgtacttcccccattaccaaagaggcctgctcttgaaaaaaccaacggtgccaccgcagtctttaacactggtattttccaataccaacaggctctagccaacatgcagttacaacagcatacagcatttctcccaccagttcccatggtgcacggtgctacgccagccactgtgtccgcagcaacaacatctgccacaagtgttcccttcgctgcaacagccacagccaaccagatccctatcatcagcgctgagcacctgactagccacaagtatgttacccagatgtacccatacgacgtaccagattacgcttagcttaagatacattgatgagtttggacaaaccacaactagaatgcagtgaaaaaaatgctttatttgtgaaatttgtgatgctattgctttatttgtaaccattataagctgcaataaacaagttttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 29 - DNA, 8175 bp, Artificial Sequence
  Synthetic
agctgaatgagttgtggcgcccacaatgctcccatgacaaggagctgacaagttccattttccgtcgcgggcatcttggaatcatgactcccacaatgccttgggcacttggtcgacagtggggccgcctctgaaaaaaaaatgtgagaggtaagtttccattttcacagtttccccgcgccgcttcattgttcggactccggcgggtctgcccgtggctgaaggaggaagtgcgaggaggtgctcgccggccgcggttctcccgggcaggggcgggccgctcgcggtagttggtttcgcgtccctcagcacctcctgcccgggggaaggcgggagggtgagccgcagccgctcctgcgcccttccctgccgcgggctctgcggacgctgtcacggcacagcccgcgcacggcttggcagcccagcgggacccagggagaaacaggggggaggctgacttgaatattacttagtctggtatcacatggcaaggttgtgatactgtaggacatcagtgaagtgctacttagtaaatcttaacctatctcttttttctacaggttggtactaagaagtgcctttcctgacgtctctgctgcttggaaccgcttctagagcagtctctgcttttgccttgcttgctgccagctagactgtgacgacagcacatccaccctccacctctagcccagacacccccatttctacttataatcaagagaaaagctctaagtatctggcattgccctaggctgctttagtgttaaaagaaaagtttgctgaaaaataagatatcttctgccaggaaatcaaggaggaaaaaaaaaatcattttctcgattttgctctaaactgctgcatctgtctatgccaaactaatcaataccgattgcaccaccaaactccattgcaaattcagctgtgaggagattccctttcagacaactttgctgaaagcagcttggaaattcggtgtcgaagggtctgccacgttttcatgcttgcattttgggctccaaattggcactgggaaggggttactgagagcacaaggctgataccaggccctacttttaaacgttcatctacttacaatcctagtatttctctaaaaaccaaaacctctttgaattaacagtttcatgctgtgaatttctagtgggagatcttttccttgatattgacgacacaattttccatgtacttttaaagcagggagtggggaaaagtattttgaggggacattttcatcatcagttcagctttttttttttggttgttgctcttttttgggggggttgggtttgttggtttcactgaaacatttaactacctgtaaaatctaaacatggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaagtgagtaactattatattcttttaaggatattcaatgattgaatgaggggtatatggacatacagttctctgcccaatgatacattcaagtgggtggtttgcattatatttttttcataatttgctgattttattcttttctaagatgatgtttgctgcttttgtttctctagggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatgggaattcctcaagctgtacttcccccattaccaaagaggcctgctcttgaaaaaaccaacggtgccaccgcagtctttaacactggtattttccaataccaacaggctctagccaacatgcagttacaacagcatacagcatttctcccaccagttcccatggtgcacggtgctacgccagccactgtgtccgcagcaacaacatctgccacaagtgttcccttcgctgcaacagccacagccaaccagatccctatcatcagcgctgagcacctgactagccacaagtatgttacccagatgtacccatacgacgtaccagattacgcttagaagggtcaagacaattctgcagatatccagcacagtggcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatccagtacccttcacc

SEQ ID NO: 30 - DNA, 8475 bp, Artificial Sequence
  Synthetic
agctgaatgagttgtggcgcccacaatgctcccatgacaaggagctgacaagttccattttccgtcgcgggcatcttggaatcatgactcccacaatgccttgggcacttggtcgacagtggggccgcctctgaaaaaaaaatgtgagaggtaagtttccattttcacagtttccccgcgccgcttcattgttcggactccggcgggtctgcccgtggctgaaggaggaagtgcgaggaggtgctcgccggccgcggttctcccgggcaggggcgggccgctcgcggtagttggtttcgcgtccctcagcacctcctgcccgggggaaggcgggagggtgagccgcagccgctcctgcgcccttccctgccgcgggctctgcggacgctgtcacggcacagcccgcgcacggcttggcagcccagcgggacccagggagaaacaggggggcgagaagagaaacaggtgcgtgcaagtgtactcacaacttaatgacctttgtcgggggaaggtgtgagtccctctgagtcgagcggcgcgcgggtcttcccggcggctcccgcatccctggccccggggctgccatcccgcccggttttcatctgggccaaatatgtgcacgcaaaggttaattctctgcttgctacccataaattacgcatgacatgtttagctgggctgccttatatggagagtgctgtatttcaggccagtttgccttttgccaagtttacagggcggaagacgcccaggctgacttgaatattacttagtctggtatcacatggcaaggttgtgatactgtaggacatcagtgaagtgctacttagtaaatcttaacctatctcttttttctacaggttggtactaagaagtgcctttcctgacgtctctgctgcttggaaccgcttctagagcagtctctgcttttgccttgcttgctgccagctagactgtgacgacagcacatccaccctccacctctagcccagacacccccatttctacttataatcaagagaaaagctctaagtatctggcattgccctaggctgctttagtgttaaaagaaaagtttgctgaaaaataagatatcttctgccaggaaatcaaggaggaaaaaaaaaatcattttctcgattttgctctaaactgctgcatctgtctatgccaaactaatcaataccgattgcaccaccaaactccattgcaaattcagctgtgaggagattccctttcagacaactttgctgaaagcagcttggaaattcggtgtcgaagggtctgccacgttttcatgcttgcattttgggctccaaattggcactgggaaggggttactgagagcacaaggctgataccaggccctacttttaaacgttcatctacttacaatcctagtatttctctaaaaaccaaaacctctttgaattaacagtttcatgctgtgaatttctagtgggagatcttttccttgatattgacgacacaattttccatgtacttttaaagcagggagtggggaaaagtattttgaggggacattttcatcatcagttcagctttttttttttggttgttgctcttttttgggggggttgggtttgttggtttcactgaaacatttaactacctgtaaaatctaaacatggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaagtgagtaactattatattcttttaaggatattcaatgattgaatgaggggtatatggacatacagttctctgcccaatgatacattcaagtgggtggtttgcattatatttttttcataatttgctgattttattcttttctaagatgatgtttgctgcttttgtttctctagggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatgggaattcctcaagctgtacttcccccattaccaaagaggcctgctcttgaaaaaaccaacggtgccaccgcagtctttaacactggtattttccaataccaacaggctctagccaacatgcagttacaacagcatacagcatttctcccaccagttcccatggtgcacggtgctacgccagccactgtgtccgcagcaacaacatctgccacaagtgttcccttcgctgcaacagccacagccaaccagatccctatcatcagcgctgagcacctgactagccacaagtatgttacccagatgtacccatacgacgtaccagattacgcttagaagggtcaagacaattctgcagatatccagcacagtggcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatccagtacccttcacc

SEQ ID NO: 31 - DNA, 9075 bp, Artificial Sequence
  Synthetic
agctgaatgagttgtggcgcccacaatgctcccatgacaaggagctgacaagttccattttccgtcgcgggcatcttggaatcatgactcccacaatgccttgggcacttggtcgacagtggggccgcctctgaaaaaaaaatgtgagaggtaagtttccattttcacagtttccccgcgccgcttcattgttcggactccggcgggtctgcccgtggctgaaggaggaagtgcgaggaggtgctcgccggccgcggttctcccgggcaggggcgggccgctcgcggtagttggtttcgcgtccctcagcacctcctgcccgggggaaggcgggagggtgagccgcagccgctcctgcgcccttccctgccgcgggctctgcggacgctgtcacggcacagcccgcgcacggcttggcagcccagcgggacccagggagaaacaggggggcgagaagagaaacaggtgcgtgcaagtgtactcacaacttaatgacctttgtcgggggaaggtgtgagtccctctgagtcgagcggcgcgcgggtcttcccggcggctcccgcatccctggccccggggctgccatcccgcccggttttcatctgggccaaatatgtgcacgcaaaggttaattctctgcttgctacccataaattacgcatgacatgtttagctgggctgccttatatggagagtgctgtatttcaggccagtttgccttttgccaagtttacagggcggaagacgccctgtaggagagggctgcctcctccttttcttcttcttcttttttttttcatcgttttgtagtgcgtcactcaaaaactgtaagaaaagtaagtatagaagctctttgggtggagccgcagttggttgaattgctttcatgaaaggttgaaaagaatgaaaagtagttagtacttgggttctggtttttagacaaaagcaggacaaatatgtagccaccccccaacttttttttttttttttaaacggagccggaatctgttattggtagtaatgtgtcatgtgagaatactttgattccagagcctgtaacacgagcaggtatatggtggcgcttatgccttcccctcgcacagcactttacaactacaggggtgttgctgccagagtaatattgtattcttaaggctctctggggccgggagagtatgcggctgttgggattctggggtgtatttccgtggagtatgtctccccaggattaaagttaagaagctgccggaaaggttgttgccgctgacttcactttgtgtgcccaccagcacaggcacaagtcatagcagatggatggcaaactctgccatatactctgaggagtttcttaggctgacttgaatattacttagtctggtatcacatggcaaggttgtgatactgtaggacatcagtgaagtgctacttagtaaatcttaacctatctcttttttctacaggttggtactaagaagtgcctttcctgacgtctctgctgcttggaaccgcttctagagcagtctctgcttttgccttgcttgctgccagctagactgtgacgacagcacatccaccctccacctctagcccagacacccccatttctacttataatcaagagaaaagctctaagtatctggcattgccctaggctgctttagtgttaaaagaaaagtttgctgaaaaataagatatcttctgccaggaaatcaaggaggaaaaaaaaaatcattttctcgattttgctctaaactgctgcatctgtctatgccaaactaatcaataccgattgcaccaccaaactccattgcaaattcagctgtgaggagattccctttcagacaactttgctgaaagcagcttggaaattcggtgtcgaagggtctgccacgttttcatgcttgcattttgggctccaaattggcactgggaaggggttactgagagcacaaggctgataccaggccctacttttaaacgttcatctacttacaatcctagtatttctctaaaaaccaaaacctctttgaattaacagtttcatgctgtgaatttctagtgggagatcttttccttgatattgacgacacaattttccatgtacttttaaagcagggagtggggaaaagtattttgaggggacattttcatcatcagttcagctttttttttttggttgttgctcttttttgggggggttgggtttgttggtttcactgaaacatttaactacctgtaaaatctaaacatggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaagtgagtaactattatattcttttaaggatattcaatgattgaatgaggggtatatggacatacagttctctgcccaatgatacattcaagtgggtggtttgcattatatttttttcataatttgctgattttattcttttctaagatgatgtttgctgcttttgtttctctagggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatgggaattcctcaagctgtacttcccccattaccaaagaggcctgctcttgaaaaaaccaacggtgccaccgcagtctttaacactggtattttccaataccaacaggctctagccaacatgcagttacaacagcatacagcatttctcccaccagttcccatggtgcacggtgctacgccagccactgtgtccgcagcaacaacatctgccacaagtgttcccttcgctgcaacagccacagccaaccagatccctatcatcagcgctgagcacctgactagccacaagtatgttacccagatgtacccatacgacgtaccagattacgcttagaagggtcaagacaattctgcagatatccagcacagtggcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatccagtacccttcacc

SEQ ID NO: 32 - DNA, 5659 bp, Artificial Sequence
  Synthetic
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagccgtgttttagagctagaaatagcaagttaaaataaggctagtccgtagcgcgtgcgccaattctgcagacaaattctagaggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtcaccatggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaaggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatggtgagtagagatatcagctctctccttgttagcagtcagaaaagcaaagtgagcaactatatctgactacaagctattcatttagtaacctttttaaaaaaattgctgaagatatgtttgttcaggtatcccagaccacgagagagatcttttctgtgtttatggatacttgagcaaaaatacagaaggcagactctctcctcctctcttcctttcactcttttttttttctgttagagtatcttgtttgtaattaactacaaagaggagttatcctcccaataacaactcagtagtgcctttattgtgcatgcttagtcttgttattcgttgtatatggcattccgatgatttgtttttttatttgttttttctcacctacccaaaaatgcactgctgcccccatgatgcacctctgcttgctgtttatgttaatgcgcttgaaccccactggcccattgccatcatgtgctcgctgcctgctaattaagactcagtcggctgtcaaatcactgaagcgacccctcgaggcaacctttgacctggtactatgacctttcaccttttagcttggcatgtagctttattgtaggccttcgactgatttttcttttttctttttctctttttactggtatttgttttttatactcattcactaaacagggaattcctcaagctgtacttcccccattaccaaagaggcctgctcttgaaaaaaccaacggtgccaccgcagtctttaacactggtattttccaataccaacaggctctagccaacatgcagttacaacagcatacagcatttctcccaccagttcccatggtgcacggtgctacgccagccactgtgtccgcagcaacaacatctgccacaagtgttcccttcgctgcaacagccacagccaaccagatacccataatatctgccgaacatctgactagccacaagtatgttacccagatgtacccatacgacgtaccagattacgcttagcttaagatacattgatgagtttggacaaaccacaactagaatgcagtgaaaaaaatgctttatttgtgaaatttgtgatgctattgctttatttgtaaccattataagctgcaataaacaagttctgattttgtagttaacacgtgcgaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 33 - DNA, 5662 bp, Artificial Sequence
  Synthetic
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagccgtgttttagagctagaaatagcaagttaaaataaggctagtccgtagcgcgtgcgccaattctgcagacaaattctagaggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtcaccatggagtacccatacgacgtaccagattacgctgctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaaggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatggtgagtagagatatcagctctctccttgttagcagtcagaaaagcaaagtgagcaactatatctgactacaagctattcatttagtaacctttttaaaaaaattgctgaagatatgtttgttcaggtatcccagaccacgagagagatcttttctgtgtttatggatacttgagcaaaaatacagaaggcagactctctcctcctctcttcctttcactcttttttttttctgttagagtatcttgtttgtaattaactacaaagaggagttatcctcccaataacaactcagtagtgcctttattgtgcatgcttagtcttgttattcgttgtatatggcattccgatgatttgtttttttatttgttttttctcacctacccaaaaatgcactgctgcccccatgatgcacctctgcttgctgtttatgttaatgcgcttgaaccccactggcccattgccatcatgtgctcgctgcctgctaattaagactcagtcggctgtcaaatcactgaagcgacccctcgaggcaacctttgacctggtactatgacctttcaccttttagcttggcatgtagctttattgtaggccttcgactgatttttcttttttctttttctctttttactggtatttgttttttatactcattcactaaacagggaattcctcaagctgtacttcccccattaccaaagaggcctgctcttgaaaaaaccaacggtgccaccgcagtctttaacactggtattttccaataccaacaagctctagccaacatgcagttacaacagcatacagcatttctcccaccagttcccatggtgcacggtgctacgccagccactgtgtccgcagcaacaacatctgccacaagtgttcccttcgctgcaacagccacagccaaccagatacccataatatctgccgaacatctgactagccacaagtatgttacccagatgtagcttaagatacattgatgagtttggacaaaccacaactagaatgcagtgaaaaaaatgctttatttgtgaaatttgtgatgctattgctttatttgtaaccattataagctgcaataaacaagttctgattttgtagttaacacgtgcgaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 34 - DNA, 5675 bp, Artificial Sequence
  Synthetic
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagccgtgttttagagctagaaatagcaagttaaaataaggctagtccgtagcgcgtgcgccaattctgcagacaaattctagaggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtcaccatggagtacccatacgacgtaccagattacgctgctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaaggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatggtgagtagagatatcagctctctccttgttagcagtcagaaaagcaaagtgagcaactatatctgactacaagctattcatttagtaacctttttaaaaaaattgctgaagatatgtttgttcaggtatcccagaccacgagagagatcttttctgtgtttatggatacttgagcaaaaatacagaaggcagactctctcctcctctcttcctttcactcttttttttttctgttagagtatcttgtttgtaattaactacaaagaggagttatcctcccaataacaactcagtagtgcctttattgtgcatgcttagtcttgttattcgttgtatatggcattccgatgatttgtttttttatttgttttttctcacctacccaaaaatgcactgctgcccccatgatgcacctctgcttgctgtttatgttaatgcgcttgaaccccactggcccattgccatcatgtgctcgctgcctgctaattaagactcagtcggctgtcaaatcactgaagcgacccctcgaggcaacctttgacctggtactatgacctttcaccttttagcttggcatgtagctttattgtatcctaacaattttagccttcgactgatttttcttttttctttttctctttttactggtatttgttttttatactcattcactaaacagggaattcctcaagctgtacttcccccattaccaaagaggcctgctcttgaaaaaaccaacggtgccaccgcagtctttaacactggtattttccaataccaacaagctctagccaacatgcagttacaacagcatacagcatttctcccaccagttcccatggtgcacggtgctacgccagccactgtgtccgcagcaacaacatctgccacaagtgttcccttcgctgcaacagccacagccaaccagatacccataatatctgccgaacatctgactagccacaagtatgttacccagatgtagcttaagatacattgatgagtttggacaaaccacaactagaatgcagtgaaaaaaatgctttatttgtgaaatttgtgatgctattgctttatttgtaaccattataagctgcaataaacaagttctgattttgtagttaacacgtgcgaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 35 - DNA, 5685 bp, Artificial Sequence
  Synthetic
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagccgtgttttagagctagaaatagcaagttaaaataaggctagtccgtagcgcgtgcgccaattctgcagacaaattctagaggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtcaccatggagtacccatacgacgtaccagattacgctgctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaaggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatggtgagtagagatatcagctctctccttgttagcagtcagaaaagcaaagtgagcaactatatctgactacaagctattcatttagtaacctttttaaaaaaattgctgaagatatgtttgttcaggtatcccagaccacgagagagatcttttctgtgtttatggatacttgagcaaaaatacagaaggcagactctctcctcctctcttcctttcactcttttttttttctgttagagtatcttgtttgtaattaactacaaagaggagttatcctcccaataacaactcagtagtgcctttattgtgcatgcttagtcttgttattcgttgtatatggcattccgatgatttgtttttttatttgttttttctcacctacccaaaaatgcactgctgcccccatgatgcacctctgcttgctgtttatgttaatgcgcttgaaccccactggcccattgccatcatgtgctcgctgcctgctaattaagactcagtcggctgtcaaatcactgaagcgacccctcgaggcaacctttgacctggtactatgacctttcaccttttagcttggcatgtagctttattgtatgctcatgcttcctaacaattttagccttcgactgatttttcttttttctttttctctttttactggtatttgttttttatactcattcactaaacagggaattcctcaagctgtacttcccccattaccaaagaggcctgctcttgaaaaaaccaacggtgccaccgcagtctttaacactggtattttccaataccaacaagctctagccaacatgcagttacaacagcatacagcatttctcccaccagttcccatggtgcacggtgctacgccagccactgtgtccgcagcaacaacatctgccacaagtgttcccttcgctgcaacagccacagccaaccagatacccataatatctgccgaacatctgactagccacaagtatgttacccagatgtagcttaagatacattgatgagtttggacaaaccacaactagaatgcagtgaaaaaaatgctttatttgtgaaatttgtgatgctattgctttatttgtaaccattataagctgcaataaacaagttctgattttgtagttaacacgtgcgaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 36 - DNA, 5693 bp, Artificial Sequence
  Synthetic
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagccgtgttttagagctagaaatagcaagttaaaataaggctagtccgtagcgcgtgcgccaattctgcagacaaattctagaggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtcaccatggagtacccatacgacgtaccagattacgctgctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaaggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatggtgagtagagatatcagctctctccttgttagcagtcagaaaagcaaagtgagcaactatatctgactacaagctattcatttagtaacctttttaaaaaaattgctgaagatatgtttgttcaggtatcccagaccacgagagagatcttttctgtgtttatggatacttgagcaaaaatacagaaggcagactctctcctcctctcttcctttcactcttttttttttctgttagagtatcttgtttgtaattaactacaaagaggagttatcctcccaataacaactcagtagtgcctttattgtgcatgcttagtcttgttattcgttgtatatggcattccgatgatttgtttttttatttgttttttctcacctacccaaaaatgcactgctgcccccatgatgcacctctgcttgctgtttatgttaatgcgcttgaaccccactggcccattgccatcatgtgctcgctgcctgctaattaagactcagtcggctgtcaaatcactgaagcgacccctcgaggcaacctttgacctggtactatgacctttcaccttttagcttggcatgtagctttattgtatgcttgcttgctcatgcttcctaacaattttagccttcgactgatttttcttttttctttttctctttttactggtatttgttttttatactcattcactaaacagggaattcctcaagctgtacttcccccattaccaaagaggcctgctcttgaaaaaaccaacggtgccaccgcagtctttaacactggtattttccaataccaacaagctctagccaacatgcagttacaacagcatacagcatttctcccaccagttcccatggtgcacggtgctacgccagccactgtgtccgcagcaacaacatctgccacaagtgttcccttcgctgcaacagccacagccaaccagatacccataatatctgccgaacatctgactagccacaagtatgttacccagatgtagcttaagatacattgatgagtttggacaaaccacaactagaatgcagtgaaaaaaatgctttatttgtgaaatttgtgatgctattgctttatttgtaaccattataagctgcaataaacaagttctgattttgtagttaacacgtgcgaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 37 - DNA, 7397 bp, Artificial Sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatcccaggtgagtagagatatcagctctctccttgttagcagtcagaaaagcaaagtgagcaactatatctgactacaagctattcatttagtaacctttttaaaaaaattgctgaagatatgtttgttcaggtatcccagaccacgagagagatcttttctgtgtttatggatacttgagcaaaaatacagaaggcagactctctcctcctctcttcctttcactcttttttttttctgttagagtatcttgtttgtaattaactacaaagaggagttatcctcccaataacaactcagtagtgcctttattgtgcatgcttagtcttgttattcgttgtatatggcattccgatgatttgtttttttatttgttttttctcacctacccaaaaatgcactgctgcccccatgatgcacctctgcttgctgtttatgttaatgcgcttgaaccccactggcccattgccatcatgtgctcgctgcctgctaattaagactcagtcggctgtcaaatcactgaagcgacccctcgaggcaacctttgaccatggtactatgacctttcaccttttagcttggcatgtagctttattgtatgcttgcttgctcatgcttcctaacaattttagccttcgactgatttttcttttttctttttctctttttactggtatttgttttttatactcattcactaaacaggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaaggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgccaatgttttcagttgcaccaagcttagccaccaatgcatcagcagccgcctttaatccctatctgggacctgtttctccaagcctggtcccggcagagatcttgccgactgcaccaatgttggttacagggaatccgggtgtccctgtacctgcagctgctgcagctgctgcacagaaattaatgcgaacagacagacttgaggtatgtcgagagtaccaacgtggcaattgcaaccgaggagaaaatgattgtcggtttgctcatcctgctgacagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatgggaattcctcaagctgtacttcccccattaccaaagaggcctgctcttgaaaaaaccaacggtgccaccgcagtctttaacactggtattttccaataccaacaggctctagccaacatgcagttacaacagcatacagcatttctcccaccagttcccatggtgcacggtgctacgccagccactgtgtccgcagcaacaacatctgccacaagtgttcccttcgctgcaacagccacagccaaccagatccctatcatcagcgctgagcacctgactagccacaagtatgttacccagatgtacccatacgacgtaccagattacgcttagcttaagatacattgatgagtttggacaaaccacaactagaatgcagtgaaaaaaatgctttatttgtgaaatttgtgatgctattgctttatttgtaaccattataagctgcaataaacaagttttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 38 - DNA, 7243 bp, Artificial Sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatccactagtccagtgtggtggaattgcccttcaccagctgaatgagttgtggcgcccacaatgctcccatgacaaggagctgacaagttccattttccgtcgcgggcatcttggaatcatgactcccacaatgccttgggcacttggtcgacagtggggccgcctctgaaaaaaaaatgtgagaggtaagtttccattttcacagtttccccgcgccgcttcattgttcggactccggcgggtctgcccgtggctgaaggaggaagtgcgaggaggtgctcgccggccgcggttctcccgggcaggggcgggccgctcgcggtagttggtttcgcaggctgacttgaatattacttagtctggtatcacatggcaaggttgtgatactgtaggacatcagtgaagtgctacttagtaaatcttaacctatctcttttttctacaggttggtactaagaagtgcctttcctgacgtctctgctgcttggaaccgcttctagagcagtctctgcttttgccttgcttgctgccagctagactgtgacgacagcacatccaccctccacctctagcccagacacccccatttctacttataatcaagagaaaagctctaagtatctggcattgccctaggctgctttagtgttaaaagaaaagtttgctgaaaaataagatatcttctgccaggaaatcaaggaggaaaaaaaaaatcattttctcgattttgctctaaactgctgcatctgtctatgccaaactaatcaataccgattgcaccaccaaactccattgcaaattcagctgtgaggagattccctttcagacaactttgctgaaagcagcttggaaattcggtgtcgaagggtctgccacgttttcatgcttgcattttgggctccaaattggcactgggaaggggttactgagagcacaaggctgataccaggccctacttttaaacgttcatctacttacaatcctagtatttctctaaaaaccaaaacctctttgaattaacagtttcatgctgtgaatttctagtgggagatcttttccttgatattgacgacacaattttccatgtacttttaaagcagggagtggggaaaagtattttgaggggacattttcatcatcagttcagctttttttttttggttgttgctcttttttgggggggttgggtttgttggtttcactgaaacatttaactacctgtaaaatctaaacatggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaagtgagtaactattatattcttttaaggatattcaatgattgaatgaggggtatatggacatacagttctctgcccaatgatacattcaagtgggtggtttgcattatatttttttcataatttgctgattttattcttttctaagatgatgtttgctgcttttgtttctctagggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgaagggcaattctgcagatatccagcacagtggcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 39 - DNA, 6829 bp, Artificial Sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatccactagtccagtgtggtggaattgcccttcaccagctgaatgagttgtggcgcccacaatgctcccatgacaaggagctgacaagttccattttccgtcgcgggcatcttggaatcatgactcccacaatgccttgggcacttggtcgacagtggggccgcctctgaaaaaaaaatgtgagaggtaagtttccattttcacagtttccccgcgccgcttcattgttcggactccggcgggtctgcccgtggctgaaggaggaagtgcgaggaggtgctcgccggccgcggttctcccgggcaggggcgggccgctcgcggtagttggtttcgcaggctgacttgaatattacttagtctggtatcacatggcaaggttgtgatactgtaggacatcagtgaagtgctacttagtaaatcttaacctatctcttttttctacaggttggtactaagaagtgcctttcctgacgtctctgctgcttggaaccgcttctagagcagtctctgcttttgccttgcttgctgccagctagactgtgacgacagcacatccaccctccacctctagcccagacacccccatttctacttataatcaagagaaaagctctaagtatctggcattgccctaggctgctttagtgttaaaagaaaagtttgctgaaaaagtaagatatcttctgccaggaaatcaaggaggaaaaaaaaaatcattttctcgattttgctctaaactgctgcatctgtctatgccaaactaatcaataccgattgcaccacccactgaaacatttaactacctgtaaaatctaaacatggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaagtgagtaactattatattcttttaaggatattcaatgattgaatgaggggtatatggacatacagttctctgcccaatgatacattcaagtgggtggtttgcattatatttttttcataatttgctgattttattcttttctaagatgatgtttgctgcttttgtttctctagggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgaagggcaattctgcagatatccagcacagtggcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 40 - DNA, 6829 bp, Artificial Sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatccactagtccagtgtggtggaattgcccttcaccagctgaatgagttgtggcgcccacaatgctcccatgacaaggagctgacaagttccattttccgtcgcgggcatcttggaatcatgactcccacaatgccttgggcacttggtcgacagtggggccgcctctgaaaaaaaaatgtgagaggtaagtttccattttcacagtttccccgcgccgcttcattgttcggactccggcgggtctgcccgtggctgaaggaggaagtgcgaggaggtgctcgccggccgcggttctcccgggcaggggcgggccgctcgcggtagttggtttcgcaggctgacttgaatattacttagtctggtatcacatggcaaggttgtgatactgtaggacatcagtgaagtgctacttagtaaatcttaacctatctcttttttctacaggttggtactaagaagtgcctttcctgacgtctctgctgcttggaaccgcttctagagcagtctctgcttttgccttgcttgctgccagctagactgtgacgacagcacatccaccctccacctctagcccagacacccccatttctacttataatcaagagaaaagctctaagtatctggcattgccctaggctgctttagtgttaaaagaaaagtttgctgaaaaactaagatatcttctgccaggaaatcaaggaggaaaaaaaaaatcattttctcgattttgctctaaactgctgcatctgtctatgccaaactaatcaataccgattgcaccacccactgaaacatttaactacctgtaaaatctaaacatggctgttagtgtcacaccaattcgggacacaaaatggctaacactggaagtctgtagagagttccagagggggacttgctcacggccagacacggaatgtaaatttgcacatccttcgaaaagctgccaagttgaaaatggacgagtaatcgcctgctttgattcattgaaagtgagtaactattatattcttttaaggatattcaatgattgaatgaggggtatatggacatacagttctctgcccaatgatacattcaagtgggtggtttgcattatatttttttcataatttgctgattttattcttttctaagatgatgtttgctgcttttgtttctctagggccgttgctccagggagaactgcaaatatcttcatccacccccacatttaaaaacgcagttggagataaatggacgcaataacttgattcagcagaagaacatggccatgttggcccagcaaatgcaactagccaatgccatgatgcctggtgccccattacaacccgtgaagggcaattctgcagatatccagcacagtggcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 41 - DNA, 9135 bp, Artificial Sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttccatggattacaaggatgacgatgacaagggggtacctgccccaaaaaaaaaacgcaaagtggaggacccagtacccggatctagcagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatggtgagtagagatatcagctctctccttgttagcagtcagaaaagcaaagtgagcaactatatctgactacaagctattcatttagtaacctttttaaaaaaattgctgaagatatgtttgttcaggtatcccagacaatatataaagaagttactttcacagaagtgagggtaactcctcaaatggttcagattgctcctactcttggcctaactcttaatctgtcttattgtggatgtagggtttctgaaatctctgctttgtaaagaaccacctgtctccttttcaacttaacgatattgccattttcagtcttcttatttctgtgtgccatctaatcagccattgttttcctttggtgtgttcttttgcacatcaaggctcttctttacatatatcttgcttttaacaataacatgatctggaactatttaggagaattgttcaacaaaattgttcttagaattagctacaggattttgagtagtatactatctagattgtctgaaataatattgtggcataaacataatagtaacactagagaagcttgactatgccattatatagacaggagagagacagcagtactcaaggagttagaaattggatcattaagaatagctactaaactatattctcaaggtaaattttaatgtgatctagtagctgattttaatgagcccattctttgttctttgcttacctgttttaatgttgtcatttttttacataaatatatgacttaaatcaatgaaatactcattttaaaagtcactgtgtagtatttcataacacagtgaaataatcacgagagagatcttttctgtgtttatggatacttgagcaaaaatacagaaggcagactctctcctcctctcttcctttcactcttttttttttctgttagagtatcttgtttgtaattaactacaaagaggagttatcctcccaataacaactcagtagtgcctttattgtgcatgcttagtcttgttattcgttgtatatggcattccgatgatttgtttttttatttgttttttctcacctacccaaaaatgcactgctgcccccatgatgcacctctgcttgctgtttatgttaatgcgcttgaaccccactggcccattgccatcatgtgctcgctgcctgctaattaagactcagtcggctgtcaaaatcactgaagcgacccctcgaggcaacctttcacctggtactatgacctttcaccttttagcttggcatgtagctttattgtagatacaagttttttttttaaatcaactttaaaatatatatccttttttctgttatagagttgtaaagtacaatgaaaaaactgagtgtggtttcctgacaaaattagtagaaagactataatctaagtacatagatggatatcatacaataaaagattctgaaagcccagcagcccacattcagtttaactacattgtagaatgttctgaagaaatagtcggaggacataaagagtgtatttttgactgtatatttatgtacctgtttggttagcatggctttagtggtagaattatttacattttaatatttatagttcagtatttaaggaaaaaaacttgcatgctctgggacatttgcatgtctaggtagatctggggggaggagcagagtgggttagtttttattttatctgtttcagtcactctgtacaattaactattaacattgcttatacttgtcaatcctcatggagctcgcagtgcggcttttcttcttttcccaaatcaatggcttctcacagaaagcctgaagcattatgggacataaagcactttaacacattataacatgtgctcttgtactagtgagattttgttttggacaaaaataatgcttcctttaaagcttttatcttgctttttttttttttttcctccttaattttttgttcattggattttttccctcgggtagttaagtgctctgctgcttgcttgctcatgcttcctaacaattttagccttcgactgatttttcttttttctttttctctttttactggtatttgttttttatactcattcactaaacagggaattcctcaagctgtacttaccggtgtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaagactcctccctgcaggacggctgcttcatctacaaggtgaagttcatcggcgtgaacttcccctccgacggccccgtaatgcagaagaagaccatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcaccgagggccgccaccacctgttcctgtagaccgcggtgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 42 - DNA, 8462 bp, Artificial Sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttccatggattacaaggatgacgatgacaagggggtacctgccccaaaaaaaaaacgcaaagtggaggacccagtacccggatctagcagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatggtgagtagagatatcagctctctccttgttagcagtcagaaaagcaaagtgagcaactatatctgactacaagctattcatttagtaacctttttaaaaaaattgctgaagatatgtttgttcaggtatcccagaccacgagagagatcttttctgtgtttatggatacttgagcaaaaatacagaaggcagactctctcctcctctcttcctttcactcttttttttttctgttagagtatcttgtttgtaattaactacaaagaggagttatcctcccaataacaactcagtagtgcctttattgtgcatgcttagtcttgttattcgttgtatatggcattccgatgatttgtttttttatttgttttttctcacctacccaaaaatgcactgctgcccccatgatgcacctctgcttgctgtttatgttaatgcgcttgaaccccactggcccattgccatcatgtgctcgctgcctgctaattaagactcagtcggctgtcaaaatcactgaagcgacccctcgaggcaacctttcacctggtactatgacctttcaccttttagcttggcatgtagctttattgtagatacaagttttttttttaaatcaactttaaaatatatatccttttttctgttatagagttgtaaagtacaatgaaaaaactgagtgtggtttcctgacaaaattagtagaaagactataatctaagtacatagatggatatcatacaataaaagattctgaaagcccagcagcccacattcagtttaactacattgtagaatgttctgaagaaatagtcggaggacataaagagtgtatttttgactgtatatttatgtacctgtttggttagcatggctttagtggtagaattatttacattttaatatttatagttcagtatttaaggaaaaaaacttgcatgctctgggacatttgcatgtctaggtagatctggggggaggagcagagtgggttagtttttattttatctgtttcagtcactctgtacaattaactattaacattgcttatacttgtcaatcctcatggagctcgcagtgcggcttttcttcttttcccaaatcaatggcttctcacagaaagcctgaagcattatgggacataaagcactttaacacattataacatgtgctcttgtactagtgagattttgttttggacaaaaataatgcttcctttaaagcttttatcttgctttttttttttttttcctccttaattttttgttcattggattttttccctcgggtagttaagtgctctgctgcttgcttgctcatgcttcctaacaattttagccttcgactgatttttcttttttctttttctctttttactggtatttgttttttatactcattcactaaacagggaattcctcaagctgtacttaccggtgtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaagactcctccctgcaggacggctgcttcatctacaaggtgaagttcatcggcgtgaacttcccctccgacggccccgtaatgcagaagaagaccatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcaccgagggccgccaccacctgttcctgtagaccgcggtgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 43 - DNA, 8394 bp, Artificial Sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttccatggattacaaggatgacgatgacaagggggtacctgccccaaaaaaaaaacgcaaagtggaggacccagtacccggatctagcagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatggtgagtagagatatcagctctctccttgttagcagtcagaaaagcaaagtgagcaactatatctgactacaagctattcatttagtaacctttttaaaaaaattgctgaagatatgtttgttcaggtatcccagacaatatataaagaagttactttcacagaagtgagggtaactcctcaaatggttcagattgctcctactcttggcctaactcttaatctgtcttattgtggatgtagggtttctgaaatctctgctttgtaaagaaccacctgtctccttttcaacttaacgatattgccattttcagtcttcttatttctgtgtgccatctaatcagccattgttttcctttggtgtgttcttttgcacatcaaggctcttctttacatatatcttgcttttaacaataacatgatctggaactatttaggagaattgttcaacaaaattgttcttagaattagctacaggattttgagtagtatactatctagattgtctgaaataatattgtggcataaacataatagtaacactagagaagcttgactatgccattatatagacaggagagagacagcagtactcaaggagttagaaattggatcattaagaatagctactaaactatattctcaaggtaaattttaatgtgatctagtagctgattttaatgagcccattctttgttctttgcttacctgttttaatgttgtcatttttttacataaatatatgacttaaatcaatgaaatactcattttaaaagtcactgtgtagtatttcataacacagtgaaataatcacgagagagatcttttctgtgtttatggatacttgagcaaaaatacagaaggcagactctctcctcctctcttcctttcactcttttttttttctgttagagtatcttgtttgtaattaactacaaagaggagttatcctcccaataacaactcagtagtgcctttattgtgcatgcttagtcttgttattcgttgtatatggcattccgatgatttgtttttttatttgttttttctcacctacccaaaaatgcactgctgcccccatgatgcacctctgcttgctgtttatgttaatgcgcttgaaccccactggcccattgccatcatgtgctcgctgcctgctaattaagactcagtcggctgtcaaaatcactgaagcgacccctcgaggcaacctttcacctggtactatgacctttcaccttttagcttggcatgtagctttattgtaggccttcgactgatttttcttttttctttttctctttttactggtatttgttttttatactcattcactaaacagggaattcctcaagctgtacttaccggtgtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaagactcctccctgcaggacggctgcttcatctacaaggtgaagttcatcggcgtgaacttcccctccgacggccccgtaatgcagaagaagaccatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcaccgagggccgccaccacctgttcctgtagaccgcggtgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 44 - DNA, 7721 bp, Artificial Sequence
  Synthetic
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttccatggattacaaggatgacgatgacaagggggtacctgccccaaaaaaaaaacgcaaagtggaggacccagtacccggatctagcagcacaatgattgacaccaatgacaacacagtcactgtgtgtatggattacatcaaagggagatgctctcgggaaaagtgcaaatactttcatccccctgcacatttgcaagccaagatcaaggctgcccaataccaggtcaaccaggctgcagctgcacaggctgcagccaccgcagctgccatggtgagtagagatatcagctctctccttgttagcagtcagaaaagcaaagtgagcaactatatctgactacaagctattcatttagtaacctttttaaaaaaattgctgaagatatgtttgttcaggtatcccagaccacgagagagatcttttctgtgtttatggatacttgagcaaaaatacagaaggcagactctctcctcctctcttcctttcactcttttttttttctgttagagtatcttgtttgtaattaactacaaagaggagttatcctcccaataacaactcagtagtgcctttattgtgcatgcttagtcttgttattcgttgtatatggcattccgatgatttgtttttttatttgttttttctcacctacccaaaaatgcactgctgcccccatgatgcacctctgcttgctgtttatgttaatgcgcttgaaccccactggcccattgccatcatgtgctcgctgcctgctaattaagactcagtcggctgtcaaaatcactgaagcgacccctcgaggcaacctttcacctggtactatgacctttcaccttttagcttggcatgtagctttattgtaggccttcgactgatttttcttttttctttttctctttttactggtatttgttttttatactcattcactaaacagggaattcctcaagctgtacttaccggtgtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaagactcctccctgcaggacggctgcttcatctacaaggtgaagttcatcggcgtgaacttcccctccgacggccccgtaatgcagaagaagaccatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcaccgagggccgccaccacctgttcctgtagaccgcggtgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 45 - DNA, 85 bp, Artificial Sequence
  Synthetic
gtaaaatctaaacatggctgttagtgtcacaccaattcgggacacaaaatggctaacactggaagtatgtagagagttccagagg

SEQ ID NO: 46 - DNA, 85 bp, Artificial Sequence
  Synthetic
gtaaaatctaaacatggctgttcttgtcacaccaattcgggacacaaaatggctaacactggaagtatgtagagagttccagagg

SEQ ID NO: 47 - protein, 7 aa, Artificial Sequence
  Synthetic
MANTGSM

SEQ ID NO: 48 - DNA, 8949 bp, Artificial Sequence
  Synthetic
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagccgtgttttagagctagaaatagcaagttaaaataaggctagtccgtagcgcgtgcgccaattctgcagacaaattctagaggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtcaccatgtacccatacgacgtaccagattacgctgcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacagctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggccgcggcaggggtggcagtggtggcggcggcggcggcggcggtggtggttacaaccgcagcagtggtggctatgaacccagaggtcgtggaggtggccgtggaggcagaggtggcatggggtaggtgtctcatgagccagggagtatctttggtggggagtgtggaggattgcatgaatctccctgaagccagtccctagtgcatggtttagtattcttgttgtctagggatctgtgagggctttgatttgggggcagtgactttctttttacatccccattttatttttgtgagaacttgggagcctgaactcccatccataccactgaatagagattttgagtaatgatacttgtttccaaaaaaaaagaaaccatacatagatacgtatggattggagtcattaatatcctaggcaagaaacatggaagtgaagacttctttctctgcaagggaaaccgatgatcccactcctgggaaatagtagggaaacttggtatgtgtattcccatgtgtcctctagggagttggtaatggttaacctgacttcagcttccaggaattggctactcttcccgttttctatagtcatttgaatccacgagcttgatttgcactaatttgaccgacattgattttgtgtgtgacttggtttatggggccagctgactgaagtaagcagaccttttgggcaaaaatatgctttgacagtggtctcccacctatttgttccactgtctgccttcccctggttacttaaaattcatcagcttgtccaactggaccttctttccttcctgctgaagttgatttgaagtaaaaccttagatttgatgttaaaacagttgtcaaatctgttggtaaataagatttgaaggaccctactctgtctcccttgaaaaaggggaggaatgtcagtgttactgtttttggaaaaagtagatttttaaaccgagtttggaaatggtaagtatgcagaggtgggtgggggcaatctcaaaaacgtgcaaaaatgaggaaaacaaaaatgaggaaatgtgtgcgtgtgtttaatgcaaaactttaaaaagaaaaacaactgttatgtgactgttaacttgctctgcattttatgtgccacaggtatgaaaggtgacattgcaaaatactccgctcttctcgcagtgtagaaggggtgaccccgggggttgggggagatcaaaaacagctcagtagttaggacagagcttagctaagtttgtcttgctttaaggggaagttgcctttggttttgactttttatggaatggggttgggtctgcttgctgctttcaaagcaaaaaccacaaaaatgtgttcaaggctaccccagcctggtgtgaaatgtcttctgggtaaattggggtagggtttttaaaccaactacttggttgtcaaccacttgcgacaagaggaaaaaaaaacatctgctccatcggaagaacgaccaaggaaaatgggttattttttttccagaggaaatagataacgtaaccttttaaagcaaaatctttataaactgtgtctgagaaattgcacacgtgtgtgtgacatgctcaaaggtcagacaaggggtggtcaggaagggatgtattttagtagccacttgtatctttttccaaaaacacctacccatgtttggggaatgttaaacaaaatcaaaaaacaaccttttgtagccgttggaagcttcatgtcctttcttctaacttgtcttctccagcggaagtgaccgtggtggcttcaataaatttggtggtaagtgaacagagtttccaaaattcccaactcccagcaatgctttgtctgattgttcatttgcagatgtcttagcgtgttaatttaaatgtcaaaggttttgaggtgtccagaaccacctccagaaaggggtagggtagaatgccacctgttgcctggtgtgtgctaacctggagcaggtaggggtaagactcaatagtcatcttttaccaaatgggtttgccccaggttaataagaggggtctagtaggccttggactgggccgttgccacacctggcacttagtgaccatcatcatgagaaactggagagtgcgtgctggaacacgtggtgccatcttggctttaggatccttttgatcgttgtgtccaaggcttgtgtgtgtgtgagtgtgtgggagacaactccgaatgtttaattctggaagagggatgtaacattgccctgaggatggtgaagttggtatacatttataaagtacggaatggtgtcaatgaatgcaattctatgtatatggacttaactgagatgggcaaatagaaactagctctgggaaggaacatgtgcactacttcaagaaagattggaagcatgtgtggctcatgggaaataaccaggtcttaaacagcacaaactgaattcgtggaccaggaaggtcttaaacagcacaaactgaattcatggaaaaatgacaaatttgagaagtctcccagtaagctggaacttttctggtttggttaacaaaaggtttcttgatttgtttcaagatttaaagccaaaggtgtgggttcatgacttaggtgtcattgcgtgtgggtacaatatttatatatggcgaattcagataaacattggtcaaagatggtctctggaaaaacaaaatagaggctgcattacggaaataagatttctggtctgttccctgggacatgcttaaaaaatacaatagctattatgtatggtttttattttcatgtggtttcggggaaacaacacggttttaaggatggtttctaaagatgaaattaaaaattgttccacaagggttaagtgtctggtggtaaagttgggagaaactggatggatgcacatcgcatggctggtggcgagcccatctctcttctctcgggtgagagaaccgggccaagctgagttggtttgttcactttaatgggtctccgtttcccctgccacctgtgctgaggacatttcccagcctgagctgggggaggcagcatttgctgaagtgtggagttgtctctgtggagactcaagttacagatcttaaggggcctgcctagaattttctcctctgggcaggcgacccaggaaagggtttggagtgaggctgtgagcacttacttgatattttacaagtttggatttggtgttaatttttttccttgtccgttttttcctgttgactaacggctcatcttttccttgtttttgttttttttttgttctttttttccatgtcactaaaggccctcgggaccaaggatcacgtcatgactccgaacaggataattcagacaacaacaccatctttgtgcaaggcctgggtgagaatgttacaattgagtctgtggctgattacttcaagcagattggtattattaagacaaacaagaaaacgggacagcccatgattaatttgtacacagacagggaaactggcaagctgaagggagaggcaacggtctcttttgatgacccaccttcagctaaagcagctattgactggtttgatggtaaagaattctccggaaatcctatcaaggtctcatttgctactcgccgggcagactttaatcggggtggtggcaatggtcgtggaggccgagggcgaggaggacccatgggccgtggaggctatggaggtggtggcagtggtggtggtggccgaggaggatttcccagtggaggtggtggcggtggaggacagcagcgagctggtgactggaagtgtcctaatcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgtattaattagcctggctccccaggttctggaacagctttttgtcctgtacccagtgttaccctcgttattttgtaaccttccaattcctgatcacccaagggtttttttgtgtcggactatgtaattgtaactatacctctggttcccattaaaagtgaccattttagttcctcttcccccttttcactttcctggaagatcgatgtcccgatcaggaaggtagagagttttcctgttcagattaccctgcccagcaggaactggaatacagtgttcggggagaaggccaaatgatatccttgagagcagagattaaacttttctgtcatggggaaagttggtgtataaatgagaaatgaagaacatgggatgtcatgagtgttggccttaagatacattgatgagtttggacaaaccacaactagaatgcagtgaaaaaaatgctttatttgtgaaatttgtgatgctattgctttatttgtaaccattataagctgcaataaacaagttctgattttgtagttaacacgtgcgaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 49 - DNA, 6815 bp, Artificial Sequence
  Synthetic
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcagccgtgttttagagctagaaatagcaagttaaaataaggctagtccgtagcgcgtgcgccaattctgcagacaaattctagaggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtcaccatgtacccatacgacgtaccagattacgctttagcggcgatggcggcggcggcggcggcggcagcagcagcgggggctgcgggcggtcggggctccgggccggggcggcggcgccatcttgtgcccggggccggtggggaggccggggagggggccccggggggcgcaggggactacgggaacggcctggagtctgaggaactggagcctgaggagctgctgctggagcccgagccggagcccgagcccgaagaggagccgccccggccccgcgcccccccgggagctccgggccctgggcctggttcgggagcccccggcagccaagaggaggaggaggagccgggactggtcgagggtgacccgggggacggcgccattgaggacccggagctggaagctatcaaagctcgagtcagggagatggaggaagaagctgagaagctaaaggagctacagaacgaggtagagaagcagatgaatatgagtccacctccaggcaatgctggcccggtgatcatgtccattgaggagaagatggaggctgatgcccgttccatctatgttggcaatgtggactatggtgcaacagcagaagagctggaagctcactttcatggctgtggttcagtcaaccgtgttaccatactgtgtgacaaatttagtggccatcccaaagggtttgcgtatatagagttctcagacaaagagtcagtgaggacttccttggccttagatgagtccctatttagaggaaggcaaatcaaggtgatcccaaaacgaaccaacagaccaggcatcagcacaacagaccggggttttccacgagcccgctaccgcgcccggaccaccaactacaacagctcccgctctcgattctacagtggttttaacagcaggccccggggtcgcgtctacaggtcaggatagatgggctgctcctctttcccccgcctcccgtgagccccgtatgcttcctcctctctggtctgaggaacctccctccccccacccctccccgtggtcttcaggaactttgtctcctgcctgtgcaggttgaggaaggtagttgcaggccaggccagaaggccagcctcatcatcttttctgcagtagaaattggtgataagggctgcatccctcccttggttcaaagaggcttccacccccagccttttttttcttgggagttggtggcatttgaaggtgtttgcggacaaaactgggaggaacagggcctccaggcagtgaaagcactgcttggacatttgttacttttttcggagttagggagggattgaagactgaacctcccttggaagaataccagaggctagctagttgatcctccccaacagccttgtgggaggattttgagatacttattctttatttgagccagtcttgcaaggttaacttctcactgggcctagtgtggtgcccaggtttttgccttgcttcacttctgtctctacatttaaatagacgggttaggcatataaaccttggcttttcataagctctacctgcctatccccaggagttagggaggatctatttgtgatggcctagggtttaagagctgtggaggactgaaaactggataaaaagggggtccttttccttgcccctgtctctcactcagatgcgcttctttttcgccactgtttggcagttttctgttaagcccccctccccctgccccagttctccaggtgcgttactatttctgggatcatggggtcagttttaggacacttgaacacttcttttccccccttcccttcacagtaactggggcaggggcctacggggaggggcttgtactgaactatctagtgatcacgttaacacctaactctccttctttcttccaggggccgggctagagcgacatcatggtattccccttactaaaaaaagtgtgtattaggaggagagagaggaaaaaaagaggaaagaaggaaaaaaaaaagaattaaaaaaaaaaaaaagaaaaacagaagatgaccttgatggaaaaaaaatattttttaaaaaaaagatatactgtggaaggggggagaatcccataactaactgctgaggagggacctgctttggggagtaggggaaggcccagggagtggggcagggggctgcttattcactctggggattcgccatggacacgtctcaactgcgcaagctgctgcccatgtttccctgccccacttcacccccttgggggctgctcaagggtaggtgggcgtgggtggtaggaggtttttttttttacccagggctctggaaggacaccaaactgttctgcttgttaccttccctccgtcttctcctcgcctttcacagtcccctcctgcctgctcctgtccagccaggtctaccacccaccccacccctcttctccggctccctgcccctccagattgcctggtgatctattttgtttccttttgtgtttctttttctgttttgagtgtctttctttgcaggtttctgtagccggaagatctccgttccgctcccagcggctccagtgtaaattccccttccccctggggaaatgcactaccttgttttggggggtttaggggtgtttttgtttttcagttgttttgtttttttgttttttttttttttcctttgccttttttcccttttatttggagggaatgggaggaagtgggaacagggaggtgggaggtggattttgtttatttttttagctcatttccaggggtgggaatttttttttaatatgtgtcatgaataaagttgtttttgaaaataaaaattgtttggccttttgggtgtaaggattatttatcttcctattctctcttagatcgcagatggctattaattctgccttgtggtagaagtactggtgttgtggaagggaggttgccacttgtgaattaaggagtaggaattacattacaactagcaggaacattatgtgcttaagatacattgatgagtttggacaaaccacaactagaatgcagtgaaaaaaatgctttatttgtgaaatttgtgatgctattgctttatttgtaaccattataagctgcaataaacaagttctgattttgtagttaacacgtgcgaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga